(19) United States
(12) Patent Application Publication
(10) Pub. No.: US 2026/0062487 A1
(43) Pub. Date: Mar. 5, 2026

(54) MULTI-SPECIFIC ANTIBODIES AND METHODS OF USE

(71) Applicant: Gritstone bio, Inc., Jackson, WY (US)

(72) Inventors: Isaac J. RONDON, Jackson, WY (US); Christine JANSON, Jackson, WY (US); Manankumar Anilkumar SHAH, Jackson, WY (US); Karin JOOSS, San Diego, CA (US)

(21) Appl. No.: 19/284,186

(22) Filed: Jul. 29, 2025

Related U.S. Application Data
(63) Continuation of application No. 18/351,184, filed on Jul. 12, 2023.
Application No. 18/351,184 is a continuation of application No. PCT/US2022/012573, filed on Jan. 14, 2022.
(60) Provisional application No. 63/137,702, filed on Jan. 14, 2021.

(51) Int. Cl. C07K 16/28; A61K 39/00; A61P 35/00; C07K 16/46

(52) U.S. Cl. CPC C07K 16/2833; A61P 35/00; C07K 16/2809; C07K 16/468; A61K 2039/505; C07K 2317/31; C07K 2317/622; C07K 2317/626; C07K 2317/732; C07K 2317/92; C07K 2317/94

(57) ABSTRACT

Provided herein are multispecific antigen binding proteins that selectively bind a particular KKLC-1 (CT-83) shared antigen and CD3, as well as related methods, kits, and compositions. Included herein are multispecific antigen binding proteins with a circularized conformation.

### CROSS REFERENCE TO RELATED APPLICATIONS

This application is a continuation of U.S. application Ser. No. 18/351,184, filed on Jul. 12, 2023, which is a continuation of International Application No. PCT/US2022/012573, filed on Jan. 14, 2022, which claims the benefit of U.S. Provisional Application No. 63/137,702, filed on Jan. 14, 2021, the disclosure of each of which is incorporated by reference herein in its entirety.

### SEQUENCE LISTING

The instant application contains a Sequence Listing which has been submitted via Patent Center and is hereby incorporated by reference in its entirety. Said XML copy, created on Aug. 1, 2023, is named GSO-099WOC1_SL.txt, and is 213,200 bytes in size.

### BACKGROUND

Specific antigen recognition is essential for antibodies to function in the adaptive immune system. The specificity of antibodies and antibody fragments for a particular antigen or antigens makes antibodies desirable therapeutic agents. Antibodies and antibody fragments can be used to target specific tissues, for example, tumor tissue or infected tissue, thereby minimizing potential side effects of non-specific targeting. Thousands of antigens are capable of eliciting responses, each almost exclusively directed to the particular antigen which elicited it.

Major histocompatibility complex class I molecules are expressed on the surface of virtually all nucleated cells in the body and are dimeric molecules comprising a transmembrane heavy chain, comprising the peptide antigen binding cleft, and a smaller extracellular chain termed beta2-microglobulin. MHC class I molecules present peptides derived from the degradation of cytosolic proteins by the proteasome, a multi-unit structure in the cytoplasm, (Niedermann G., 2002. Curr Top Microbiol Immunol. 268:91-136; for processing of bacterial antigens, refer to Wick M J, and Ljunggren H G., 1999. Immunol Rev. 172:153-62). Cleaved peptides are transported into the lumen of the endoplasmic reticulum (ER) by the transporter associated with antigen processing (TAP) where they are bound to the groove of the assembled class I molecule, and the resultant MHC/peptide complex is transported to the cell membrane to enable antigen presentation to T lymphocytes (Yewdell J W., 2001. Trends Cell Biol. 11:294-7; Yewdell J W. and Bennink J R., 2001. Curr Opin Immunol. 13:13-8). Alternatively, cleaved peptides can be loaded onto MHC class I molecules in a TAP-independent manner and can also present extracellularly-derived proteins through a process of cross-presentation. As such, a given MHC/peptide complex presents a novel protein structure on the cell surface that can be targeted by a novel antigen-binding protein (e.g., antibodies or TCRs) once the identity of the complex's structure (peptide sequence and MHC subtype) is determined.

Tumor cells can express antigens and may display such antigens on the surface of the tumor cell. Such tumor-associated antigens can be used for development of novel immunotherapeutic reagents for the specific targeting of tumor cells. For example, tumor-associated antigens can be used to identify therapeutic antigen binding proteins, e.g., antibodies or antigen-binding fragments thereof.

Normal cells also display restricted peptides on their surface. In some cases, restricted peptides displayed by normal cells can have sequence overlap to the tumor-specific antigens. Such sequence-overlapping restricted peptides therefore represent potential off-target liabilities for therapeutic cancer immunotherapy.

Therefore, there exists a need for antigen-binding proteins that selectively bind tumor-specific antigens displayed on the surface of tumor cells, preferably with minimal or no off-target liability.

### SUMMARY

Provided herein are antigen binding proteins that specifically bind to KKLC-1 and a cell surface molecule (e.g., CD3) on an effector cell.

In one aspect, provided herein are isolated antigen binding proteins (ABP) that comprises: a first antigen binding region (ABR) and a second ABR that each specifically bind a first target antigen, a Fab that specifically binds an additional target antigen that is distinct from the first target antigen, and an Fc domain, wherein the ABP comprises a first polypeptide, a second polypeptide, and a third polypeptide; wherein the first polypeptide comprises, in an N to C direction, the first ABR-a first hinge-CH2-CH3; wherein the second polypeptide comprises, in an N to C direction, the second ABR-a variable heavy chain (VH) domain of the Fab-a CH1 domain of the Fab-a second hinge-CH2-CH3; wherein the third polypeptide comprises, in an N to C direction, a variable light chain (VL) domain of the Fab-a CL domain of the Fab; wherein the first ABR and second ABR each comprise, in an N to C direction: (i) a VH domain-a VL domain or (ii) a VL domain-VH domain; wherein the VH domain of the first ABR is attached to the VL domain of the first ABR via a first linker; wherein the VH domain of the second ABR is attached to the VL domain of the second ABR via a second linker; wherein the first linker and second linker are each about 5-15 amino acids in length; and wherein the first target antigen is an HLA-PEPTIDE target comprising an HLA-restricted peptide complexed with an HLA Class I molecule, wherein the HLA Class I molecule is HLA subtype HLA-A*01:01 and the HLA-restricted peptide comprises the sequence NTDNNLAVY (SEQ ID NO: 214); and wherein the VH domains of the first and second ABRs each comprise complementarity-determining regions (CDR) H1 (CDR-H1), CDR-H2, and CDR-H3 comprising the sequences set forth in SEQ ID NOS:18, 19, and 20, respectively and wherein the VL comprises CDR-L1, CDR-L2, and CDR-L3 comprising the sequences set forth in SEQ ID NOS:21, 22, and 23, respectively.

In one aspect, provided herein are isolated antigen binding proteins (ABP) that comprises: a first antigen binding region (ABR) the specifically binds a first target antigen and a second ABR that specifically binds a second target antigen, a Fab that specifically binds an additional target antigen that is distinct from the first target antigen, and an Fc domain, wherein the ABP comprises a first polypeptide, a second polypeptide, and a third polypeptide; wherein the first polypeptide comprises, in an N to C direction, a variable heavy chain (VH) domain or variable light chain (VL) domain of the first ABR-a VH domain or a VL domain of the second ABR-a first hinge-CH2-CH3; wherein the second polypeptide comprises, in an N to C direction, the VH domain or VL domain of the second ABR-the VH domain or VL domain of the first ABR-a VH domain of the Fab-a CH1 domain of the Fab-a second hinge-CH2-CH3, wherein the third polypeptide comprises, in an N to C direction, a VL domain of the Fab-a CL domain of the Fab; wherein the VH domain or VL domain of the first ABR of the first polypeptide is attached to the VL domain or VH domain of the second ABR of the first polypeptide via a first linker; wherein the VH domain or VL domain of the second ABR of the second polypeptide is attached to the VL domain or VH domain of the first ABR via a second linker; wherein the first linker and second linker are each about 5-15 amino acids in length.

In some embodiments, the first target antigen is an HLA-PEPTIDE target comprising an HLA-restricted peptide complexed with an HLA Class I molecule, wherein the HLA Class I molecule is HLA subtype HLA-A*01:01 and the HLA-restricted peptide comprises the sequence NTDNNLAVY (SEQ ID NO: 214); and wherein the VH domains of the first and/or second ABRs each comprise complementarity-determining regions (CDR) H1 (CDR-H1), CDR-H2, and CDR-H3 comprising the sequences set forth in SEQ ID NOS:18, 19, and 20, respectively and wherein the VL comprises CDR-L1, CDR-L2, and CDR-L3 comprising the sequences set forth in SEQ ID NOS:21, 22, and 23, respectively.

In some embodiments, the first linker and second linker are each individually 5, 6, 7, 8, 9, 10, 11, 12, 13, 14 or 15 amino acids in length.

In some embodiments, the first linker and second linker are each individually 5-15, 5-14, 5-13, 5-12, 5-11, 5-10, 5-9, 5-8, 5-7, 5-6, 6-15, 6-14, 6-13, 6-12, 6-11, 6-10, 6-9, 6-8, 6-7, 7-15, 7-14, 7-13, 7-12, 7-11, 7-10, 7-9, 7-8, 8-15, 8-14, 8-13, 8-12, 8-11, 8-10, 8-9, 9-15, 9-14, 9-13, 9-12, 9-11 or 9-10 amino acids in length.

In some embodiments, the first linker and second linker are each individually 5-11 amino acids in length.

In some embodiments, the first linker and second linker each consist of 5, 6, 7, 8, 9, 10 or 11 amino acids.

In some embodiments, the first linker and second linker each consist of 5, 8 or 10 amino acids.

In some embodiments, the first linker and second linker each consist of 10 amino acids.

In some embodiments, the additional target antigen is CD3.

In some embodiments, the VH and VL domains of the Fab comprise CDR-H1, CDR-H2, CDR-H3, CDR-L1, CDR-L2, and CDR-L3 comprising the sequences set forth in SEQ ID NOS: 36, 37, 38, 39, 40, and 41; 26, 27, 28, 29, 30, and 31; or 42, 43, 44, 45, 46, and 47 respectively.

In some embodiments, the VH of the first and second ABRs comprises the sequence set forth in SEQ ID NO: 6 and wherein the VL of the first and second ABR comprises the sequence set forth in SEQ ID NO: 4.

In some embodiments, the VH and VL of the Fab comprises the sequence set forth in SEQ ID NOS: 32 and 33; 24 and 25; or 34 and 35, respectively.

In some embodiments, the first polypeptide comprises the sequence set forth in SEQ ID NO: 55.

In some embodiments, the first polypeptide comprises the sequences set forth in SEQ ID NO: 55 and 48.

In some embodiments, the second polypeptide comprises the sequence set forth in SEQ ID NOs: 55.

In some embodiments, the second polypeptide comprises the sequences set forth in SEQ ID NOs: 55 and 51, 49, or 53.

In some embodiments, the third polypeptide comprises the sequence set forth in SEQ ID NOs: 52, 50, or 54.

In some embodiments, the first polypeptide comprises the sequence set forth in SEQ ID NO: 55 and 48, the second polypeptide comprises the sequence set forth in SEQ ID NO: 55 and 51, and the third polypeptide comprises the sequence set forth in SEQ ID NOs: 52.

In some embodiments, the VH domain of the first ABR comprise complementarity-determining regions (CDR) H1 (CDR-H1), CDR-H2, and CDR-H3 comprising the sequences set forth in SEQ ID NOs: 18, 19, and 20, respectively, and wherein the VL comprises CDR-L1, CDR-L2, and CDR-L3 comprising the sequences set forth in SEQ ID NOs: 21, 22, and 23, respectively.

In some embodiments, the VH domain of the second ABR comprise CDR-H1, CDR-H2, CDR-H3 comprising the sequences set forth in SEQ ID NOS: 36, 37, and 38; 26, 27, and 28; or 42, 43, and 44, respectively, and wherein the VL comprises CDR-L1, CDR-L2, and CDR-L3 comprising the sequences set forth in SEQ ID NOS: 39, 40, and 41; 29, 30, and 31; or 45, 46, and 47, respectively.

In some embodiments, the VH and VL domains of the Fab comprise CDR-H1, CDR-H2, CDR-H3, CDR-L1, CDR-L2, and CDR-L3 comprising SEQ ID NOS: 18, 19, 20, 21, 22, and 23, respectively.

In some embodiments, the VH of the first ABR comprises SEQ ID NO:6 and wherein the VL of the first ABR comprises SEQ ID NO:4.

In some embodiments, the VH of the second ABR comprises SEQ ID NO: 32, 24, or 34 and wherein the VL of the second ABR comprises SEQ ID NO: 33, 25, or 35.

In some embodiments, the first polypeptide comprises the sequence set forth in SEQ ID NO: 48 and 58 or 59.

In some embodiments, the second polypeptide comprises the sequence set forth in SEQ ID NO: 56 and 58 or 59.

In some embodiments, the third polypeptide comprises the sequence set forth in SEQ ID NO: 57.

In some embodiments, the first polypeptide comprises the sequence set forth in SEQ ID NO: 48 and 58 or 59, the second polypeptide comprises the sequence set forth in SEQ ID NO: 56 and 58 or 59, and wherein the third polypeptide comprises the sequence set forth in SEQ ID NO: 57.

In some embodiments, the first linker and second linker each consist of (GGGGS)N, wherein N=1-3 (SEQ ID NO: 215).

In some embodiments, the first linker and second linker each consist of (GSGGG)N, wherein N=2 (SEQ ID NO: 216).

In some embodiments, the first linker and second linker each comprise glycine, serine, or glycine and serine amino acids.

In some embodiments, the first linker and second linker each consist of glycine and serine amino acids.

In some embodiments, the VH domain of the first ABR interacts with the VL domain of the second ABR.

In some embodiments, the VL domain of the first ABR interacts with the VH domain of the second ABR.

In some embodiments, the VL domain of the first ABR interacts with the VH domain of the second ABR and wherein the VH domain of the first ABR interacts with the VL domain of the second ABR.

In some embodiments, the interaction of the VL domain of the first ABR with the VH domain of the second ABR and the interaction of the VH domain of the first ABR with the VL domain of the second ABR results in a circularized conformation.

In one aspect, provided herein are isolated antigen binding proteins (ABP) that comprises: (a) a first antigen binding site that specifically binds to a human leukocyte antigen (HLA)-PEPTIDE target, wherein the HLA-PEPTIDE target comprises an HLA-restricted peptide complexed with an HLA Class I molecule, wherein the HLA-restricted peptide is located in the peptide binding groove of an α1/α2 heterodimer portion of the HLA Class I molecule, wherein the HLA Class I molecule is HLA subtype HLA-A*01:01 and the HLA-restricted peptide comprises the sequence NTDNNLAVY (SEQ ID NO: 214); and (b) a second antigen binding site that specifically binds to a CD3 target on an effector cell; wherein the first antigen binding site comprises a variable heavy chain domain (VH) and a variable light chain domain (VL), wherein the VH of the first antigen binding site comprises complementarity-determining regions (CDR) H1 (CDR-H1), CDR-H2, and CDR-H3 comprising SEQ ID NOS: 18, 19, and 20, respectively and wherein the VL comprises a CDR-L1, CDR-L2, and CDR-L3 comprising SEQ ID NOS: 21, 22, and 23, respectively.

In some embodiments, the second antigen binding site comprises a VH and a VL, wherein the VH and VL comprise a CDR-H1, CDR-H2, CDR-H3, CDR-L1, CDR-L2, and CDR-L3 comprising SEQ ID NOS: 36, 37, 38, 39, 40, and 41; 26, 27, 28, 29, 30, and 31; or 42, 43, 44, 45, 46, and 47, respectively.

In some embodiments, the VH and VL of the first antigen binding site comprises SEQ ID NOS: 6 and 4, respectively.

In some embodiments, the VH and VL of the second antigen binding site comprises SEQ ID NOS: 32 and 33, respectively.

In some embodiments, the effector cell is a T cell or an NK cell.

In some embodiments, the ABP binds the HLA-PEPTIDE target with greater affinity as compared to an off-target HLA-PEPTIDE comprising an off-target restricted peptide complexed with an HLA Class I molecule.

In some embodiments, contacting the ABP with cancer cells results in at least 20%, 30%, 40%, 50%, 60%, 70%, 80%, 90% or 95% cytotoxicity.

In some embodiments, the cytotoxicity that results from the ABP contacting cancer cells is greater than a reference antigen binding protein.

In some embodiments, the cancer cells have an A*01:01_NTDNNLAVY (SEQ ID NO: 214) copy number of less than about 500, about 1000, about 1500, about 2000, about 2500, about 3000, about 3500, about 4000, about 4500, about 5000, about 5500, about 6000, about 6500, about 7000, about 7500, about 8000, about 8500, about 9000, about 9500, about 10000, about 20000, about 30000, and about 40000 copies/cell.

In some embodiments, the cancer cells have an A*01:01_NTDNNLAVY (SEQ ID NO: 214) copy number of about 500, about 1000, about 1500, about 2000, about 2500, about 3000, about 3500, about 4000, about 4500, about 5000, about 5500, about 6000, about 6500, about 7000, about 7500, about 8000, about 8500, about 9000, about 9500, about 10000, about 20000, about 30000, and about 40000 copies/cell.

In some embodiments, contacting the ABP with a tumor results in a reduction in tumor growth, relative to before contacting the ABP with a tumor.

In some embodiments, contacting the ABP with a tumor results in decreased tumor volume relative to contacting with a reference antigen binding protein.

In some embodiments, the ABP comprises an antibody or antigen-binding fragment thereof.

In some embodiments, the antigen binding protein is linked to a scaffold, optionally wherein the scaffold comprises serum albumin or Fc, optionally wherein Fc is human Fc and is an IgG (IgG1, IgG2, IgG3, IgG4), an IgA (IgA1, IgA2), an IgD, an IgE, or an IgM.

In some embodiments, the antigen binding protein is linked to a scaffold via a linker, optionally wherein the linker is a peptide linker, optionally wherein the peptide linker is a hinge region of a human antibody.

In some embodiments, the antigen binding protein comprises an Fv fragment, a Fab fragment, a F(ab′)2 fragment, a Fab′ fragment, an scFv fragment, an scFv-Fc fragment, and/or a single-domain antibody or antigen binding fragment thereof.

In some embodiments, the antigen binding protein comprises a single-domain antibody fragment.

In some embodiments, the antigen binding protein comprises an scFv fragment.

In some embodiments, the antigen binding protein comprises an VHH fragment.

In some embodiments, the antigen binding protein comprises an antibody.

In some embodiments, the antigen binding protein is a monoclonal antibody.

In some embodiments, the antigen binding protein is a humanized, human, or chimeric antibody.

In some embodiments, the antigen binding protein is multispecific, optionally bispecific.

In some embodiments, the antigen binding protein binds greater than one antigen or greater than one epitope on a single antigen.

In some embodiments, the antigen binding protein comprises a heavy chain constant region of a class selected from IgG, IgA, IgD, IgE, and IgM.

In some embodiments, the antigen binding protein comprises a heavy chain constant region of the class human IgG and a subclass selected from IgG1, IgG4, IgG2, and IgG3.

In some embodiments, the antigen binding protein comprises a modified Fc, optionally wherein the modified Fc comprises one or more mutations that extend half-life, optionally wherein the one or more mutations that extend half-life is YTE.

In some embodiments, a sequence comprising the CH2-CH3 domains of the first polypeptide is distinct from a sequence comprising the CH2-CH3 domains of the second polypeptide.

In some embodiments, the APB comprises a variant CH2-CH3 domain.

In some embodiments, the variant CH2-CH3 domain comprises a modification that alters an affinity of the ABP for an Fc receptor as compared to an ABP with a non-variant Fc region.

In some embodiments, the variant CH2-CH3 domain comprises one or more amino acid substitutions that reduce at least one Fc effector function.

In some embodiments, the variant CH2-CH3 domain comprises one or more amino acid substitutions that reduce binding to an Fc receptor on the cell surface of an effector cell.

In some embodiments, the Fc receptor on the cell surface of an effector cell is selected from: FcγRI; FcγRIIA; FcγRIIB1; FcγRIIIB2; FcγRIIIA; and FcγRIIIB receptors.

In some embodiments, the Fc effector function that is reduced comprises one or more functions selected from: complement-dependent cytotoxicity (CDC), antibody-dependent cellular cytotoxicity (ADCC), and complement fixation.

In some embodiments, the one or more amino acid substitutions is selected from: L234, L235, P331, L234F, L235E, and P331S, according to the EU numbering system.

In some embodiments, the variant CH2-CH3 domain comprises the amino acid substitutions of L234F, L235E, and P331S, according to the EU numbering system.

In some embodiments, the variant CH2-CH3 domain of the first polypeptide comprises a knob-in-hole modification.

In some embodiments, the variant CH2-CH3 domain of the second polypeptide comprises a knob-in-hole modification.

In some embodiments, one CH2-CH3 domain-bearing chain of the ABP comprises a T366W mutation, and the other CH2-CH3 domain-bearing chain of the ABP comprises a T366S, L368A, and Y407V mutation, according to EU numbering.

In some embodiments, the APB comprises an S354C and T366W mutation in one CH2-CH3 domain and a Y349C, T366S, L368A and Y407V mutation in the other CH2-CH3 domain, according to EU numbering.

In some embodiments, one CH2-CH3 domain comprises a H435, Y436, H435R, Y436F, or H435R_Y436F mutation, according to EU numbering.

In some embodiments, the variant CH2-CH3 domain comprises a set of mutations that renders homodimerization electrostatically unfavorable but heterodimerization favorable.

In some embodiments, the first hinge comprises a C220S mutation, according to EU numbering.

In some embodiments, the antigen binding protein is a portion of a chimeric antigen receptor (CAR) comprising: an extracellular portion comprising the antigen binding protein; and an intracellular signaling domain.

In one aspect, provided herein are isolated polynucleotides or sets of polynucleotides encoding the antigen binding protein as disclosed herein or an antigen-binding portion thereof.

In one aspect, provided herein are vectors or sets of vectors comprising the polynucleotide or set of polynucleotides as disclosed herein.

In one aspect, provided herein are host cells comprising the polynucleotide or set of polynucleotides as disclosed herein or the vector or set of vectors as disclosed herein, optionally wherein the host cell is CHO or HEK293, or optionally wherein the host cell is a T cell.

In one aspect, provided herein are methods of producing an antigen binding protein comprising expressing the antigen binding protein with the host cell as disclosed herein and isolating the expressed antigen binding protein.

In one aspect, provided herein are pharmaceutical compositions comprising the antigen binding protein as disclosed herein and a pharmaceutically acceptable excipient.

In one aspect, provided herein are methods of increasing an immune response in a subject, comprising administering to the subject the ABP as disclosed herein or a pharmaceutical composition as disclosed herein, optionally wherein the subject has cancer.

In some embodiments, the cancer is selected from a solid tumor and a hematological tumor.

In some embodiments, the cancer is selected from: esophageal cancer, gastric adenocarcinoma, lung adenocarcinoma, and lung squamous cancer.

In one aspect, provided herein are methods of treating cancer in a subject, comprising administering to the subject an effective amount of the antigen binding protein as disclosed herein or a pharmaceutical composition as disclosed herein.

In some embodiments, the cancer is selected from a solid tumor and a hematological tumor.

In some embodiments, the cancer is selected from: esophageal cancer, gastric adenocarcinoma, lung adenocarcinoma, and lung squamous cancer.

In some embodiments, the cancer expresses or is predicted to express the HLA-PEPTIDE target.

In some embodiments, the method comprises, prior to the administering, determining or having determined the presence of any one or more of the HLA-PEPTIDE target, the restricted peptide of the HLA-PEPTIDE target, and the HLA molecule of the HLA-PEPTIDE target in a biological sample obtained from the subject.

In some embodiments, the biological sample is a blood sample or a tumor sample.

In some embodiments, the blood sample is a plasma or serum sample.

In some embodiments, after having determined the presence of the HLA-PEPTIDE target, restricted peptide, or HLA in the biological sample obtained from the subject, administering to the subject an ABP that selectively binds to the HLA-PEPTIDE antigen.

In one aspect, provided herein are kits comprising the antigen binding protein as disclosed herein or a pharmaceutical composition as disclosed herein and instructions for use.

### BRIEF DESCRIPTION OF THE SEVERAL VIEWS OF THE DRAWINGS

These and other features, aspects, and advantages of the present invention will become better understood with regard to the following description, and accompanying drawings, where:

FIG. 1A depicts levels of expression (in transcript per million (TPM)) of the KKLC-1 gene in normal tissue. FIG. 1B depicts the levels of expression (in transcript per million (TPM)) of the KKLC-1 gene in the indicated tumor cells.

FIG. 2 depicts the copy numbers for tumor antigen targets, e.g., the copy number for *A01:01 target in the CT-83 gene (arrow).

FIG. 3 is a flow chart showing the selection and screening process for identifying lead clones for use in the described antigen binding proteins.

FIG. 4A depicts a Format 4 antibody in dual scFv conformation. FIG. 4B depicts Format 4, 5, and 6 antibody structures.

FIG. 5 depicts the equilibrium/unstable conformation state that the Format 4 antibody can exist in when in solution, which can depend on linker length and/or other types of modifications to the antibody.

FIG. 6 depicts potential modifications to the Format 4 antibody that may result in stable conformation (either dual-scFv conformation or diabody conformation).

FIGS. 7A and 7B include size exclusion chromatograms of the modified Format 4 antibodies. The two arrows (FIG. 7A) show two antibodies that stably formed diabodies (Format 41 antibody formed). The two antibodies in FIG. 7B also stably formed diabodies.

FIG. 8A depicts the stable conformation that resulted from shortening the L1 and L2 linkers to 10 amino acids. FIG. 8B includes a flow chart showing the screening process to identify preferred clones that were incorporated into Format 41 antibodies for further experimentation.

FIG. 9 depicts greater cytotoxicity resulting from the E07-CD3 diabody compared to a Format 41 antibody that incorporated the VH and VL sequences from D5 clone.

FIG. 10A shows the cytotoxicity measurements of cells having the indicated target antigen copy number levels when contacted with the E07-CD3 diabody. FIG. 10B shows the cytotoxicity measurements of cells having the indicated target antigen copy number levels when contacted with the E07-CD3 diabody. FIG. 10C shows the cytotoxicity measurements of cells having the indicated target antigen copy number levels when contacted with the E07-CD3 diabody. FIG. 10D shows the cytotoxicity measurements of cells having the indicated target antigen copy number levels when contacted with the E07-CD3 diabody

FIG. 11 depicts the VH and VL sequences for the E07 clone with protein liabilities shown. FIG. 11 discloses SEQ ID NOS 235 and 4, respectively, in order of appearance.

FIG. 12 depicts the VH and VL sequences for the hOKT3a clone with protein liabilities shown. FIG. 12 discloses SEQ ID NOS 24 and 25, respectively, in order of appearance.

FIG. 13 depicts the procedures for conducting the off-target liability assessment (OTLA) with EDGE™ to identify OTLA sequences.

FIG. 14 includes a schematic showing the evaluation of K562 cells pulsed with the target antigen or off-target OTLAs and evaluated by flow cytometry.

FIG. 15 depicts the flow cytometry results from the cell-based off-target liability assessment using K562-pulsed cells. FIG. 15 discloses SEQ ID NOS 214 and 204-206, respectively, in order of appearance.

FIG. 16A depicts the study design for NOD/SCID/γcommon^{-/-} mice treated with the indicated doses of E07-CD3 diabody for a pharmacokinetic assessment. FIG. 16B shows the plasma levels of the different doses of E07-CD3 diabody in the mice.

FIG. 17 depicts the study design for Tg32 mice treated with the indicated doses of E07-CD3 diabody for a pharmacokinetic assessment.

FIG. 18 depicts the study design for an in vivo efficacy assessment using NOD/SCID/γcommon^{-/-} mice injected with tumor cells and PBMCs at Day 1 followed by E07-CD3 diabody at Day 4.

FIG. 19 shows the correlation between the drug (E07-CD3 diabody) concentration and trimer formation.

FIG. 20 depicts the study design for an in vivo efficacy assessment using NOD/SCID/γcommon^{-/-} mice injected with tumor cells and PBMCs at Day 1 followed by E07-CD3 diabody at Day 4

FIG. 21A shows the tumor volume measurements from the indicated effector:target (E:T) ratio and E07-CD3 diabody dose levels. FIG. 21B shows the tumor volume measurements from the indicated effector:target (E:T) ratio and E07-CD3 diabody dose levels. FIG. 21C shows the tumor volume measurements from the indicated effector:target (E:T) ratio and E07-CD3 diabody dose levels. FIG. 21D shows the tumor volume measurements from the indicated effector:target (E:T) ratio and E07-CD3 diabody dose levels. FIG. 21E shows the tumor volume measurements from the indicated effector:target (E:T) ratio and E07-CD3 diabody dose levels. FIG. 21F shows the tumor volume measurements from the indicated effector:target (E:T) ratio and E07-CD3 diabody dose levels.

FIG. 22A depicts the tumor volume measurements from the indicated E:T ratios and E07-CD3 diabody dose levels. FIG. 22B depicts the tumor volume measurements from the indicated E:T ratios and E07-CD3 diabody dose levels

FIG. 23 depicts the study design for an in vivo efficacy assessment using NOD/SCID/γcommon^{-/-} mice injected with tumor cells at Day 1, PBMCs at Day 3 followed by E07-CD3 diabody at Day 4.

FIG. 24 shows schematics of the 41-E07-hOKT3a, 41-E07-hUCHT1v9, and 41-E07-hSP34x bispecific antibodies.

FIG. 25 shows binding of the 41-E07 hOKT3, 41-E07 hUCHT1v9, or 41-E07 hSP34x antibodies to the cells expressing 1×10⁶ copies of the pHLA 9mer or control cells (A375 Luc).

FIG. 26 shows binding of the 41-E07 hOKT3, 41-E07 hUCHT1v9, or 41-E07 hSP34x antibodies to CD3− and CD3+ Jurkat cells.

FIG. 27 shows binding of the 41-E07 hOKT3, 41-E07 hUCHT1v9, or 41-E07 hSP34x antibodies to the indicated cell lines.

FIG. 28 provides schematics of Format 43 antibodies.

FIG. 29 shows binding of the 41-E07 hUCHT1v9 or 43-E07 hUCHT1v9 antibodies to the cells expressing 1×10⁶ copies of the pHLA 9mer or control cells (A375 Luc).

FIG. 30 shows binding of the 41-E07 hUCHT1v9 or 43-E07 hUCHT1v9 antibodies to CD3− and CD3+ Jurkat cells.

FIG. 31 shows binding of the 41-E07 UCHT1v9 or 43-E07 UCHT1v9 antibodies to the indicated cell lines.

FIG. 32 shows binding of the 41-E07 hOKT3a or 41-G5(1C12) hOKT3a to the cells expressing 1×10⁶ copies of the G2 pHLA or control cells (A375 Luc).

FIG. 33 shows binding of the 41-G5(1C12) hUCHT1v9 to the cells expressing 4500 copies of the G5 pHLA.

FIG. 34 shows binding of the 41-E07 hOKT3a or 41-G5(1C12) hOKT3a antibodies to CD3− and CD3+ Jurkat cells.

FIG. 35 shows binding of the 41-G5(1C12) hOKT3a or 41-G5(1C12) UCHT1v9 antibodies to the cells expressing 30,000 copies of the G2 pHLA or control cells (A375 Luc).

FIG. 36A shows tumor volume in mice treated with 0.01 mg/kg, 0.1 mg/kg, or 1 mg/kg of the indicated antibody. FIG. 36B shows tumor volume in mice treated with 1 mg/kg of the indicated antibody. FIG. 36C shows tumor volume in mice treated with 0.1 mg/kg of the indicated antibody. FIG. 36D shows tumor volume in mice treated with 0.01 mg/kg of the indicated antibody.

FIG. 37A shows tumor volume in mice treated with 0.01 mg/kg, 0.1 mg/kg, or 1 mg/kg of the indicated antibody. FIG. 37B shows tumor volume in mice treated with 1 mg/kg of the indicated antibody. FIG. 37C shows tumor volume in mice treated with 0.1 mg/kg of the indicated antibody. FIG. 37D shows tumor volume in mice treated with 0.01 mg/kg of the indicated antibody.

FIG. 38A shows tumor volume in mice treated with 0.01 mg/kg, 0.1 mg/kg, or 1 mg/kg of the indicated antibody. FIG. 38B shows tumor volume in mice treated with 1 mg/kg of the indicated antibody. FIG. 38C shows tumor volume in mice treated with 0.1 mg/kg of the indicated antibody. FIG. 38D shows tumor volume in mice treated with 0.01 mg/kg of the indicated antibody.

FIG. 39A shows tumor volume in mice treated with 2 mg/kg of the indicated antibody. FIG. 39B shows tumor volume in mice treated with 1 mg/kg of the indicated antibody. FIG. 39C shows tumor volume in mice treated with 0.1 mg/kg of the indicated antibody. FIG. 39D shows tumor volume in mice treated with 0.01 mg/kg of the indicated antibody.

FIG. 40A shows binding of the 41-E07 hOKT3a antibody to cells pulsed with the indicated peptide complex. FIG. 40A discloses SEQ ID NOS 214 and 128-133, respectively, in order of appearance. FIG. 40B shows binding of the 41-E07 hOKT3a antibody to cells pulsed with the indicated peptide complex. FIG. 40B discloses SEQ ID NOS 214 and 120-127, respectively, in order of appearance.

FIG. 41 shows binding of the 41-E07 hOKT3a antibody to cells pulsed with the indicated peptide complex. FIG. 41 discloses SEQ ID NOS 214, 204-206, 214, 236, 214, and 233, respectively, in order of appearance.

FIG. 42A shows binding of the 41-E07 UCHT1v9 antibody to cells expressing the indicated peptide complex. FIG. 42B shows binding of the 41-E07 UCHT1v9 antibody to the G2-pHLA complex or the ROBO1 pHLA complex.

FIG. 43A shows the SDS-PAGE results from the FabALACTICA digestion of the format 41 diabody. FIG. 43A shows the SDS-PAGE results from the FabALACTICA digestion of the control Format 3 antibody.

FIG. 44A shows SEC-HPLC results from a product quality screening of antibodies using a TSKgel SuperSW mAb HTP column (top panel), where a peak tailing between 4.5-5.5 minutes suggested presence of an additional antibody moiety that either interacts more with the SEC column, or is more compacted and thus migrates slower than the main antibody conformation. FIG. 44A also shows SEC-HPLC results from a TSKgel G3000SWx1 column (bottom panel) which resolved the tailing into a “split peak.” FIG. 44B includes a schematic showing two conformations of Format 4 antibodies that may exist in solution: dual scFv and diabody conformation.

FIG. 45A shows expected protein digestion fragments of “standard” Format 4 antibodies and a “diabody” isomer of Format 4. FIG. 45B shows SEC-HPLC results from a FabALACTICA digestion experiment, where Format 4 antibodies were treated with a cysteine protease that digests human IgG1 at one specific site in the upper hinge (KSCDKT/HTCPPC (SEQ ID NO: 211)).

FIG. 46 includes a schematic representation of the undigested Format 4 “dual scFv” conformation (left), the diabody conformation without digestion (middle), and the diabody conformation after proteolytic digestion (right).

FIG. 47 shows results from an electron microscopy study of a representative Format 4 antibody, Format 4-hOKT3-G5(1C12).

FIG. 48 includes SEC-HPLC chromatograms from a Format 4 G2(1H11) bispecific antibody with an engineered VH44/VL100 disulfide bond (top panel), and without the engineered disulfide bond (bottom panel).

FIG. 49 includes SEC-HPLC chromatograms from a Format 4 G5(1C12) bispecific antibody with an engineered VH44/VL100 disulfide bond (top panel), and without the engineered disulfide bond (bottom panel).

FIG. 50A includes overlayed SEC-HPLC chromatograms for digested Format 4 G5(1C12) bispecific antibody with an engineered VH44/VL100 disulfide bond. FIG. 50B includes reduced and non-reduced gels (CE-SDS) for digested Format 4 G5(1C12) bispecific antibody with an engineered VH44/VL100 disulfide bond.

FIG. 51A includes overlayed SEC-HPLC chromatograms for digested Format 4 G2(1H11) bispecific antibody with an engineered VH44/VL100 disulfide bond. FIG. 51B includes reduced and non-reduced gels (CE-SDS) for digested Format 4 G2(1H11) bispecific antibody with an engineered VH44/VL100 disulfide bond.

FIG. 52 includes plots showing the BLI results from representative bispecific Format 4 antibodies with and without the engineered VH44/VL100 disulfide bond.

FIG. 53 includes plots showing MSD results from representative bispecific Format 4 antibodies with and without the engineered VH44/VL100 disulfide bond.

FIG. 54 includes plots showing cell binding results from representative bispecific Format 4 antibodies with and without the engineered VH44/VL100 disulfide bond.

FIG. 55 includes plots showing 2D cytotoxicity and spheroid toxicity results from representative G2 Format 4 antibodies with and without the engineered VH44/VL100 disulfide bond.

FIG. 56 includes overlayed SEC-HPLC chromatograms and reduced and non-reduced gels (SDS-PAGE) from digestion of a Format 4 G5(1C12) ABP having a non-shortened linker.

FIG. 57 includes overlayed SEC-HPLC chromatograms and reduced and non-reduced gels (SDS-PAGE) from digestion of a Format 4 G2(1H11) ABP having a non-shortened linker.

FIG. 58 includes overlayed SEC-HPLC chromatograms and reduced and non-reduced gels (SDS-PAGE) from digestion of a Format 4 G5(1C12) ABP having shortened first and second linkers (10 amino acids long).

FIG. 59 includes overlayed SEC-HPLC chromatograms and reduced and non-reduced gels (SDS-PAGE) from digestion of a Format 4 G2(1H11) ABP having shortened first and second linkers (10 amino acids long).

FIG. 60 includes an SEC-HPLC plot and reduced and non-reduced gels (CE-SDS) from digestion of a Format 4 G2(1H11) ABP having (i) shortened first and second linkers (10 amino acids long) and (ii) engineered VH44/VL100 disulfide bond.

FIG. 61 includes plots showing the BLI results from representative bispecific Format 4 antibodies with (“DAB”) and without (2×scFv) shortened first and second linkers (10 amino acids long).

FIG. 62 shows the cell binding results from the indicated G2 and G5 Format 4 ABPs.

FIG. 63 shows the cytotoxicity results for the indicated G2 and G5 Format 4 ABPs.

FIG. 64 includes schematic of Format 4 diabody constructs with disulfide bridge stabilization outside variable domains.

### DETAILED DESCRIPTION

Unless otherwise defined, all terms of art, notations and other scientific terminology used herein are intended to have the meanings commonly understood by those of skill in the art. In some cases, terms with commonly understood meanings are defined herein for clarity and/or for ready reference, and the inclusion of such definitions herein should not necessarily be construed to represent a difference over what is generally understood in the art. The techniques and procedures described or referenced herein are generally well understood and commonly employed using conventional methodologies by those skilled in the art, such as, for example, the widely utilized molecular cloning methodologies described in Sambrook et al., Molecular Cloning: A Laboratory Manual 4th ed. (2012) Cold Spring Harbor Laboratory Press, Cold Spring Harbor, NY. As appropriate, procedures involving the use of commercially available kits and reagents are generally carried out in accordance with manufacturer-defined protocols and conditions unless otherwise noted.

As used herein, the singular forms “a,” “an,” and “the” include the plural referents unless the context clearly indicates otherwise. The terms “include,” “such as,” and the like are intended to convey inclusion without limitation, unless otherwise specifically indicated.

As used herein, the term “comprising” also specifically includes embodiments “consisting of” and “consisting essentially of” the recited elements, unless specifically indicated otherwise. For example, a multispecific ABP “comprising a diabody” includes a multispecific ABP “consisting of a diabody” and a multispecific ABP “consisting essentially of a diabody.”

The term “about” indicates and encompasses an indicated value and a range above and below that value. In certain embodiments, the term “about” indicates the designated value ±10%, ±5%, or ±1%. In certain embodiments, where applicable, the term “about” indicates the designated value(s)±one standard deviation of that value(s).

The term “immunoglobulin” refers to a class of structurally related proteins generally comprising two pairs of polypeptide chains: one pair of light (L) chains and one pair of heavy (H) chains. In an “intact immunoglobulin,” all four of these chains are interconnected by disulfide bonds. The structure of immunoglobulins has been well characterized. See, e.g., Paul, Fundamental Immunology 7th ed., Ch. 5 (2013) Lippincott Williams & Wilkins, Philadelphia, PA. Briefly, each heavy chain typically comprises a heavy chain variable region (VH) and a heavy chain constant region (C_{H}). The heavy chain constant region typically comprises three domains, abbreviated C_{H1}, C_{H2}, and C_{H3}. Each light chain typically comprises a light chain variable region (V_{L}) and a light chain constant region. The light chain constant region typically comprises one domain, abbreviated C_{L}.

The term “antigen binding protein” or “ABP” is used herein in its broadest sense and includes certain types of molecules comprising one or more antigen-binding domains that specifically bind to an antigen or epitope.

In some embodiments, the ABP comprises an antibody. In some embodiments, the ABP consists of an antibody. In some embodiments, the ABP consists essentially of an antibody. An ABP specifically includes intact antibodies (e.g., intact immunoglobulins), antibody fragments, ABP fragments, and multispecific antibodies. In some embodiments, the ABP comprises an alternative scaffold. In some embodiments, the ABP consists of an alternative scaffold. In some embodiments, the ABP consists essentially of an alternative scaffold. In some embodiments, the ABP comprises an antibody fragment. In some embodiments, the ABP consists of an antibody fragment. In some embodiments, the ABP consists essentially of an antibody fragment. In some embodiments, a CAR comprises an ABP provided herein. An “HLA-PEPTIDE ABP,” “anti-HLA-PEPTIDE ABP,” or “HLA-PEPTIDE-specific ABP” is an ABP, as provided herein, which specifically binds to the antigen HLA-PEPTIDE. An ABP includes proteins comprising one or more antigen-binding domains that specifically bind to an antigen or epitope via a variable region, such as a variable region derived from a B cell (e.g., antibody).

The term “antibody” herein is used in the broadest sense and includes polyclonal and monoclonal antibodies, including intact antibodies and functional (antigen-binding) antibody fragments, including fragment antigen binding (Fab) fragments, F(ab′)2 fragments, Fab′ fragments, Fv fragments, recombinant IgG (rIgG) fragments, variable heavy chain (VH) regions capable of specifically binding the antigen, single chain antibody fragments, including single chain variable fragments (scFv), and single domain antibodies (e.g., sdAb, sdFv, nanobody, camelid VHH, engineered or evolved human VH that does not require pairing to VL for solubility or activity) fragments. The term encompasses genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific, antibodies, diabodies, triabodies, and tetrabodies, tandem di-scFv, tandem tri-scFv. Unless otherwise stated, the term “antibody” should be understood to encompass functional antibody fragments thereof. The term also encompasses intact or full-length antibodies, including antibodies of any class or sub-class, including IgG and sub-classes thereof, IgM, IgE, IgA, and IgD.

“Reference antigen binding protein” or “reference ABP” refers to an ABP that, for example, having specificity for a target antigen other than HLA-PEPTIDE target, wherein the HLA Class I molecule is HLA-A*01:01 and the HLA-restricted peptide comprises the sequence NTDNNLAVY (SEQ ID NO: 214). In some embodiments, a reference ABP refers to an ABP having specificity for an HLA-PEPTIDE target, wherein the HLA Class I molecule is HLA-A*01:01 and the HLA-restricted peptide comprises the sequence NTDNNLAVY (SEQ ID NO: 214), however the same ABP has affinity for validate OTLAs (see Examples). In some embodiments, a reference ABP is a Format 4 ABP having an L1 linker and/or L2 linker of a length outside of the range 10-15. In some embodiments, a reference ABP refers to a Format 4 ABP having an L1 linker and/or L2 linker less than 10 amino acids or less than 9 amino acids. In some embodiments, a reference ABP refers to a Format 4 ABP having an L1 linker and/or L2 linker greater than 15 amino acids.

As used herein, “variable region” refers to a variable nucleotide sequence that arises from a recombination event, for example, it can include a V, J, and/or D region of an immunoglobulin.

The term “antigen-binding domain” means the portion of an ABP that is capable of specifically binding to an antigen or epitope. One example of an antigen-binding domain is an antigen-binding domain formed by an antibody V_{H}-V_{L} dimer of an ABP. Another example of an antigen-binding domain is an antigen-binding domain formed by diversification of certain loops from the tenth fibronectin type III domain of an Adnectin. An antigen-binding domain can include antibody CDRs 1, 2, and 3 from a heavy chain in that order; and antibody CDRs 1, 2, and 3 from a light chain in that order.

The antibody V_{H} and V_{L} regions may be further subdivided into regions of hypervariability (“hypervariable regions (HVRs);” also called “complementarity determining regions” (CDRs)) interspersed with regions that are more conserved. The more conserved regions are called framework regions (FRs). Each V_{H} and V_{L} generally comprises three antibody CDRs and four FRs, arranged in the following order (from N-terminus to C-terminus): FR1-CDR1-FR2-CDR2-FR3-CDR3-FR4. The antibody CDRs are involved in antigen binding, and influence antigen specificity and binding affinity of the ABP. See Kabat et al., Sequences of Proteins of Immunological Interest 5th ed. (1991) Public Health Service, National Institutes of Health, Bethesda, MD, incorporated by reference in its entirety.

The light chain from any vertebrate species can be assigned to one of two types, called kappa (κ) and lambda (λ), based on the sequence of its constant domain.

The heavy chain from any vertebrate species can be assigned to one of five different classes (or isotypes): IgA, IgD, IgE, IgG, and IgM. These classes are also designated α, δ, ε, γ, and μ, respectively. The IgG and IgA classes are further divided into subclasses on the basis of differences in sequence and function. Humans express the following subclasses: IgG1, IgG2, IgG3, IgG4, IgA1, and IgA2.

The amino acid sequence boundaries of an antibody CDR can be determined by one of skill in the art using any of a number of known numbering schemes, including those described by Kabat et al., supra (“Kabat” numbering scheme); Al-Lazikani et al., 1997, J. Mol. Biol., 273:927-948 (“Chothia” numbering scheme); MacCallum et al., 1996, J. Mol. Biol. 262:732-745 (“Contact” numbering scheme); Lefranc et al., Dev. Comp. Immunol., 2003, 27:55-77 (“IMGT” numbering scheme); and Honegge and Pluckthun, J. Mol. Biol., 2001, 309:657-70 (“AHo” numbering scheme); each of which is incorporated by reference in its entirety.

Table A provides the positions of antibody LCDR1, LCDR2, LCDR3, HCDR1, HCDR2, and HCDR3 as identified by the Kabat and Chothia schemes. For HCDR1, residue numbering is provided using both the Kabat and Chothia numbering schemes.

Antibody CDRs may be assigned, for example, using ABP numbering software, such as Abnum, available at www.bioinf.org.uk/abs/abnum/, and described in Abhinandan and Martin, Immunology, 2008, 45:3832-3839, incorporated by reference in its entirety.

TABLE A
Residues in CDRs according to Kabat
and Chothia numbering schemes

| CDR | Kabat | Chothia |
|---|---|---|
| L1 | L24-L34 | L24-L34 |
| L2 | L50-L56 | L50-L56 |
| L3 | L89-L97 | L89-L97 |
| H1 (Kabat Numbering) | H31-H35B | H26-H32 or H34* |
| H1 (Chothia Numbering) | H31-H35 | H26-H32 |
| H2 | H50-H65 | H52-H56 |
| H3 | H95-H102 | H95-H102 |

*The C-terminus of CDR-H1, when numbered using the Kabat numbering convention, varies between H32 and H34, depending on the length of the CDR.

The “EU numbering scheme” is generally used when referring to a residue in an ABP heavy chain constant region (e.g., as reported in Kabat et al., supra). Unless stated otherwise, the EU numbering scheme is used to refer to residues in ABP heavy chain constant regions described herein.

The terms “full length antibody,” “intact antibody,” and “whole antibody” are used herein interchangeably to refer to an antibody having a structure substantially similar to a naturally occurring antibody structure and having heavy chains that comprise an Fc region. For example, when used to refer to an IgG molecule, a “full length antibody” is an antibody that comprises two heavy chains and two light chains.

An “ABP fragment” comprises a portion of an intact ABP, such as the antigen-binding or variable region of an intact ABP. ABP fragments include, for example, Fv fragments, Fab fragments, F(ab′)₂ fragments, Fab′ fragments, scFv (sFv) fragments, and scFv-Fc fragments. ABP fragments include antibody fragments. Antibody fragments can include Fv fragments, Fab fragments, F(ab′)₂ fragments, Fab′ fragments, scFv (sFv) fragments, and scFv-Fc fragments

“Fv” fragments comprise a non-covalently-linked dimer of one heavy chain variable domain and one light chain variable domain.

“Fab” fragments comprise, in addition to the heavy and light chain variable domains, the constant domain of the light chain and the first constant domain (C_{H1}) of the heavy chain. Fab fragments may be generated, for example, by recombinant methods or by papain digestion of a full-length ABP.

“F(ab′)₂” fragments contain two Fab′ fragments joined, near the hinge region, by disulfide bonds. F(ab′)₂ fragments may be generated, for example, by recombinant methods or by pepsin digestion of an intact ABP. The F(ab′) fragments can be dissociated, for example, by treatment with β-mercaptoethanol.

“Single-chain Fv” or “sFv” or “scFv” fragments comprise a VH domain and a V_{L} domain in a single polypeptide chain. The V_{H} and V_{L} are generally linked by a peptide linker. See Pluckthun A. (1994). Any suitable linker may be used. In some embodiments, the linker is a (GGGGS)ₙ (SEQ ID NO: 217). In some embodiments, n=1, 2, 3, 4, 5, or 6. See ABPs from Escherichia coli. In Rosenberg M. & Moore G. P. (Eds.), The Pharmacology of Monoclonal ABPs vol. 113 (pp. 269-315). Springer-Verlag, New York, incorporated by reference in its entirety.

“scFv-Fc” fragments comprise an scFv attached to an Fc domain. For example, an Fc domain may be attached to the C-terminal of the scFv. The Fc domain may follow the VH or VL, depending on the orientation of the variable domains in the scFv (i.e., V_{H}-V_{L} or V_{L}-V_{H}). Any suitable Fc domain known in the art or described herein may be used. In some cases, the Fc domain comprises an IgG4 Fc domain.

The term “Fc region” or “Fc” refers to the C-terminal region of an immunoglobulin heavy chain that, in naturally occurring antibodies, interacts with Fc receptors and certain proteins of the complement system. The structures of the Fc regions of various immunoglobulins, and the glycosylation sites contained therein, are known in the art. See Schroeder and Cavacini, J. Allergy Clin. Immunol., 2010, 125:S41-52, incorporated by reference in its entirety. The Fc region may be a naturally occurring Fc region, or an Fc region modified as described in the art or elsewhere in this disclosure.

The term “alternative scaffold” refers to a molecule in which one or more regions may be diversified to produce one or more antigen-binding domains that specifically bind to an antigen or epitope. In some embodiments, the antigen-binding domain binds the antigen or epitope with specificity and affinity similar to that of an ABP. Exemplary alternative scaffolds include those derived from fibronectin (e.g., Adnectins™), the β-sandwich (e.g., iMab), lipocalin (e.g., Anticalins®), EETI-II/AGRP, BPTI/LACI-D1/ITI-D2 (e.g., Kunitz domains), thioredoxin peptide aptamers, protein A (e.g., Affibody®), ankyrin repeats (e.g., DARPins), gamma-B-crystallin/ubiquitin (e.g., Affilins), CTLD₃ (e.g., Tetranectins), Fynomers, and (LDLR-A module) (e.g., Avimers). Additional information on alternative scaffolds is provided in Binz et al., Nat. Biotechnol., 2005 23:1257-1268; Skerra, Current Opin. in Biotech., 2007 18:295-304; and Silacci et al., J. Biol. Chem., 2014, 289:14392-14398; each of which is incorporated by reference in its entirety. An alternative scaffold is one type of ABP.

A “multispecific ABP” is an ABP that comprises two or more different antigen-binding domains that collectively specifically bind two or more different epitopes. The two or more different epitopes may be epitopes on the same antigen (e.g., a single HLA-PEPTIDE molecule expressed by a cell) or on different antigens (e.g., different HLA-PEPTIDE molecules expressed by the same cell, or a HLA-PEPTIDE molecule and a non-HLA-PEPTIDE molecule). In some aspects, a multispecific ABP binds two different epitopes (i.e., a “bispecific ABP”). In some aspects, a multispecific ABP binds three different epitopes (i.e., a “trispecific ABP”).

The term “monoclonal antibody” refers to an antibody from a population of substantially homogeneous antibodies. A population of substantially homogeneous antibodies comprises antibodies that are substantially similar and that bind the same epitope(s), except for variants that may normally arise during production of the monoclonal antibody. Such variants are generally present in only minor amounts. A monoclonal antibody is typically obtained by a process that includes the selection of a single antibody from a plurality of antibodies. For example, the selection process can be the selection of a unique clone from a plurality of clones, such as a pool of hybridoma clones, phage clones, yeast clones, bacterial clones, or other recombinant DNA clones. The selected antibody can be further altered, for example, to improve affinity for the target (“affinity maturation”), to humanize the antibody, to improve its production in cell culture, and/or to reduce its immunogenicity in a subject.

As used, the term “diabody” refers to a dimerized antigen binding region (ABR) comprising a heavy chain variable domain (VH) and a light chain variable domain (V_{L}). Diabodies have two antigen binding sites and can be bispecific or monospecific. (See, for example, Holliger and Winter, Cancer Immunol Immunother, 1997, 45:128-130 and Proc. Natl. Acad. Sci. USA, 1993, 90:6444-6448, each of which is incorporated by reference in its entirety).

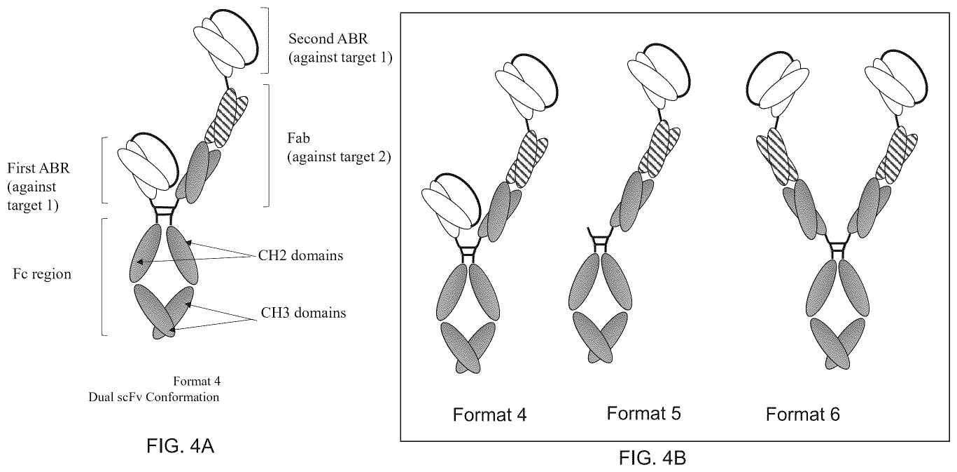

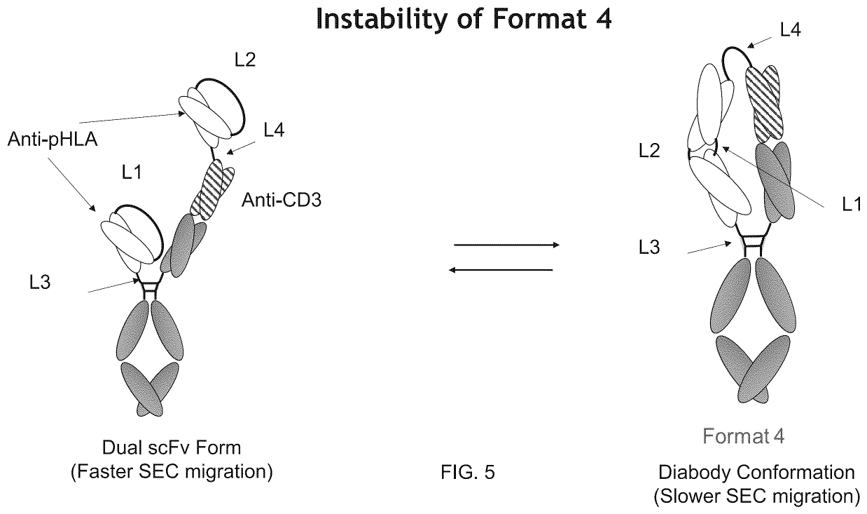

As used, the term “interacts” refers to the non-covalent pairing of VH and VL sequences either within an scFv or between a VH domain and VL domain of an ABR or set of ABRs, e.g., to form an antigen binding site. It is also contemplated that the VH domain from a first ABR can interact with the VL domain from another ABR. For example, in FIG. 4A, in each of the ABRs, a V_{H} is shown interacting with a V_{L} from the same polypeptide (intramolecular interaction). In the diabody of FIG. 5 (right), a V_{H} from the first ABR (in the first polypeptide) is shown interacting with a V_{L} from the second ABR (in the second polypeptide), while a V_{L} from the first ABR (in the first polypeptide) is shown interacting with a V_{H} from the second ABR (in the second polypeptide). These noncovalent interactions that facilitate the pairing can consist of hydrophobic, electrostatic, and van der Waals interactions. Further, in some embodiments, these noncovalent interactions may be stabilized by introduction of 2 Cys residues to form a disulfide bond (DSB) holding VH and VL together covalently. In some embodiments, this involves a Cys introduced in VH at position 44 according to Kabat numbering and a second Cys at VL100 according to Kabat numbering.

The term “chimeric antibody” refers to an antibody in which a portion of the heavy and/or light chain is derived from a particular source or species, while the remainder of the heavy and/or light chain is derived from a different source or species.

“Humanized” forms of non-human antibodies are chimeric antibodies that contain minimal sequence derived from the non-human antibody. A humanized antibody is generally a human antibody (recipient antibody) in which residues from one or more CDRs are replaced by residues from one or more CDRs of a non-human antibody (donor antibody). The donor antibody can be any suitable non-human antibody, such as a mouse, rat, rabbit, chicken, or non-human primate antibody having a desired specificity, affinity, or biological effect. In some instances, selected framework region residues of the recipient antibody are replaced by the corresponding framework region residues from the donor antibody. Humanized antibodies may also comprise residues that are not found in either the recipient antibody or the donor antibody. Such modifications may be made to further refine antibody function. For further details, see Jones et al., Nature, 1986, 321:522-525; Riechmann et al., Nature, 1988, 332:323-329; and Presta, Curr. Op. Struct. Biol., 1992, 2:593-596, each of which is incorporated by reference in its entirety.

A “human antibody” is one which possesses an amino acid sequence corresponding to that of an antibody produced by a human or a human cell, or derived from a non-human source that utilizes a human antibody repertoire or human antibody-encoding sequences (e.g., obtained from human sources or designed de novo). Human antibodies specifically exclude humanized antibodies.

As used, the term “tumor antigen” refers to refers to an antigen or portion thereof expressed only by a tumor or at a level that is higher than that expressed by normal tissue. In some embodiments, tumor antigens are exclusively expressed on tumor cells. In some embodiments, the presence or expression of a tumor antigen on normal cells is negligible. In some embodiments, these tumor antigens are expressed in a significantly higher amount on tumor cells than on normal cells. In some embodiments, the tumor antigen is an HLA-PEPTIDE.

As used, the term “target antigen” refers to an antigen or portion thereof capable of stimulating an immune response and/or being bound by a binding domain of an immune cell. Target antigens can be bound by the antigen binding site of an antibody or antibody fragment. The term target antigen encompasses, for example, cell surface molecules present on effector cells such as T cells or NK cells. In some embodiments, the target antigen is CD3. The term target antigen also encompasses tumor antigens, as described supra.

“T cells” refer to a type of lymphocyte that naturally expresses a T-cell receptor on its cell surface and plays a central role in the immune response (e.g., immune-related cell death). They differentiate into several distinct types of T cells (e.g., helper, regulatory, or cytotoxic T cells, and memory T cells). Effector T cells, for example, refer to the subset of cytotoxic T cells which are actively involved in eliminating (e.g., killing) different types of cells that are infected with pathogens, or are otherwise damaged or dysfunctional.

“Natural killer cells” or “NK cells” are a component of the innate immune system and make up approximately 15% of circulating lymphocytes. NK cells infiltrate virtually all tissues, killing target cells by means similar to cytotoxic T cells—i.e., via cytolytic granules that contain perforin and granzymes as well as via death receptor pathways. Activated NK cells also secrete inflammatory cytokines and chemokines that promote the recruitment of other leukocytes to the target tissue.

As used herein, the term “cytotoxicity” refers to the ability of antibodies, antibody fragments, and ABPs as described herein to mediate or facilitate cell death or elimination through an effector cell of the immune system (e.g., T cells and/or NK cells). For example, the term “cytotoxicity” can refer to a process by which an ABP binds an effector cell (e.g., an anti-CD3 binding domain of the ABP binds to CD3 present on an effector cell (e.g., a T cell)) and a tumor antigen binding domain of the ABP binds a target cell expressing an antigen such as a tumor antigen (e.g., a pHLA binding domain binds to a target cell expressing pHLA). Thereafter, the effector cell facilitates cell death and/or destruction (e.g., via apoptosis or lysis) of the target cell. Cytotoxic T cells, for example, can destroy the target cell through release of various molecules such as cytokines, perforin, granzymes, and proteases, which cause the target cell to undergo cell death (e.g., apoptosis). In some embodiments, the term cytotoxicity also encompasses antibody-dependent cellular cytotoxicity (also referred to as antibody-dependent cell-mediated cytotoxicity), which is an immune defense mechanism whereby effector cells of the immune system actively lyse a target cell. It is typically driven by Fc bind to Fc receptors.

“Affinity” refers to the strength of the sum total of non-covalent interactions between a single binding site of a molecule (e.g., an ABP) and its binding partner (e.g., an antigen or epitope). Unless indicated otherwise, as used herein, “affinity” refers to intrinsic binding affinity, which reflects a 1:1 interaction between members of a binding pair (e.g., ABP and antigen or epitope). The affinity of a molecule X for its partner Y can be represented by the dissociation equilibrium constant (K_{D}). The kinetic components that contribute to the dissociation equilibrium constant are described in more detail below. Affinity can be measured by common methods known in the art, including those described herein, such as surface plasmon resonance (SPR) technology (e.g., BIACORE®) or biolayer interferometry (e.g., FORTEBIO®).

With regard to the binding of an ABP to a target molecule, the terms “bind,” “specific binding,” “specifically binds to,” “specific for,” “selectively binds,” and “selective for” a particular antigen (e.g., a polypeptide target) or an epitope on a particular antigen mean binding that is measurably different from a non-specific or non-selective interaction (e.g., with a non-target molecule). Specific binding can be measured, for example, by measuring binding to a target molecule and comparing it to binding to a non-target molecule. Specific binding can also be determined by competition with a control molecule that mimics the epitope recognized on the target molecule. In that case, specific binding is indicated if the binding of the ABP to the target molecule is competitively inhibited by the control molecule. In some aspects, the affinity of a HLA-PEPTIDE ABP for a non-target molecule is less than about 50% of the affinity for HLA-PEPTIDE. In some aspects, the affinity of a HLA-PEPTIDE ABP for a non-target molecule is less than about 40% of the affinity for HLA-PEPTIDE. In some aspects, the affinity of a HLA-PEPTIDE ABP for a non-target molecule is less than about 30% of the affinity for HLA-PEPTIDE. In some aspects, the affinity of a HLA-PEPTIDE ABP for a non-target molecule is less than about 20% of the affinity for HLA-PEPTIDE. In some aspects, the affinity of a HLA-PEPTIDE ABP for a non-target molecule is less than about 10% of the affinity for HLA-PEPTIDE. In some aspects, the affinity of a HLA-PEPTIDE ABP for a non-target molecule is less than about 1% of the affinity for HLA-PEPTIDE. In some aspects, the affinity of a HLA-PEPTIDE ABP for a non-target molecule is less than about 0.1% of the affinity for HLA-PEPTIDE.

The term “k_{d}” (sec⁻¹), as used herein, refers to the dissociation rate constant of a particular ABP-antigen interaction. This value is also referred to as the k_{off} value.

The term “kₐ” (M⁻¹×sec⁻¹), as used herein, refers to the association rate constant of a particular ABP-antigen interaction. This value is also referred to as the kₒₙ value.

The term “K_{D}” (M), as used herein, refers to the dissociation equilibrium constant of a particular ABP-antigen interaction. K_{D}=k_{d}/kₐ. In some embodiments, the affinity of an ABP is described in terms of the K_{D} for an interaction between such ABP and its antigen. For clarity, as known in the art, a smaller K_{D} value indicates a higher affinity interaction, while a larger K_{D} value indicates a lower affinity interaction.

The term “K_{A}” (M⁻¹), as used herein, refers to the association equilibrium constant of a particular ABP-antigen interaction. K_{A}=kₐ/k_{d}.

An “immunoconjugate” is an ABP conjugated to one or more heterologous molecule(s), such as a therapeutic (cytokine, for example) or diagnostic agent.

“Fc effector functions” refer to those biological activities mediated by the Fc region of an ABP having an Fc region, which activities may vary depending on isotype. Examples of ABP effector functions include C1q binding to activate complement dependent cytotoxicity (CDC), Fc receptor binding to activate ABP-dependent cellular cytotoxicity (ADCC), and ABP dependent cellular phagocytosis (ADCP).

When used herein in the context of two or more ABPs, the term “competes with” or “cross-competes with” indicates that the two or more ABPs compete for binding to an antigen (e.g., HLA-PEPTIDE).

The term “epitope” means a portion of an antigen that specifically binds to an ABP. Epitopes frequently consist of surface-accessible amino acid residues and/or sugar side chains and may have specific three dimensional structural characteristics, as well as specific charge characteristics. Conformational and non-conformational epitopes are distinguished in that the binding to the former but not the latter may be lost in the presence of denaturing solvents. An epitope may comprise amino acid residues that are directly involved in the binding, and other amino acid residues, which are not directly involved in the binding. The epitope to which an ABP binds can be determined using known techniques for epitope determination such as, for example, testing for ABP binding to HLA-PEPTIDE variants with different point-mutations, or to chimeric HLA-PEPTIDE variants.

Percent “identity” between a polypeptide sequence and a reference sequence, is defined as the percentage of amino acid residues in the polypeptide sequence that are identical to the amino acid residues in the reference sequence, after aligning the sequences and introducing gaps, if necessary, to achieve the maximum percent sequence identity. Alignment for purposes of determining percent amino acid sequence identity can be achieved in various ways that are within the skill in the art, for instance, using publicly available computer software such as BLAST, BLAST-2, ALIGN, MEGALIGN (DNASTAR), CLUSTALW, CLUSTAL OMEGA, or MUSCLE software. Those skilled in the art can determine appropriate parameters for aligning sequences, including any algorithms needed to achieve maximal alignment over the full length of the sequences being compared.

In some embodiments, the VH and VL of the antibody binding regions (ABRs) in the ABPs of the present disclosure are at least about 90%, about 91%, about 92%, about 93%, about 94%, about 95%, about 96%, about 97%, about 98%, about 99%, about 100% identical to the VH and VL for 31E07 (Table 19). In some embodiments, the VH and VL of the Fab region in the ABP of the present disclosure are at least about 90%, about 91%, about 92%, about 93%, about 94%, about 95%, about 96%, about 97%, about 98%, about 99%, about 100% identical to the VH and VL for hOKT3a (Table 21).

A “conservative substitution” or a “conservative amino acid substitution,” refers to the substitution an amino acid with a chemically or functionally similar amino acid. Conservative substitution tables providing similar amino acids are well known in the art. By way of example, the groups of amino acids provided in Tables 10, 15 and 16 are, in some embodiments, considered conservative substitutions for one another.

TABLE B
Selected groups of amino acids that are considered conservative
substitutions for one another, in certain embodiments.

| | |
|---|---|
| Acidic Residues | D and E |
| Basic Residues | K, R, and H |
| Hydrophilic Uncharged Residues | S, T, N, and Q |
| Aliphatic Uncharged Residues | G, A, V, L, and I |
| Non-polar Uncharged Residues | C, M, and P |
| Aromatic Residues | F, Y, and W |

TABLE C
Additional selected groups of amino acids that
are considered conservative substitutions
for one another, in certain embodiments.

| | |
|---|---|
| Group 1 | A, S, and T |
| Group 2 | D and E |
| Group 3 | N and Q |
| Group 4 | R and K |
| Group 5 | I, L, and M |
| Group 6 | F, Y, and W |

TABLE D
Further selected groups of amino acids that are considered conservative
substitutions for one another, in certain embodiments.

| | |
|---|---|
| Group A | A and G |
| Group B | D and E |
| Group C | N and Q |
| Group D | R, K, and H |
| Group E | I, L, M, V |
| Group F | F, Y, and W |
| Group G | S and T |
| Group H | C and M |

Additional conservative substitutions may be found, for example, in Creighton, Proteins: Structures and Molecular Properties 2nd ed. (1993) W. H. Freeman & Co., New York, NY. An ABP generated by making one or more conservative substitutions of amino acid residues in a parent ABP is referred to as a “conservatively modified variant.”

The term “amino acid” refers to the twenty common naturally occurring amino acids. Naturally occurring amino acids include alanine (Ala; A), arginine (Arg; R), asparagine (Asn; N), aspartic acid (Asp; D), cysteine (Cys; C); glutamic acid (Glu; E), glutamine (Gln; Q), Glycine (Gly; G); histidine (His; H), isoleucine (Ile; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V).

The term “protein liability” refers to a possible chemical modification at a particular amino acid, or sequence of amino acids, in a protein that could lead to alterations of biophysical and/or biological properties.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

The terms “host cell,” “host cell line,” and “host cell culture” are used interchangeably and refer to cells into which an exogenous nucleic acid has been introduced, and the progeny of such cells. Host cells include “transformants” (or “transformed cells”) and “transfectants” (or “transfected cells”), which each include the primary transformed or transfected cell and progeny derived therefrom. Such progeny may not be completely identical in nucleic acid content to a parent cell, and may contain mutations.

The term “treating” (and variations thereof such as “treat” or “treatment”) refers to clinical intervention in an attempt to alter the natural course of a disease or condition in a subject in need thereof. Treatment can be performed both for prophylaxis and during the course of clinical pathology. Desirable effects of treatment include preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis.

As used herein, the term “therapeutically effective amount” or “effective amount” refers to an amount of an ABP or pharmaceutical composition provided herein that, when administered to a subject, is effective to treat a disease or disorder.

As used herein, the term “subject” means a mammalian subject. Exemplary subjects include humans, monkeys, dogs, cats, mice, rats, cows, horses, camels, goats, rabbits, and sheep. In certain embodiments, the subject is a human. In some embodiments the subject has a disease or condition that can be treated with an ABP provided herein. In some aspects, the disease or condition is a cancer. In some aspects, the disease or condition is a viral infection.

The term “package insert” is used to refer to instructions customarily included in commercial packages of therapeutic or diagnostic products (e.g., kits) that contain information about the indications, usage, dosage, administration, combination therapy, contraindications and/or warnings concerning the use of such therapeutic or diagnostic products.

The term “tumor” refers to all neoplastic cell growth and proliferation, whether malignant or benign, and all pre-cancerous and cancerous cells and tissues. The terms “cancer,” “cancerous,” “cell proliferative disorder,” “proliferative disorder” and “tumor” are not mutually exclusive as referred to herein. The terms “cell proliferative disorder” and “proliferative disorder” refer to disorders that are associated with some degree of abnormal cell proliferation. In some embodiments, the cell proliferative disorder is a cancer. In some aspects, the tumor is a solid tumor. In some aspects, the tumor is a hematologic malignancy.

The term “pharmaceutical composition” refers to a preparation which is in such form as to permit the biological activity of an active ingredient contained therein to be effective in treating a subject, and which contains no additional components which are unacceptably toxic to the subject in the amounts provided in the pharmaceutical composition.

The terms “modulate” and “modulation” refer to reducing or inhibiting or, alternatively, activating or increasing, a recited variable.

The terms “increase” and “activate” refer to an increase of 10%, 20%, 30%, 40%, 50%, 60%, 70%, 75%, 80%, 85%, 90%, 95%, 100%, 2-fold, 3-fold, 4-fold, 5-fold, 10-fold, 20-fold, 50-fold, 100-fold, or greater in a recited variable.

The terms “reduce” and “inhibit” refer to a decrease of 10%, 20%, 30%, 40%, 50%, 60%, 70%, 75%, 80%, 85%, 90%, 95%, 2-fold, 3-fold, 4-fold, 5-fold, 10-fold, 20-fold, 50-fold, 100-fold, or greater in a recited variable.

The terms “nucleic acids” and “polynucleotides” may be used interchangeably herein to refer to polymeric form of nucleotides of any length, either deoxyribonucleotides or ribonucleotides, or analogs thereof. Polynucleotides can include, but are not limited to coding or non-coding regions of a gene or gene fragment, loci (locus) defined from linkage analysis, exons, introns, messenger RNA (mRNA), cDNA, recombinant polynucleotides, branched polynucleotides, plasmids, vectors, isolated DNA, isolated RNA, nucleic acid probes, and primers. A polynucleotide may comprise modified nucleotides, such as methylated nucleotides and nucleotide analogs. Exemplary modified nucleotides include, e.g., 5-fluorouracil, 5-bromouracil, 5-chlorouracil, 5-iodouracil, hypoxanthine, xanthine, 4-acetylcytosine, 5-(carboxyhydroxymethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-substituted adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthioN6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, 3-(3-amino-3-N-2-carboxypropyl) uracil, and 2,6-diaminopurine.

### Formats, Dual ScFv Conformation and Diabody Conformation in ABPs

This application is related to PCT/US2020/015736, filed on Jan. 29, 2020, published as WO2020160189A1; U.S. application Ser. No. 17/426,627, filed Jul. 28, 2021; and PCT/US2021/043796, filed on Jul. 29, 2021, each of which are hereby incorporated by reference in their entirety for all purposes.

The inventors of the present disclosure identified that antibodies (e.g., Format 4 antibodies) can exist in two conformations: (i) dual scFv (Format 4) and (ii) diabody conformation (Format 41). In solution, these antibodies may exist in equilibrium between the two conformations and these two conformations may have different relative properties. For example, the two conformations in a solution may differ in terms of, but not limited to, antibody affinity to a target (e.g., to tumor-associated antigens, receptors expressed on tumor cells, receptors highly expressed on tumor cells, pHLA, etc.), cytotoxicity to diseased cells, pharmacokinetic profiles, immunogenicity, stimulation of anti-drug antibodies, etc. The present disclosure identifies modifications, as described herein, to drive the antibodies towards a single conformation, i.e., either dual scFv conformation or diabody conformation.

Provided herein are Format 4 antibodies, as shown in FIG. 4A. A Format 4 antibody can be described as an ABP comprising three polypeptides, wherein the first polypeptide comprises, in an N→C direction, a first ABR (antigen binding region), a first hinge, a CH2 domain and a CH3 domain. The second polypeptide comprises, in an N→C direction, a VH domain of a Fab region, a CH1 domain of a Fab region, a second hinge, a CH2 domain, and a CH3 domain. The third polypeptide comprises a light chain comprising, in an N→C direction, a VL domain of the Fab region and a CL domain of the Fab region. The first and second ABRs each comprise a VH domain and a VL domain. A second ABR is attached, directly or indirectly, to the N-terminus of the second polypeptide or the third polypeptide. The first and second ABRs each comprise in an N→C direction: (i) a VH domain-a VL domain or (ii) a VL domain-VH domain. In some embodiments, the VH and VL domains of the first ABR interact with each other (shown in FIG. 4A). In some embodiments, the VH and VL domains of the second ABR interact with each other (shown in FIG. 4A). The hinge-CH2-CH3 domains on the first and second polypeptide constitute the Fc region of the ABP. This ABP is referred to herein as a Format 4 antibody in extended conformation, dual scFv conformation or 2×scFv conformation. (See FIG. 4A).

In some embodiments, a Format 4 antibody can be described as an ABP comprising three polypeptides, wherein the first polypeptide comprises, in an N→C direction, a first ABR (antigen binding region), a first hinge, a CH2 domain and a CH3 domain. The second polypeptide comprises, in an N→C direction, a second ABR, a VH domain of a Fab region, a CH1 domain of a Fab region, a second hinge, a CH2 domain, and a CH3 domain. The third polypeptide comprises a light chain comprising, in an N→C direction, a VL domain of the Fab region and a CL domain of the Fab region. The first and second ABRs each comprise a VH domain and a VL domain. The first and second ABRs each comprise in an N→C direction: (i) a VH domain-a VL domain or (ii) a VL domain-VH domain. In some embodiments, the VH and VL domains of the first ABR interact with each other (shown in FIG. 4A). In some embodiments, the VH and VL domains of the second ABR interact with each other (shown in FIG. 4A). The hinge-CH2-CH3 domains on the first and second polypeptide constitute the Fc region of the ABP. This ABP is referred to herein as a Format 4 antibody in extended conformation, dual scFv conformation or 2×scFv conformation. (See FIG. 4A).

In some embodiments, the first ABR comprises, in an N→C direction, a VH domain of the first ABR and a VL domain of the first ABR. In some embodiments, the first ABR comprises, in an N→C direction, a VL domain of the first ABR and a VH domain of the first ABR. In some embodiments, the second ABR comprises, in an N→C direction, a VH domain of the second ABR and a VL domain of the second ABR. In some embodiments, the second ABR comprises in an N→C direction, a VL domain of the second ABR and a VH domain of the second ABR.

In certain embodiments, wherein the ABP is a Format 4 antibody in extended conformation, the ABPs each comprise a first ABR and a second ABR that each specifically bind an epitope of a first target antigen (e.g., G2), a Fab that specifically binds an epitope of an additional target antigen (e.g., a cell surface molecule on an effector cell, e.g. CD3) that is distinct from the first target antigen, and an Fc domain, wherein the ABP comprises a first polypeptide, a second polypeptide, and a third polypeptide, wherein the first polypeptide comprises, in an N→C direction, a first ABR-a hinge-CH2-CH3, wherein the second polypeptide comprises, in an N→C direction, a second ABR-a VH domain of the Fab-a CH1 domain of the Fab-a hinge-CH2-CH3, and wherein the third polypeptide comprises, in an N→C direction, a VL domain of the Fab-a CL domain of the Fab. The VH domain of the first ABR is attached to the VL domain of the first ABR via a first linker (e.g. see L1 of the 2×scFv form in FIG. 5) and the VH domain of the second ABR is attached to the VL domain of the second ABR via a second linker (e.g., see L2 of the 2×scFv form in FIG. 5). Additional linkers shown in FIG. 5 are linker 3 (L3) and linker 4 (L4). L3 attaches the first ABR to the Fc domain. L4 attaches the second ABR to the Fab.

In some embodiments, the first target antigen is an HLA-PEPTIDE target. In some embodiments, the additional target antigen is expressed on an effector cell (e.g. T cells or NK cells). In some embodiments, the additional target antigen is a cell surface molecule expressed on an effector cells (e.g. T cells or NK cells). In some embodiments, the cell surface molecule is CD3, optionally CD38.

The present disclosure also provides ABPs comprising three polypeptides, wherein the first polypeptide comprises, in an N→C direction, a first ABR, a first hinge, a CH2 domain, and a CH3 domain. The second polypeptide comprises, in an N→C direction, a second ABR, a VH domain of a Fab region, a CH1 domain of a Fab region, a second hinge, a CH2 domain, and a CH3 domain. The third polypeptide comprises a light chain comprising, in an N→C direction, a VL domain of the Fab region and a CL domain of the Fab region. In this conformation of ABP, the VH domain of the first ABR interacts with the VL domain of the second ABR, while the VH domain of the second ABR interacts with the VL domain of the first ABR, thereby forming a diabody (see FIG. 5). The hinge-CH2-CH3 domains on the first and second polypeptide constitute the Fc region of the ABP. This ABP is referred to herein as a Format 4 antibody in compact conformation or diabody conformation. The diabody conformation is also referred to herein as “circularized conformation” and is shown in FIG. 5.

In some embodiments, the first ABR comprises, in an N→C direction, a VH domain of the first ABR and a VL domain of the first ABR. In some embodiments, the first ABR comprises, in an N→C direction, a VL domain of the first ABR and a VH domain of the first ABR. In some embodiments, the second ABR comprises, in an N→C direction, a VH domain of the second ABR and a VL domain of the second ABR. In some embodiments, the second ABR comprises in an N→C direction, a VL domain of the second ABR and a VH domain of the second ABR.

In certain embodiments, wherein the ABP is a Format 4 antibody in diabody conformation, the VH domain of the first ABR is attached to the VL domain of the second ABR via a first linker (e.g., L1 of the diabody form in FIG. 5) and the VH domain of the second ABR is attached to the VL domain of the first ABR via a second linker (e.g., L2 of the diabody form in FIG. 5).

Also described herein, are Format 5 and Format 6 antibodies, as shown in FIG. 4B and described in International Application No. PCT/US2015/033076, which is incorporated by reference in its entirety.

In some embodiments, Format 4 antibodies exist in equilibrium between two conformations: (i) an extended conformation referred to as dual scFv (or 2×scFv) conformation, and (ii) compact conformation (or diabody conformation).

When a Format 4 antibody is in the dual scFv conformation (shown in FIG. 4A), the VH of the first ABR interacts (pairs) with the VL of the first ABR and the VH of the second ABR interacts with the VL of the second ABR. Alternatively, when the Format 4 antibody is in compact conformation (or diabody conformation), the VH of the first ABR interacts (pairs) with the VL of the second ABR, while the VH of the second ABR interacts with the VL of the first ABR and forms a diabody.

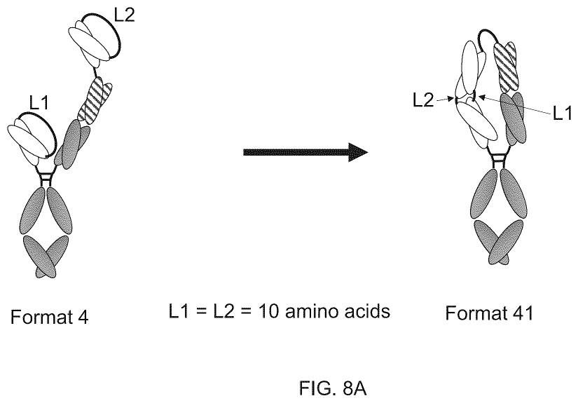

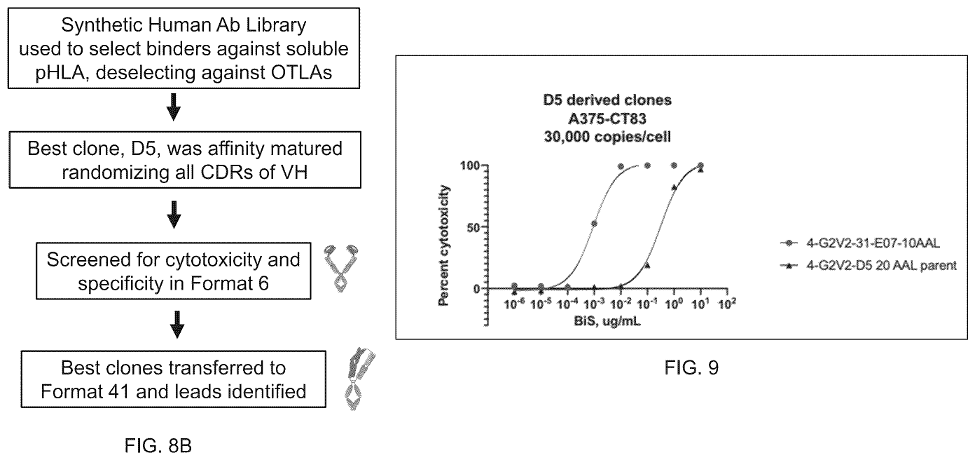

Format 41 antibodies (shown in FIG. 8A) exist stably in diabody conformation in solution (i.e. they do not form dual scFv conformation in solution or there is a negligible fraction that will form dual scFv conformation) and they have shortened L1 and L2 linkers. In some embodiments, the shortened L1 and L2 linkers in Format 41 antibodies can range from 5-15 amino acids in length. In some embodiments, the L1 and L2 linkers consist of 5, 6, 7, 8, 9, 10, 11, 12, 13, 14 or 15 amino acids. In some embodiments, the L1 and L2 linkers consist of 5, 8 or 10 amino acids. In some embodiments, the L1 and L2 linkers consist of 5, 8 or 10 amino acids. In some embodiments, the L1 and L2 linkers consist of 5, 6, 7, 8, 9 or 10 amino acids. In some embodiments, the L1 and L2 linkers consist of a range of amino acids selected from: 5-15, 5-14, 5-13, 5-12, 5-11, 5-10, 5-9, 5-8, 5-7, 5-6, 6-15, 6-14, 6-13, 6-12, 6-11, 6-10, 6-9, 6-8, 6-7, 7-15, 7-14, 7-13, 7-12, 7-11, 7-10, 7-9, 7-8, 8-15, 8-14, 8-13, 8-12, 8-11, 8-10, 8-9, 9-15, 9-14, 9-13, 9-12, 9-11, 9-10, 10-15, 10-14, 10-13, 10-12, 10-11, 11-15, 11-14, 11-13, 11-12, 12-15, 12-14, 12-13, 13-15, and 13-14 amino acids in length. In some embodiments, the L1 an L2 linkers consist of less than 5, 6, 7, 8, 9, 10, 11, 12, 13, 14 or 15 amino acids.

Described herein is an isolated ABP comprising a first antigen binding region (ABR) and a second ABR that each specifically bind a first target antigen, a Fab that specifically binds an additional target antigen that is distinct from the first target antigen, and an Fc domain, wherein the ABP comprises a first polypeptide, a second polypeptide, and a third polypeptide, wherein the first polypeptide comprises, in an N→C direction, the first ABR-a first hinge-CH2-CH3, wherein the second polypeptide comprises, in an N→C direction, the second ABR-a variable heavy chain (VH) domain of the Fab-a CH1 domain of the Fab-a second hinge-CH2-CH3, wherein the third polypeptide comprises, in an N→C direction, a variable light chain (VL) domain of the Fab-a CL domain of the Fab; wherein the first ABR and second ABR each comprise, in an N→C direction: (i) a VH domain-a VL domain or (ii) a VL domain-VH domain; wherein the VH domain of the first ABR is attached to the VL domain of the first ABR via a first linker; wherein the VH domain of the second ABR is attached to the VL domain of the second ABR via a second linker; and wherein the first linker and second linker are each about 5-15 amino acids in length.

It is also contemplated that the first and second linker are of different lengths (number of amino acids). See Hudson, P. J., and Kortt, A. A., Journal of immunological methods 231.1-2 (1999): 177-189, which is incorporated by reference in its entirety.

Examples of diabodies described in the art are provided in Hollinger et al., Proc. Natl. Acad. Sci. USA, 1993, 90:6444-6448; Olafsen, T. et al. Protein Eng Des Sel., 2004, 17(1):21-27; Wu, A. et al. Protein Engineering, 2001, 14(2): 1025-1033; Asano et al., 2004, Abstract 3P-683, J. Biochem. 76(8):992; Takemura, S. et al. Protein Eng., 2000, 13(8):583-588; Baeuerle, P. A. et al. Cancer Res., 2009, 69(12):4941-4944; U.S. Pat. No. 7,129,330; and International Application No. PCT/US2015/033076, each of which is incorporated by reference in its entirety.

Exemplary sequences of Format 41 diabodies are provided in Table 23. Table 23 provides the linker-Fc sequence of an exemplary first polypeptide chain, minus the first ABR sequence (SEQ ID NO: 48); the linker-Fab-Fc sequence of an exemplary second polypeptide with an OKT3, UCHT1v9 or SP34 CD3 binding fragment (SEQ ID NOs: 49, 51, 53); and the light chain of an exemplary OKT3, UCHT1v9 or SP34 CD3 binding fragment (SEQ ID NOS: 50, 52, 54). The 31E07 ABR (scFv format) is provided as SEQ ID NO: 55. In some embodiments, the first polypeptide comprises the sequence as set forth in SEQ ID NO: 55. In some embodiments, the first polypeptide comprises the sequence as set forth in SEQ ID NOs: 55 and 48. In some embodiments, the second polypeptide comprises the sequence as set forth in SEQ ID NO: 51, 49, or 53. In some embodiments, the second polypeptide comprises the sequence as set forth in SEQ ID NO: 55 and one of 51, 49, or 53. In some embodiments, the second polypeptide comprises the sequence as set forth in SEQ ID NO: 55 and 51. In some embodiments, the third polypeptide comprises the sequence as set forth in SEQ ID NO: 52, 50, or 54. In some embodiments, the isolated ABP comprising a first antigen binding region (ABR) and a second ABR that each specifically bind a first target antigen, a Fab that specifically binds an additional target antigen that is distinct from the first target antigen, and an Fc domain comprises the sequences as set forth in SEQ ID NOs: 48, 49, 50, and 55; 48, 51, 52, and 55, or 48, 53, 54, and 55. In some embodiments, the isolated ABP comprising a first antigen binding region (ABR) and a second ABR that each specifically bind a first target antigen, a Fab that specifically binds an additional target antigen that is distinct from the first target antigen, and an Fc domain comprises the sequences as set forth in SEQ ID NOs: 48, 51, 52, and 55.

Described herein is an isolated ABP comprising a first antigen binding region (ABR) and a second ABR that each specifically bind distinct target antigens, (e.g., a first target antigen and a second target antigen wherein the second target antigen is distinct from the first target antigen), a Fab that specifically binds the first target antigen, and an Fc domain, wherein the ABP comprises a first polypeptide, a second polypeptide, and a third polypeptide, wherein the first polypeptide comprises, in an N→C direction, a variable heavy chain (VH) domain or variable light chain (VL) domain of the second ABR-a VH domain or the VL domain of the first ABR-a first hinge-CH2-CH3, wherein the second polypeptide comprises, in an N→C direction, the VH domain or VL domain of the first ABR-the VH or VL of the second ABR-the VH domain of the Fab-a CH1 domain of the Fab-a second hinge-CH2-CH3, wherein the third polypeptide comprises, in an N→C direction, the VL domain of the Fab-a CL domain of the Fab; wherein the first ABR and second ABR each comprise: (i) a VH domain-a VL domain or (ii) a VL domain-VH domain; wherein the VH domain or VL domain of the first ABR is attached to the VL domain or VH domain of the second ABR via a first linker; wherein the VH domain or VL domain of the second ABR is attached to the VL domain or VH domain of the first ABR via a second linker; and wherein the first linker and second linker are each about 5-15 amino acids in length.

The VH or VL domain of the first ABR on the first polypeptide interacts with the VL or VH domain of the first ABR on the second polypeptide to form a complete ABR molecule. The VH or VL domain of the second ABR on the first polypeptide interacts with the VL or VH domain of the second ABR on the second polypeptide to form a complete ABR molecule. The first ABR binds a first target antigen and the second ABR binds a second target antigen that is distinct from the first target antigen. The Fab binds an additional target antigen that can be the first target antigen and is distinct from the second antigen target.

The VH or VL domain of the first ABR on the first polypeptide interacts with the VL or VH domain of the first ABR on the second polypeptide to form a complete ABR molecule. The VH or VL domain of the second ABR on the first polypeptide interacts with the VL or VH domain of the second ABR on the second polypeptide to form a complete ABR molecule. The first ABR binds a first target antigen and the second ABR binds a second target antigen that is distinct from the first target antigen. The Fab binds the first target antigen.

Exemplary sequences of Format 43 antibodies as described in the paragraph above are provided in Table 25. Table 25 provides the sequence of an exemplary first polypeptide chain comprising the linker-Fc (SEQ ID NO: 48); the linker-Fab-Fc sequence of an exemplary second comprising the VH domains of the E07 antigen binding fragment (SEQ ID NO: 56); the light chain of an exemplary 31E07 antibody (SEQ ID NO: 57); and the mixed ABR comprising the VH or VL of the E07 antigen binding fragment and the VL or VH of the UCHT1V9 CD3 binding fragment (SEQ ID NOs: 58 and 59). In some embodiments, the first polypeptide comprises the sequence as set forth in SEQ ID NO: 48. In some embodiments, the first polypeptide comprises the sequence as set forth in SEQ ID NO: 48 and one of 58 or 59. In some embodiments, the second polypeptide comprises the sequence as set forth in SEQ ID NO: 56. In some embodiments, the second polypeptide comprises the sequence as set forth in SEQ ID NO: 56 and one of 58 or 59. In some embodiments, the third polypeptide comprises the sequence as set forth in SEQ ID NO: 57. In some embodiments, the isolated ABP comprising a first antigen binding region (ABR) that specifically binds a first target antigen and a second ABR that specifically bind a second target antigen, a Fab that specifically binds the first target antigen, and an Fc domain comprises the sequences as set forth in SEQ ID NOs: 48, 56, 57, 58, and 59.

In some aspects, provided herein are isolated antigen binding proteins (ABP) that comprises: a first antigen binding region (ABR) the specifically binds a first target antigen and a second ABR that specifically binds a second target antigen, a Fab that specifically binds an additional target antigen that is distinct from the first target antigen, and an Fc domain, wherein the ABP comprises a first polypeptide, a second polypeptide, and a third polypeptide; wherein the first polypeptide comprises, in an N→C direction, a variable heavy chain (VH) domain or variable light chain (VL) domain of the first ABR-a VH domain or a VL domain of the second ABR-a first hinge-CH2-CH3; wherein the second polypeptide comprises, in an N→C direction, the VH domain or VL domain of the second ABR-the VH domain or VL domain of the first ABR-a VH domain of the Fab-a CH1 domain of the Fab-a second hinge-CH2-CH3, wherein the third polypeptide comprises, in an N→C direction, a VL domain of the Fab-a CL domain of the Fab; wherein the VH domain or VL domain of the first ABR of the first polypeptide is attached to the VL domain or VH domain of the second ABR of the first polypeptide via a first linker; wherein the VH domain or VL domain of the second ABR of the second polypeptide is attached to the VL domain or VH domain of the first ABR via a second linker; wherein the first linker and second linker are each about 5-15 amino acids in length; wherein the first target antigen is an HLA-PEPTIDE target comprising an HLA-restricted peptide complexed with an HLA Class I molecule, wherein the HLA Class I molecule is HLA subtype HLA-A*01:01 and the HLA-restricted peptide comprises the sequence NTDNNLAVY (SEQ ID NO: 214); and wherein the VH domains of the first ABR and Fab each comprise complementarity-determining regions (CDR) H1 (CDR-H1), CDR-H2, and CDR-H3 comprising SEQ ID NOS:18, 19, and 20, respectively and wherein the VL domain of the first ABR comprises CDR-L1, CDR-L2, and CDR-L3 comprising SEQ ID NOS:21, 22, and 23, respectively.

Described herein is an isolated ABP comprising a first antigen binding region (ABR) and a second ABR that each specifically bind distinct target antigens, (e.g., a first target antigen and a second target antigen wherein the second target antigen is distinct from the first target antigen), a Fab that specifically binds the first target antigen, and an Fc domain, wherein the ABP comprises a first polypeptide, a second polypeptide, and a third polypeptide, wherein the first polypeptide comprises, in an N→C direction, a variable heavy chain (VH) domain or variable light chain (VL) domain of the first ABR-a VH domain or the VL domain of the second ABR-a first hinge-CH2-CH3, wherein the second polypeptide comprises, in an N→C direction, the VH domain or VL domain of the second ABR-the VH domain or VL domain of the first ABR-the VH domain of the Fab-a CH1 domain of the Fab-a second hinge-CH2-CH3, wherein the third polypeptide comprises, in an N→C direction, the VL domain of the Fab-a CL domain of the Fab; wherein the first ABR and second ABR each comprise: (i) a VH domain-a VL domain or (ii) a VL domain-VH domain; wherein the VH domain or VL domain of the first ABR is attached to the VL domain or VH domain of the second ABR via a first linker; wherein the VH domain or VL domain of the second ABR is attached to the VL domain or VH domain of the first ABR via a second linker; and wherein the first linker and second linker are each about 5-15 amino acids in length.

The VH or VL domain of the first ABR on the first polypeptide interacts with the VL or VH domain of the first ABR on the second polypeptide to form a complete ABR molecule. The VH or VL domain of the second ABR on the first polypeptide interacts with the VL or VH domain of the second ABR on the second polypeptide to form a complete ABR molecule. The first ABR binds a first target antigen and the second ABR binds a second target antigen that is distinct from the first target antigen. The Fab binds an additional target antigen that can be the first target antigen and is distinct from the second antigen target.

Exemplary sequences of Format 42 antibodies as described in the paragraph above are provided in Table 24. Table 24 provides the linker-Fc sequence of an exemplary first polypeptide chain (SEQ ID NO: 48); the linker-Fab-Fc sequence of an exemplary second polypeptide with a Fab comprising the VH domains of the E07 antigen binding fragment (SEQ ID NO: 56); and the light chain of the E07 antibody (SEQ ID NO: 57). Table 24 also provides the mixed ABR sequence comprising the VH or VL of the E07 antigen binding fragment and the VL or VH of the UCHT1v9 CD3 binding fragment (SEQ ID NO: 59 and 58). In some embodiments, the first polypeptide comprises the sequence as set forth in SEQ ID NO: 48 and 59 or 58. In some embodiments, the second polypeptide comprises the sequence as set forth in SEQ ID NO: 56 and 59 or 58. In some embodiments, the third polypeptide comprises the sequence as set forth in SEQ ID NO: 58. In some embodiments, the isolated ABP comprising a first antigen binding region (ABR) that specifically binds a first target antigen and a second ABR that specifically bind a second target antigen, a Fab that specifically binds the first target antigen, and an Fc domain comprises the sequences as set forth in SEQ ID NOs: 48, 56, 57, 58, and 59.

### Linkers

Various linkers are contemplated for use in the ABPs described herein, particularly between the variable domains (variable heavy and variable light domains), between the variable regions and N-terminus of the VH domain of the Fab, and/or between the variable regions and hinge of the first polypeptide. In some embodiments, the linker is a polypeptide linker. In some embodiments, the amino acids in the polypeptide linker are selected with properties that confer flexibility and resist cleavage from proteases (e.g., glycine and serine). In some embodiments, the polypeptide linker comprises one or more glycine and/or serine residues.

In some embodiments, the linker includes one or more glycines. In some embodiments, the linker includes one or more serines. In some embodiments, the linker comprises or consists of glycines and serines. In some embodiments, the linker comprises or consists of a (GS)ₙ (SEQ ID NO: 218), (GGS)ₙ(SEQ ID NO: 219), (GGGS)ₙ (SEQ ID NO: 220), (GGSG)ₙ (SEQ ID NO: 221), (GGSGG)ₙ (SEQ ID NO: 222), and (GGGGS)ₙ (SEQ ID NO: 223) sequence, wherein n is 1, 2, 3, 4, 5, 6, 7, 8, 9, 10, 11, 12, 13, 14, 15, 16, 17, 18, 19, or 20. In some embodiments, the linker comprises or consists of a (GGGGS)ₙ (SEQ ID NO: 223) sequence, wherein n is 1, 2, 3, 4, 5, 6, 7, 8, 9, 10, 11, 12, 13, 14, 15, 16, 17, 18, 19, or 20. In some embodiments, the n values are 1, 2, 3 or 4. In some embodiments, the n values are 1, 2, or 3. Any combination of glycines and serines in the linker is contemplated. In some embodiments, the linker comprises or consists of a (GSGGG)ₙ (SEQ ID NO: 224), (GGSGG)ₙ (SEQ ID NO: 222) or (GGGSG)ₙ (SEQ ID NO: 225) sequence, wherein n is 1, 2, 3, 4, 5, 6, 7, 8, 9, 10, 11, 12, 13, 14, 15, 16, 17, 18, 19, or 20. In some embodiments, the n values are 1, 2, 3 or 4. In some embodiments, the n values are 1, 2 or 3. In some embodiments, the linker comprises or consists of a (GGGGG)ₙ (SEQ ID NO: 226) sequence, wherein n is 1, 2, 3, 4, 5, 6, 7, 8, 9, 10, 11, 12, 13, 14, 15, 16, 17, 18, 19, or 20. In some embodiments, the n values are 1, 2, 3 or 4. In some embodiments, the n values are 1, 2, or 3. In some embodiments, n is 1. In some embodiments, n is 2. In some embodiments, n is 3. In some embodiments, n is 4. In some embodiments, n is 5. In some embodiments, n is 6. In some embodiments, n is 7. In some embodiments, n is 8. In some embodiments, n is 9. In some embodiments, n is 10. In some embodiments, n is 11. In some embodiments, n is 12. In some embodiments, n is 13. In some embodiments, n is 14. In some embodiments, n is 15. In some embodiments, n is 16. In some embodiments, n is 17. In some embodiments, n is 18. In some embodiments, n is 19. In some embodiments, n is 20. In some embodiments, n is 1-5; 1-4, 1-3, 1-2, 2-5, 2-4, 2-3, 3-5, 3-4, or 4-5. In some embodiments, n is 1-3. In some embodiments, n is 2-3.

In some embodiments, the VH domain of the first ABR is attached to the VL domain of the second ABR via a first linker (“L1” in FIG. 5); wherein the VH domain of the second ABR is attached to the VL domain of the second ABR via a second linker (“L2” in FIG. 5) and the first and second linkers are polypeptide linkers. In some embodiments, the first and second linkers each consist of (GGGGS)_{N}, wherein _{N}=1-3 (SEQ ID NO: 215). In some embodiments, the first and second linkers each consist of (GGGGS)_{N}, wherein _{N}=2 (SEQ ID NO: 111).

In some embodiments, wherein the VH domain of the first ABR is attached to the VL domain of the second ABR via a first linker (“L1” in FIG. 5), wherein the VH domain of the second ABR is attached to the VL domain of the second ABR via a second linker (“L2” in FIG. 5), wherein the first and second linkers each consist of (GGGGS)_{N}, and wherein _{N}=2 (SEQ ID NO: 111), the resulting conformation is a Format 41 antibody having a diabody conformation (see FIG. 8A).

The present disclosure provides methods of producing a multispecific ABP with a diabody conformation, by creating a Format 4 antibody having shortened linkers between the VH and VL domains of the ABRs, wherein a shortened linker is a peptide linker having less than 16 amino acids (e.g., (GGGGS)_{N}, wherein _{N}=1-3 (SEQ ID NO: 215)), optionally having greater than 4 amino acids or optionally having 5-10 amino acids.

In some embodiments, the present disclosure provides methods of producing a multispecific ABP with a diabody conformation by creating a Format 41 antibody having shortened linkers between the VH and VL domains of the ABRs, wherein a shortened linker is a peptide linker having 10 amino acids. In some embodiments, the 10 amino-acid peptide linker is (GGGGS)₂ (SEQ ID NO: 111). One of ordinary skill in the art would appreciate that (GGGGS)₂ (SEQ ID NO: 111) can also be referred to as GGGGSGGGGS (SEQ ID NO: 111) or (G4S)₂ (SEQ ID NO: 111).

In some embodiments, the present disclosure provides methods of producing an ABP with a diabody conformation by creating a Format 4 antibody having shortened linkers between the VH and VL domains of the ABRs, wherein a shortened linker is a peptide linker having 10 amino acids. In some embodiments, the 10 amino-acid peptide linker is (GGGGS)₂ (SEQ ID NO: 111). One of ordinary skill in the art would appreciate that (GGGGS)₂ (SEQ ID NO: 111) can also be referred to as GGGGSGGGGS (SEQ ID NO: 111) or (G4S)₂ (SEQ ID NO: 111).

Introduction of a shortened linker, as described supra, at the first linker (L1 in FIG. 5) and second linker (L2 in FIG. 5) can shift the equilibrium of 2×scFv-conformed antibodies and diabody conformed antibody towards a higher proportion of diabody conformed antibodies. (See Examples).

Stabilizing Dual scFv Conformation with Disulfide Bonds (DSBs)

The present disclosure provides compostions of APBs internal disulfide bonds (internal DSB) that stabilize the 2×scFv conformation of Format 4 APBs. As used herein, the term “internal DSB” refers to a DSB resulting from cysteines present in an ABP, e.g., in certain variable domains of the ABP.

In some embodiments, the ABP is a Format 4 antibody, wherein the VH domain of the first ABR is attached to the VL domain of the first ABR via a first linker (L1 in FIG. 5); wherein the VH domain of the second ABR is attached to the VL domain of the second ABR via a second linker (L2 in FIG. 5); wherein the VL domain of the first ABR is attached to the hinge in the first polypeptide via a third linker (L3 in FIG. 5); wherein the VL domain of the second ABR is attached to the N-terminus of the second polypeptide or the third polypeptide via a fourth linker (L4 in FIG. 5). In some embodiments, the first linker and the second linker each comprise 20 amino acids. In some embodiments, the first linker and the second linker each comprise 14 amino acids. For example, the first and second linker can each consist of 14, 15, 16, 17, 18, 19, 20, 21, 22, 23, 24, 25, 26, 27, 28, 29 or 30 amino acids. The first and second linker can consist of (GGGGS)_{N}, wherein _{N}=3-10 (SEQ ID NO: 227). The first and second linker can consist of (GGGGS)_{N}, wherein _{N}=4 (SEQ ID NO: 228). In some embodiments, the third linker and the fourth linker each consist of 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 amino acids. In some embodiments, the third linker and the fourth linker each consist of 10 amino acids. For example, the third and fourth linker can consist of (GGGGS)_{N}, wherein _{N}=2 (SEQ ID NO: 111). In such embodiments, the ABPs exist at an equilibrium between the extended conformation of Format 4 antibody and the diabody conformation of Format 4 antibody.

In some embodiments, when the first and second linker comprise 20 amino acids (optionally comprise 14, 15, or 16 amino acids), the VH domain of the first ABR and/or second ABR further comprises a cysteine (Cys) at amino acid residue 44 of the VH domain according to the Kabat numbering system and wherein the VL domain of the first ABR and/or second ABR comprises a cysteine residue at amino acid residue 100 of the VL domain according to the Kabat numbering system (referred to as H44-L100 in reference to the VH (“H”) and VL (“L”) domains of the ABRs). By introducing Cys residues at both of these positions, a disulfide bond (DSB) forms that stabilizes the VH/VL interactions within each ABR. This reduces the probability that the two ABRs of the Format 4 antibodies interact to form the alternative diabody conformation. (See Examples). As a result, the 2×scFv conformation is “stabilized”. In some embodiments, this results in the absence of diabody-conformed ABPs or a negligible amount of diabody-conformed ABPs.

Other internal disulfide bonds are contemplated in the present ABPs. Non-limiting examples of other disulfide bridge mutations/positions are H105-L43, H110b-L49, H100-L50 and H101-L46. See Weatherill, E. E., et al., Protein Engineering, Design & Selection 25.7 (2012): 321-329, which is incorporated by reference in its entirety. Additional potential DSBs that are useful in the ABPs described herein can be calculated using computational modeling.

In some embodiments, in these ABPs with internal DSBs, the first ABR and second ABR bind to an HLA-peptide target with a dissociation constant (K_{D}) less than or equal to 100 nM, as measured by surface plasmon resonance (SPR) technology (e.g., BIACORE®), biolayer interferometry (e.g., FORTEBIO®) or other methods known in the art for measuring affinity. In some embodiments, the first ABR and second ABR bind to an HLA-peptide target with a dissociation constant (K_{D}) less than or equal to about 80 nM, about 82 nM, about 84 nM, about 86 nM, about 88 nM, about 90 nM, about 92 nM, about 94 nM, about 96 nM, about 98 nM, about 100 nM, about 102 nM, about 104 nM, about 106 nM, about 108 nM, about 110 nM, about 112 nM, about 114 nM, about 116 nM, about 118 nM, about 120 nM, as measured by surface plasmon resonance (SPR) technology (e.g., BIACORE®), biolayer interferometry (e.g., FORTEBIO®) or other methods known in the art for measuring affinity. In some embodiments, the antibody that binds to an HLA peptide target has a dissociation constant (K_{D}) of ≤1 μM, ≤100 nM, ≤10 nM, ≤1 nM, ≤0.1 nM, ≤0.01 nM, or ≤0.001 nM (e.g., 10⁻⁸M or less, e.g., from 10⁻⁸M to 10⁻¹³M, e.g., from 10⁻⁹M to 10⁻¹³ M).

In some embodiments, in these ABPs with internal DSBs, the Fab binds to a cell surface molecule on an effector cell (e.g. a CD3 target) with a dissociation constant (K_{D}) less than or equal to 500 nM, preferably 20-100 nM, as measured by surface plasmon resonance (SPR) technology (e.g., BIACORE®), biolayer interferometry (e.g., FORTEBIO®) or other methods known in the art for measuring affinity. In some embodiments, the antibody that binds to CD3 target has a dissociation constant (K_{D}) of ≤1 μM, ≤100 nM, ≤10 nM, ≤1 nM, ≤0.1 nM, ≤0.01 nM, or ≤0.001 nM (e.g., 10⁻⁸M or less, e.g., from 10⁻⁸M to 10⁻¹³M, e.g., from 10⁻⁹M to 10⁻¹³ M).

In some embodiments, the ABP binds to HLA-peptide targets (.e.g. in vitro) at a higher affinity than a reference ABP. In some embodiment, the ABP binds to HLA-peptide targets (e.g. in vitro) at a the same affinity or a lower affinity than a reference ABP. In some embodiments, the ABP binds to HLA-peptide targets (e.g. in vitro) at a the same affinity or a lower affinity than a reference ABP, but has greater product homogeneity and/or stability. Stability, as used here, refers to drug stability as known in the art (e.g., stability in terms of long term storage, serum stability, through freeze-thaw cycle stability, etc.). Stability, as used here, refers to drug stability as known in the art (e.g., stability in terms of long term storage, serum stability, through freeze-thaw cycle stability, etc.). In some embodiments, the ABP binds to a CD3 target on cells at a higher affinity than a reference ABP. In some embodiments, the ABP binds to a CD3 target on cells at a the same or a lower affinity than a reference ABP. In some embodiments, the ABP binds to a CD3 target on cells at a the same or a lower affinity than a reference ABP, but as greater product homogeneity and/or stability. Stability, as used here, refers to drug stability as known in the art (e.g., stability in terms of long term storage, serum stability, through freeze-thaw cycle stability, etc.). As used herein, the term “reference ABP” can refer to a Format 4 antibody similar to the ABP, but without the internal DSB(s). In some embodiments, the reference ABP refers to another monospecific ABP, a Format 3 antibody (as disclosed in International application No. PCT/US2020/15736, incorporated by reference in its entirety), a Format 5 antibody (as shown in FIG. 4B and as disclosed in International application No. PCT/US2020/015736, incorporated by reference in its entirety), a Format 6 antibody (as shown in FIG. 4B), a different type of bispecific or multispecific antibody.

In some embodiments, an ABP described herein having at least one internal DSB results in cytotoxicity once the ABP contacts a cell expressing a tumor antigen (e.g. HLA-peptide). In some embodiments, contacting the ABP with cancer cells results in at least about 10%, 20%, 30%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%, cytotoxicity. In some embodiments, a concentration of 0.1 nM, 1 nM, 5 nM or 10 nM of the ABP is sufficient to result in that cytotoxicity upon contacting the ABP with a cancer cell that expresses a tumor antigen (e.g., HLA-peptide) and an effector cell.

In some embodiments, an ABP described herein results in greater cytotoxicity than a reference ABP, as described supra. In some embodiments, an ABP results in similar cytotoxicity to a reference ABP or less cytotoxicity than a reference ABP, as described supra. In some embodiments, the ABP described herein results in similar cytotoxicity to a reference ABP (or less cytotoxicity), but has greater product homogeneity and/or stability. Stability, as used here, refers to drug stability as known in the art (e.g., stability in terms of long term storage, serum stability, through freeze-thaw cycle stability, etc.).

Non-limiting examples of cancer cells that express tumor antigen (e.g. HLA-peptide) include A375 cells and LN229 cells.

Stabilizing Diabodies with Disulfide Bonds and Shortened Linkers

As described supra, the introduction of shortened linkers at the first linker of the first ABR and second linker of the second ABR of the Format 4 antibodies drives diabody formation. In addition, the introduction of an internal DSB can stabilize diabody formation. This benefit of combining the shortened linker with internal DSBs is most notable when the ABP type “breathes” and is prone to fall apart under non-reduced denaturing conditions during proteolytic digestion. As used, the term “breathing” refers to large-scale movements of secondary structures, subunits or domains or the rapid association and disassociation of antibody domains. The introduction of DSBs in the protein can reduces this breathing. (See Makowski L, et al., J Mol Biol. 2008; 375(2):529-546, which is incorporated herein by reference in its entirety).

In some embodiments, where the ABP comprises a first linker and a second linker that are each a shortened linker, as described supra, the VH domain of the first ABR and/or second ABR further comprises a cysteine (Cys) at amino acid residue 44 of the VH domain according to the Kabat numbering system and wherein the VL domain of the first ABR and/or second ABR comprises a cysteine residue at amino acid residue 100 of the VL domain according to the Kabat numbering system. The cysteine mutations that form this disulfide bond are herein referred to as “H44/L100,” “VH44/VL100,” “DSB44/100,” “H44-L100” or any other term known in the art to describe that mutation. By introducing Cys residues at both of these positions, a disulfide bond (DSB) forms that stabilizes the diabody conformation. This reduces the probability that the complexes or fragments will dissociate under non-reducing denaturing conditions during proteolytic digestion (See Examples). As a result, the diabody conformation is “stabilized”. In some embodiments, this results in the absence of 2×scFv-conformed ABPs or a negligible amount of 2×scFv-conformed ABPs.

In some embodiments, in these ABPs that combine the shortened first and second linkers with internal DSBs, the first ABR and second ABR bind to an HLA-peptide target with a dissociation constant (K_{D}) less than or equal to 100 nM, as measured by surface plasmon resonance (SPR) technology (e.g., BIACORE®), biolayer interferometry (e.g., FORTEBIO®) or other methods known in the art for measuring affinity. In some embodiments, the first ABR and second ABR bind to an HLA-peptide target with a dissociation constant (K_{D}) less than or equal to about 80 nM, about 82 nM, about 84 nM, about 86 nM, about 88 nM, about 90 nM, about 92 nM, about 94 nM, about 96 nM, about 98 nM, about 100 nM, about 102 nM, about 104 nM, about 106 nM, about 108 nM, about 110 nM, about 112 nM, about 114 nM, about 116 nM, about 118 nM, about 120 nM, as measured by surface plasmon resonance (SPR) technology (e.g., BIACORE®), biolayer interferometry (e.g., FORTEBIO®) or other methods known in the art for measuring affinity. In some embodiments, the antibody that binds to an HLA peptide target has a dissociation constant (K_{D}) of ≤1 μM, ≤100 nM, ≤10 nM, ≤1 nM, ≤0.1 nM, ≤0.01 nM, or ≤0.001 nM (e.g., 10⁻⁸M or less, e.g., from 10⁻⁸M to 10⁻¹³M, e.g., from 10⁻⁹M to 10⁻¹³ M).

In some embodiments, in these ABPs that combine the shortened first and second linkers with internal DSBs, the Fab binds to CD3 target with a dissociation constant (K_{D}) less than or equal to 500 nM, preferably 20-100 nM, as measured by surface plasmon resonance (SPR) technology (e.g., BIACORE®), biolayer interferometry (e.g., FORTEBIO®) or other methods known in the art for measuring affinity. In some embodiments, the antibody that binds to CD3 target has a dissociation constant (K_{D}) of ≤1 μM, ≤100 nM, ≤10 nM, ≤1 nM, ≤0.1 nM, ≤0.01 nM, or ≤0.001 nM (e.g., 10⁻⁸M or less, e.g., from 10⁻⁸M to 10⁻¹³M, e.g., from 10⁻⁹M to 10⁻¹³ M).

In some embodiments, the ABP binds to HLA-peptide targets (.e.g. in vitro) at a higher affinity than a reference ABP. In some embodiment, the ABP binds to HLA-peptide targets (e.g. in vitro) at a the same affinity or a lower affinity than a reference ABP. In some embodiments, the ABP binds to HLA-peptide targets (e.g. in vitro) at a the same affinity or a lower affinity than a reference ABP, but has greater product homogeneity and/or stability. Stability, as used here, refers to drug stability as known in the art (e.g., stability in terms of long term storage, serum stability, through freeze-thaw cycle stability, etc.). In some embodiments, the ABP binds to a CD3 target on cells at a higher affinity than a reference ABP. In some embodiments, the ABP binds to a CD3 target on cells at a the same or a lower affinity than a reference ABP. In some embodiments, the ABP binds to a CD3 target on cells at a the same or a lower affinity than a reference ABP, but as greater product homogeneity and/or stability. Stability, as used here, refers to drug stability as known in the art (e.g., stability in terms of long term storage, serum stability, through freeze-thaw cycle stability, etc.). As used herein, the term “reference ABP” can refer to a Format 4 antibody similar to the ABP, but without the internal DSB(s) and/or the shortened first and second linkers. In some embodiments, the reference ABP refers to another monospecific ABP, a Format 3 antibody (as disclosed in International application No. PCT/US2020/15736, incorporated by reference in its entirety), a Format 5 antibody (as shown in FIG. 4B and as disclosed in International application No. PCT/US2020/15736, incorporated by reference in its entirety), a Format 6 antibody (as shown in FIG. 4B), a different type of bispecific or multispecific antibody.

In some embodiments, the ABP described herein has similar affinity to CD3 and/or pHLA to a reference ABP, but has greater product homogeneity and/or stability. Stability, as used here, refers to drug stability as known in the art (e.g., stability in terms of long term storage, serum stability, through freeze-thaw cycle stability, etc.).

In some embodiments, an ABP described herein having shortened first and second linkers and internal DSBs results in cytotoxicity once the ABP contacts a cell expressing a tumor antigen (e.g. HLA-peptide) and an effector cell. In some embodiments, contacting the ABP with cancer cells results in at least about 10%, 20%, 30%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%, cytotoxicity. In some embodiments, a concentration of 0.1 nM, 1 nM, 5 nM or 10 nM of the ABP is sufficient to result in that cytotoxicity upon contacting the ABP with the a cancer cell that expresses a tumor antigen (e.g., HLA-peptide) and an effector cell.

In some embodiments, an ABP described herein results in greater cytotoxicity than a reference ABP, as described supra. In some embodiments, an ABP results in similar cytotoxicity to a reference ABP or less cytotoxicity than a reference ABP, as described supra. In some embodiments, the ABP described herein results in similar cytotoxicity to a reference ABP (or less cytotoxicity), but has greater product homogeneity and/or stability. Stability, as used here, refers to drug stability as known in the art (e.g., stability in terms of long term storage, serum stability, through freeze-thaw cycle stability, etc.).

Non-limiting examples of cancer cells that express tumor antigen (e.g. HLA-peptide) include A375 cells and LN229 cells.

### External DSBs

It is also contemplated that the ABPs described supra comprise an external DSB in place of or in addition to the internal DSBs. In some embodiments, this external DSB confers the same benefits as the internal DSBs (e.g. homogeneity, stability, affinity to target antigens, cytotoxicity, etc.). As used, the term “external DSB” refers to DSBs that result from Cys residues outside of the ABRs, for example in linkers 3 and 4, in the Fab region or in the Fc region of the ABPs. Non-limiting examples of cysteine mutations that result in external DSBs in the ABPs described herein are provided in Table 17 and 18 (see Examples).

In some embodiments, the introduction of external DSBs stabilizes a Format 4 ABP, as described herein, in either the 2×scFv conformation or the diabody conformation. In some embodiments, the introduction of an external DSB increases the affinity of the ABP comprising that external DSB to a target antigen (e.g. pHLA, CD3) relative to a reference ABP. In some embodiments, the affinity of the ABP with the external DSB(s) is similar to that of the reference ABP, but the ABP having the external DSB(s) exhibits greater product homogeneity and/or stability. Stability, as used here, refers to drug stability as known in the art (e.g., stability in terms of long term storage, serum stability, through freeze-thaw cycle stability, etc.).

In some embodiments, the introduction of an external DSB in an ABP increases the cytotoxicity of the ABP relative to a reference ABP. For example, when the ABP having an external DSB contacts a cancer cell or a virally-infected cell, in the presence of an effector cell, the contacting results in greater cytotoxicity than a reference ABP. In some embodiments, the cytotoxicity of the ABP with the external DSB(s) is similar or lower compared to that of the reference ABP, but the ABP having the external DSB(s) exhibits greater product homogeneity and/or stability. Stability, as used here, refers to drug stability as known in the art (e.g., stability in terms of long term storage, serum stability, through freeze-thaw cycle stability, etc.).

As used herein, the term “reference ABP” refers to a Format 4 ABP lacking an external DSB or having a different number of external DSBs than the ABP claimed or as disclosed herein. In some embodiments, the reference ABP refers to another monospecific ABP, a Format 3 antibody (see International application No. PCT/US2020/15736, incorporated by reference in its entirety), a Format 5 antibody (see FIG. 4B and as disclosed in International application No. PCT/US2020/15736, incorporated by reference in its entirety), a Format 6 antibody (as shown in FIG. 4B), a different type of bispecific or multispecific antibody.

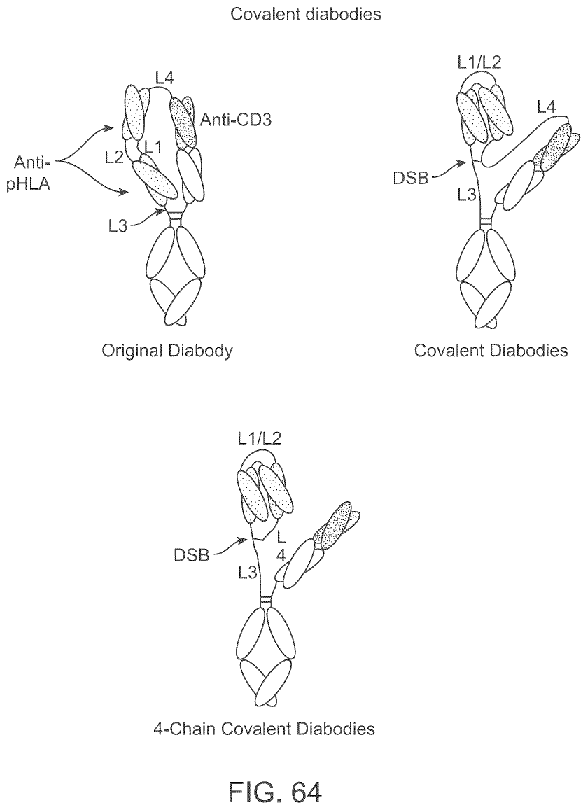

In some embodiments, the ABP having an external DSB is a covalent diabody or 4-chain covalent diabody (e.g., as shown in FIG. 64; see Examples).

### Cluster of Differentiation 3 (CD3)

In some embodiments, the present disclosure provides an antigen binding protein (ABPs) comprising variable regions (e.g., in the Fab region) that bind to a cell surface molecule present on T cells, for example, cluster of differentiation 3 (CD3). CD3 is a protein complex and T cell co-receptor that is involved in activating both the cytotoxic T cell (CD8+ naive T cells) and T helper cells (CD4+ naive T cells). As used herein, the term “cluster of differentiation 3” or “CD3” refers to any native CD3 from any vertebrate source, including mammals such as primates (e.g. humans) and rodents (e.g., mice and rats), unless otherwise indicated, including, for example, CD3ε, CD3γ, CD3α, and CD3β chains. The term encompasses “full-length,” unprocessed CD3 (e.g., unprocessed or unmodified CD3ε or CD3γ), as well as any form of CD3 that results from processing in the cell. The term also encompasses naturally occurring variants of CD3, including, for example, splice variants or allelic variants. CD3 includes, for example, human CD3ε protein (NCBI RefSeq No: NP_000724), which is 207 amino acids in length, and human CD3γ protein (NCBI RefSeq No: NP_000064), which is 182 amino acids in length.

### HLA-Peptide ABPs

Provided herein are ABPs, e.g., ABPs that specifically bind to an HLA-PEPTIDE target, wherein the HLA-PEPTIDE target comprises an HLA-restricted peptide complexed with an HLA Class I molecule, wherein the HLA-restricted peptide is located in the peptide binding groove of an α1/α2 heterodimer portion of the HLA Class I molecule, and wherein the HLA-PEPTIDE target corresponds to tumor-specific gene product KKLC-1.

In some embodiments of the HLA-PEPTIDE target, the HLA Class I molecule is HLA-A*01:01 and the HLA-restricted peptide comprises the sequence NTDNNLAVY (SEQ ID NO: 214). In some embodiments, the HLA Class I molecule is HLA-A*01:01 and the HLA-restricted peptide consists essentially of the sequence NTDNNLAVY (SEQ ID NO: 214). In some embodiments, the HLA Class I molecule is HLA-A*01:01 and the HLA-restricted peptide consists of the sequence NTDNNLAVY (SEQ ID NO: 214).

In some embodiments, the ABP is an ABP that selectively binds HLA-PEPTIDE target A*01:01_NTDNNLAVY (SEQ ID NO: 214). HLA-PEPTIDE target A*01:01_NTDNNLAVY (SEQ ID NO: 214), also referred to herein as “G2”, refers to an HLA-PEPTIDE target comprising the HLA-restricted peptide NTDNNLAVY (SEQ ID NO: 214) complexed with the HLA Class I molecule A*01:01, wherein the HLA-restricted peptide is located in the peptide binding groove of an α1/α2 heterodimer portion of the HLA Class I molecule. In some embodiments, the restricted peptide is from tumor-specific gene product KKLC-1.

The HLA-PEPTIDE target may be expressed on the surface of any suitable target cell including a tumor cell.

In some embodiments, the ABP does not bind HLA class I in the absence of the HLA-restricted peptide. In some embodiments, the ABP does not bind the HLA-restricted peptide in the absence of human MHC class I. In some embodiments, the ABP binds tumor cells presenting human MHC class I being complexed with the HLA-restricted peptide. In some embodiments, the HLA restricted peptide is a tumor antigen characterizing the cancer.

An ABP can bind to each portion of an HLA-PEPTIDE complex (i.e., HLA and peptide representing each portion of the complex), which when bound together form a novel target and protein surface for interaction with and binding by the ABP, distinct from a surface presented by the peptide alone or HLA subtype alone. Generally the novel target and protein surface formed by binding of HLA to peptide does not exist in the absence of each portion of the HLA-PEPTIDE complex.

In some embodiments, an ABP specific for HLA-PEPTIDE target A*01:01_NTDNNLAVY (SEQ ID NO: 214) (G2) selectively binds G2 with greater affinity as compared to an off-target HLA-PEPTIDE complex. The off-target HLA-PEPTIDE complex may comprise an off-target restricted peptide complexed with an HLA Class I molecule, wherein the HLA-restricted peptide is located in the peptide binding groove of an α1/α2 heterodimer portion of the HLA Class I molecule.

In some embodiments, the HLA Class I molecule of the off-target HLA-PEPTIDE is HLA subtype A*01:01.

In some embodiments, the restricted peptide of the off-target HLA-PEPTIDE comprises a sequence that has no more than 5 amino acid mismatches from the G2 target restricted peptide NTDNNLAVY (SEQ ID NO: 214).

In some embodiments, the restricted peptide of the off-target HLA-PEPTIDE is 5-14 amino acids in length. In some embodiments, the restricted peptide of the off-target HLA-PEPTIDE is 7-12 amino acids in length. In some embodiments, the restricted peptide of the off-target HLA-PEPTIDE is 8-10 amino acids in length. In some embodiments, the restricted peptide of the off-target HLA-PEPTIDE is 9 amino acids in length.

In some embodiments, the restricted peptide of the off-target HLA-PEPTIDE is expressed in normal human tissue as indicated by the public GTEx database.

In some embodiments, the restricted peptide of the off-target HLA-PEPTIDE is derived from the gene product PTS, DSG3, DSG4, KDM7A, or ICE1. In particular embodiments, the restricted peptide of the off-target HLA-PEPTIDE is derived from the gene product PTS.

In some embodiments, the restricted peptide of the off-target HLA-PEPTIDE comprises the sequence ETDNNIVVY (SEQ ID NO: 204), YTDNWLAVY (SEQ ID NO: 152), GTDNWLAQY (SEQ ID NO: 203), PTDENLARY (SEQ ID NO: 205), or NTDNLLTEY (SEQ ID NO: 206). In some embodiments, the restricted peptide of the off-target HLA-PEPTIDE consists essentially of the sequence ETDNNIVVY (SEQ ID NO: 204), YTDNWLAVY (SEQ ID NO: 152), GTDNWLAQY (SEQ ID NO: 203), PTDENLARY (SEQ ID NO: 205), or NTDNLLTEY (SEQ ID NO: 206). In some embodiments, the restricted peptide of the off-target HLA-PEPTIDE consists of the sequence ETDNNIVVY (SEQ ID NO: 204), YTDNWLAVY (SEQ ID NO: 152), GTDNWLAQY (SEQ ID NO: 203), PTDENLARY (SEQ ID NO: 205), or NTDNLLTEY (SEQ ID NO: 206).

In some embodiments, the off-target HLA-PEPTIDE is HLA-A*01:01_ETDNNIVVY (SEQ ID NO: 204), HLA-A*01:01_YTDNWLAVY (SEQ ID NO: 152), HLA-A*01:01_GTDNWLAQY (SEQ ID NO: 203), HLA-A*01:01_PTDENLARY (SEQ ID NO: 205), or HLA-A*01:01_NTDNLLTEY (SEQ ID NO: 206).

In some embodiments, the ABP binds to the HLA-PEPTIDE target with more than 10-fold, 20-fold, 50-fold stronger affinity as compared to the off-target HLA-PEPTIDE. In some embodiments, the ABP binds to the HLA-PEPTIDE target with 100×-10,000× stronger affinity as compared to the off-target HLA-PEPTIDE.

In particular embodiments, the ABP binds to the HLA-PEPTIDE target with more than 10-fold, 20-fold, 50-fold stronger affinity as compared to the off-target HLA-PEPTIDE A*01:01_ETDNNIVVY (SEQ ID NO: 204). In particular embodiments, the ABP binds to the HLA-PEPTIDE target with 100×-10,000× stronger affinity as compared to the off-target HLA-PEPTIDE A*01:01_ETDNNIVVY (SEQ ID NO: 204).

In some embodiments, the ABP exhibits little or weak binding to the off-target HLA-PEPTIDE. For example, in some embodiments, the ABP binds to the off-target HLA-PEPTIDE with a Kd that is at least 1 μM or higher, 5 μM or higher, 10 μM or higher, 20 μM or higher, 50 μM or higher, 100 μM or higher, or 1000 μM or higher.

In particular embodiments, the ABP binds to the off-target HLA-PEPTIDE A*01:01_ETDNNIVVY (SEQ ID NO: 204) with a Kd that is at least 1 μM or higher, 5 μM or higher, 10 μM or higher, 20 μM or higher, 50 μM or higher, 100 μM or higher, or 1000 μM or higher.

In some embodiments, the ABP does not exhibit detectable binding to the off-target HLA-PEPTIDE. In some embodiments, the ABP does not bind to the off-target HLA-PEPTIDE.

In some embodiments, the ABP does not exhibit detectable binding to the off-target HLA-PEPTIDE A*01:01_ETDNNIVVY (SEQ ID NO: 204). In some embodiments, the ABP does not bind to the off-target HLA-PEPTIDE A*01:01_ETDNNIVVY (SEQ ID NO: 204).

The ABP can be capable of specifically binding a complex comprising the HLA-PEPTIDE target, e.g., derived from a tumor. In some embodiments, the ABP does not bind HLA in an absence of the HLA-restricted peptide derived from the tumor. In some embodiments, the ABP does not bind the HLA-restricted peptide derived from the tumor in an absence of HLA. In some embodiments, the ABP binds a complex comprising HLA and HLA-restricted peptide when naturally presented on a cell such as a tumor cell.

In some embodiments, an ABP provided herein modulates binding of the HLA-PEPTIDE to one or more ligands of the HLA-PEPTIDE.

Also provided herein is an ABP is an ABP that competes with an illustrative ABP disclosed herein. In some aspects, the ABP that competes with the illustrative ABP provided herein binds the same epitope as an illustrative ABP provided herein.

In some aspects, provided herein are ABPs referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining ABPs. In some embodiments, a variant is derived from any of the sequences provided herein, wherein one or more conservative amino acid substitutions are made. In some embodiments, a variant is derived from any of the sequences provided herein, wherein one or more nonconservative amino acid substitutions are made. Conservative amino acid substitutions are described herein. Exemplary nonconservative amino acid substitutions include those described in J Immunol. 2008 May 1; 180(9):6116-31, which is hereby incorporated by reference in its entirety. In preferred embodiments, the non-conservative amino acid substitution does not interfere with or inhibit the biological activity of the functional variant. In yet more preferred embodiments, the non-conservative amino acid substitution enhances the biological activity of the functional variant, such that the biological activity of the functional variant is increased as compared to the parent ABP.

In some embodiments, either the first ABR or the second ABR binds to an additional target antigen that is not an HLA-Peptide target. In some embodiments, either the first ABR and the second ABR binds to an additional target antigen.

The first ABR, second ABR, and Fab can be referred to as three different binders. In some of such embodiments, one of the binders binds to CD3. In some embodiments, the other two binders bind to an HLA-PEPTIDE target (different or the same). In alternate embodiments, one binder binds to CD3, a second binder binds to an HLA-peptide target, and a third binder binds to a receptor on a effector cells (e.g., T cell) that is not CD3, for example, CD28. In some embodiments, where two binders bind to a receptor on an effector cell (e.g., T cell), one but not both binders will bind CD3. Without being bound by theory or mechanism, this is to avoid overstimulation of the T cells.

### Sequences of Exemplary ABPs

The ABP specific for A*01:01_NTDNNLAVY (SEQ ID NO: 214) may comprise one or more sequences, as described in further detail below.

### CDRs

The ABP specific for A*01:01_NTDNNLAVY (SEQ ID NO: 214) may comprise one or more antibody complementarity determining region (CDR) sequences, e.g., may comprise three heavy chain CDRs (HCDR1, HCDR2, HCDR3) and three light chain CDRs (LCDR1, LCDR2, LCDR3). For example, the ABP specific for A*01:01_NTDNNLAVY (SEQ ID NO: 214) may comprise one or more antibody complementarity determining region (CDR) sequences from the clone designated C11, D5, or E07 (Table 20).

The ABP specific for A*01:01_NTDNNLAVY (SEQ ID NO: 214) may comprise a particular HCDR3 sequence. In some embodiments, the ABP comprises the HCDR3 from the clone designated C11, D5, or E07 (Table 20).

The ABP specific for A*01:01_NTDNNLAVY (SEQ ID NO: 214) may comprise a particular light chain CDR3 sequence. The LCDR3 sequence may be selected from the clone designated C11, D5, or E07 (Table 20).

The ABP specific for A*01:01_NTDNNLAVY (SEQ ID NO: 214) may comprise a particular heavy chain CDR3 (HCDR3) sequence and a particular light chain CDR3 (LCDR3) sequence. In some embodiments, the ABP comprises the HCDR3 and the LCDR3 from the clone designated C11, D5, or E07 (Table 20).

The ABP specific for A*01:01_NTDNNLAVY (SEQ ID NO: 214) may comprise all six CDRs from the clone designated C11, D5, or E07 (Table 20).

In some embodiments, an ABP provided herein comprises a CDR-H3 of SEQ ID NO: 20, a CDR-H2 of SEQ ID NO: 19, a CDR-H1 of SEQ ID NO: 18, a CDR-L3 of SEQ ID NO: 23, a CDR-L2 of SEQ ID NO: 22, and a CDR-L1 of SEQ ID NO: 21. In some embodiments, an ABP provided herein comprises a CDR-H3 of SEQ ID NO: 9, a CDR-H2 of SEQ ID NO: 8, a CDR-H1 of SEQ ID NO: 7, a CDR-L3 of SEQ ID NO: 11, a CDR-L2 of SEQ ID NO: 10, and a CDR-L1 of SEQ ID NO: 21. In some embodiments, an ABP provided herein comprises a CDR-H3 of SEQ ID NO: 14, a CDR-H2 of SEQ ID NO: 13, a CDR-H1 of SEQ ID NO: 12, a CDR-L3 of SEQ ID NO: 23, a CDR-L2 of SEQ ID NO: 22, and a CDR-L1 of SEQ ID NO: 21. In some embodiments, an ABP provided herein comprises a CDR-H3 of SEQ ID NO: 17, a CDR-H2 of SEQ ID NO: 16, a CDR-H1 of SEQ ID NO: 15, a CDR-L3 of SEQ ID NO: 11, a CDR-L2 of SEQ ID NO: 10, and a CDR-L1 of SEQ ID NO: 21. In some embodiments, the CDR-H3 has at least about 50%, 75%, 80%, 85%, 90%, or 95% identity with a CDR-H3 of SEQ ID NO: 20, 17, 14, or 9, the CDR-H2 has at least about 50%, 75%, 80%, 85%, 90%, or 95% identity with a CDR-H2 of SEQ ID NO: 19, 16, 13, or 8, the CDR-H1 has at least about 50%, 75%, 80%, 85%, 90%, or 95% identity with a CDR-H1 of SEQ ID NO: 18, 15, 12, or 7, the CDR-L3 has at least about 50%, 75%, 80%, 85%, 90%, or 95% identity with a CDR-L3 of SEQ ID NO: 23 or 11, the CDR-L2 has at least about 50%, 75%, 80%, 85%, 90%, or 95% identity with a CDR-L2 of SEQ ID NO: 22 or 10, and the CDR-L1 has at least about 50%, 75%, 80%, 85%, 90%, or 95% identity with a CDR-L1 of SEQ ID NO: 21. In some embodiments, the CDR-H3 is a CDR-H3 of SEQ ID NO: 20, 17, 14, or 9, with up to 1, 2, 3, 4, 5, 6, 7, or 8 amino acid substitutions; the CDR-H2 is a CDR-H2 of SEQ ID NO: 19, 16, 13, or 8, with up to 1, 2, 3, 4, 5, 6, 7, or 8 amino acid substitutions; the CDR-H1 is a CDR-H1 of SEQ ID NO: 18, 15, 12, or 7, with up to 1, 2, 3, 4, or 5 amino acid substitutions; the CDR-L3 is a CDR-L3 of SEQ ID NO: 23 or 11, with up to 1, 2, 3, 4, or 5 amino acid substitutions; the CDR-L2 is a CDR-L2 of SEQ ID NO: 22 or 10, with up to 1, 2, 3, or 4 amino acid substitutions; and the CDR-L1 is a CDR-L1 of SEQ ID NO: 21 with up to 1, 2, 3, 4, 5, or 6 amino acid substitutions.

The CD3 antibody or antigen binding fragment may comprise three heavy chain CDRs (HCDR1, HCDR2, HCDR3) and three light chain CDRs (LCDR1, LCDR2, LCDR3). For example, the CD3 antibody or antigen binding fragment may comprise one or more antibody complementarity determining region (CDR) sequences from the clone designated OKT3, UCHT1v9 or SP34 (Table 21).

In some embodiments, a CD3 antibody or antigen binding fragment provided herein comprises a CDR-H3 of SEQ ID NO: 38, a CDR-H2 of SEQ ID NO: 37, a CDR-H1 of SEQ ID NO: 36, a CDR-L3 of SEQ ID NO: 41, a CDR-L2 of SEQ ID NO: 40, and a CDR-L1 of SEQ ID NO: 39. In some embodiments, a CD3 antibody or antigen binding fragment provided herein comprises a CDR-H3 of SEQ ID NO: 44, a CDR-H2 of SEQ ID NO: 43, a CDR-H1 of SEQ ID NO: 42, a CDR-L3 of SEQ ID NO: 47, a CDR-L2 of SEQ ID NO: 46, and a CDR-L1 of SEQ ID NO: 45. In some embodiments, a CD3 antibody or antigen binding fragment provided herein comprises a CDR-H3 of SEQ ID NO: 28, a CDR-H2 of SEQ ID NO: 27, a CDR-H1 of SEQ ID NO: 26, a CDR-L3 of SEQ ID NO: 31, a CDR-L2 of SEQ ID NO: 30, and a CDR-L1 of SEQ ID NO: 29. In some embodiments, the CDR-H3 has at least about 50%, 75%, 80%, 85%, 90%, or 95% identity with a CDR-H3 of SEQ ID NO: 38, 44, or 28, the CDR-H2 has at least about 50%, 75%, 80%, 85%, 90%, or 95% identity with a CDR-H2 of SEQ ID NO: 37, 43, or 27, the CDR-H1 has at least about 50%, 75%, 80%, 85%, 90%, or 95% identity with a CDR-H1 of SEQ ID NO: 36, 42, or 26, the CDR-L3 has at least about 50%, 75%, 80%, 85%, 90%, or 95% identity with a CDR-L3 of SEQ ID NO: 41, 47, or 31, the CDR-L2 has at least about 50%, 75%, 80%, 85%, 90%, or 95% identity with a CDR-L2 of SEQ ID NO: 40, 46, or 30, and the CDR-L1 has at least about 50%, 75%, 80%, 85%, 90%, or 95% identity with a CDR-L1 of SEQ ID NO: 39, 45, or 29. In some embodiments, the CDR-H3 is a CDR-H3 of SEQ ID NO: 38, 44, or 28, with up to 1, 2, 3, 4, 5, 6, 7, or 8 amino acid substitutions; the CDR-H2 is a CDR-H2 of SEQ ID NO: 37, 43, or 27, with up to 1, 2, 3, 4, 5, 6, 7, or 8 amino acid substitutions; the CDR-H1 is a CDR-H1 of SEQ ID NO: 36, 42, or 26, with up to 1, 2, 3, 4, or 5 amino acid substitutions; the CDR-L3 is a CDR-L3 of SEQ ID NO: 41, 47, or 31, with up to 1, 2, 3, 4, or 5 amino acid substitutions; the CDR-L2 is a CDR-L2 of SEQ ID NO: 40, 46, or 30, with up to 1, 2, 3, or 4 amino acid substitutions; and the CDR-L1 is a CDR-L1 of SEQ ID NO: 39, 45, or 29 with up to 1, 2, 3, 4, 5, or 6 amino acid substitutions.

### VH and VL

The ABP specific for A*01:01_NTDNNLAVY (SEQ ID NO: 214) may comprise a particular VL sequence. The VL sequence may be from the clone designated C11, D5, or E07 (Table 19).

The ABP specific for A*01:01_NTDNNLAVY (SEQ ID NO: 214) may comprise a VH sequence. The VH sequence may be from the clone designated C11, D5, or E07 (Table 19).

In some embodiments, an ABP provided herein comprises a VH sequence selected from SEQ ID NOs: 6, 1, 3, or 5. In some embodiments, an ABP provided herein comprises a VH sequence of SEQ ID NO: 6. In some embodiments, an ABP provided herein comprises a VH sequence of SEQ ID NO: 1. In some embodiments, an ABP provided herein comprises a VH sequence of SEQ ID NO: 3. In some embodiments, an ABP provided herein comprises a VH sequence of SEQ ID NO: 5.

In some embodiments, the VH sequence has at least 70%, 80%, or 90% identity with SEQ ID NO: 1, wherein any variation from SEQ ID NO: 1 does not occur within CDR-H1, CDR-H2, or CDR-H3. In some embodiments, the VH sequence has at least 70%, 80%, or 90% identity with SEQ ID NO: 6, wherein any variation from SEQ ID NO: 6 does not occur within CDR-H1, CDR-H2, or CDR-H3. In some embodiments, the VH sequence has at least 70%, 80%, or 90% identity with SEQ ID NO: 3, wherein any variation from SEQ ID NO: 3 does not occur within CDR-H1, CDR-H2, or CDR-H3. In some embodiments, the VH sequence has at least 70%, 80%, or 90% identity with SEQ ID NO: 5, wherein any variation from SEQ ID NO: 5 does not occur within CDR-H1, CDR-H2, or CDR-H3.

In some embodiments, an antibody provided herein comprises a VH sequence having at least about 50%, 60%, 70%, 80%, 90%, 95%, or 99% identity to SEQ ID NO: 1. In some embodiments, an antibody provided herein comprises a VH sequence having at least about 50%, 60%, 70%, 80%, 90%, 95%, or 99% identity to SEQ ID NO: 3. In some embodiments, an antibody provided herein comprises a VH sequence having at least about 50%, 60%, 70%, 80%, 90%, 95%, or 99% identity to SEQ ID NO: 6. In some embodiments, an antibody provided herein comprises a VH sequence having at least about 50%, 60%, 70%, 80%, 90%, 95%, or 99% identity to SEQ ID NO: 5.

In some embodiments, an antibody provided herein comprises a VH sequence provided in SEQ ID NOs: 1, 3, 5, or 6, with up to 1, 2, 3, 4, 5, 6, 7, 8, 9, 10, 11, 12, 13, 14, 15, 16, 17, 18, 19, 20, 21, 22, 23, 24, or 25 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In some embodiments, an ABP thereof provided herein comprises a VL sequence selected from SEQ ID NOs: 4 or 2. In some embodiments, an ABP provided herein comprises a VL sequence of SEQ ID NO: 4. In some embodiments, an antibody provided herein comprises a VL sequence of SEQ ID NO: 2.

In some embodiments, the VL sequence has at least 70%, 80%, or 90% identity with SEQ ID NO: 4, wherein any variation from SEQ ID NO: 4 does not occur within CDR-L1, CDR-L2, or CDR-L3. In some embodiments, the VL sequence has at least 70%, 80%, or 90% identity with SEQ ID NO: 2, wherein any variation from SEQ ID NO: 2 does not occur within CDR-L1, CDR-L2, or CDR-L3.

In some embodiments, an ABP provided herein comprises a VL sequence having at least about 50%, 60%, 70%, 80%, 90%, 95%, or 99% identity to SEQ ID NO: 4. In some embodiments, an antibody provided herein comprises a VL sequence having at least about 50%, 60%, 70%, 80%, 90%, 95%, or 99% identity to SEQ ID NO: 2.

In some embodiments, an ABP provided herein comprises a VL sequence provided in SEQ ID NOs: 4 or 2, with up to 1, 2, 3, 4, 5, 6, 7, 8, 9, 10, 11, 12, 13, 14, 15, 16, 17, 18, 19, 20, 21, 22, 23, 24, or 25 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In some embodiments, a CD3 antibody or antigen binding fragment provided herein comprises a VH sequence selected from SEQ ID NOs: 32, 24, or 34. In some embodiments, a CD3 antibody or antigen binding fragment provided herein comprises a VH sequence of SEQ ID NO: 32. In some embodiments, a CD3 antibody or antigen binding fragment provided herein comprises a VH sequence of SEQ ID NO: 24. In some embodiments, a CD3 antibody or antigen binding fragment provided herein comprises a VH sequence of SEQ ID NO: 34.

In some embodiments, the CD3 antibody or antigen binding fragment VH sequence has at least 70%, 80%, or 90% identity with SEQ ID NO: 32, 24, or 34, wherein any variation from SEQ ID NO: 32, 24, or 34 does not occur within CDR-H1, CDR-H2, or CDR-H3.

In some embodiments, a CD3 antibody or antigen binding fragment provided herein comprises a VH sequence having at least about 50%, 60%, 70%, 80%, 90%, 95%, or 99% identity to SEQ ID NO: 32, 24, or 34.

In some embodiments, a CD3 antibody or antigen binding fragment provided herein comprises a VH sequence provided in SEQ ID NOs: 32, 24, or 34, with up to 1, 2, 3, 4, 5, 6, 7, 8, 9, 10, 11, 12, 13, 14, 15, 16, 17, 18, 19, 20, 21, 22, 23, 24, or 25 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In some embodiments, a CD3 antibody or antigen binding fragment thereof provided herein comprises a VL sequence selected from SEQ ID NOs: 33, 25, or 35. In some embodiments, an ABP provided herein comprises a VL sequence of SEQ ID NO: 33.

In some embodiments, the CD3 antibody or antigen binding fragment VL sequence has at least 70%, 80%, or 90% identity with SEQ ID NO: 33, wherein any variation from SEQ ID NO: 33 does not occur within CDR-L1, CDR-L2, or CDR-L3.

In some embodiments, a CD3 antibody or antigen binding fragment provided herein comprises a VL sequence having at least about 50%, 60%, 70%, 80%, 90%, 95%, or 99% identity to SEQ ID NO: 33, 25, or 35.

In some embodiments, a CD3 antibody or antigen binding fragment provided herein comprises a VL sequence provided in SEQ ID NOs: 33, 25, or 35, with up to 1, 2, 3, 4, 5, 6, 7, 8, 9, 10, 11, 12, 13, 14, 15, 16, 17, 18, 19, 20, 21, 22, 23, 24, or 25 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

### VH-VL Combinations

The ABP specific for A*01:01_NTDNNLAVY (SEQ ID NO: 214) may comprise a particular VH sequence and a particular VL sequence. In some embodiments, the ABP specific for A*01:01_NTDNNLAVY (SEQ ID NO: 214) comprises the VH sequence and the VL sequence from the clone designated C11, D5, or E07 (Table 19).

In some embodiments, an ABP provided herein comprises a VH sequence selected from SEQ ID NOs: 6, 1, 3, or 5; and a VL sequence selected from SEQ ID NOs: 2 or 4. In some embodiments, an antibody provided herein comprises a VH sequence of SEQ ID NO: 6 and a VL sequence of SEQ ID NO: 4. In some embodiments, an antibody provided herein comprises a VH sequence of SEQ ID NO: 1 and a VL sequence of SEQ ID NO: 2. In some embodiments, an antibody provided herein comprises a VH sequence of SEQ ID NO: 3 and a VL sequence of SEQ ID NO: 4. In some embodiments, an antibody provided herein comprises a VH sequence of SEQ ID NO: 5 and a VL sequence of SEQ ID NO: 2.

In some embodiments, a CD3 antibody or antigen binding fragment thereof provided herein comprises a VH sequence selected from SEQ ID NOs: 32, 24, or 34; and a VL sequence selected from SEQ ID NOs: 33, 25, or 35. In some embodiments, an antibody provided herein comprises a VH sequence of SEQ ID NO: 32 and a VL sequence of SEQ ID NO: 33. In some embodiments, an antibody provided herein comprises a VH sequence of SEQ ID NO: 24 and a VL sequence of SEQ ID NO: 25. In some embodiments, an antibody provided herein comprises a VH sequence of SEQ ID NO: 34 and a VL sequence of SEQ ID NO: 35.

### Fc Region and Variants

An Fc region (also referred to herein as an Fc domain) can be an integral part of an antibody or Fc-fusion molecule, and can play a role in mediating effector functions such as, e.g., antibody-dependent cell-mediated cytotoxicity (ADCC), complement dependent cytotoxicity (CDC), opsonization and transcytosis.

In certain embodiments, a multispecific ABP provided herein comprises an Fc region (e.g., a CH2-CH3 domain). An Fc region can be wild-type or a variant thereof. A “wild-type Fc” or “wild-type CH2-CH3 domain” refers to one comprising an amino acid sequence identical to the amino acid sequence of an Fc region found in nature. For example, wild-type human Fc regions include a wild-type-sequence human IgG1 Fc region (non-A and A allotypes); wild-type-sequence human IgG2 Fc region; wild-type sequence human IgG3 Fc region; and wild-type-sequence human IgG4 Fc region, as well as naturally occurring variants thereof. In certain embodiments, an ABP provided herein comprises an Fc region with one or more amino acid substitutions, insertions, or deletions in comparison to a naturally occurring Fc region. In some aspects, such substitutions, insertions, or deletions yield ABP with altered stability, glycosylation, or other characteristics. In some aspects, such substitutions, insertions, or deletions yield a glycosylated ABP.

A “variant Fc region,” “engineered Fc region” or “variant CH2-CH3 domain” comprises an amino acid sequence that differs from that of a native-sequence Fc region by virtue of at least one, relative amino acid modification, e.g., one or more amino acid substitution(s). Preferably, the variant Fc region has at least one amino acid substitution compared to a native-sequence Fc region or to the Fc region of a parent polypeptide, e.g., from about one to about ten amino acid substitutions, and preferably from about one to about five amino acid substitutions in a native-sequence Fc region or in the Fc region of the parent polypeptide. The variant Fc region herein will preferably possess at least about 80% homology with a native-sequence Fc region and/or with an Fc region of a parent polypeptide, and most preferably at least about 90% homology therewith, more preferably at least about 95% homology therewith.

The term “Fe-region-comprising ABP” refers to an ABP that comprises an Fe region. The C-terminal lysine (residue 447 according to the EU numbering system) of the Fe region may be removed, for example, during purification of the ABP or by recombinant engineering the nucleic acid encoding the ABP. Accordingly, an ABP having an Fc region can comprise an ABP with or without K447.

In some aspects, the Fc region (e.g., the CH2-CH3 domain) of an ABP provided herein is modified to yield an ABP with altered affinity for an Fc receptor, or an ABP that is more immunologically inert. In some embodiments, the ABP variants provided herein possess some, but not all, effector functions. Such ABPs may be useful, for example, when the half-life of the ABP is important in vivo, but when certain effector functions (e.g., complement activation and ADCC) are unnecessary or deleterious.

In some embodiments, an ABP provided herein has one or more mutations to reduce an effector function. For example, an ABP may have mutations in the Fc of human IgG1 that result in reduced, substantial loss or complete loss of the ABP binding to CD64, CD32A, CD16 and C1q (FcγRI, FcγRII, FcγRIII and C1q) relative to an unmodified version of the Fe. In some embodiments, an ABP provided herein comprises a variant CH2-CH3 domain comprising one or more amino acid substitutions that reduce binding to an Fe receptor on the cell surface of an effector cell, e.g., FcγRI; FcγRIIA; FcγRIIB1; FcγRIIIB2; FcγRIIIA; FcγRIIIB receptors. In some embodiments, the reduced effector functions can include one or more of reduced complement-dependent cytotoxicity (CDC), reduced antibody-dependent cellular cytotoxicity (ADCC), and reduced complement fixation. These modifications to the Fe can prevent multispecific ABPs from causing target cell death (e.g., T cell death) or, e.g., unwanted cytokine secretion. The modification(s) can also help reduce inter-individual variation in patient response to an ABP provided herein. Disabling productive Fe receptor engagement by reducing binding to one or more Fc receptors other than FcRn, where the Fe receptor binds monomeric IgG and/or multimeric immune complexes, can restore activity to the antibody and provide an improved therapeutic profile.

Examples of Fe effector functions that can be reduced through modification include, without limitation: ability to activate classical complement; ADCC; opsonization; ability to bind FcγRI (CD64) at, e.g., a high affinity of 1×10⁻⁹ M; ability to bind FcγRIIIa,b (CD16), e.g., at an affinity 5×10⁻⁵ M or higher; and ability to bind FcγRIIa,b (CD32), e.g., at an affinity of 2×10⁻⁶ M or higher. Properties of antibodies having reduced effector function via Fe silencing are described, for example, in An et al. mAbs vol. 1,6 (2009): 572-9 and Wang, et al. Protein & cell 9.1 (2018): 63-73, the relevant disclosures of each of which are herein incorporated by reference.

In some embodiments, the ABP comprises a variant CH2-CH3 domain comprising one or more amino acid substitutions which reduce Fc effector functions. In some embodiments, the one or more amino acid substitutions are in the CH2 domain at one or more of EU index positions: 234, 235, and/or 331. In some embodiments, the one or more amino acid substitutions are in the CH2 domain at EU index positions: 234, 235, and 331. In some embodiments, the one or more amino acid substitutions are selected from: L234F, L235E, and P331S, according to the EU numbering system. In some embodiments, the variant CH2-CH3 domain comprises the amino acid substitutions of L234F, L235E, and P331S (dubbed “TM” modifications or mutations), according to the EU numbering system.

Binding of IgG to the FcγRs or C1q depends on residues located in the hinge region and the CH2 domain. Substitutions in human IgG1 or IgG2 residues at positions 233-236 and IgG4 residues at positions 327, 330 and 331 have been shown to greatly reduce ADCC and CDC. The triple amino acid substitution L234A, L235A, and G237A largely eliminates FcγR and complement effector functions (see, for example, U.S. Pat. No. 9,644,025, the relevant disclosures of which are herein incorporated by reference). The LALA variant, L234A/L235A, also has significantly reduced FcγR binding; as does E233P/L234V/L235A/G236+A327G/A330S/P331S. See, for example, Armour et al. (1999) Eur J Immunol. 29(8):2613-24. The set of mutations: K322A, L234A and L235A are sufficient to almost completely abolish FcγR and C1q binding.

Additional modifications to silence the Fc region or reduce effector function may include three amino acid substitutions in the CH2 region to reduce FcγRI binding at EU index positions 234, 235, and 237 (see Duncan et al., (1988) Nature 332:563). Two amino acid substitutions in the complement C1q binding site at EU index positions 330 and 331 reduce complement fixation (see Tao et al., J. Exp. Med. 178:661 (1993) and Canfield and Morrison, J. Exp. Med. 173:1483 (1991)). Substitution into human IgG1 of IgG2 residues at positions 233-236 and IgG4 residues at positions 327, 330 and 331 greatly reduces ADCC and CDC (see, for example, Armour K L. et al., 1999 Eur J Immunol. 29(8):2613-24; and Shields R L. et al., 2001. J Biol Chem. 276(9):6591-604).

Additional mutations that reduce binding to FcγR include, without limitation, modification of the glycosylation on asparagine 297 of the Fc domain, which is known to be required for optimal FcR interaction. For example known amino acid substitutions include N297 mutations, for example N297A/Q/D/H/G/C, which changes result in the loss of a glycosylation site on the protein. Enzymatically deglycosylated Fc domains, recombinantly expressed antibodies in the presence of a glycosylation inhibitor and the expression of Fc domains in bacteria have a similar loss of glycosylation and consequent binding to FcγRs.

Additional examples of Fc silencing are known to those of ordinary skill in the art and are provided, for example, in U.S. Pat. No. 10,611,842, the relevant disclosures of which are herein incorporated by reference.

As used herein, a “silenced Fc” or “silenced CH2-CH3 domain” refers to one that has been mutagenized to retain activity with respect to, for example, prolonging serum half-life through interaction with, e.g., FcRn, or while retaining its PK profile, but which has reduced or absent binding to one or more other Fc receptor(s), including without limitation a human FcγR as listed supra.

In some embodiments, the Fc region or CH2-CH3 domain of an ABP provided herein is a human IgG4 Fc region comprising one or more of the hinge stabilizing mutations S228P and L235E, according to EU numbering. See Aalberse et al., Immunology, 2002, 105:9-19, incorporated by reference in its entirety. In some embodiments, the IgG4 Fc region comprises one or more of the following mutations: E233P, F234V, and L235A, according to EU numbering. See Armour et al., Mol. Immunol., 2003, 40:585-593, incorporated by reference in its entirety. In some embodiments, the IgG4 Fc region comprises a deletion at position G236.

In some embodiments, the Fc region or CH2-CH3 domain of an ABP provided herein is a human IgG1 Fc region comprising one or more mutations to reduce Fc receptor binding. In some aspects, the one or more mutations are in residues selected from S228 (e.g., S228A), L234 (e.g., L234A), L235 (e.g., L235A), D265 (e.g., D265A), and N297 (e.g., N297A), according to EU numbering. In some aspects, the ABP comprises a PVA236 mutation. PVA236 means that the amino acid sequence ELLG (SEQ ID NO: 212), from amino acid position 233 to 236 of IgG1 or EFLG (SEQ ID NO: 213) of IgG4, is replaced by PVA, according to EU numbering. See U.S. Pat. No. 9,150,641, incorporated by reference in its entirety.

In some embodiments, the Fc region of an ABP provided herein is modified as described in Armour et al., Eur J. Immunol., 1999, 29:2613-2624; WO 1999/058572; and/or U.K. Pat. App. No. 98099518; each of which is incorporated by reference in its entirety.

In some embodiments, a sequence comprising the CH2-CH3 domains of the first polypeptide is distinct from a sequence comprising the CH2-CH3 domains of the second polypeptide. In some embodiments, the CH2-CH3 domains of the first polypeptide and/or the CH2-CH3 domains of the second polypeptide comprise a variant CH2-CH3 domain. In some embodiments, the variant CH2-CH3 domain comprises a modification that alters an affinity of the ABP for an Fc receptor as compared to a multispecific ABP with a non-variant CH2-CH3 domain. In some embodiments, the first hinge comprises a C220S mutation, according to EU numbering. In some embodiments, the variant CH2-CH3 domain comprises a human IgG4 Fc region comprising one or more of the hinge stabilizing mutations S228P and L235E, or comprising one or more of the following mutations: E233P, F234V, and L235A, according to EU numbering. In some embodiments, the variant CH2-CH3 domain is a human IgG1 Fc region comprising one or more mutations to reduce Fc receptor binding, optionally wherein the one or more mutations are in residues selected from S228 (e.g., S228A), L234 (e.g., L234A), L235 (e.g., L235A), D265 (e.g., D265A), and N297 (e.g., N297A or N297Q), or optionally wherein the amino acid sequence ELLG (SEQ ID NO: 212), from amino acid position 233 to 236 of IgG1 or EFLG (SEQ ID NO: 213) of IgG4, is replaced by PVA, according to EU numbering. In some embodiments, the variant CH2-CH3 domain is a human IgG2 Fc region comprising one or more of mutations A330S and P331S, according to EU numbering. In some embodiments, the variant CH2-CH3 domain comprises an amino acid substitution at one or more positions selected from 238, 265, 269, 270, 297, 327 and 329, optionally wherein the variant CH2-CH3 domain comprises substitutions at two or more of amino acid positions 265, 269, 270, 297 and 327, optionally wherein the variant CH2-CH3 domain comprises substitution of residues 265 or 297 with alanine, optionally wherein the variant CH2-CH3 domain comprises substitution of residues 265 and 297 with alanine, according to EU numbering

In some embodiments, the variant CH2-CH3 domain of an ABP provided herein is a human IgG2 Fc region comprising one or more of mutations A330S and P331S, according to EU numbering.

In some embodiments, the Fc region (variant CH2-CH3 domain) of an ABP provided herein has an amino acid substitution at one or more positions selected from 238, 265, 269, 270, 297, 327 and 329, according to EU numbering. See U.S. Pat. No. 6,737,056, incorporated by reference in its entirety. Such Fc mutants include Fc mutants with substitutions at two or more of amino acid positions 265, 269, 270, 297 and 327, including the so-called “DANA” Fc mutant with substitution of residues 265 and 297 with alanine, according to EU numbering. See U.S. Pat. No. 7,332,581, incorporated by reference in its entirety. In some embodiments, the ABP comprises an alanine at amino acid position 265. In some embodiments, the ABP comprises an alanine at amino acid position 297.

In certain embodiments, an ABP provided herein comprises an Fe region (a variant CH2-CH3 domain) with one or more amino acid substitutions that reduce at least one Fc effector function. In certain embodiments, an ABP provided herein comprises a CH2-CH3 domain comprising one or more amino acid substitutions that reduce binding to an Fc receptor on the cell surface of an effector cell.

In certain embodiments, the Fc receptor on the cell surface of an effector cell is selected from: FcγRI; FcγRIIA; FcγRIIB1; FcγRIIIB2; FcγRIIIA; and FcγRIIIB receptors. In certain embodiments, the one or more amino acid substitutions is selected from: L234, L235, P331, L234F, L235E, and P331S, according to the EU numbering system. In certain embodiments, the variant CH2-CH3 domain comprises the amino acid substitutions of L234F, L235E, and P331S, according to the EU numbering system. In certain embodiments, the Fc effector function that is reduced comprises one or more functions selected from: complement-dependent cytotoxicity (CDC), antibody-dependent cellular cytotoxicity (ADCC), and complement fixation.

In some embodiments, the variant CH2-CH3 domain comprises one or more amino acid substitutions which improve ADCC, such as a substitution at one or more of positions 298, 333, and 334 of the variant CH2-CH3 domain, or a substitution at one or more of positions 239, 332, and 330 of the variant CH2-CH3 domain, according to EU numbering. In some embodiments, the variant CH2-CH3 domain comprises one or more modifications to increase half-life, optionally wherein the variant CH2-CH3 domain comprises substitutions at one or more of the variant CH2-CH3 domain residues: 238, 250, 256, 265, 272, 286, 303, 305, 307, 311, 312, 314, 317, 340, 356, 360, 362, 376, 378, 380, 382, 413, 424, 428, and 434 of an IgG, according to EU numbering. In some embodiments, the ABP comprises a G1m17,1 allotype.

In certain embodiments, an ABP provided herein comprises a variant CH2-CH3 domain with one or more amino acid substitutions which improve ADCC, such as a substitution at one or more of positions 298, 333, and 334 of the Fc region, according to EU numbering. In some embodiments, an ABP provided herein comprises a variant CH2-CH3 domain with one or more amino acid substitutions at positions 239, 332, and 330, as described in Lazar et al., Proc. Natl. Acad. Sci. USA, 2006,103:4005-4010, incorporated by reference in its entirety, according to EU numbering.

In some embodiments, an ABP provided herein comprises one or more alterations that improves or diminishes C1q binding and/or CDC. See U.S. Pat. No. 6,194,551; WO 99/51642; and Idusogie et al., J. Immunol., 2000, 164:4178-4184; each of which is incorporated by reference in its entirety.

In some embodiments, an ABP provided herein comprises one or more alterations to increase half-life. ABPs with increased half-lives and improved binding to the neonatal Fc receptor (FcRn) are described, for example, in Hinton et al., J. Immunol., 2006, 176:346-356; and U.S. Pat. Pub. No. 2005/0014934; each of which is incorporated by reference in its entirety. Such Fc variants include those with substitutions at one or more of Fc region residues: 238, 250, 256, 265, 272, 286, 303, 305, 307, 311, 312, 314, 317, 340, 356, 360, 362, 376, 378, 380, 382, 413, 424, 428, and 434 of an IgG, according to EU numbering. In some embodiments, the ABP comprises one or more non-Fc modifications that extend half-life. Exemplary non-Fc modifications that extend half-life are described in, e.g., US20170218078, which is hereby incorporated by reference in its entirety.

In some embodiments, an ABP provided herein comprises a G1m17,1 allotype. Such allotype is described in, e.g., Lefranc G, Lefranc M-P. Gm allotype and Gm haplotypes> Allotypes. In IMGT Repertoire (IG and TR). IMGT®, the international ImMunoGeneTics information System®. www.imgt.org/IMGTrepertoire/Proteins/allotypes/human/IGH/IGHC/G1m_allotypes.html, which is hereby incorporated by reference in its entirety.

In some embodiments, an ABP provided herein comprises one or more variant CH2-CH3 domains as described in U.S. Pat. Nos. 7,371,826 5,648,260, and 5,624,821; Duncan and Winter, Nature, 1988, 322:738-740; and WO 94/29351; each of which is incorporated by reference in its entirety.

In some embodiments, the multispecific ABP comprises one or more Fc (CH2-CH3 domain) modifications that promote heteromultimerization. In some embodiments, the Fc modification comprises a knob-in-hole modification. Knob-in-hole modifications are described in, e.g., U.S. Pat. No. 7,695,936, Merchant et al., Nature Biotechnology 1998 July; 16(7):677-81; Ridgway et al., Protein Engineering 1996 July; 9(7):617-21; and Atwell et al., J Mol Biol. 1997 Jul. 4; 270(1):26-35, each of which is incorporated by reference in its entirety.

In some embodiments, the hinge region on the knob side has a C220 mutation, e.g., C220S, according to EU numbering. This C220S mutation is added in the antibody arm that does not have a light-chain because a free Cys can cause challenges with folding.

In some embodiments, one Fc-bearing chain (CH2-CH3 domain) of the multispecific ABP comprises a T366W mutation, and the other Fc-bearing chain (CH2-CH3 domain) of the multispecific ABP comprises a T366S, L368A, and Y407V mutation, according to EU numbering. In some embodiments, the multispecific ABP comprising a knob-in-hole modification further comprises an engineered disulfide bridge in the Fe region. In some embodiments, the engineered disulfide bridge comprises a K392C mutation in one Fe-bearing chain (CH2-CH3 domain) of the multispecific ABP, and a D399C in the other Fc-bearing chain (CH2-CH3 domain) of the multispecific ABP, according to EU numbering. In some embodiments, the engineered disulfide bridge comprises a S354C mutation in one Fc-bearing chain (CH2-CH3 domain) of the multispecific ABP, and a Y349C mutation in the other Fc-bearing chain of the multispecific ABP, according to EU numbering.

In some embodiments, the ABP comprises a T366W mutation, and the other Fc-bearing chain (CH2-CH3 domain) of the ABP comprises a T366S, L368A, and Y407V mutation, according to EU numbering; and the ABP comprises an engineered disulfide bridge, wherein the engineered disulfide bridge comprises a S354C mutation in one Fe-bearing chain (CH2-CH3 domain) of the ABP, and a Y349C mutation in the other Fe-bearing chain (CH2-CH3 domain) of the ABP, according to EU numbering.

In some embodiments, the engineered disulfide bridge comprises a 447C mutation in both Fe-bearing chains of the ABP, which 447C mutations are provided by extension of the C-terminus of a CH3 domain incorporating a KSC tripeptide sequence. In some embodiments, the ABP comprises an S354C and T366W mutation in one Fe-bearing chain and a Y349C, T366S, L368A and Y407V mutation in the other Fe-bearing chain, according to EU numbering.

In some embodiments, the Fe modification comprises a set of mutations described in Von Kreudenstein T S, Escobar-Carbrera E, Lario P I, et al. Improving biophysical properties of a bispecific antibody scaffold to aid developability: quality by molecular design. MAbs. 2013; 5(5):646-54, which is hereby incorporated by reference in its entirety. In some embodiments, the Fe modification comprises a set of mutations as provided in the following

TABLE E
(numbering is according to EU numbering).
Table E

| Chain-A | Chain-B |
|---|---|
| F405A_Y407V | T394W |
| F405A_Y407V | T366I_T394W |
| F405A_Y407V | T366L_T394W |
| F405A_Y407V | T366L_K392M_T394W |
| L351Y_F405A_Y407V | T366L_K392M_T394W |
| T350V_L351Y_F405A_Y407V | T350V_T366L_K392M_T394W |
| T350V_L351Y_F405A_Y407V | T350V_T366L_K392L_T394W |

In some embodiments, the Fe modification comprises a set of mutations described in Labrijn A F, et al., Proc Natl Acad Sci USA. 2013 Mar. 26; 110(13):5145-50. doi: 10.1073/pnas. In some embodiments, the Fc region is an IgG1 Fc, and the Fe modification comprises a K409R mutation in one Fe-bearing chain and a mutation selected from a Y407, L368, F405, K370, and D399 mutation in the other Fe-bearing chain, according to EU numbering. In some embodiments, the Fe modification comprises a K409R mutation in one Fe-bearing chain and a F405L mutation in the other Fe-bearing chain, according to EU numbering.

In some embodiments, the Fe modification comprises a set of mutations that renders homodimerization electrostatically unfavorable but heterodimerization favorable. An exemplary set of mutations is described in U.S. Pat. No. 8,592,562, and in Gunasekaran K et al., The Journal of Biological Chemistry 285, 19637-19646, doi: 10.1074/jbc.M110.117382, which are each incorporated by reference in its entirety. In some embodiments, the Fe modification comprises a K409D_K392D mutation in one Fe-bearing chain and a D399K_E356K mutation in the other Fe-bearing chain, according to EU numbering.

In some embodiments, the engineered disulfide bridge comprises a K392C mutation in one CH2-CH3 domain (Fc region) of the ABP, and a D399C in the other CH2-CH3 domain (Fc region) of the ABP, according to EU numbering. In some embodiments, the engineered disulfide bridge comprises a S354C mutation in one CH2-CH3 domain of the ABP, and a Y349C mutation in the other CH2-CH3 domain of the ABP, according to EU numbering. In some embodiments, the engineered disulfide bridge comprises a 447C mutation in both CH2-CH3 domains of the ABP, which 447C mutations are provided by extension of the C-terminus of a CH3 domain incorporating a KSC tripeptide sequence, according to EU numbering

In some embodiments, the Fe modification comprises a set of mutations described in WO2011143545, which is hereby incorporated by reference in its entirety. In some embodiments, the Fe modification comprises a K409R mutation in one Fe-bearing chain and a L368E or L368D mutation in the other Fe-bearing chain, according to EU numbering. In some embodiments, the Fe modification comprises a set of mutations described in Strop P et al., J. Mol. Biol., 420 (2012), pp. 204-219, which is hereby incorporated by reference in its entirety. In some embodiments, the Fe modification comprises a D221E, P228E, and L368E mutation in one Fe-bearing chain and a D221R, P228R, and K409R in the other Fe-bearing chain, according to EU numbering.

In some embodiments, the Fc modification comprises a set of mutations described in Moore G L, et al., mAbs, 3 (2011), pp. 546-557, which is hereby incorporated by reference in its entirety. In some embodiments, the Fc modification comprises an S364H and F405A mutation in one Fc-bearing chain and a Y349T and T394F mutation in the other Fc-bearing chain, according to EU numbering. In some embodiments, the Fc modification comprises a set of mutations described in U.S. Pat. No. 9,822,186, which is hereby incorporated by reference in its entirety. In some embodiments, the Fc modification comprises an E375Q and S364K mutation in one Fc-bearing chain and a L368D and K370S mutation in the other Fc-bearing chain, according to EU numbering.

In some embodiments, the Fc modification comprises strand-exchange engineered domain (SEED) CH3 heterodimers. Such SEED CH3 heterodimers are described in, e.g., Davis J H et al., Protein Eng Des Sel. 2010 April; 23(4):195-202. doi: 10.1093/protein/gzp094, which is hereby incorporated by reference in its entirety.

In some embodiments, the Fc modification comprises a modification in the CH3 sequence that affects the ability of the CH3 domain to bind an affinity agent, e.g., Protein A. In some embodiments, one of the variant CH2-CH3 domains is capable of binding Protein A and the other variant CH2-CH3 domain comprises a mutation that reduces binding affinity of such CH2-CH3 domain to Protein A as compared to the first CH2-CH3 domain. Such modifications, and methods of producing multispecific ABPs comprising the modifications, are described in U.S. Pat. No. 8,586,713, US20160024147A1, and Smith E J, et al., Scientific Reports 2015 Dec. 11; 5:17943. doi: 10.1038/srep17943., each of which is hereby incorporated by reference in its entirety. In some embodiments, the Fc modification comprises a H435 and/or Y436 mutation (e.g., H435R and/or Y436F mutation) in at least one Fc-bearing chain, according to EU numbering. In some embodiments, the other Fc-bearing chain does not comprise an amino acid mutation. In some embodiments, the other CH2-CH3 domain comprises a H435, Y436, H435R, Y436F, or H435R_Y436F mutation, according to EU numbering.

### Isolated HLA-Peptide Targets

The major histocompatibility complex (MHC) is a complex of antigens encoded by a group of linked loci, which are collectively termed H-2 in the mouse and HLA in humans. The two principal classes of the MHC antigens, class I and class II, each comprise a set of cell surface glycoproteins which play a role in determining tissue type and transplant compatibility. In transplantation reactions, cytotoxic T-cells (CTLs) respond mainly against class I glycoproteins, while helper T-cells respond mainly against class II glycoproteins.

Human major histocompatibility complex (MHC) class I molecules, referred to interchangeably herein as HLA Class I molecules, are expressed on the surface of nearly all cells. These molecules function in presenting peptides which are mainly derived from endogenously synthesized proteins to, e.g., CD8+ T cells via an interaction with the alpha-beta T-cell receptor. The class I MHC molecule comprises a heterodimer composed of a 46-kDa a chain which is non-covalently associated with the 12-kDa light chain beta-2 microglobulin. The a chain generally comprises α1 and u2 domains which form a groove for presenting an HLA-restricted peptide, and an α3 plasma membrane-spanning domain which interacts with the CD8 co-receptor of T-cells. See, e.g., Kerry S E, Buslepp J, Cramer L A, et al. Interplay between TCR Affinity and Necessity of Coreceptor Ligation: High-Affinity Peptide-MHC/TCR Interaction Overcomes Lack of CD8 Engagement. Journal of immunology (Baltimore, Md: 1950). 2003; 171(9):4493-4503.)

Class I MHC-restricted peptides (also referred to interchangeably herein as HLA-restricted antigens, HLA-restricted peptides, MHC-restricted antigens, restricted peptides, or peptides) generally bind to the heavy chain alpha1-alpha2 groove via about two or three anchor residues that interact with corresponding binding pockets in the MHC molecule. The beta-2 microglobulin chain plays an important role in MHC class I intracellular transport, peptide binding, and conformational stability. For most class I molecules, the formation of a heterotrimeric complex of the MHC class I heavy chain, peptide (self, non-self, and/or antigenic) and beta-2 microglobulin leads to protein maturation and export to the cell-surface.

Binding of a given HLA subtype to an HLA-restricted peptide forms a complex with a unique and novel surface that can be specifically recognized by an ABP such as, e.g., a TCR on a T cell or an antibody or antigen-binding fragment thereof. HLA complexed with an HLA-restricted peptide is referred to herein as an HLA-PEPTIDE or HLA-PEPTIDE target. In some cases, the restricted peptide is located in the α1/α2 groove of the HLA molecule. In some cases, the restricted peptide is bound to the α1/α2 groove of the HLA molecule via about two or three anchor residues that interact with corresponding binding pockets in the HLA molecule.

The HLA-PEPTIDE targets described herein is useful for cancer immunotherapy. In some embodiments, the HLA-PEPTIDE targets identified herein are presented on the surface of a tumor cell. The HLA-PEPTIDE targets identified herein may be expressed by tumor cells in a human subject. The HLA-PEPTIDE targets identified herein may be expressed by tumor cells in a population of human subjects. For example, the HLA-PEPTIDE targets identified herein may be shared antigens which are commonly expressed in a population of human subjects with cancer.

The HLA-PEPTIDE targets identified herein may have a prevalence with an individual tumor type The prevalence with an individual tumor type may be about 0.1%, 0.2%, 0.3%, 0.4%, 0.5%, 0.6%, 0.7%, 0.8%, 0.9%, 1%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%. The prevalence with an individual tumor type may be about 0.1%-100%, 0.2-50%, 0.5-25%, 2-20% or 1-10%.

Preferably, HLA-PEPTIDE targets are not generally expressed in most normal tissues. For example, the HLA-PEPTIDE targets may in some cases not be expressed in tissues in the Genotype-Tissue Expression (GTEx) Project, or may in some cases be expressed only in immune privileged or non-essential tissues. Exemplary immune privileged or non-essential tissues include testis, minor salivary glands, the endocervix, and the thyroid. In some cases, an HLA-PEPTIDE target may be deemed to not be expressed on essential tissues or non-immune privileged tissues if the median expression of a gene from which the restricted peptide is derived is less than 0.5 RPKM (Reads Per Kilobase of transcript per Million mapped reads) across GTEx samples, if the gene is not expressed with greater than 10 RPKM across GTEX samples, if the gene was expressed at >=5 RPKM in no more two samples across all essential tissue samples, or any combination thereof.

Also provided herein are off-target HLA-PEPTIDES. Such off-target HLA-PEPTIDES may be useful for identifying a cancer therapeutic, e.g., an ABP disclosed herein.

### HLA-Restricted Peptides

The HLA-restricted peptides of an HLA-PEPTIDE target disclosed herein (referred to interchangeably herein) as “restricted peptides” can be peptide fragments of tumor-specific genes, e.g., cancer-specific genes. Preferably, the cancer-specific genes are expressed in cancer samples. The restricted peptide contemplated herein is from tumor-specific gene product CT83 (Cancer/testis antigen 83; also referred to as KKLC-1). Genes which are aberrantly expressed in cancer samples can be identified through a database. Exemplary databases include, by way of example only, The Cancer Genome Atlas (TCGA) Research Network: cancergenome.nih.gov/; the International Cancer Genome Consortium: dcc.icgc.org/. In some embodiments, the cancer-specific gene has an observed expression of at least 10 RPKM in at least 5 samples from the TCGA database. The cancer-specific gene may have an observable bimodal distribution.

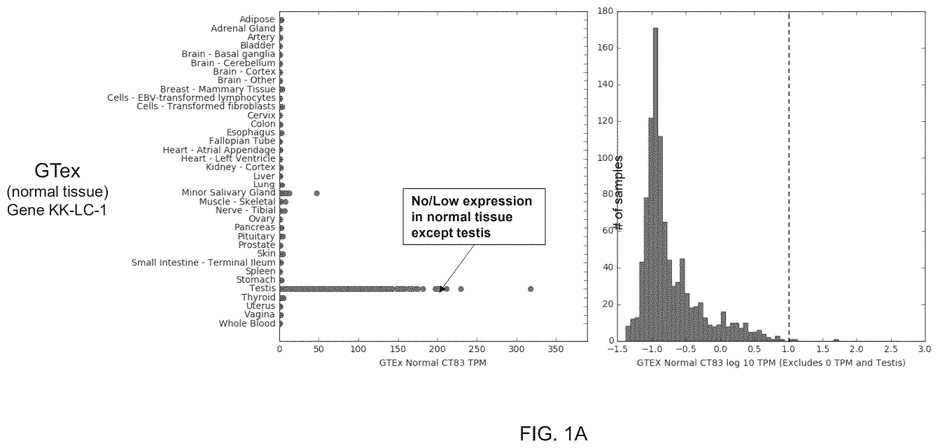

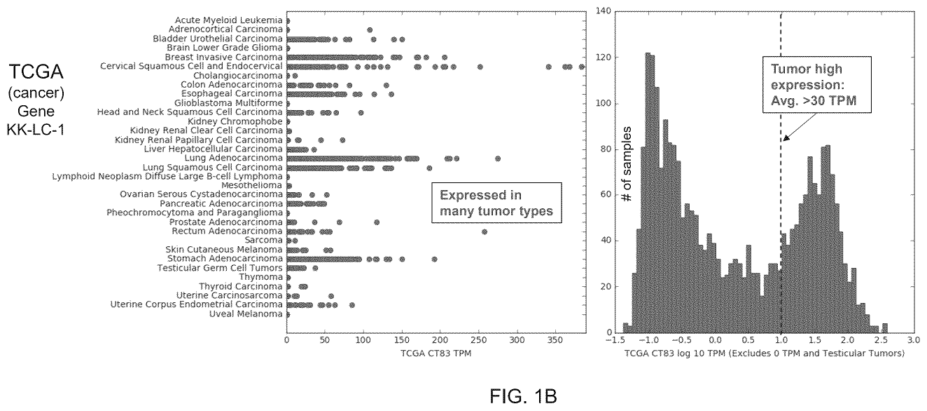

The cancer-specific gene may have an observed expression of greater than 10, 20, 30, 40, 50, 60, 70, 80, 90, or 100 transcripts per million (TPM) in at least one TCGA tumor tissue. See FIGS. 1A and 1B. In some embodiments, the cancer-specific gene has an observed expression of greater than 100 TPM in at least one TCGA tumor tissue. In some cases, the cancer specific gene has an observed bimodal distribution of expression across TCGA samples. Without wishing to be bound by theory, such bimodal expression pattern is consistent with a biological model in which there is minimal expression at baseline in all tumor samples and higher expression in a subset of tumors experiencing epigenetic dysregulation.

Preferably, the cancer-specific gene is not generally expressed in most normal tissues. For example, the cancer-specific gene may in some cases not be expressed in tissues in the Genotype-Tissue Expression (GTEx) Project, or may in some cases be expressed in immune privileged or non-essential tissues. Exemplary immune privileged or non-essential tissues include testis, minor salivary glands, the endocervix, and thyroid. In some cases, an cancer-specific gene may be deemed to not be expressed an essential tissues or non-immune privileged tissue if the median expression of the cancer-specific gene is less than 0.5 RPKM (Reads Per Kilobase of transcript per Million mapped reads) across GTEx samples, if the gene is not expressed with greater than 10 RPKM across GTEX samples, if the gene was expressed at >=5 RPKM in no more two samples across all essential tissue samples, or any combination thereof.

In some embodiments, the cancer-specific gene meets the following criteria by assessment of the GTEx: (1) median GTEx expression in brain, heart, or lung is less than 0.1 transcripts per million (TPM), with no one sample exceeding 5 TPM, (2) median GTEx expression in other essential organs (excluding testis, thyroid, minor salivary gland) is less than 2 TPM with no one sample exceeding 10 TPM.

In some embodiments, the cancer-specific gene is not likely expressed in immune cells generally, e.g., is not an interferon family gene, is not an eye-related gene, not an olfactory or taste receptor gene, and is not a gene related to the circadian cycle (e.g., not a CLOCK, PERIOD, CRY gene).

The restricted peptide preferably may be presented on the surface of a tumor.

The restricted peptides may have a size of about 5, about 6, about 7, about 8, about 9, about 10, about 11, about 12, about 13, about 14, or about 15 amino molecule residues, and any range derivable therein. In particular embodiments, the restricted peptide has a size of about 8, about 9, about 10, about 11, or about 12 amino molecule residues. The restricted peptide may be about 5-15 amino acids in length, preferably may be about 7-12 amino acids in length, or more preferably may be about 8-11 amino acids in length.

A restricted peptide of an off-target HLA-PEPTIDE can be a fragment of a protein expressed in normal, e.g., non-tumor tissue. In some embodiments, a restricted peptide of an off-target HLA-PEPTIDE is indicated as being expressed in normal tissues according to the public GTEX database.

HLA Class I molecules which do not associate with a restricted peptide ligand are generally unstable. Accordingly, the association of the restricted peptide with the α1/α2 groove of the HLA molecule may stabilize the non-covalent association of the β2-microglobulin subunit of the HLA subtype with the α-subunit of the HLA subtype.

Stability of the non-covalent association of the β2-microglobulin subunit of the HLA subtype with the α-subunit of the HLA subtype can be determined using any suitable means. For example, such stability may be assessed by dissolving insoluble aggregates of HLA molecules in high concentrations of urea (e.g., about 8M urea), and determining the ability of the HLA molecule to refold in the presence of the restricted peptide during urea removal, e.g., urea removal by dialysis. Such refolding approaches are described in, e.g., Proc. Natl. Acad. Sci. USA Vol. 89, pp. 3429-3433, April 1992, hereby incorporated by reference in its entirety.

For other example, such stability may be assessed using conditional HLA Class I ligands. Conditional HLA Class I ligands are generally designed as short restricted peptides which stabilize the association of the β2 and a subunits of the HLA Class I molecule by binding to the α1/α2 groove of the HLA molecule, and which contain one or more amino acid modifications allowing cleavage of the restricted peptide upon exposure to a conditional stimulus. Upon cleavage of the conditional ligand, the β2 and α-subunits of the HLA molecule dissociate, unless such conditional ligand is exchanged for a restricted peptide which binds to the α1/α2 groove and stabilizes the HLA molecule. Conditional ligands can be designed by introducing amino acid modifications in either known HLA peptide ligands or in predicted high-affinity HLA peptide ligands. For HLA alleles for which structural information is available, water-accessibility of side chains may also be used to select positions for introduction of the amino acid modifications. Use of conditional HLA ligands may be advantageous by allowing the batch preparation of stable HLA-peptide complexes which may be used to interrogate test restricted peptides in a high throughput manner. Conditional HLA Class I ligands, and methods of production, are described in, e.g., Proc Natl Acad Sci USA. 2008 Mar. 11; 105(10): 3831-3836; Proc Natl Acad Sci USA. 2008 Mar. 11; 105(10): 3825-3830; J Exp Med. 2018 May 7; 215(5): 1493-1504; Choo, J. A. L. et al. Bioorthogonal cleavage and exchange of major histocompatibility complex ligands by employing azobenzene-containing peptides. Angew Chem Int Ed Engl 53, 13390-13394 (2014); Amore, A. et al. Development of a Hypersensitive Periodate-Cleavable Amino Acid that is Methionine- and Disulfide-Compatible and its Application in MHC Exchange Reagents for T Cell Characterisation. ChemBioChem 14, 123-131 (2012); Rodenko, B. et al. Class I Major Histocompatibility Complexes Loaded by a Periodate Trigger. J Am Chem Soc 131, 12305-12313 (2009); and Chang, C. X. L. et al. Conditional ligands for Asian HLA variants facilitate the definition of CD8+ T-cell responses in acute and chronic viral diseases. Eur J Immunol 43, 1109-1120 (2013). These references are incorporated by reference in their entirety.

Accordingly, in some embodiments, the ability of an HLA-restricted peptide described herein to stabilize the association of the β2- and α-subunits of the HLA molecule, is assessed by performing a conditional ligand mediated-exchange reaction and assay for HLA stability. HLA stability can be assayed using any suitable method, including, e.g., mass spectrometry analysis, immunoassays (e.g., ELISA), size exclusion chromatography, and HLA multimer staining followed by flow cytometry assessment of T cells.

Other exemplary methods for assessing stability of the non-covalent association of the β2-microglobulin subunit of the HLA subtype with the α-subunit of the HLA subtype include peptide exchange using dipeptides. Peptide exchange using dipeptides has been described in, e.g., Proc Natl Acad Sci USA. 2013 Sep. 17, 110(38):15383-8; Proc Natl Acad Sci USA. 2015 Jan. 6, 112(1):202-7, which is hereby incorporated by reference in its entirety.

Provided herein are useful antigens comprising an HLA-PEPTIDE target. The HLA-PEPTIDE targets may comprise a specific HLA-restricted peptide having a defined amino acid sequence complexed with a specific HLA subtype allele.

The HLA-PEPTIDE target or off-target HLA-PEPTIDE may be isolated and/or in substantially pure form. For example, the HLA-PEPTIDE targets or off-target HLA-PEPTIDEs may be isolated from their natural environment, or may be produced by means of a technical process. In some cases, the HLA-PEPTIDE target or off-target HLA-PEPTIDE is provided in a form which is substantially free of other peptides or proteins.

THE HLA-PEPTIDE targets or off-target HLA-PEPTIDEs may be presented in soluble form, and optionally may be a recombinant HLA-PEPTIDE target complex. The skilled artisan may use any suitable method for producing and purifying recombinant HLA-PEPTIDE targets or off-target HLA-PEPTIDEs. Suitable methods include, e.g., use of E. coli expression systems, insect cells, and the like. Other methods include synthetic production, e.g., using cell free systems. An exemplary suitable cell free system is described in WO2017089756, which is hereby incorporated by reference in its entirety.

Also provided herein are compositions comprising an HLA-PEPTIDE target or off-target HLA-PEPTIDE.

In some cases, the composition comprises an HLA-PEPTIDE target or off-target HLA-PEPTIDE attached to a solid support. Exemplary solid supports include, but are not limited to, beads, wells, membranes, tubes, columns, plates, sepharose, magnetic beads, and chips. Exemplary solid supports are described in, e.g., Catalysts 2018, 8, 92; doi:10.3390/cata18020092, which is hereby incorporated by reference in its entirety.

The HLA-PEPTIDE target may be attached to the solid support by any suitable methods known in the art. In some cases, the HLA-PEPTIDE target is covalently attached to the solid support.

In some cases, the HLA-PEPTIDE target is attached to the solid support by way of an affinity binding pair. Affinity binding pairs generally involved specific interactions between two molecules. A ligand having an affinity for its binding partner molecule can be covalently attached to the solid support, and thus used as bait for immobilizing Common affinity binding pairs include, e.g., streptavidin and biotin, avidin and biotin; polyhistidine tags with metal ions such as copper, nickel, zinc, and cobalt; and the like.

The HLA-PEPTIDE target may comprise a detectable label.

### ABPs Comprising an Antibody or Antigen-Binding Fragment Thereof

In some embodiments, the ABP comprises an antibody or antigen-binding fragment thereof.

In some embodiments, the ABPs provided herein comprise a light chain. In some aspects, the light chain is a kappa light chain. In some aspects, the light chain is a lambda light chain.

In some embodiments, the ABPs provided herein comprise a heavy chain. In some aspects, the heavy chain is an IgA. In some aspects, the heavy chain is an IgD. In some aspects, the heavy chain is an IgE. In some aspects, the heavy chain is an IgG. In some aspects, the heavy chain is an IgM. In some aspects, the heavy chain is an IgG1. In some aspects, the heavy chain is an IgG2. In some aspects, the heavy chain is an IgG3. In some aspects, the heavy chain is an IgG4. In some aspects, the heavy chain is an IgA1. In some aspects, the heavy chain is an IgA2.

In some embodiments, the ABPs provided herein comprise an antibody fragment. In some embodiments, the ABPs provided herein consist of an antibody fragment. In some embodiments, the ABPs provided herein consist essentially of an antibody fragment. In some aspects, the ABP fragment is an Fv fragment. In some aspects, the ABP fragment is a Fab fragment. In some aspects, the ABP fragment is a F(ab′)₂ fragment. In some aspects, the ABP fragment is a Fab′ fragment. In some aspects, the ABP fragment is an scFv (sFv) fragment. In some aspects, the ABP fragment is an scFv-Fc fragment. In some aspects, the ABP fragment is a fragment of a single domain ABP.

In some embodiments, an ABP fragment provided herein is derived from an illustrative ABP provided herein. In some embodiments, an ABP fragments provided herein is not derived from an illustrative ABP provided herein and may, for example, be isolated de novo according to the methods provided herein for obtaining ABP fragments.

In some embodiments, an ABP fragment provided herein retains the ability to bind the HLA-PEPTIDE target, as measured by one or more assays or biological effects described herein. In some embodiments, an ABP fragment provided herein retains the ability to prevent HLA-PEPTIDE from interacting with one or more of its ligands, as described herein.

The ABP fragments provided herein may be made by any suitable method, including the illustrative methods described herein or those known in the art. Suitable methods include recombinant techniques and proteolytic digestion of whole ABPs.

In some embodiments, the ABPs provided herein are monoclonal ABPs. Monoclonal ABPs may be obtained, for example, using a hybridoma method or using phage or yeast-based libraries.

DNA encoding the monoclonal ABPs may be readily isolated and sequenced using conventional procedures.

In some embodiments, the ABPs provided herein are polyclonal ABPs.

In some embodiments, the ABPs provided herein comprise a chimeric ABP. In some embodiments, the ABPs provided herein consist of a chimeric ABP. In some embodiments, the ABPs provided herein consist essentially of a chimeric ABP. Chimeric ABPs can be made by any methods known in the art. In some embodiments, a chimeric ABP is made by using recombinant techniques to combine a non-human variable region (e.g., a variable region derived from a mouse, rat, hamster, rabbit, or non-human primate, such as a monkey) with a human constant region.

In some embodiments, the ABPs provided herein comprise a humanized ABP. In some embodiments, the ABPs provided herein consist of a humanized ABP. In some embodiments, the ABPs provided herein consist essentially of a humanized ABP. Humanized ABPs may be generated by replacing most, or all, of the structural portions of a non-human monoclonal ABP with corresponding human ABP sequences.

In some embodiments, the ABPs provided herein comprise a human ABP. In some embodiments, the ABPs provided herein consist of a human ABP. In some embodiments, the ABPs provided herein consist essentially of a human ABP. Human ABPs can be generated by a variety of techniques known in the art, for example by using transgenic animals (e.g., humanized mice), can be derived from phage-display libraries, can be generated by in vitro activated B cells, or can be derived from yeast-based libraries

In some embodiments, the ABPs provided herein comprise an alternative scaffold. In some embodiments, the ABPs provided herein consist of an alternative scaffold. In some embodiments, the ABPs provided herein consist essentially of an alternative scaffold. Any suitable alternative scaffold may be used. In some aspects, the alternative scaffold is selected from an Adnectin™, an iMab, an Anticalin®, an EETI-II/AGRP, a Kunitz domain, a thioredoxin peptide aptamer, an Affibody®, a DARPin, an Affilin, a Tetranectin, a Fynomer, and an Avimer. The alternative scaffolds provided herein may be made by any suitable method, including the illustrative methods described herein or those known in the art.

Also disclosed herein is an isolated humanized, human, or chimeric ABP that competes for binding to an HLA-PEPTIDE with an ABP disclosed herein.

Also disclosed herein is an isolated humanized, human, or chimeric ABP that binds an HLA-PEPTIDE epitope bound by an ABP disclosed herein.

In certain aspects, an ABP may comprise a human Fc region comprising at least one modification that reduces binding to a human Fc receptor.

It is known that when an ABP is expressed in cells, the ABP is modified after translation. Examples of the posttranslational modification include cleavage of lysine at the C terminus of the heavy chain by a carboxypeptidase; modification of glutamine or glutamic acid at the N terminus of the heavy chain and the light chain to pyroglutamic acid by pyroglutamylation; glycosylation; oxidation; deamidation; and glycation, and it is known that such posttranslational modifications occur in various ABPs (See Journal of Pharmaceutical Sciences, 2008, Vol. 97, p. 2426-2447, incorporated by reference in its entirety). In some embodiments, an ABP is an ABP or antigen-binding fragment thereof which has undergone posttranslational modification. Examples of an ABP or antigen-binding fragment thereof which have undergone posttranslational modification include an ABP or antigen-binding fragments thereof which have undergone pyroglutamylation at the N terminus of the heavy chain variable region and/or deletion of lysine at the C terminus of the heavy chain. It is known in the art that such posttranslational modification due to pyroglutamylation at the N terminus and deletion of lysine at the C terminus does not have any influence on the activity of the ABP or fragment thereof (Analytical Biochemistry, 2006, Vol. 348, p. 24-39, incorporated by reference in its entirety).

In some embodiments, the ABPs provided herein are multispecific ABPs.

In some embodiments, a multispecific ABP provided herein binds more than one antigen. In some embodiments, a multispecific ABP binds 2 antigens. In some embodiments, a multispecific ABP binds 3 antigens. In some embodiments, a multispecific ABP binds 4 antigens. In some embodiments, a multispecific ABP binds 5 antigens.

In some embodiments, a multispecific ABP provided herein binds more than one epitope on the HLA-PEPTIDE target. In some embodiments, a multispecific ABP binds 2 epitopes on the HLA-PEPTIDE target. In some embodiments, a multispecific ABP binds 3 epitopes on the HLA-PEPTIDE target.

In some embodiments, the multispecific ABP comprises an antigen-binding domain (ABD) that specifically binds to an HLA-PEPTIDE target disclosed herein and an additional ABD that binds to an additional target antigen.

Many multispecific ABP constructs are known in the art, and the ABPs provided herein may be provided in the form of any suitable multispecific construct.

The multispecific ABPs provided herein may be made by any suitable method, including the illustrative methods described herein or those known in the art.

In certain embodiments, an ABP provided herein comprises an Fe region. An Fe region can be wild-type or a variant thereof. In certain embodiments, an ABP provided herein comprises an Fc region with one or more amino acid substitutions, insertions, or deletions in comparison to a naturally occurring Fc region. In some aspects, such substitutions, insertions, or deletions yield ABP with altered stability, glycosylation, or other characteristics. In some aspects, such substitutions, insertions, or deletions yield a glycosylated ABP.

In some embodiments, the Fc region is a variant Fc region. A “variant Fc region” or “engineered Fc region” comprises an amino acid sequence that differs from that of a native-sequence Fc region by virtue of at least one amino acid modification, preferably one or more amino acid substitution(s). Preferably, the variant Fc region has at least one amino acid substitution compared to a native-sequence Fc region or to the Fc region of a parent polypeptide, e.g., from about one to about ten amino acid substitutions, and preferably from about one to about five amino acid substitutions in a native-sequence Fc region or in the Fc region of the parent polypeptide. The variant Fc region herein will preferably possess at least about 80% homology with a native-sequence Fc region and/or with an Fc region of a parent polypeptide, and most preferably at least about 90% homology therewith, more preferably at least about 95% homology therewith.

The term “Fc-region-comprising ABP” refers to an ABP that comprises an Fc region. The C-terminal lysine (residue 447 according to the EU numbering system) of the Fc region may be removed, for example, during purification of the ABP or by recombinant engineering the nucleic acid encoding the ABP. Accordingly, an ABP having an Fc region can comprise an ABP with or without K447.

In some aspects, the Fc region of an ABP provided herein is modified to yield an ABP with altered affinity for an Fc receptor, or an ABP that is more immunologically inert. In some embodiments, the ABP variants provided herein possess some, but not all, effector functions. Such ABPs may be useful, for example, when the half-life of the ABP is important in vivo, but when certain effector functions (e.g., complement activation and ADCC) are unnecessary or deleterious.

In some embodiments, an ABP provided herein comprises one or more alterations that improves or diminishes C1q binding and/or CDC.

In some embodiments, an ABP provided herein comprises one or more alterations to increase half-life. In some embodiments, the ABP comprises one or more non-Fc modifications that extend half-life.

In some embodiments, the multispecific ABP comprises one or more Fc modifications that promote heteromultimerization. In some embodiments, the Fc modification comprises a set of mutations that renders homodimerization electrostatically unfavorable but heterodimerization favorable.

In some embodiments, the Fc modification comprises a modification in the CH3 sequence that affects the ability of the CH3 domain to bind an affinity agent, e.g., Protein A.

Among the provided ABPs, e.g., HLA-PEPTIDE ABPs, are receptors. The receptors can include antigen receptors and other chimeric receptors that specifically bind an HLA-PEPTIDE target disclosed herein. The receptor may be a chimeric antigen receptor (CAR).

Also provided are cells expressing the receptors and uses thereof in adoptive cell therapy, such as treatment of diseases and disorders associated with HLA-PEPTIDE expression, including cancer.

The chimeric receptors, such as CARs, generally include an extracellular antigen binding domain that includes, is, or is comprised within, one of the provided anti-HLA-PEPTIDE ABPs such as anti-HLA-PEPTIDE antibodies. Thus, the chimeric receptors, e.g., CARs, typically include in their extracellular portions one or more HLA-PEPTIDE-ABPs, such as one or more antigen-binding fragment, domain, or portion, or one or more antibody variable domains, and/or antibody molecules, such as those described herein. In some embodiments, the CAR includes a HLA-PEPTIDE-binding portion or portions of the ABP (e.g., antibody) molecule, such as a variable heavy (VH) chain region and/or variable light (VL) chain region of the antibody, e.g., an scFv antibody fragment.

In some embodiments, the CAR is a recombinant CAR.

The recombinant CAR may be a human CAR, comprising fully human sequences, e.g., natural human sequences.

Also provided are cells such as cells that contain an antigen receptor, e.g., that contains an extracellular domain including an anti-HLA-PEPTIDE ABP (e.g., a CAR), described herein. Also provided are populations of such cells, and compositions containing such cells. In some embodiments, compositions or populations are enriched for such cells, such as in which cells expressing the HLA-PEPTIDE ABP make up at least 1, 5, 10, 20, 30, 40, 50, 60, 70, 80, 90, 91, 92, 93, 94, 95, 96, 97, 98, 99, or more than 99 percent of the total cells in the composition or cells of a certain type such as T cells or CD8+ or CD4+ cells. In some embodiments, a composition comprises at least one cell containing an antigen receptor disclosed herein. Among the compositions are pharmaceutical compositions and formulations for administration, such as for adoptive cell therapy. Also provided are therapeutic methods for administering the cells and compositions to subjects, e.g., patients.

Thus also provided are genetically engineered cells expressing an ABP comprising a receptor, e.g., a CAR.

### Nucleotides, Vectors, Host Cells, and Related Methods

Also provided are isolated nucleic acids encoding HLA-PEPTIDE ABPs, vectors comprising the nucleic acids, and host cells comprising the vectors and nucleic acids, as well as recombinant techniques for the production of the ABPs.

The nucleic acids may be recombinant. The recombinant nucleic acids may be constructed outside living cells by joining natural or synthetic nucleic acid segments to nucleic acid molecules that can replicate in a living cell, or replication products thereof. For purposes herein, the replication can be in vitro replication or in vivo replication.

For recombinant production of an ABP, the nucleic acid(s) encoding it may be isolated and inserted into a replicable vector for further cloning (i.e., amplification of the DNA) or expression. In some aspects, the nucleic acid may be produced by homologous recombination, for example as described in U.S. Pat. No. 5,204,244, incorporated by reference in its entirety.

Many different vectors are known in the art. The vector components generally include one or more of the following: a signal sequence, an origin of replication, one or more marker genes, an enhancer element, a promoter, and a transcription termination sequence, for example as described in U.S. Pat. No. 5,534,615, incorporated by reference in its entirety.

Exemplary vectors or constructs suitable for expressing an ABP, e.g., a CAR, antibody, or antigen binding fragment thereof, include, e.g., the pUC series (Fermentas Life Sciences), the pBluescript series (Stratagene, LaJolla, CA), the pET series (Novagen, Madison, WI), the pGEX series (Pharmacia Biotech, Uppsala, Sweden), and the pEX series (Clontech, Palo Alto, CA). Bacteriophage vectors, such as AGTlO, AGTl 1, AZapII (Stratagene), AEMBL4, and ANM1 149, are also suitable for expressing an ABP disclosed herein.

Illustrative examples of suitable host cells are provided below. These host cells are not meant to be limiting, and any suitable host cell may be used to produce the ABPs provided herein.

Suitable host cells include any prokaryotic (e.g., bacterial), lower eukaryotic (e.g., yeast), or higher eukaryotic (e.g., mammalian) cells. Suitable prokaryotes include eubacteria, such as Gram-negative or Gram-positive organisms, for example, Enterobacteriaceae such as Escherichia (E. coli), Enterobacter, Erwinia, Klebsiella, Proteus, Salmonella (S. typhimurium), Serratia (S. marcescans), Shigella, Bacilli (B. subtilis and B. licheniformis), Pseudomonas (P. aeruginosa), and Streptomyces. One useful E. coli cloning host is E. coli 294, although other strains such as E. coli B, E. coli X1776, and E. coli W3110 are also suitable.

In addition to prokaryotes, eukaryotic microbes such as filamentous fungi or yeast are also suitable cloning or expression hosts for HLA-PEPTIDE ABP-encoding vectors. Saccharomyces cerevisiae, or common baker's yeast, is a commonly used lower eukaryotic host microorganism. However, a number of other genera, species, and strains are available and useful, such as Schizosaccharomyces pombe, Kluyveromyces (K. lactis, K fragilis, K bulgaricus K wickeramii, K. waltii, K drosophilarum, K. thermotolerans, and K. marxianus), Yarrowia, Pichia pastoris, Candida (C. albicans), Trichoderma reesia, Neurospora crassa, Schwanniomyces (S. occidentalis), and filamentous fungi such as, for example Penicillium, Tolypocladium, and Aspergillus (A. nidulans and A. niger).

Useful mammalian host cells include COS-7 cells, HEK293 cells; baby hamster kidney (BHK) cells; Chinese hamster ovary (CHO); mouse sertoli cells; African green monkey kidney cells (VERO-76), and the like.

The host cells used to produce the HLA-PEPTIDE ABP may be cultured in a variety of media. Commercially available media such as, for example, Ham's F10, Minimal Essential Medium (MEM), RPMI-1640, and Dulbecco's Modified Eagle's Medium (DMEM) are suitable for culturing the host cells. In addition, any of the media described in Ham et al., Meth. Enz., 1979, 58:44; Barnes et al., Anal. Biochem., 1980, 102:255; and U.S. Pat. Nos. 4,767,704, 4,657,866, 4,927,762, 4,560,655, and 5,122,469; or WO 90/03430 and WO 87/00195 may be used. Each of the foregoing references is incorporated by reference in its entirety.

Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics, trace elements (defined as inorganic compounds usually present at final concentrations in the micromolar range), and glucose or an equivalent energy source. Any other necessary supplements may also be included at appropriate concentrations that would be known to those skilled in the art.

The culture conditions, such as temperature, pH, and the like, are those previously used with the host cell selected for expression, and will be apparent to the ordinarily skilled artisan.

When using recombinant techniques, the ABP can be produced intracellularly, in the periplasmic space, or directly secreted into the medium. If the ABP is produced intracellularly, as a first step, the particulate debris, either host cells or lysed fragments, is removed, for example, by centrifugation or ultrafiltration. For example, Carter et al. (Bio/Technology, 1992, 10:163-167, incorporated by reference in its entirety) describes a procedure for isolating ABPs which are secreted to the periplasmic space of E. coli. Briefly, cell paste is thawed in the presence of sodium acetate (pH 3.5), EDTA, and phenylmethylsulfonylfluoride (PMSF) over about 30 min. Cell debris can be removed by centrifugation.

In some embodiments, the ABP is produced in a cell-free system. In some aspects, the cell-free system is an in vitro transcription and translation system as described in Yin et al., mAbs, 2012, 4:217-225, incorporated by reference in its entirety. In some aspects, the cell-free system utilizes a cell-free extract from a eukaryotic cell or from a prokaryotic cell. In some aspects, the prokaryotic cell is E. coli. Cell-free expression of the ABP may be useful, for example, where the ABP accumulates in a cell as an insoluble aggregate, or where yields from periplasmic expression are low.

Where the ABP is secreted into the medium, supernatants from such expression systems are generally first concentrated using a commercially available protein concentration filter, for example, an Amicon® or Millipore® Pellcon® ultrafiltration unit. A protease inhibitor such as PMSF may be included in any of the foregoing steps to inhibit proteolysis and antibiotics may be included to prevent the growth of adventitious contaminants.

The ABP composition prepared from the cells can be purified using, for example, hydroxylapatite chromatography, gel electrophoresis, dialysis, and affinity chromatography, with affinity chromatography being a particularly useful purification technique. The suitability of protein A as an affinity ligand depends on the species and isotype of any immunoglobulin Fc domain that is present in the ABP. Protein A can be used to purify ABPs that comprise human γ1, γ2, or γ4 heavy chains (Lindmark et al., J. Immunol. Meth., 1983, 62:1-13, incorporated by reference in its entirety). Protein G is useful for all mouse isotypes and for human γ3 (Guss et al., EMBO J., 1986, 5:1567-1575, incorporated by reference in its entirety).

The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the ABP comprises a C_{H3} domain, the BakerBond ABX® resin is useful for purification.

Other techniques for protein purification, such as fractionation on an ion-exchange column, ethanol precipitation, Reverse Phase HPLC, chromatography on silica, chromatography on heparin Sepharose®, chromatofocusing, SDS-PAGE, and ammonium sulfate precipitation are also available, and can be applied by one of skill in the art.

Following any preliminary purification step(s), the mixture comprising the ABP of interest and contaminants may be subjected to low pH hydrophobic interaction chromatography using an elution buffer at a pH between about 2.5 to about 4.5, generally performed at low salt concentrations (e.g., from about 0 to about 0.25 M salt).

### Methods of Identifying ABPs

Identification and/or preparation of an ABP described herein may comprise use of an HLA-PEPTIDE target or off-target HLA-PEPTIDE.

Such antigens may comprise intact HLA-PEPTIDE complexes or fragments thereof. Such antigen may be, for example, in the form of isolated protein or a protein expressed on the surface of a cell.

In some embodiments, the HLA-PEPTIDE antigen is a non-naturally occurring variant of HLA-PEPTIDE, such as a HLA-PEPTIDE protein having an amino acid sequence or post-translational modification that does not occur in nature.

In some embodiments, the HLA-PEPTIDE antigen is truncated by removal of, for example, intracellular or membrane-spanning sequences, or signal sequences. In some embodiments, the HLA-PEPTIDE antigen is fused at its C-terminus to a human IgG1 Fc domain or a polyhistidine tag.

ABPs that bind HLA-PEPTIDE can be identified using any method known in the art, e.g., phage display or immunization of a subject.

One method of identifying an antigen binding protein includes binding a target disclosed herein with an antigen binding protein, contacting the antigen binding protein with one or more off-target HLA-PEPTIDEs disclosed herein, and identifying the antigen binding protein if the antigen binding protein does not bind to the one or more off-target HLA-PEPTIDEs. The antigen binding protein can be present in a library comprising a plurality of distinct antigen binding proteins.

In some embodiments, the library is a phage display library. The phage display library can be developed so that it is substantially free of antigen binding proteins that non-specifically bind the HLA of the HLA-PEPTIDE target. The antigen binding protein can be present in a yeast display library comprising a plurality of distinct antigen binding proteins. The yeast display library can be developed so that it is substantially free of antigen binding proteins that non-specifically bind the HLA of the HLA-PEPTIDE target.

In some embodiments, the library is a yeast display library.

Another method of identifying an antigen binding protein can include obtaining at least one HLA-PEPTIDE target; administering the HLA-PEPTIDE target to a subject (e.g., a mouse, rabbit or a llama), optionally in combination with an adjuvant; and isolating the antigen binding protein from the subject.

In some aspects, isolating the antigen binding protein comprises isolating a B cell from the subject that expresses the antigen binding protein. The B cell can be used to create a hybridoma. The B cell can also be used for cloning one or more of its CDRs. The B cell can also be immortalized, for example, by using EBV transformation. Sequences encoding an antigen binding protein can be cloned from immortalized B cells or can be cloned directly from B cells isolated from an immunized subject. A library that comprises the antigen binding protein of the B cell can also be created, optionally wherein the library is phage display or yeast display.

### Engineered Cells

Also provided are engineered cells such as cells that contain an antigen receptor, e.g., that contains an extracellular domain including an anti-HLA-PEPTIDE ABP (e.g., a CAR), described herein. Also provided are populations of such cells, and compositions containing such cells. In some embodiments, compositions or populations are enriched for such cells, such as in which cells expressing the HLA-PEPTIDE ABP make up at least 1, 5, 10, 20, 30, 40, 50, 60, 70, 80, 90, 91, 92, 93, 94, 95, 96, 97, 98, 99, or more than 99 percent of the total cells in the composition or cells of a certain type such as T cells or CD8+ or CD4+ cells. In some embodiments, a composition comprises at least one cell containing an antigen receptor disclosed herein. Among the compositions are pharmaceutical compositions and formulations for administration, such as for adoptive cell therapy. Also provided are therapeutic methods for administering the cells and compositions to subjects, e.g., patients.

Thus also provided are genetically engineered cells expressing an ABP comprising a receptor, e.g., an anti-HLA-PEPTIDE ABP CAR. The cells generally are eukaryotic cells, such as mammalian cells, and typically are human cells. In some embodiments, the cells are derived from the blood, bone marrow, lymph, or lymphoid organs, are cells of the immune system, such as cells of the innate or adaptive immunity, e.g., myeloid or lymphoid cells, including lymphocytes, typically T cells and/or NK cells. Other exemplary cells include stem cells, such as multipotent and pluripotent stem cells, including induced pluripotent stem cells (iPSCs). The cells typically are primary cells, such as those isolated directly from a subject and/or isolated from a subject and frozen. In some embodiments, the cells include one or more subsets of T cells or other cell types, such as whole T cell populations, CD4+ cells, CD8+ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation. With reference to the subject to be treated, the cells may be allogeneic and/or autologous. Among the methods include off-the-shelf methods. In some aspects, such as for off-the-shelf technologies, the cells are pluripotent and/or multipotent, such as stem cells, such as induced pluripotent stem cells (iPSCs). In some embodiments, the methods include isolating cells from the subject, preparing, processing, culturing, and/or engineering them, as described herein, and re-introducing them into the same patient, before or after cryopreservation.

Among the sub-types and subpopulations of T cells and/or of CD4+ and/or of CD8+ T cells are naive T (TN) cells, effector T cells (TEFF), memory T cells and sub-types thereof, such as stem cell memory T (TSCM), central memory T (TCM), effector memory T (TEM), or terminally differentiated effector memory T cells, tumor-infiltrating lymphocytes (TIL), immature T cells, mature T cells, helper T cells, cytotoxic T cells, mucosa-associated invariant T (MALT) cells, naturally occurring and adaptive regulatory T (Treg) cells, helper T cells, such as TH1 cells, TH2 cells, TH3 cells, TH17 cells, TH9 cells, TH22 cells, follicular helper T cells, alpha/beta T cells, and delta/gamma T cells.

In some embodiments, the cells are natural killer (NK) cells. In some embodiments, the cells are monocytes or granulocytes, e.g., myeloid cells, macrophages, neutrophils, dendritic cells, mast cells, eosinophils, and/or basophils.

The cells may be genetically modified to reduce expression or knock out endogenous TCRs. Such modifications are described in Mol Ther Nucleic Acids. 2012 December; 1(12): e63; Blood. 2011 Aug. 11; 118(6):1495-503; Blood. 2012 Jun. 14; 119(24): 5697-5705; Torikai, Hiroki et al “HLA and TCR Knockout by Zinc Finger Nucleases: Toward “off-the-Shelf” Allogeneic T-Cell Therapy for CD19+ Malignancies.” Blood 116.21 (2010): 3766; Blood. 2018 Jan. 18; 131(3):311-322. doi: 10.1182/blood-2017-05-787598; and WO2016069283, which are incorporated by reference in their entirety.

The cells may be genetically modified to promote cytokine secretion. Such modifications are described in Hsu C, Hughes M S, Zheng Z, Bray R B, Rosenberg S A, Morgan R A. Primary human T lymphocytes engineered with a codon-optimized IL-15 gene resist cytokine withdrawal-induced apoptosis and persist long-term in the absence of exogenous cytokine. J Immunol. 2005; 175:7226-34; Quintarelli C, Vera J F, Savoldo B, Giordano Attianese G M, Pule M, Foster A E, Co-expression of cytokine and suicide genes to enhance the activity and safety of tumor-specific cytotoxic T lymphocytes. Blood. 2007; 110:2793-802; and Hsu C, Jones S A, Cohen C J, Zheng Z, Kerstann K, Zhou J, Cytokine-independent growth and clonal expansion of a primary human CD8+ T-cell clone following retroviral transduction with the IL-15 gene. Blood. 2007; 109:5168-77.

Mismatching of chemokine receptors on T cells and tumor-secreted chemokines has been shown to account for the suboptimal trafficking of T cells into the tumor microenvironment. To improve efficacy of therapy, the cells may be genetically modified to increase recognition of chemokines in tumor micro environment. Examples of such modifications are described in Moon et al., Expression of a functional CCR2 receptor enhances tumor localization and tumor eradication by retargeted human T cells expressing a mesothelin-specific chimeric antibody receptor. Clin Cancer Res. 2011; 17: 4719-4730; and Craddock et al., Enhanced tumor trafficking of GD2 chimeric antigen receptor T cells by expression of the chemokine receptor CCR2b. J Immunother. 2010; 33: 780-788.

The cells may be genetically modified to enhance expression of costimulatory/enhancing receptors, such as CD28 and 41BB.

Adverse effects of T cell therapy can include cytokine release syndrome and prolonged B-cell depletion. Introduction of a suicide/safety switch in the recipient cells may improve the safety profile of a cell-based therapy. Accordingly, the cells may be genetically modified to include a suicide/safety switch. The suicide/safety switch may be a gene that confers sensitivity to an agent, e.g., a drug, upon the cell in which the gene is expressed, and which causes the cell to die when the cell is contacted with or exposed to the agent. Exemplary suicide/safety switches are described in Protein Cell. 2017 August; 8(8): 573-589. The suicide/safety switch may be HSV-TK. The suicide/safety switch may be cytosine deaminase, purine nucleoside phosphorylase, or nitroreductase. The suicide/safety switch may be RapaCIDe™, described in U.S. Patent Application Pub. No. US20170166877A1. The suicide/safety switch system may be CD20/Rituximab, described in Haematologica. 2009 September; 94(9): 1316-1320. These references are incorporated by reference in their entirety.

The anti-HLA-PEPTIDE ABP CAR may be introduced into the recipient cell as a split receptor which assembles only in the presence of a heterodimerizing small molecule. Such systems are described in Science. 2015 Oct. 16; 350(6258): aab4077, and in U.S. Pat. No. 9,587,020, which are hereby incorporated by reference in its entirety.

In some embodiments, the cells include one or more nucleic acids, e.g., a polynucleotide encoding an anti-HLA-PEPTIDE ABP CAR disclosed herein, wherein the polynucleotide is introduced via genetic engineering, and thereby express recombinant or genetically engineered receptors, e.g., anti-HLA-PEPTIDE ABP CARs, as disclosed herein. In some embodiments, the nucleic acids are heterologous, i.e., normally not present in a cell or sample obtained from the cell, such as one obtained from another organism or cell, which for example, is not ordinarily found in the cell being engineered and/or an organism from which such cell is derived. In some embodiments, the nucleic acids are not naturally occurring, such as a nucleic acid not found in nature, including one comprising chimeric combinations of nucleic acids encoding various domains from multiple different cell types.

The nucleic acids may include a codon-optimized nucleotide sequence. Without being bound to a particular theory or mechanism, it is believed that codon optimization of the nucleotide sequence increases the translation efficiency of the mRNA transcripts. Codon optimization of the nucleotide sequence may involve substituting a native codon for another codon that encodes the same amino acid, but can be translated by tRNA that is more readily available within a cell, thus increasing translation efficiency. Optimization of the nucleotide sequence may also reduce secondary mRNA structures that would interfere with translation, thus increasing translation efficiency.

Methods for Engineering Cells with ABPs

Also provided are methods, nucleic acids, compositions, and kits, for expressing the ABPs, including receptors comprising antibodies, and anti-HLA-PEPTIDE ABP CARs, and for producing genetically engineered cells expressing such ABPs. The genetic engineering generally involves introduction of a nucleic acid encoding the recombinant or engineered component into the cell, such as by retroviral transduction, transfection, or transformation.

### Preparation of Engineered Cells

In some embodiments, preparation of the engineered cells includes one or more culture and/or preparation steps. The cells for introduction of the HLA-PEPTIDE-ABP, e.g., CAR, can be isolated from a sample, such as a biological sample, e.g., one obtained from or derived from a subject. In some embodiments, the subject from which the cell is isolated is one having the disease or condition or in need of a cell therapy or to which cell therapy will be administered. The subject in some embodiments is a human in need of a particular therapeutic intervention, such as the adoptive cell therapy for which cells are being isolated, processed, and/or engineered.

In some embodiments, the cells are incubated and/or cultured prior to or in connection with genetic engineering.

### Assays

A variety of assays known in the art may be used to identify and characterize an HLA-PEPTIDE ABP provided herein.

### Pharmaceutical Compositions

An ABP, cell, or HLA-PEPTIDE target provided herein can be formulated in any appropriate pharmaceutical composition and administered by any suitable route of administration. Suitable routes of administration include, but are not limited to, the intra-arterial, intradermal, intramuscular, intraperitoneal, intravenous, nasal, parenteral, pulmonary, and subcutaneous routes. These compositions can comprise, in addition to one or more of the antibodies disclosed herein, a pharmaceutically acceptable excipient, carrier, buffer, stabilizer or other materials well known to those skilled in the art. Such materials should be non-toxic and should not interfere with the efficacy of the active ingredient. The precise nature of the carrier or other material can depend on the route of administration, e.g, oral, intravenous, cutaneous or subcutaneous, nasal, intramuscular, intraperitoneal routes.

### Therapeutic Applications

For therapeutic applications, ABPs and/or cells are administered to a mammal, generally a human, in a pharmaceutically acceptable dosage form such as those known in the art and those discussed above. For example, ABPs and/or cells may be administered to a human intravenously as a bolus or by continuous infusion over a period of time, by intramuscular, intraperitoneal, intra-cerebrospinal, subcutaneous, intra-articular, intrasynovial, intrathecal, or intratumoral routes. The ABPs also are suitably administered by peritumoral, intralesional, or perilesional routes, to exert local as well as systemic therapeutic effects. The intraperitoneal route may be particularly useful, for example, in the treatment of ovarian tumors.

The ABPs and/or cells provided herein can be useful for the treatment of any disease or condition involving HLA-PEPTIDE. In some embodiments, the disease or condition is a disease or condition that can benefit from treatment with an anti-HLA-PEPTIDE ABP and/or cell. In some embodiments, the disease or condition is a tumor. In some embodiments, the disease or condition is a cell proliferative disorder. In some embodiments, the disease or condition is a cancer.

In some embodiments, the ABPs and/or cells provided herein are provided for use as a medicament. In some embodiments, the ABPs and/or cells provided herein are provided for use in the manufacture or preparation of a medicament. In some embodiments, the medicament is for the treatment of a disease or condition that can benefit from an anti-HLA-PEPTIDE ABP and/or cell. In some embodiments, the disease or condition is a tumor. In some embodiments, the disease or condition is a cell proliferative disorder. In some embodiments, the disease or condition is a cancer.

In some embodiments, provided herein is a method of treating a disease or condition in a subject in need thereof by administering an effective amount of an ABP and/or cell provided herein to the subject. In some aspects, the disease or condition is a cancer.

In some embodiments, provided herein is a method of treating a disease or condition in a subject in need thereof by administering an effective amount of an ABP and/or cell provided herein to the subject, wherein the disease or condition is a cancer, and the cancer is selected from a solid tumor and a hematological tumor. In some embodiments, the cancer is selected from any one of the tumor types shown in FIG. 1B. In some embodiments, the cancer is selected from

In some embodiments, provided herein is a method of modulating an immune response in a subject in need thereof, comprising administering to the subject an effective amount of an ABP and/or cell or a pharmaceutical composition disclosed herein. In some embodiments, the modulating of the immune response comprises increasing the immune response. Stimulating the immune response may comprise stimulating an immune response or enhancing an immune response.

In some embodiments of any one of the methods described herein, the presence of an HLA-PEPTIDE target described herein has been detected in the subject or a biological sample obtained from the subject. In some embodiments of any one of the methods described herein, the presence of a restricted peptide of an HLA-PEPTIDE target described herein has been detected in the subject or a biological sample obtained from the subject. In some embodiments of any one of the methods described herein, the presence of the HLA subtype of an HLA-PEPTIDE target described herein has been detected in the subject or a biological sample obtained from the subject. In some embodiments, the method comprises administering an ABP disclosed herein to the subject after having determined the presence of the HLA-PEPTIDE target, restricted peptide, or HLA in the biological sample obtained from the subject.

In some embodiments, contacting the ABP with cancer cells results in at least about 10%, 20%, 30%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% cytotoxicity. In some embodiments, a concentration of 0.1 nM, 1 nM, 5 nM or 10 nM of the ABP is sufficient to result in that cytotoxicity upon contacting the ABP with the a cancer cell that expresses a tumor antigen (e.g., HLA-peptide) and an effector cell.

In some embodiments, the cytotoxicity resulting from contacting an ABP of the present disclosure with a cancer cell is greater than from contacting a cancer cell with a reference ABP.

In some embodiments, the cancer cells have an A*01:01_NTDNNLAVY (SEQ ID NO: 214) copy numbers of less than about 300, about 350, about 400, about 450, about 500, about 550, about 600, about 650, about 700, about 750, about 800, about 850, about 900, about 950, about 1000, about 1500, about 2000, about 2500, about 3000, about 3500, about 4000, about 4500, about 5000, about 5500, about 6000, about 6500, about 7000, about 7500, about 8000, about 8500, about 9000, about 9500, about 10000, about 20000, about 30000, and about 40000 copies/cell.

In some embodiments, the cancer cells have an A*01:01_NTDNNLAVY (SEQ ID NO: 214) copy numbers of less than about 300, about 350, about 400, about 450, about 500, about 550, about 600, about 650, about 700, about 750, about 800, about 850, about 900, about 950, about 1000, about 1500, about 2000, about 2500, about 3000, about 3500, about 4000, about 4500, about 5000, about 5500, about 6000, about 6500, about 7000, about 7500, about 8000, about 8500, about 9000, about 9500, about 10000, about 20000, about 30000, and about 40000 copies/cell.

### Diagnostic Methods

Also provided are methods for predicting and/or detecting the presence of a given HLA-PEPTIDE on a cell from a subject. Such methods may be used, for example, to predict and evaluate responsiveness to treatment with an ABP and/or cell provided herein.

In some embodiments, a blood or tumor sample is obtained from a subject and the fraction of cells expressing HLA-PEPTIDE is determined. In some aspects, the relative amount of HLA-PEPTIDE expressed by such cells is determined. The fraction of cells expressing HLA-PEPTIDE and the relative amount of HLA-PEPTIDE expressed by such cells can be determined by any suitable method. In some embodiments, flow cytometry is used to make such measurements. In some embodiments, fluorescence assisted cell sorting (FACS) is used to make such measurement. See L1 et al., J. Autoimmunity, 2003, 21:83-92 for methods of evaluating expression of HLA-PEPTIDE in peripheral blood.

In some embodiments, detecting the presence of a given HLA-PEPTIDE on a cell from a subject is performed using immunoprecipitation and mass spectrometry. This can be performed by obtaining a tumor sample (e.g., a frozen tumor sample) such as a primary tumor specimen and applying immunoprecipitation to isolate one or more peptides. The HLA alleles of the tumor sample can be determined experimentally or obtained from a third party source. The one or more peptides can be subjected to mass spectrometry (MS) to determine their sequence(s). The spectra from the MS can then be searched against a database. An example is provided in the Examples section below.

In some embodiments, predicting the presence of a given HLA-PEPTIDE on a cell from a subject is performed using a computer-based model applied to the peptide sequence and/or RNA measurements of one or more genes comprising that peptide sequence (e.g., RNA seq or RT-PCR, or nanostring) from a tumor sample. The model used can be as described in international patent application no. PCT/US2016/067159, herein incorporated by reference, in its entirety, for all purposes.

### Kits

Also provided are kits comprising an ABP and/or cell provided herein. The kits may be used for the treatment, prevention, and/or diagnosis of a disease or disorder, as described herein.

In some embodiments, the kit comprises a container and a label or package insert on or associated with the container. Suitable containers include, for example, bottles, vials, syringes, and IV solution bags. The containers may be formed from a variety of materials, such as glass or plastic. The container holds a composition that is by itself, or when combined with another composition, effective for treating, preventing and/or diagnosing a disease or disorder. The container may have a sterile access port. For example, if the container is an intravenous solution bag or a vial, it may have a port that can be pierced by a needle. At least one active agent in the composition is an ABP provided herein. The label or package insert indicates that the composition is used for treating the selected condition.

In some embodiments, the kit comprises (a) a first container with a first composition contained therein, wherein the first composition comprises an ABP and/or cell provided herein; and (b) a second container with a second composition contained therein, wherein the second composition comprises a further therapeutic agent. The kit in this embodiment can further comprise a package insert indicating that the compositions can be used to treat a particular condition, e.g., cancer.

Alternatively, or additionally, the kit may further comprise a second (or third) container comprising a pharmaceutically-acceptable excipient. In some aspects, the excipient is a buffer. The kit may further include other materials desirable from a commercial and user standpoint, including filters, needles, and syringes.

### EXAMPLES

Below are examples of specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

The practice of the present invention will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W.H. Freeman and Company, 1993); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.); Remington's Pharmaceutical Sciences, 18th Edition (Easton, Pennsylvania: Mack Publishing Company, 1990); Carey and Sundberg Advanced Organic Chemistry 3^{rd} Ed. (Plenum Press) Vols A and B (1992).

### Example 1: Determining Expression Levels of CT83 in Various Tumor Types

CT83 (also referred to as KKLC1) is a cancer testis antigen that is highly expressed in several cancer types. As shown in FIG. 1A and Table 1 there are low (and occasionally negligible) expression levels of CT83 in several normal tissues, with the exception of normal testis tissue.

TABLE 1
Expression levels of CT83 in normal tissue

| Normal Tissue | Median TPM | 95%—tile TPM |
|---|---|---|
| Brain (all GTEx areas) | 0 | 0.08 |
| Adipose | 0 | 0.11 |
| Adrenal Gland | 0 | 0.10 |
| Artery—Aorta | 0 | 0.10 |
| Artery—Coronary | 0 | 0.10 |
| Bladder | 0 | 0.32 |
| Breast—Mammary Tissue | 0 | 0.34 |
| Cervix—Ectocervix | 0.06 | 0.14 |
| Cervix—Endocervix | 0 | 0.13 |
| Colon | 0 | 0.12 |
| Esophagus—Gastroesophageal Junction | 0 | 0.00 |
| Esophagus—Mucosa | 0 | 0.12 |
| Esophagus—Muscularis | 0 | 0.11 |
| Fallopian Tube | 0 | 0.16 |
| Heart—Atrial Appendage | 0 | 0.06 |
| Heart—Left Ventricle | 0 | 0.00 |
| Kidney—Cortex | 0 | 0.15 |
| Liver | 0 | 0.02 |
| Lung | 0 | 0.32 |
| Minor Salivary Gland | 0.66 | 6.11 |
| Muscle—Skeletal | 0 | 0.08 |
| Nerve—Tibial | 0 | 0.11 |
| Ovary | 0 | 0.12 |
| Pancreas | 0 | 0.10 |
| Pituitary | 0 | 0.13 |
| Prostate | 0 | 0.18 |
| Skin | 0 | 0.12 |
| Small Intestine—Terminal Ileum | 0 | 0.12 |
| Spleen | 0 | 0.00 |
| Stomach | 0 | 0.12 |
| Testis | 74.54 | 163.97 |
| Thyroid | 0 | 0.26 |
| Uterus | 0 | 0.11 |
| Vagina | 0 | 0.24 |
| Whole Blood | 0 | 0.00 |

In contrast, as shown in FIG. 1B, CT83 is highly expressed across several type of tumor tissues, and of the tumor tissue types shown in FIG. 1B, there is an average CT83 transcript per million (TPM) of over 30 TPM.

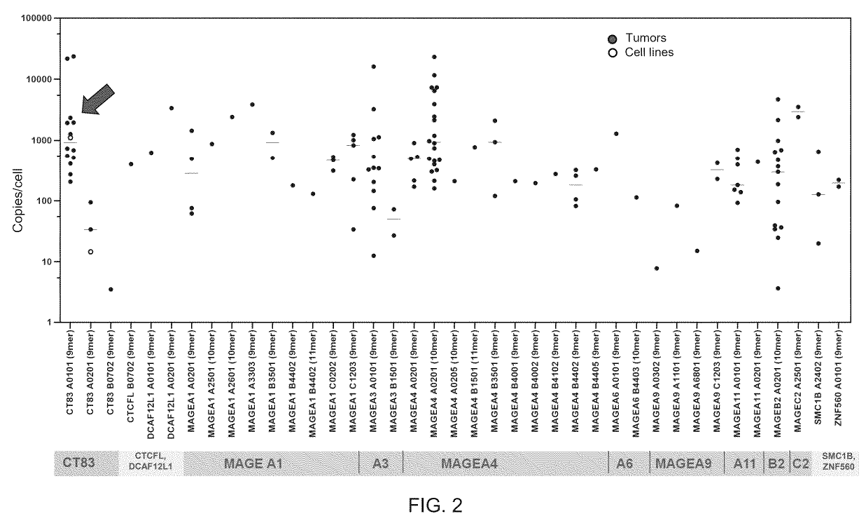

Mass spectrometry was used to determine the copies/cell of various targets within the tumor antigens indicated in FIG. 2. Due to a heavy standard deviation observed at end of the mass spectrometry process, copy numbers were recalculated assuming 20% generic recovery. The results showed that the HLA-PEPTIDE target A*01:01_NTDNNLAVY (SEQ ID NO: 214) (also referred to as G2) was detected in multiple tumor samples at a high median copy number (FIG. 2, arrow). Table 2 shows the mean and median copy numbers measured using mass spectrometry (n=14 tumors) as well as the prevalence of top tumor indications within the United States of America. The mean and median copy numbers reported in Table 2 are corrected copies per cell based on median G2 process recovery of 6% in n=18 tumor/tumor cell lines.

TABLE 2
The calculated copy numbers for A*01:01_ NTDNNLAVY
(SEQ ID NO: 214) target and top tumor indications
within which CT83 is highly expressed.

| | | | | | Preva- |
|---|---|---|---|---|---|
| | | | Calculated | | lence |
| | HLA | Gene | Copies/Cell | Top Tumor indications | (US) |
| G2 | A*01:01 | CT83 | 13000 mean | Esophageal | 10% |
| | | | 3000 median | Gastric Adenocarcinoma | 11% |
| | | | | Lung Adenocarcinoma | 9% |
| | | | | Lung squamous | 5% |

These results indicate that the G2 target is highly expressed in multiple prevalent cancer types and has a high copy number in these prevalent tumor indications.

### Example 2: Identification of Antigen-Binding Proteins (ABPs) that Selectively Bind HLA-PEPTIDE Target A*01:01 NTDNNLAVY (SEQ ID NO: 214) Over Identified G2 Off-Target Liability Peptides

To identify lead target sequences against HLA-PEPTIDE target A*01:01_NTDNNLAVY (SEQ ID NO: 214) (G2), two lead molecules: 2H03 (D5) and 2F10 (C11), were selected based on their specificity, affinity, and potency (see, e.g., WO2021168355A1, published Aug. 26, 2021, hereby incorporated by reference in its entirety). To improve the potency, an affinity maturation campaign was carried out on both of these molecules. For the affinity maturation library design, the CDR3 regions of the heavy chains (VH) were randomized to include up to 3 mutations at a time while, CDR1 and CDR2 regions of the heavy chains included single mutations. For the C11_VH library, CDR3 region was randomized using 17 of 20 amino acids (except, Cysteine, Methionine, and Proline). D5_VH affinity maturation library was designed using a tailored in vitro affinity maturation (TiAM) approach.

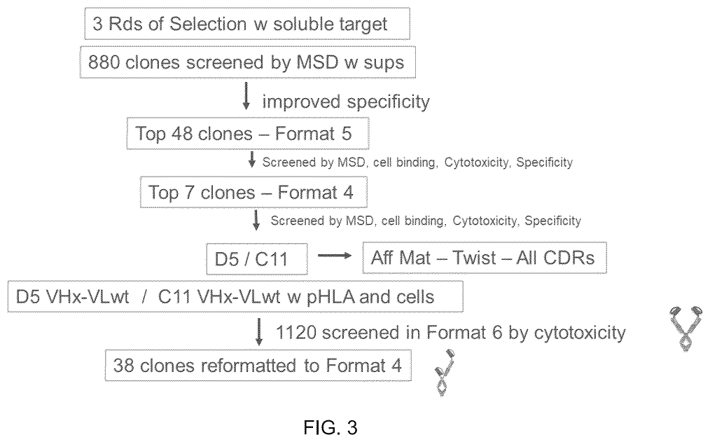

As shown in FIG. 3, through affinity maturation of D5 and C11 and cytotoxicity screening of the randomly mutated clones in Format 6, 38 clones were identified as potential leads and for screening in Format 4.

### Construction of Affinity Maturation Libraries

Affinity maturation libraries were synthesized at Twist Biosciences and delivered as double stranded V-gene fragments. To clone these libraries into pADL-23c phagemid vector, gene fragments were PCR amplified using forward (5′-CTCGCGGCCCAGCCGGCCATGG-3′ (SEQ ID NO: 229)) and reverse (5′-GTTGGCCTCCCGGGCCACTAGTTTTGATCTC-3′ (SEQ ID NO: 230)) primers. PCR was carried out using Phusion high-fidelity PCR master mix with GC buffer (NEB) using 400 ng of template in 750 μl volume and cycled 25 times at 98° C.-10 sec, 70° C.-3, 72° C.-30 sec. Next, the PCR products as well as pADL-23c phagemid vector were digested using BglI restriction enzyme (NEB) for 4 hours at 37° C. and purified using gel extraction kit (Macherey-Nagel). The digested insert and vector were ligated using T4 DNA ligase (NEB) overnight at 16° C. Ligation reactions were purified using PCR clean-up kit (Macherey-Nagel) before electroporation into TG1 electrocompetent E. coli cells (Lucigen). After electroporation, the library was pooled and plated on 2YT media with 100 μg/mL carbenicillin and 2% glucose (2YTCG) agar plates and incubated overnight at 30° C. Serial dilution plates were also generated to determine the electroporation efficiency and to calculate the library size. Following, overnight incubation, plates were scrapped into 2YTCG media, aliquoted, and stored at −80° C.

### Packaging of Affinity Maturation Libraries Employing CM13K-Trypsin Sensitive Helper Phage

Aliquots of the libraries were thawed and 500 ml of 2YTCG cultures were inoculated with the initial OD₆₀₀=0.1. Cultures were grown at 37° C., 250 rpm until OD₆₀₀ reached 0.5. At that stage, CM13K helper phage were added to the cultures to the multiplicity of infection (MOI) of 10 and incubated at 37° C. without shaking for 30 minutes followed by 30 minutes with vigorous shaking. Cultures were centrifuged and pellets were resuspended in 2YT media with 100 μg/mL carbenicillin and 50 μg/mL kanamycin (2YTCK) medium and incubated at 25° C. overnight. On the following day, cultures were centrifuged and ¼ volume of PEG/NaCl solution was added to the supernatant to precipitate phage particles. After 4 hours of incubation on ice, the mixtures were centrifuged and white pellets containing phage particles were collected and resuspended in PBS. 250 μL of PEG/NaCl was added to the phage solution and incubated on ice for 30 minutes, followed by centrifugation at 18,000×g for 10 minutes. Supernatant was discarded and phage pellets were resuspended in PBS.

### Panning of Affinity Maturation Libraries

For each of the affinity maturation libraries, three parallel panning campaigns were carried out. These were a) soluble-soluble panning, b) soluble-cell panning, c) cell-cell panning.

### Soluble-Soluble Panning:

For the first-round panning, libraries (1.0E+11) were depleted against 100 nM of a pool of five negative control pHLAs (Table 3) for 1 hour at room temperature with rotation. pHLA bound phage were pulled down using Dynabead M-280 streptavidin beads (Life Technologies), and the supernatant (depleted library) was collected for selections.

TABLE 3
List of G2 target and negative control peptides
presented on A*0101 pHLA complex.

| Peptide | Peptide Sequence | SEQ ID NO: |
|---|---|---|
| Target peptide | NTDNNLAVY | 214 |
| Control peptide-1 | YTDNWLAVY | 152 |
| Control peptide-2 | GTDNWLAQY | 203 |
| Control peptide-3 | ETDNNIVVY | 204 |
| Control peptide-4 | PTDENLARY | 205 |
| Control peptide-5 | NTDNLLTEY | 206 |

For selections, the depleted library was incubated with 100 nM of target pHLA complex for 1 hour at room temperature with rotation. Dynabead M-280 streptavidin beads (Life Technologies), blocked in 3% milk/PBS for one hour, were added to the mixture and incubated for one additional hour with rotation. Beads were collected using magnetic separator and any non-specific phages were removed by washing the beads five times with PBS with 0.05% tween-20 (PBST) followed by five additional washes with PBS. To elute the bound phages from the washed beads, 100 μl of freshly prepared 100 μg/mL TPCK-trypsin (NEB) was added and incubated for 15 minutes at room temperature. The eluted phages were then used to infect log growth TG-1 cells (OD₆₀₀=0.5) and after an hour of incubation at 37° C., cells were plated onto 2YTCG agar plates for output titer and bacterial growth for subsequent panning rounds. The next day, cells were scrapped and fresh 5 ml 2YTCG culture was started. Once OD₆₀₀ reached 0.5, phage particles were rescued using CM13K helper phage to a MOI of 10. For the second round of soluble-soluble panning, the process was repeated with lower target pHLA concentration, 50 nM.

### Soluble-Cell Panning:

For the first-round of soluble panning, libraries (1.0E+11) were depleted against 100 nM of against a pool of five negative control pHLAs (Table 3) for 1 hour at room temperature with rotation. pHLA bound phage were pulled down using Dynabead M-280 streptavidin beads (Life Technologies), and the supernatant (depleted library) was collected for selections. The depleted library was incubated with 100 nM of target pHLA complex for 1 hour at room temperature with rotation. Dynabead M-280 streptavidin beads (Life Technologies), blocked in 3% milk/PBS for one hour, were added to the mixture and incubated for one additional hour with rotation. Beads were collected using magnetic separator and any non-specific phages were removed by washing the beads five times with PBST followed by five additional washes with PBS. To elute the bound phages from the washed beads, 100 μl of freshly prepared 100 μg/mL TPCK-trypsin (NEB) was added and incubated for 15 minutes at room temperature. The eluted phages were then used to infect log growth TG-1 cells (OD₆₀₀=0.5) and after an hour of incubation at 37° C., cells were plated onto 2YTCG agar plates for output titer and bacterial growth for subsequent panning rounds. Next day, cells were scrapped and fresh 5 ml 2YTCG culture was started. Once OD₆₀₀ reached 0.5, phages were rescued using CM13K helper phage to a MOI of 10.

For the second round, cell panning was carried out using A375 cells overexpressing G2 target peptide. Approximately, 0.5×10⁷ of K562 cells, pulsed with 5 control peptides (Table 3) and 0.5×10⁷ of A375-Luciferase cells were incubated with ˜10¹¹ phage particles from the first round output of soluble panning for 1 hour at 4° C. to deplete the library against cell background. The phage/cell suspension was centrifuged at 500×g for 5 minutes, and the supernatant containing non-bound phage was incubated with 1×10⁷ of A375 cells overexpressing G2 target for 1 hour at 4° C. The cells were then washed three times with pH 5 buffer (PBS with pH lowered to 5.0 using citric acid), followed by three washes with PBS, pH 7.4. To elute cell bound phage, cells were incubated with 100 μl of freshly prepared 100 μg/mL TPCK-trypsin (NEB) for 15 minutes at room temperature, followed by centrifugation at 500×g for 5 minutes. The phage elute was used to infect 2YCG culture at OD₆₀₀ 0.5 for 1 hour followed by plating cells on onto 2YTCG agar plates.

### Cell-Cell Panning:

For the first round for cell panning, approximately, 1.0×10⁷ of A375-Luciferase cells were incubated with ˜10¹¹ phage particles for 1 hour at 4° C. to deplete the library against cell background. The phage/cell suspension was centrifuged at 500×g for 5 minutes, then the supernatant containing non-bound phage was incubated with 1×10⁷ of A375 cells overexpressing G2 target for 1 hour at 4° C. The cells were then washed three times with pH 5 buffer (PBS with pH lowered to 5.0 using citric acid), followed by three washes with PBS, pH 7.4. To elute cell bound phage, cells were incubated with 100 μl of freshly prepared 100 μg/mL TPCK-trypsin (NEB) for 15 minutes at room temperature, followed by centrifugation at 500×g for 5 minutes. The phage elute was used to infect 2YCG culture at OD₆₀₀ 0.5 for 1 hour followed by plating cells on onto 2YTCG agar plates.

For the second round, approximately, 0.5×10⁷ of K562 cells, pulsed with 5 control peptide (Table 3) and 0.5×10⁷ of A375-Luciferase cells were incubated with ˜10¹¹ phage particles from the first round output for 1 hour at 4° C. to deplete the library against cell background. The phage/cell suspension was centrifuged at 500×g for 5 minutes, then the supernatant containing non-bound phage was incubated with 1×10⁷ of A375 cells overexpressing G2 target for 1 hour at 4° C. The cells were then washed three times with pH 5 buffer (PBS with pH lowered to 5.0 using citric acid), followed by 3 washes with PBS, pH 7.4. To elute cell bound phage, cells were incubated with 100 μl of freshly prepared 100 μg/mL TPCK-trypsin (NEB) for 15 minutes at room temperature, followed by centrifugation at 500×g for 5 minutes. The phage elute was used to infect 2YCG culture at OD₆₀₀ 0.5 for 1 hour followed by plating cells on onto 2YTCG agar plates.

### Example 3: Generation of Bispecific Antibodies that Specifically Bind an HLA-PEPTIDE Target and CD3

Antigen binding domains specific for various combinations of distinct targets were formatted into six bispecific construct designs (also referred to herein as formats). See International Application No. PCT/US2020/15736, which is hereby incorporated by reference in its entirety. For clarity, for designs (formats) #2-#6, the antigen binding domains are attached, directly or indirectly, to an Fc region. Format #3, #4, and #5 optionally comprise knob-hole or other Fc heterodimerization modification(s). Format #2 and #6 optionally comprise WT IgG1 Fc or IgG1 FC with TM mutations (P331S, L234E, L235F) sequences without knob-hole modification(s). In some embodiments, target 1 is the HLA-PEPTIDE target and target 2 is a cell surface molecule present on a T cell or NK cell. In some embodiments, target 2 is CD3. The antigen binding domain specific for CD3 can comprise CDRs or variable regions from any anti-CD3 antibody or antigen binding fragment thereof. In some embodiments, target 2 is CD16. It is contemplated that target 1 is A*01:01_NTDNNLAVY (SEQ ID NO: 214) (G2).

Briefly, bispecific antibodies were generated using standard molecular cloning techniques, including restriction digestion and ligation, gene synthesis, and homology-based cloning methods such as In-fusion (Takara). Positive clones were confirmed by DNA sequencing and used to generate bispecific antibody molecules by transfecting Expi-293 cells (Thermo) according to the manufacturer's protocol. Cultures were harvested and bispecific antibodies were purified from the supernatants using protein A, Kappa-select, or IMAC (GE healthcare) based chromatography methods. If necessary, bispecific antibodies or controls were polished by SEC or mixed-mode (CHT, BIO-RAD) chromatography. Molecules were formulated in PBS by dialysis or desalting chromatography. Molecules were evaluated to confirm high monomer purity (>95%) and low endotoxin (≤1 EU/mg) prior to subsequent testing.

Examples of Formats 4, 5, and 6 are shown in FIGS. 4A and 4B. In the screening procedures for a lead anti-CT83 antibody (as outlined in FIG. 3), the first and second ABRs were the G2-targeted clones and the Fab portions were specific for CD3.

### Example 4: Modification to Stabilize Conformation of Format 4 Antibodies that Specifically Bind G2 Target and CD3

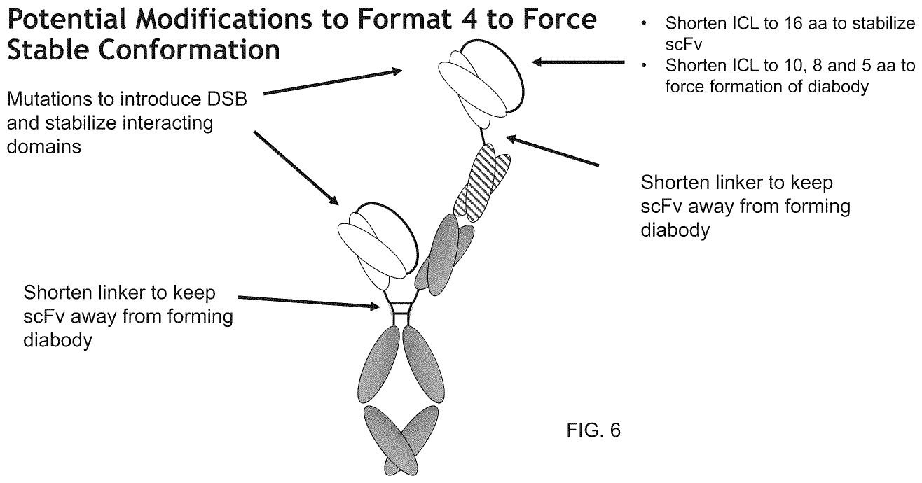

Format 4 antibodies (as shown in FIGS. 4A-5) can exist in two conformations. In solution, Format 4 antibody will be in equilibrium between (1) a dual scFv conformation and (2) a diabody conformation (see FIG. 5). The purpose of this experiment was to identify modifications to the Format 4 antibody that would stabilize the antibody in one conformation when in solution. Groups of Format 4 antibodies targeting G2-pHLA (C11, D5, 52C11, and 31E07 (also referred to as “E07” and affinity matured from D5); see Tables 19 and 20) were generated, each with one type of potential modification for Format-4 stability, as shown in FIG. 6. Examples of the groups of antibodies tested are shown in Table 4. The antibodies were evaluated for stability using size exclusion chromatography (SEC).

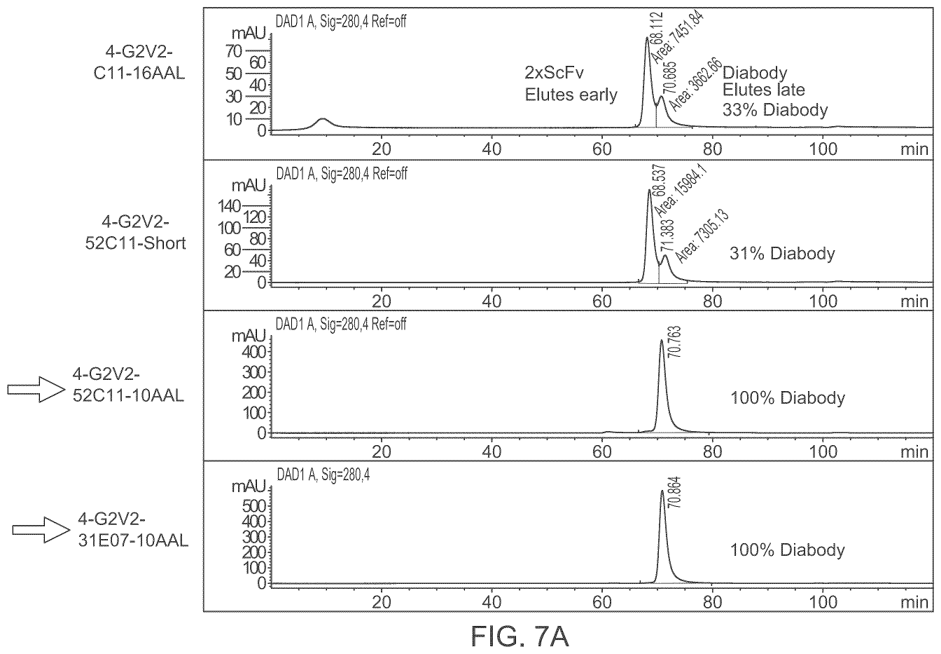

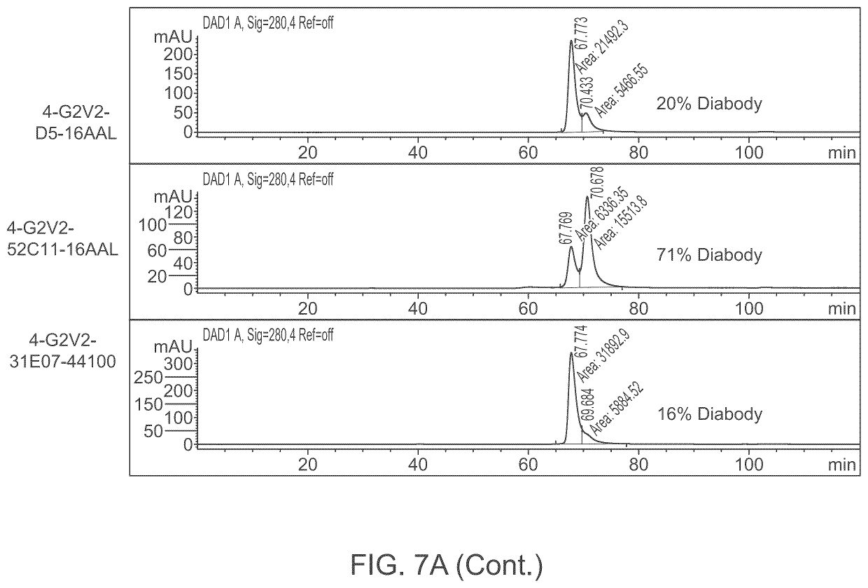

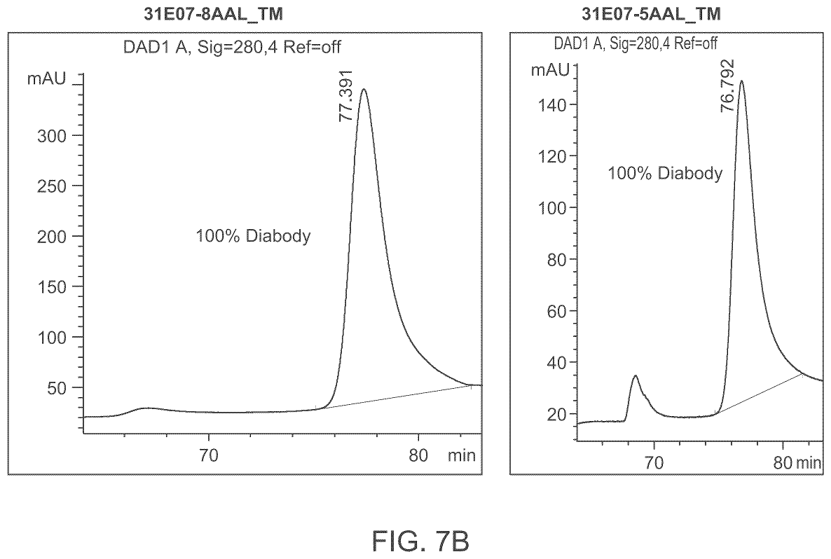

TABLE 4
Modifications tested for stability in Format-4 antibodies.

| Format 4 Antibody | Anti-pHLA | |
|---|---|---|
| (see FIGS. 7A-7B) | Clone | Modification for Stability |
| 4-G2V2-C11-16AAL | C11 | Shortened ICL; L1 = L2 = 16aa* |
| 4-G2V2-52-C11-Short | C11 | Shortened L3 and L4; L3 = GSGGGG-CP |
| | | (SEQ ID NO: 237) and L4 = GSGGGG |
| | | (SEQ ID NO: 231) |
| 4-G2V2-52-C11-10AAL | C11 | Shortened ICL; L1 = L2 = 10aa |
| 4-G2V2-31-E07-10AAL | 31E07 | Shortened ICL; L1 = L2 = 10aa |
| 4-G2V2-D5-16AAL | D5 | Shortened ICL; L1 = L2 = 16aa |
| 4-G2V2-52-C11-16AAL | C11 | Shortened ICL; L1 = L2 = 16aa |
| 4-G2V2-31-E07-44100 | 31E07 | Disulfide bond; cysteines at position 44 |
| | | in the VH and position 100 in the VL |
| | | (Kabat) |
| 31-E07-8AAL_TM | 31E07 | Shortened ICL; L1 = L2 = 8aa |
| 31-E07-5AAL_TM | 31E07 | Shortened ICL; L1 = L2 = 5aa |

*aa = amino acids

### Size Exclusion Chromatography

Purified bispecific antibody samples (50 ug) were filtered using 0.22 um centrifugal filters (VWR P/N 82031-348) and spun at 4000 g for 1 minute to remove any large particulates. Approximately 40 μg of the filtered samples were then loaded on the Agilent HPLC-SEC with TSKgel G3000SWxl column (Tosoh P/N 08541) with TSK guard column (Tosoh P/N 08543). An isocratic method using PBS with Calcium and Magnesium (Corning P/N 21-030-CM) as mobile phase was ran over 120 minutes with a flow rate of 0.125 ml/min to ensure good separation of 2×ScFv and diabody species. Responses were detected and recorded using A280 wavelength. The peaks on the chromatogram were manually integrated and the percentage of the diabody species were calculated based on the total peak areas from each sample.

The SEC chromatograms showed that most of the antibody groups had mixed populations of proteins in solution (i.e. unstable with both the dual scFv and diabody conformations present). However, four groups demonstrated a stable conformation: 4-G2V2-52-C11-10AAL, 4-G2V2-31-E07-10AAL, 31-E07-8AAL_TM, and 31-E07-5AAL_TM. These four antibody groups had shortened 5aa (5 amino acid), 8aa or 10aa linkers present at positions L1 and L2 (see FIGS. 7A-8). The four resulting chromatograms showed a single peak resulting from each group, indicating 100% diabody formation (see FIGS. 7A and 7B). The length and compositions of the ABR-region linkers for the 4-G2V2-52-C11-10AAL and 4-G2V2-31-E07-10AAL are shown in Table 5.

TABLE 5
Linker compositions for the 4-G2V2-52-C11-10AAL
and 4-G2V2-31-E07-10AAL

| | ABR-region Linkers |
|---|---|
| L1 | (GGGGS)x2 (SEQ ID NO: 111) |
| L2 | (GGGGS)x2 (SEQ ID NO: 111) |
| Knob (L3) | GGGGS-EPKSSDKTHTCP (SEQ ID NO: 232) |
| Hole (L4) | GGGGSGGGGS (SEQ ID NO: 111) |

This format having the shortened 10aa L1 and L2 linkers is referred to as “Format 41” (see FIG. 8A). Following the affinity maturation of D5 and C11, the selected clones were cytotoxicity screened in Format 6 and screened with cell binding to remove any clones that bind off-target pHLAs (see Example 2). From there, 38 of the clones (including E07) were incorporating into Format 41 for further cytotoxicity screening and cell-binding screening (see FIG. 8B).

As described herein, E07-CD3 diabody refers to the Format 41 bispecific antibody with its first and second ABRs comprising the VH and VL sequences for E07 (Table 19) and its Fab comprising the VH and VL for hOKT3a (Table 21).

### Fabalactica Digestion to Confirm Diabody Formation

The results showed that shorter amino acid linker length (e.g., 10 amino acids of (G4S)×2 (SEQ ID NO: 111)) is enough to form the diabody confirmation for affinity mature molecules. The format 4 antibodies having a linker length of 10 amino acids without DSB were named “Format 41” antibodies. Their formation of diabody was confirmed by FabALACTICA digestion experiment, using the proteolysis by FabALACTICA methods described herein.

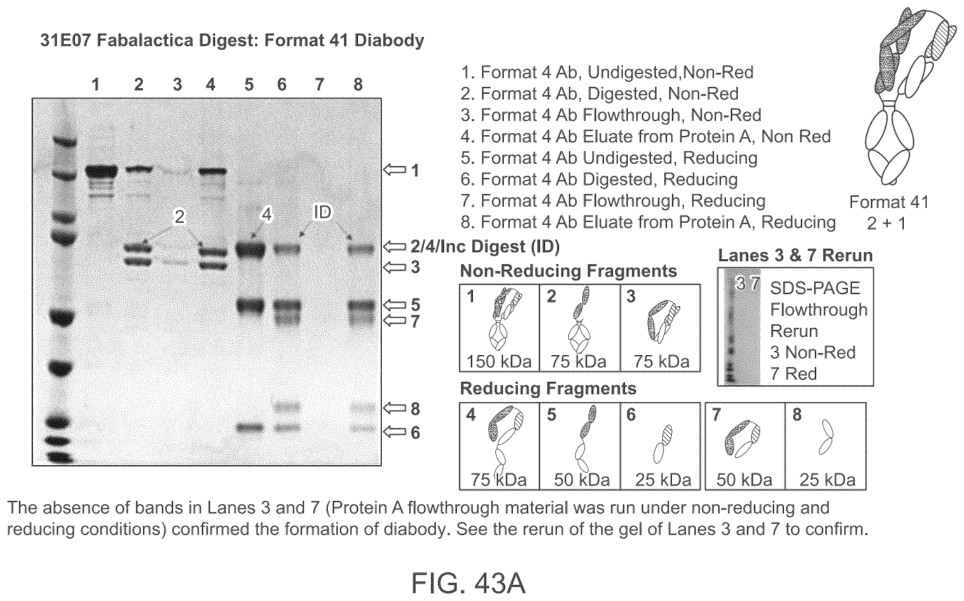

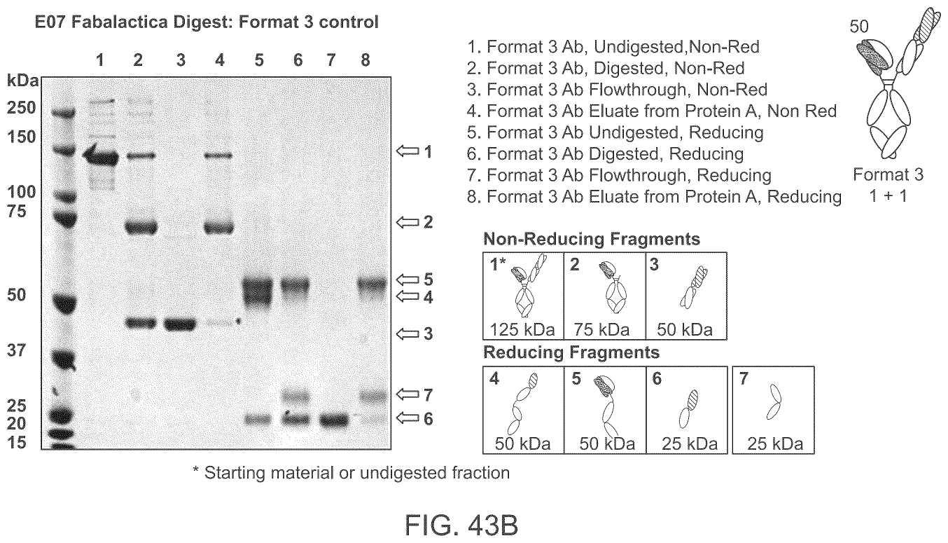

As shown in FIGS. 43A and 43B, the Format 41 antibody formed a diabody. After fabalactica digestion of format 41, whole content was passed through protein A column. The diabody formed by non-covalent interactions between VH(knob)-VL(hole) and VH(hole)-VL(Knob) resulted in retaining of digested fragments in the protein A column. A Format 3 antibody was used as a control. In case of the Format 3 antibody, after FabALACTICA digestion, the Fab portion of the molecule flows through protein A column showing the corresponding band in the SDS-PAGE gel. This can be seen by comparing lanes 3 and 7 of the FIG. 43A (Format 41) and FIG. 43B (Format 3).

### Example 5: Cytotoxicity Screening in Format 41

Following the affinity maturation and screening to the select clones from D5 and C11, selected clones were incorporated into Format 41 and tested. E07 (which had been affinity matured from D5) was identified as a preferred clone.

### Cytotoxicity Assay

Target and control cells were plated at 50,000 cells per well of 96 well plate. The target density was confirmed via mass spectrometry. These lines were transduced with luciferase. The target cell line were A375 melanoma cell lines (ATCC) transduced with CT83 and luciferase; while the A375 transduced with luciferase alone serves as the negative control. After allowing the cells to adhere for 3 hours, human T cells (All Cells) were added at a ratio of 5:1 effector to target (5:1 E:T) cells. Bispecific antibody (E07-CD3 diabody or D05 incorporate into Format 41 anti-CD3 antibody) was added to the well at indicated final concentration. Cultures were incubated for three days. Luciferase signal was assessed using Promega's Bio-Glo assay system (Cat. #G7941) according to manufacturer's instructions and read on the SpectraMax M5. Signal was normalized to control wells to determine the percent of cytotoxicity. Loss of luciferase signal is interpreted as loss of cell viability.

The results, as shown in FIG. 9, revealed significantly higher levels of cytotoxicity of the target cells (having a high copy number, e.g., 30,000 copies/cell) when using the affinity matured E07 lead compared to D05. Cytotoxicity screening revealed the E07 lead clone increased EC50 by approximately 350-fold.

Additional target cells lines were used in cytotoxicity assays. For example, the NCI-H1703 and NCI-H820, which were obtained from ATCC, were tested. The target density was confirmed via mass spectrometry.

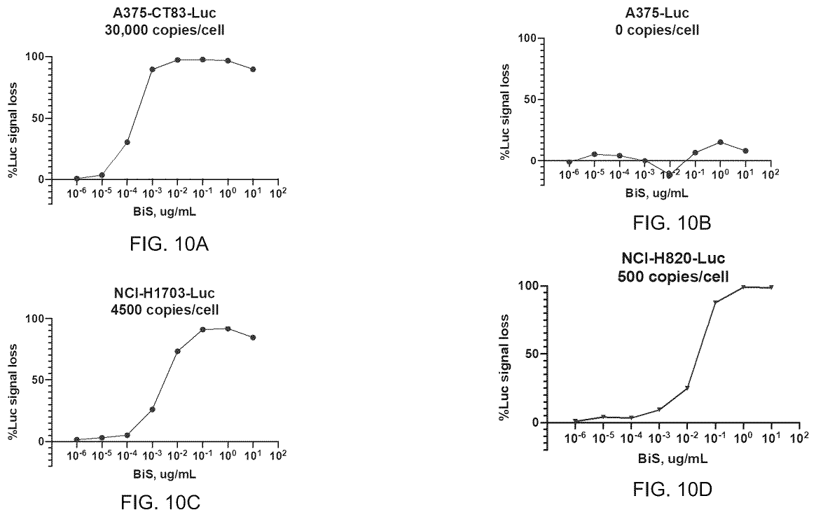

The results, as shown in FIGS. 10A-10D, revealed high potency of the 41-E07 hOKT3a antibodies even when used on cells with low copy number (e.g., NCI-H820 cells with an A*01:01_NTDNNLAVY (SEQ ID NO: 214) copy number of 500 copies/cell). Potency was demonstrated across a broad copy number range and relevant target density cell lines. See Table 6 and FIGS. 10A-10D.

TABLE 6
Luciferase-cytotoxicity assays with 41-E07 hOKT3a antibodies

| Cell line | EC50 (ug/ml) |
|---|---|
| A375-CT83 | 0.00017 |
| NCI-H1703 | 0.0025 |
| NCI-H820 | 0.077 |

### Example 6: Examination and Optimization for Liabilities

The 41-E07 hOKT3a diabody was evaluated to determine potential off-target liabilities. These potential off target liabilities are peptide sequences predicted by EDGE and presented by the A01:01 HLA and have sequence similarity to the G2 target peptide.

### Off-Target Liability Assessment (OTLA)

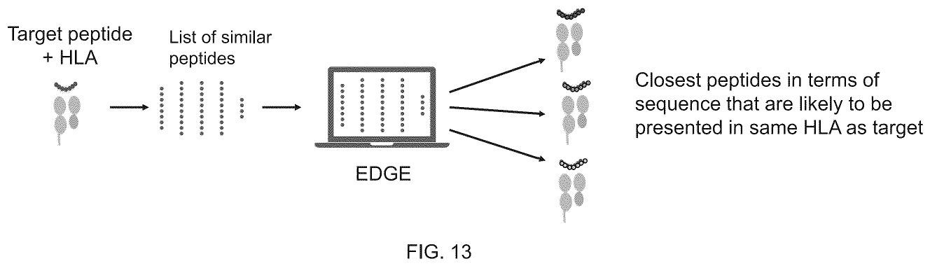

Peptides were prioritized for deselection and screening in the discovery campaign through our off-target liability analysis (OTLA) process. Briefly, EDGE™ (the proprietary presentation prediction algorithm EDGE, as described in Bulik-Sullivan, et al. Nat Biotechnol. 2018 Dec. 17. doi: 10.1038/nbt.4313, which is hereby incorporated by reference in its entirety) generated a large list of off-target liabilities for CT83 target sequence. This list was further prioritized based on their EDGE score (likelihood of being presented) and the number of mismatched amino acids (≤4 mismatches) from the target sequence. The reduced sequence list was then validated by mass spectrometry on relevant tumor cells yielding 3 OTLAs. See FIG. 13.

### Cell-Based Off-Target Liability Assessment

To evaluate specificity for target vs. off-target validated OTLAs, pulsed K562-HLA cells were treated with the bispecifics (41-E07 hOKT3a diabody) K562 cell line generation:

The Phoenix-AMPHO cells (ATCC®, CRL-3213™) were cultured in DMEM (Corning™, 17-205-CV) supplemented with 10% FBS (Seradigm, 97068-091) and Glutamax (Gibco™, 35050079). K-562 cells (ATCC®, CRL-243™) were cultured in IMDM (Gibco™, 31980097) supplemented with 10% FBS. Lipofectamine LTX PLUS (Fisher Scientific, 15338100) contains a Lipofectamine reagent and a PLUS reagent. Opti-MEM (Gibco™, 31985062) was purchased from Fisher Scientific.

Phoenix cells were plated at 5×10e5 cells/well in a 6 well plate and incubated overnight at 37° C. For the transfection, 10 μg plasmid, 10 μL Plus reagent and 100 μL Opti-MEM were incubated at room temperature for 15 minutes. Simultaneously, 8 μL Lipofectamine was incubated with 92 μL Opti-MEM at room temperature for 15 minutes. These two reactions were combined and incubated again for 15 minutes at room temperature after which 800 μL Opti-MEM was added. The culture media was aspirated from the Phoenix cells and they were washed with 5 mL pre-warmed Opti-MEM. The Opti-MEM was aspirated from the cells and the lipofectamine mixture was added. The cells were incubated for 3 hours at 37° C. and 3 mL complete culture medium was added. The plate was then incubated overnight at 37° C. The media was replaced with Phoenix culture medium and the plate incubated an additional 2 days at 37° C.

The media was collected and filtered through a 45 μm filter into a clean 6 well dish. 20 μL Plus reagent was added to each virus suspension and incubated at room temperature for 15 minutes followed by the addition of 8 μL/well of Lipofectamine and another 15 min room temperature incubation. K562 cells were counted and resuspended to 5×10⁶ cells/mL and 100 μL added to each virus suspension. The 6 well plate was centrifuged at 700 g for 30 minutes and then incubated at 37° C. for 5-6 hours. The cells and virus suspension were then transferred to a T25 flask and 7 mL K562 culture medium was added. The cells were then incubated for three days. The transduced K562 cells were then cultured in medium supplemented with 0.6 μg/mL Puromycin (Invivogen, ant-pr-1) and selection monitored by flow cytometry.

### Flow Cytometry Methods:

HLA-transduced K562 cells were pulsed the night before with 50 μM of peptide (Genscript) in IDMEM containing 1% FBS in 6 well plates and incubated under standard tissue culture conditions. Cells were harvested, washed in PBS, and stained with eBioscience Fixable Viability Dye eFluor 450 for 15 minutes at room temperature. Following another wash in PBS+2% FBS, cells were resuspended with 41-E07 hOKT3a diabody at varying concentrations. Cells were incubated with bispecifics (41-E07 hOKT3a diabody) for 1 hour at 4° C. After another wash, PE-conjugated goat anti-human IgG secondary antibody (Jackson ImmunoResearch) was added at 1:100. After incubating at 4° C. for 45 minutes and washing in PBS+2% FBS, cells were resuspended in PBS+2% FBS and analyzed by flow cytometry. Flow cytometric analysis was performed on the Attune NxT Flow Cytometer (ThermoFisher) using the Attune NxT Software. Data was analyzed using FlowJo.

### Results

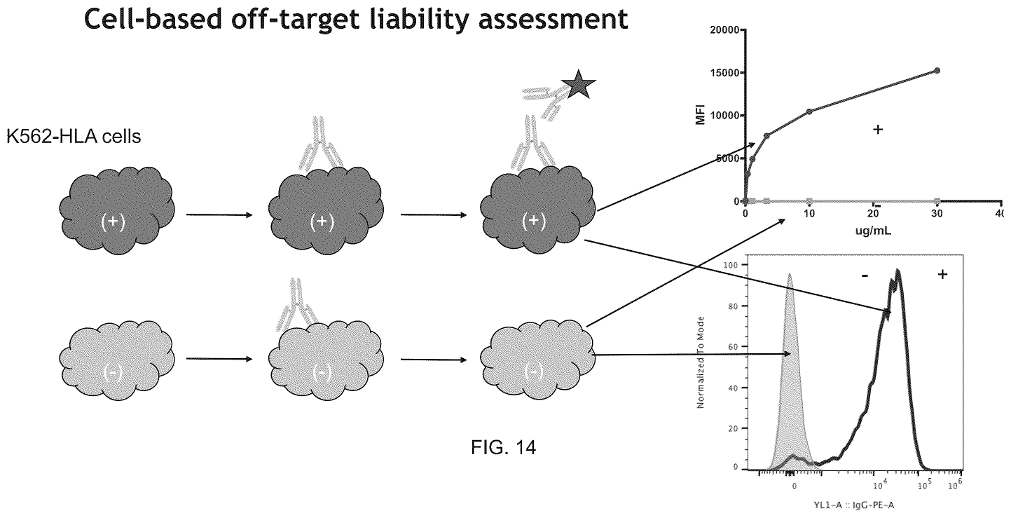

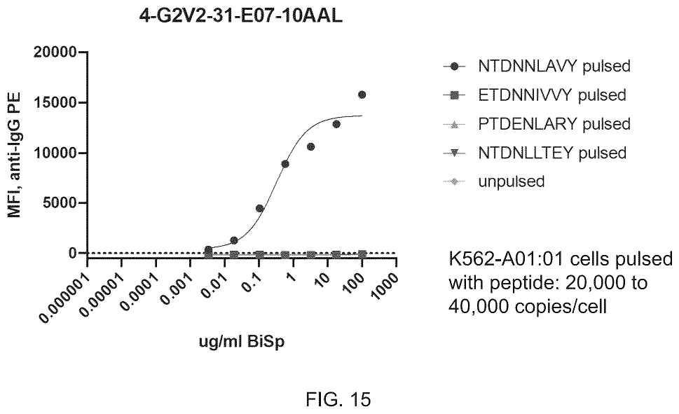

FIG. 14 is a schematic showing the binding of HLA-transduced K562 cells and expected exemplary MFI signals where there is binding specificity for the pulsed target (+) and there is no (or negligible) binding affinity to the pulsed OTLAs or unpulsed cells (−). As shown in FIG. 15, the flow cytometry results revealed 41-E07 hOKT3a diabody binds to its specific G2 target (NTDNNLAVY (SEQ ID NO: 214)) but not to the validated OTLAs.

### Example 7: Additional Molecular Assessments for Drug-Ability

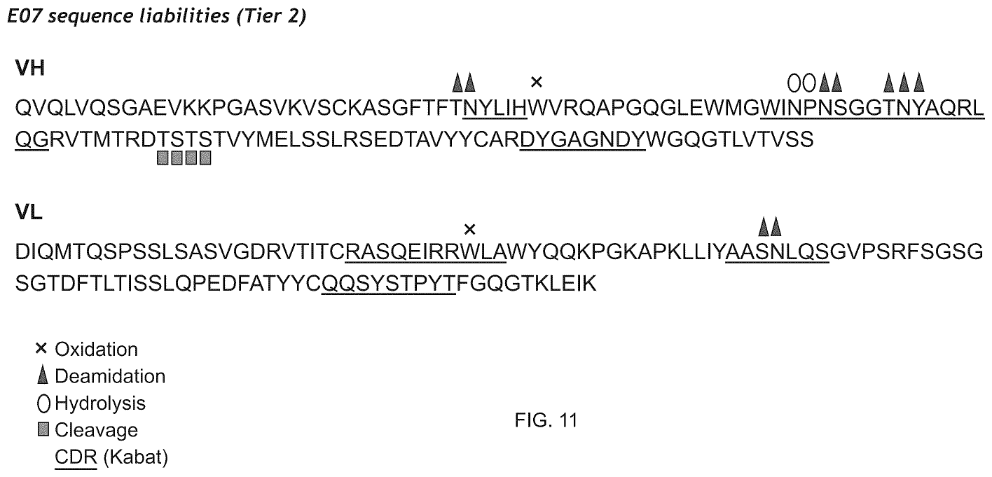

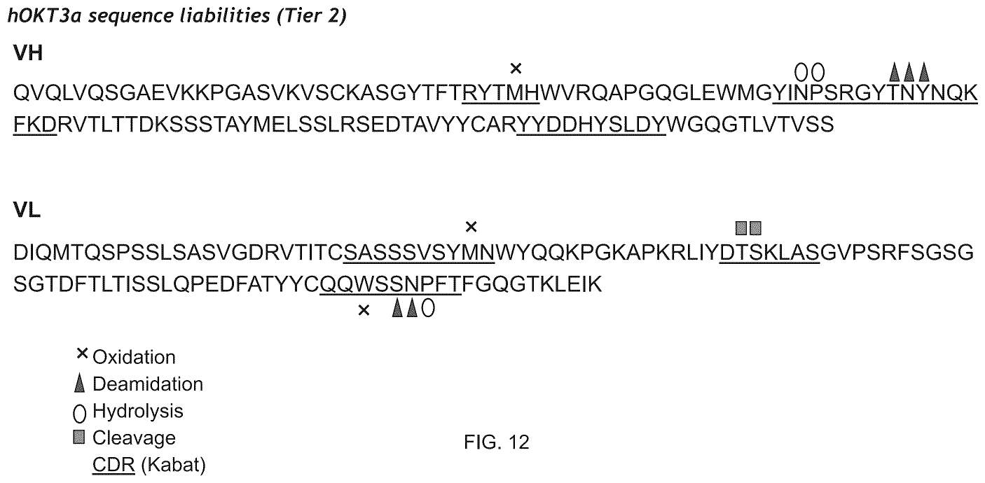

The 41-E07 hOKT3a diabody was evaluated to determine potential protein liabilities using Geneious Biologics software. See FIGS. 11 and 12.

Additional testing is conducted on the 41-E07 hOKT3a diabody to demonstrate its drug-ability and manufacturing. For example, the following assays are conducted on the 41-E07 hOKT3a diabody:
Polyreactivity assays to assess specificity
AC-SINS (affinity-capture self-interaction nanoparticle spectroscopy) to assess self-interaction
DSF (Temperature stability measurements) to determine the melting temperature
Analytical SEC to determine purity
pH Hold Assay to determine stability of the molecule at low pH
High concentration buffer screen over time (0, 1, 3, and 6 weeks) and at various temperatures (4° C., 37° C., and 40° C.) to determine best buffer to formulate and its stability
Viscosity measurements to determine at what concentrations the material stays in solution
Expression in Expi-CHO Titer and QC (quality control) to determine the level of expression and quality of material generated by CHO cells

### Example 8: In Vivo Phamacokinetic (PK) Profiling

### PK in NSG Mice

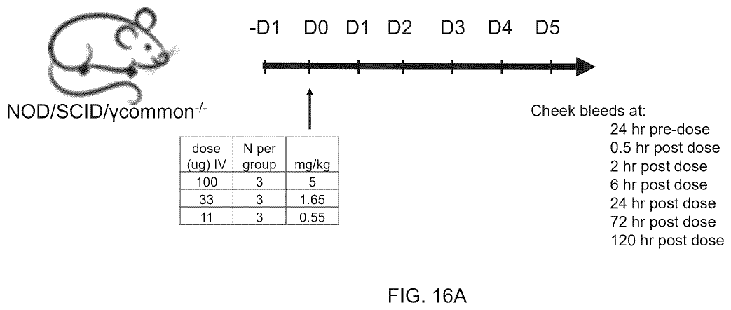

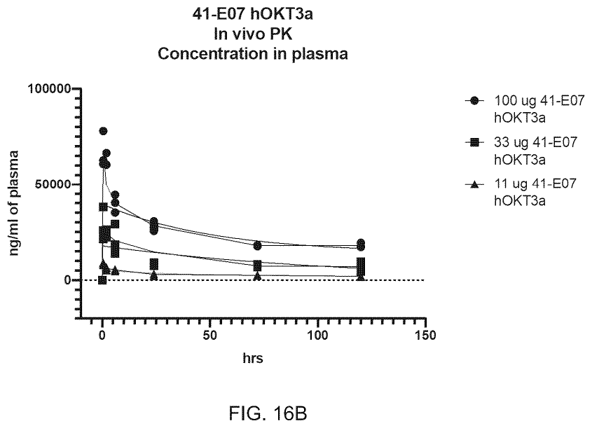

The 41-E07 hOKT3a diabody was administered to mice to examine the pharmacokinetic profile of the antibody. Briefly, eight to ten week old female NOD.Ch-Prkd^{escid}Il2rg^{tm1wjl}/SzJ (JAX) mice were used to assess PK in vivo. Blood was collected pre-dose from the submandibular vein. Mice were then dosed IV (intravitreally) with drug at varying concentrations as listed in FIG. 16A. Blood was collected from the submandibular vein at timepoints indicated. Blood samples were spun down in the presence of EDTA to collect plasma. Concentration of drug in the plasma was determined by Meso Scale Discovery (MSD). Briefly, an MSD GOLD Streptavidin plate (MSD) was coated with 1 μg/ml biotinylated pHLA antigen overnight. The plasma samples were diluted in PBS+10% FBS and mixed 1:1 with 1 μg/ml SulfoTag labeled anti-Fc antibody (Jackson ImmunoResearch) and incubated for 2 hours at room temperature. The pHLA coated plate was washed in PBS+0.1% Tween and plasma/antibody mixture was transferred to the plate and incubated for an additional 2 hours. The plate was then washed again and 2× read buffer was added before reading on MSD Qplex. Plasma concentration was determined by comparing to standard curve with purified drug on the same plate. Calculated concentrations were analyzed by two phase decay on GraphPad Prism V8.4.2 to determine half-life.

The half-life for the 41-E07 hOKT3a diabody in mice was approximately 5 days. See Table 7 and FIGS. 16A-16B.

TABLE 7

| Dose | Apparent t_{1/2} (hr) |
|---|---|
| 100 ug | 140 |
| 33 ug | 113 |
| 11 ug | LoD |

### PK in Tg32 Mice

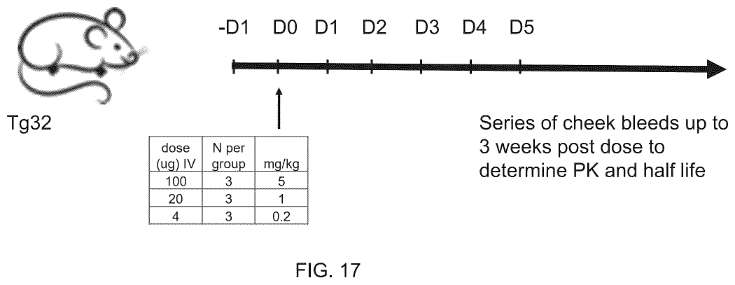

The pharmacokinetic profile for 41-E07 hOKT3a diabody is evaluated in Tg32 mice. These mice are knock-out KO for mouse FcRn receptor with knock-in of human FcRn receptor. It is a better predictor of the PK in humans. The dosing and overall procedure is illustrated in FIG. 17. Drug concentration in the plasma is determined by MSD using methods described supra.

### Example 9: In Vivo Efficacy Tests

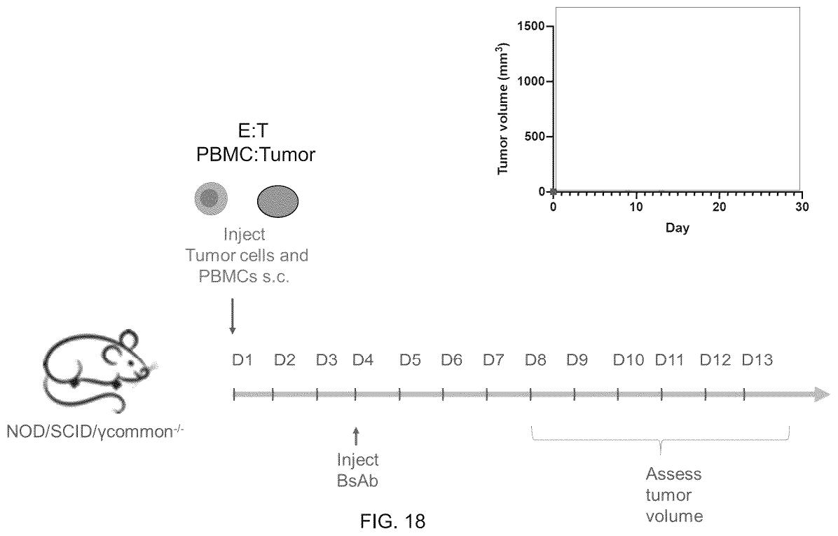

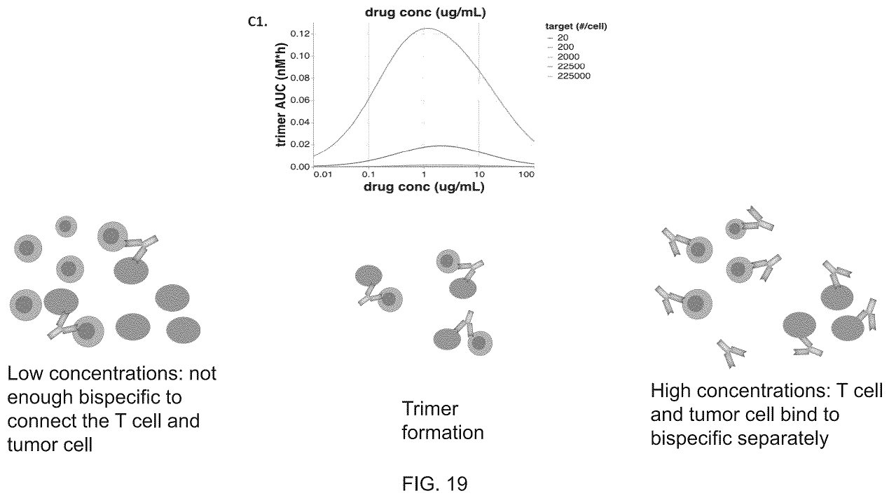

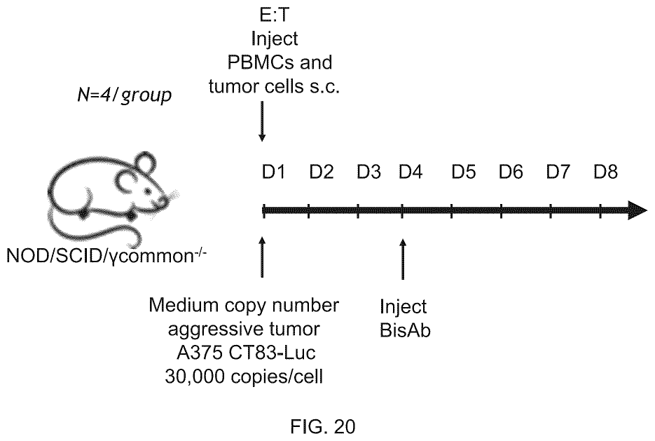

Eight to ten week old female NOD.Ch-Prkd^{escid}Il2rg^{tm1wjl}/SzJ (JAX) mice were used to assess in vivo efficacy. FIG. 18 illustrates the general study design. Modeling data from Applied Biomath analysis shows a bell-shaped curve in the relationship between drug concentration and trimer formation (see FIG. 19).

Briefly, the A375 tumor cell line (ATCC) transduced to overexpress the CT83 gene and luciferase was used. This target cell was mixed with human PBMCs (All Cells) at 1:10 or 1:5 effector to target ratio (E:T). Namely, 1×10⁶ tumor cells and 1×10⁵ PBMCs per mouse for 1:10 E:T and 1×10⁶ tumor cells and 5×10⁵ PBMCs for 1:5 E:T. The mix of tumor and effector cells were injected s.c. (subcutaneously) in the flank of mouse. Tumors were allowed to form for three days. Tumors were measured by caliper and mice were randomized into groups based on tumor volumes. Then 41-E07 hOKT3a diabody diluted in PBS was dosed IV at the concentration indicated. Mice were monitored by caliper measurements of tumors as well as body weight. Mice were sacrificed at humane endpoints. Tumor volume data measurements were analyzed by 2-way ANOVA in GraphPad Prism V8.4.2 to determine significance.

Effect of E:T Ratios with Titration Curve of E07-CD3 Diabody with High Copy Number Tumors

The 41-E07 hOKT3a diabody was dosed at day 4 and the E:T ratio was varied with titration curve of 41-E07 hOKT3a diabody to determine the correct values for each. Table 8 shows the groups and respective doses tested. This experiment was conducted with mice administered with medium copy number aggressive tumors (A375 CT83-Luc; 30,000 copies/cell).

TABLE 8

| | | Drug dose |
|---|---|---|
| Group | E:T (PBMCs:A375-CT83-Luc)* | (ug/mouse) |
| 1 | 1 to 2 | 4 |
| 2 | | 0.8 |
| 3 | | 0.16 |
| 4 | | 0.032 |
| 5 | | 0 |
| 6 | 1 to 5 | 4 |
| 7 | | 0.8 |
| 8 | | 0.16 |
| 9 | | 0.032 |
| 10 | | 0 |
| 11 | 1 to 10 | 4 |
| 12 | | 0.8 |
| 13 | | 0.16 |
| 14 | | 0.032 |
| 15 | | 0 |
| 16 | 1 to 25 | 4 |
| 17 | | 0.8 |
| 18 | | 0.16 |
| 19 | | 0.032 |
| 20 | | 0 |
| 21 | 1 to 50 | 4 |
| 22 | | 0.8 |
| 23 | | 0.16 |
| 24 | | 0.032 |
| 25 | | 0 |
| 26 | 1 to 100 | 4 |
| 27 | | 0.8 |
| 28 | | 0.16 |
| 29 | | 0.032 |
| 30 | | 0 |

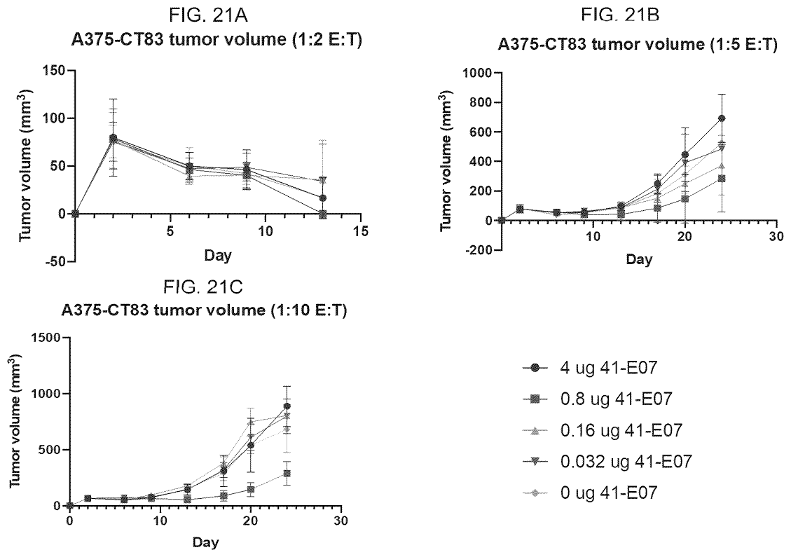

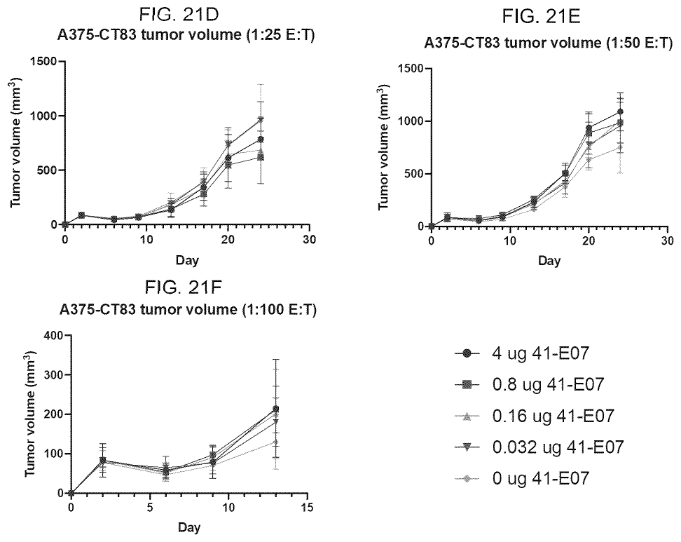

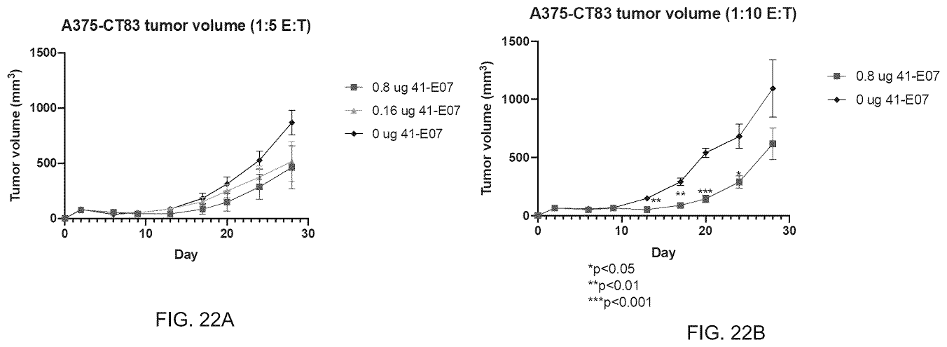

Tumor regression without the drug was observed in the 1:2 E:T ratio group. As shown in FIGS. 21A-21F, when the E07-CD3 diabody was dosed at day 4, tumor inhibition was observed at 1:10 E:T and 0.8 ug/mouse dose. There was some growth suppression observed at the 1:5 E:T with 0.16 and 0.8 ug/mouse doses. See FIGS. 21A-22B.

### Alternative Models to Test Dosing Timing and Efficacy

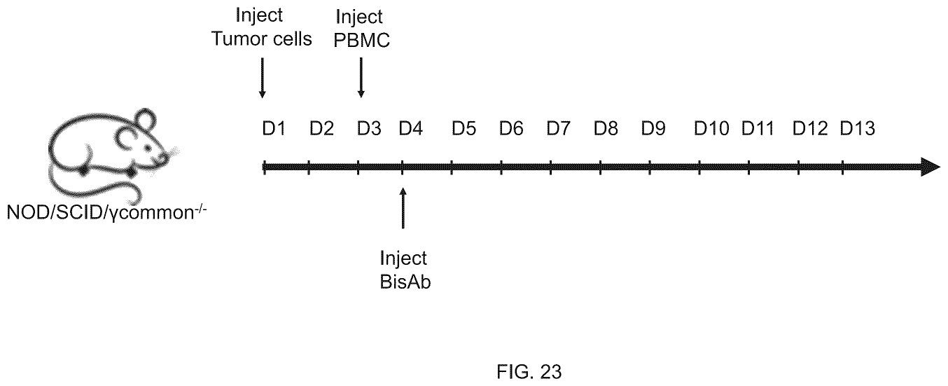

To evaluate the effect of alternative dosing strategies, the tumor cells are injected in the mice at Day 1, the PBMC injected at Day 3, and the 41-E07 hOKT3a diabody dosed (by injection) at Day 4. The E:T ratio is varied with titration curve of 41-E07 hOKT3a diabody to determine the correct values for each. Tumor volume measurements will be taken throughout the study to evaluate efficacy of treatment. See FIG. 23 for general experimental design.

### Example 10: Optimizing CD3 Antigen Binding Fragment

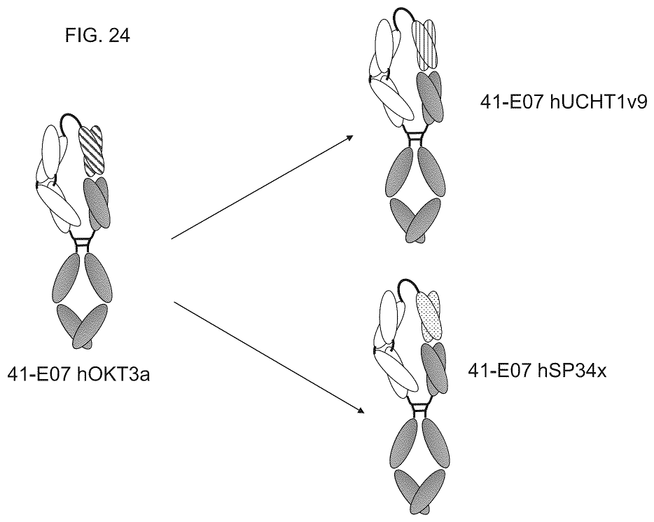

The CD3 binder was optimized in combination with the G2(31E07) antibody and the G5(1C12) antibody in the Format 41 orientation. The hOKT3a CD3 binder (Kd=2 uM) in the Format 41 diabody was replaced with UCHT1v9 (Kd=2 nM) or hSP34x (Kd=8 nM). Diagrams of the new 41-E07 binder formats are provided in FIG. 24.

Binding of each of the three 41-E07 CD3 binders (hOKT3, UCHT1v9, and hSP34x) to G2 pHLA was assessed in a A375 cell line expressing 1×10⁶ copies of the CT83 pHLA 9-mer. A375 transduced with luciferase alone served as the negative control. Cells were incubated with increasing concentrations of the different 41-E07 CD3 antibodies. Binding was determined by flow cytometery using an anti IgG-PE antibody for detection.

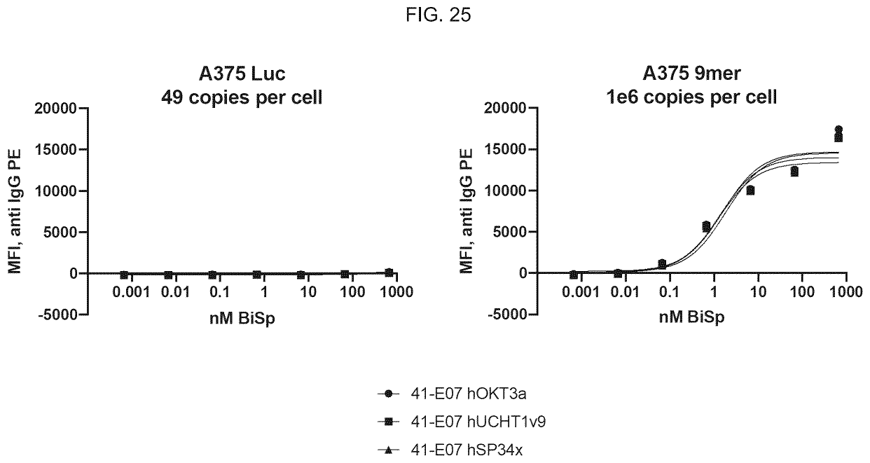

As shown in FIG. 25, there was no difference in binding of the 41-E07 hOKT3, 41-E07 UCHT1v9, or 41-E07 hSP34x antibodies to the cells expressing 1×10⁶ copies of the pHLA 9-mer.

Next, binding of the three 41-E07 CD3 binders (hOKT3, UCHT1v9, and hSP34x) to CD3 was assessed in Jurkat cells. Increasing concentrations of the antibodies were incubated with CD3(+) and CD9(−) Jurkat cells. Binding was determined by flow cytometery using an anti IgG-PE antibody for detection.

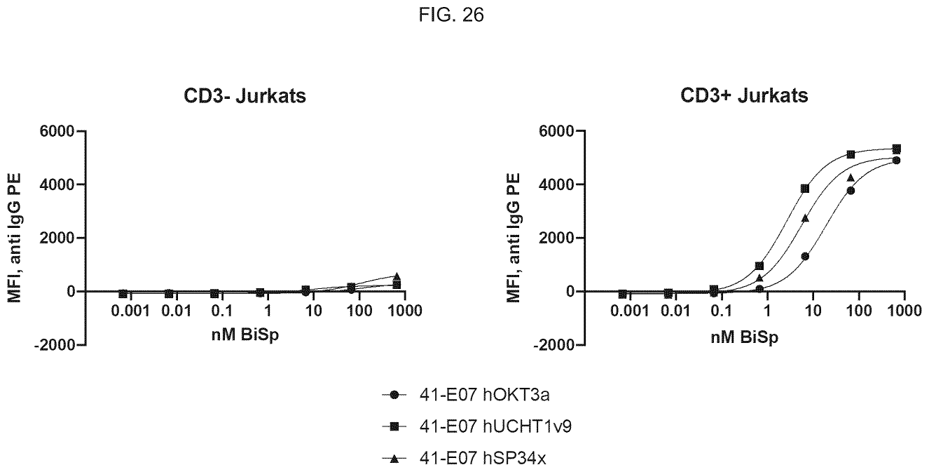

As shown in FIG. 26, 41-E07 UCHT1v9 showed the highest binding to CD3 in the CD3+ Jurkat cells. Little to no binding was observed by all antibodies in the control CD3(−) Jurkat cells.

Next, cytotoxicity of the three 41-E07 CD3 antibodies was assessed as described in Example 5. Increasing concentrations of the three antibodies were incubated with the A375 luciferase control cell line, the A375 CT83 cell line expressing 30,000 copies of the pHLA, the NCI H1703 cell line expressing 4,500 copies of the pHLA, and the NCI H820 cell line expressing 500 copies of the pHLA. Luciferase activity was assessed after incubation with the indicated antibody, and loss of luciferase signal indicated cell death, as described in Example 5.

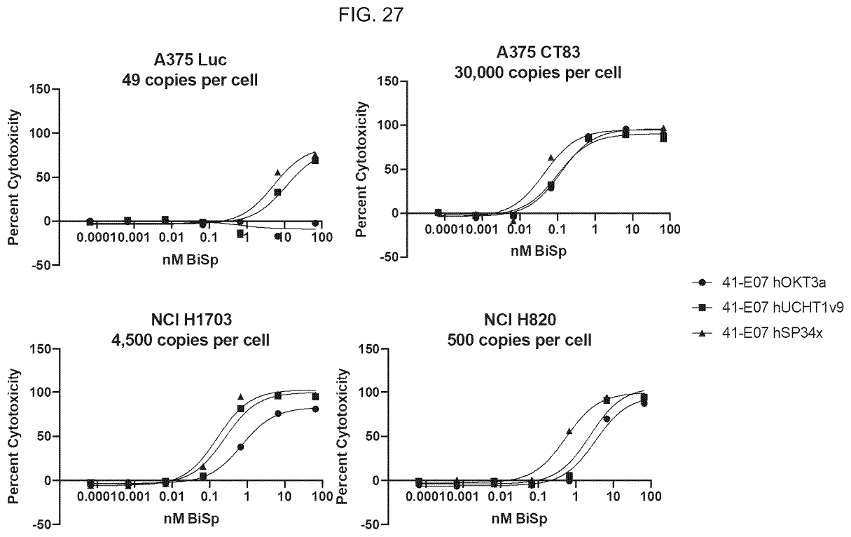

As shown in FIG. 27, 41-E07-hSP34x was more cytotoxic against tumor cells with low density pHLA expression than 41-E07 UCHT1v9 and 41-E07 hOKT3a antibodies.

In sum, similar binding of 41-E07 bispecific antibody with different CD3 antibody binding fragments to G2 pHLA was observed. The 41-E07-UCHT1v9 bispecific antibody exhibited improved binding to CD3 as compared to the 41-E07 OKT3 bispecific antibody. The 41-E07-hSP34x bispecific antibody was more potent at killing tumor cells with pHLA ranging from 500-30,000 copies per cell.

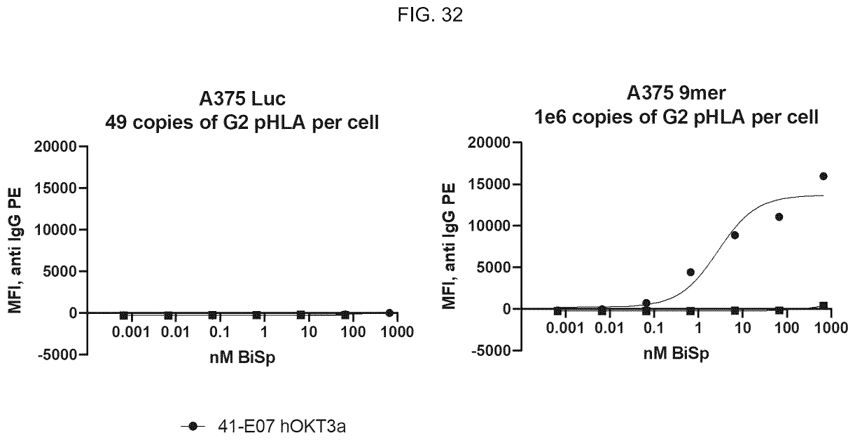

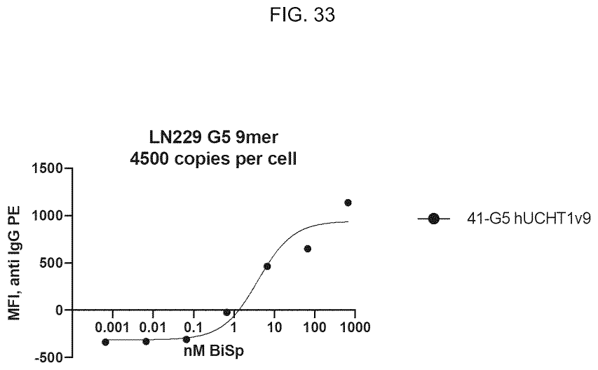

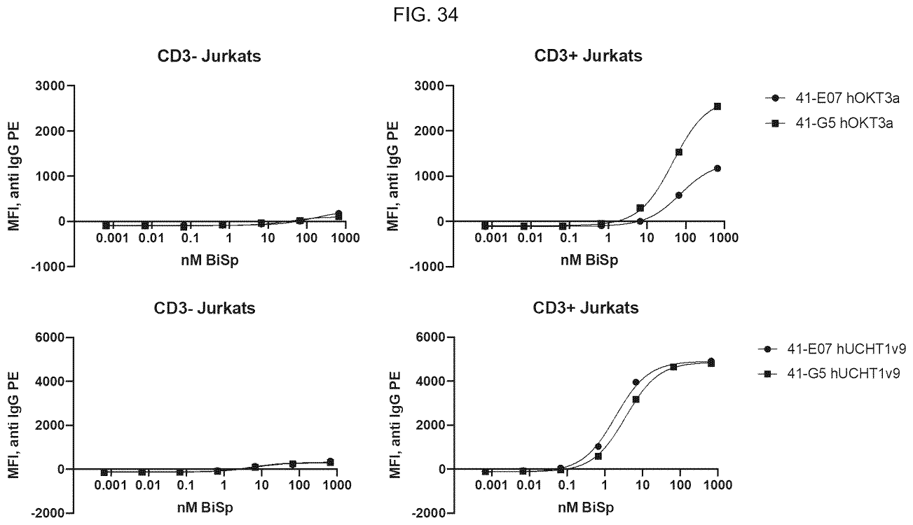

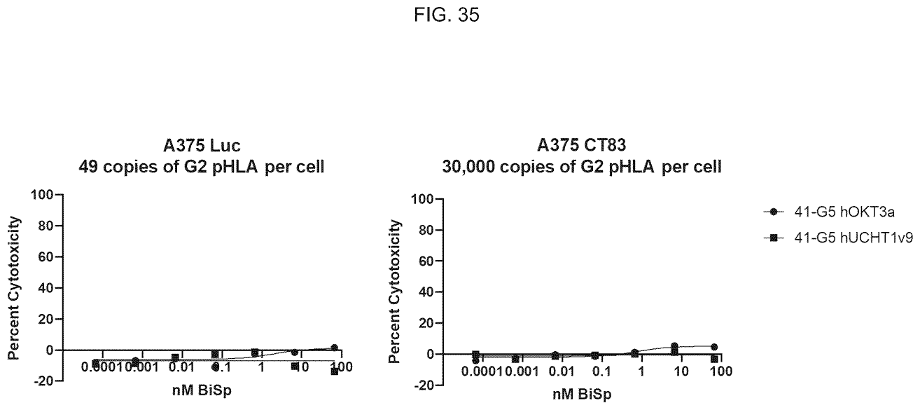

Next, the binding and cytotoxicity of the 41-G5(1C12) UCHT1v9 diabody was assessed in the A375-G2 expressing cell line. 41-G5(1C12) UCHT1v9 did not bind to cells expressing G2 pHLA (FIG. 32) but did bind to cells expressing G5 pHLA (FIG. 33). 41-G5(1C12) UCHT1v9 bound similarly to Jurkat CD3+ cells as compared to 41-E07 UCHT1v9 (FIG. 34). 41-G5(1C12) hOKT3a and 41-G5(1C12) UCHT1v9 did not induce killing of G2 pHLA positive cell lines (FIG. 35). In sum, 41-G5(1C12) UCHT1v9 exhibited G5 specific binding, did not bind to the off target G2 antigen, and did not induce off target cytotoxicity. In addition, altering the CD3 binder from OKT3 to UCHT1v9 did not adversely affect CD3 binding.

### Example 11: Alternative Antibody Formats

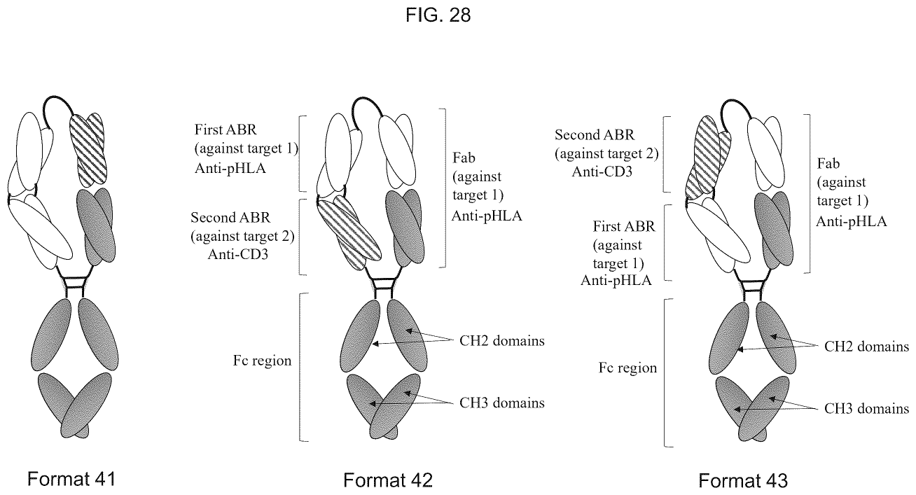

A new bispecific antibody format, 43, was constructed with the anti-CD3 and anti-pHLA binders. The format is shown in FIG. 28. In this new format, the two ABRs on the second arm (non-Fab arm) are encoded partially on the first polypeptide chain and partially on the second polypeptide chain (e.g., one VH domain and one VL domain from each ABR is encoded on each of polypeptide chain one and two). For example, a VH domain that binds target 1 can be linked to the VL domain that binds target 2 and encoded on polypeptide one while polypeptide two encodes a VL domain that binds target 1 linked to the VH domain that binds target 2. The respective paired VH and VL domains on polypeptide one and polypeptide two can interact with each other and thus form two complete ABRs to each respective target (target 1 and target 2). The Format 43 also places a Fab fragment against target 1 (e.g., an anti-pHLA Fab) on one arm. One variable region of the target 1 binder (e.g., a pHLA binder) is linked to the CH2 and CH3 regions of the Fc region on polypeptide chain one, while one variable region of the target 2 binder (e.g., a CD3 binder) is linked to the Fab heavy chain on polypeptide chain two such that the target 2 binder is sandwiched between two target 1 binders in the circularized antibody (see FIG. 28).

Binding of the 41-E07 UCHT1v9 and 43-E07 UCHT1v9 to the G2 pHLA was assessed in a A375 cell line expressing 1×10⁶ copies of the CT83 pHLA 9-mer. A375 transduced with luciferase alone served as the negative control. Cells were incubated with increasing concentrations of the 41-E07 UCHT1v9 and 43-E07 UCHT1v9 antibodies. Binding was determined by flow cytometery using an anti IgG-PE antibody for detection.

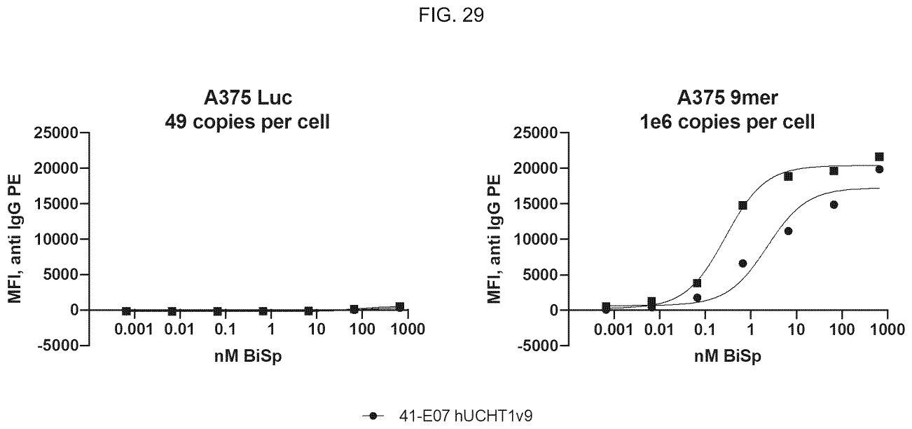

As shown in FIG. 29, 43-E07 UCHT1v9 showed improved binding (approximately 10 fold increase) to the cells expressing 1×10⁶ copies of the pHLA 9-mer as compared to 41-E07 UCHT1v9. Thus, the change in the format from Format 41 to Format 43 improved the binding ability of the bispecific antibody to the pHLA target.

Next, binding of the 41-E07 UCHT1v9 and 43-E07 UCHT1v9 to CD3 was assessed in Jurkat cells. Increasing concentrations of the antibodies were incubated with CD3(+) and CD9(−) Jurkat cells. Binding was determined by flow cytometery using an anti IgG-PE antibody for detection.

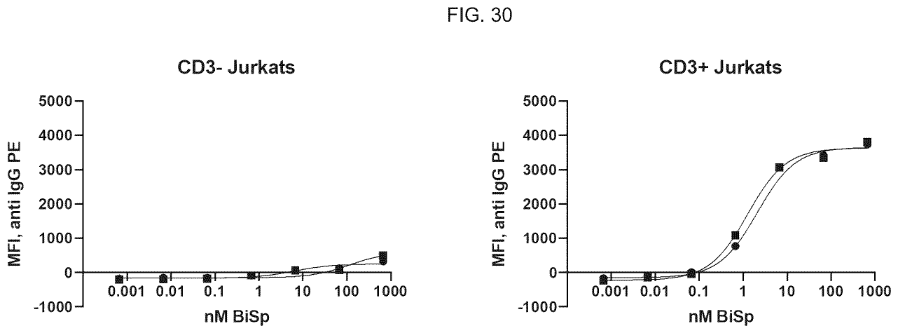

As shown in FIG. 30, 43-E07 UCHT1v9 has similar binding to CD3 as 41-E07 UCHT1v9 in the CD3+ Jurkat cells. Little to no binding was observed by both antibodies in the CD3− Jurkat cells.

Next, cytotoxicity of the 41-E07 UCHT1v9 and 43-E07 UCHT1v9 antibodies was assessed as described in Example 5. Increasing concentrations of the two antibodies were incubated with the A375 luciferase control cell line, the A375 CT83 cell line expressing 30,000 copies of the pHLA, and the NCI H820 cell line expressing 500 copies of the pHLA. Luciferase activity was assessed after incubation with the indicated antibody, and loss of luciferase signal indicated cell death, as described in Example 5.

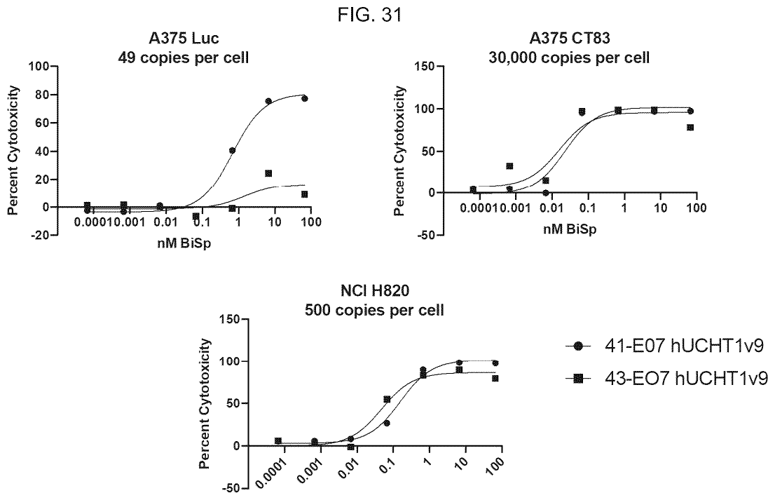

As shown in FIG. 31, 41-E07 UCHT1v9 and 43-E07 UCHT1v9 has similar cytotoxicity potency on tumor cells expressing 500-30,000 copies of pHLA.

In sum, 43-E07 UCHT1v9 showed an approximately 10-fold increase in binding to pHLA relative to 41-E07 UCHT1v9. In addition, altering the position of CD3 and E07 in the bispecific antibody did not affect CD3 binding, as the format 43-E07 UCHT1v9 showed the same level of CD3 binding as the format 41-E07 UCHT1v9. Furthermore, 41-E07 UCHT1v9 and 43-E07 UCHT1v9 had similar cytotoxicity potency on tumor cells with pHLA ranging from 500-30,000 copies per cell

### Example 12: In Vivo Efficacy of 41-E07 hOKT3a, 43-E07 hUCHT1v9 and 41-E07 hSP3 Bispecific Antibodies

### Methods

Eight-week-old female NOD.Cg-Prkd^{scid} H2-K1^{tm1Bpe} H2-Ab1^{em1Mvw} H2 D1^{tm1Bpe} Il2rg^{tm1Wjl}/SzJ (JAX) mice were used to assess in vivo efficacy of the bispecific antibodies with different CD3 binders. The A375 tumor cell line (ATCC) transduced to overexpress the CT83 gene and luciferase. 1×10⁶ A375-CT83-Luc (A375 CT83) cells were injected subcutaneously in the flank of each mouse on day 0. 1×10⁷ human PBMCs were injected intravenously (i.v.) on either day 0 (groups 2-6 and 10-12) or day 1 (groups 7-9 and 13-15). Tumors were allowed to grow to 50-100 mm³ prior to antibody dosing. Tumors were measured by caliper and mice were randomized into groups based on tumor volumes. Mice were bled on day 7 prior to antibody dosing and PBMC engraftment was monitored via flow cytometry. Mice were bled on day 14 and day 21 post PBMC injection to also assess PBMC engraftment and expansion. 41-E07 hOKT3a, 43-E07 UCHT1v9 or 41-E07 hSP34x were diluted in PBS and administered intravenously at the indicated concentration on day 7 (Table 9). 41-G5(1C12) hOKT3a, which binds to a different pHLA target, was used as a control. Mice were monitored by caliper measurements of tumors as well as body weight twice a week. Mice were sacrificed at humane endpoints or by day 40.

TABLE 9

| | | PBMC | Dugs IV | |
|---|---|---|---|---|
| Group | Tumor cells s.c. | IV | (mg/kg) | Antibody |
| 1 | 1 × 10⁶ A375 CT83-Luc | 0 PBMC | 0 | |
| 2 | 0 | 1 × 10⁷ | 0 | |
| 3 | 1 × 10⁶ A375 CT83-Luc | 1 × 10⁷ | 0 | PBS |
| 4 | 1 × 10⁶ A375 CT83-Luc | 1 × 10⁷ | 1 | 41-E07-hOKT3a |
| 5 | 1 × 10⁶ A375 CT83-Luc | 1 × 10⁷ | 0.1 | 41-E07-hOKT3a |
| 6 | 1 × 10⁶ A375 CT83-Luc | 1 × 10⁷ | 0.01 | 41-E07-hOKT3a |
| 7 | 1 × 10⁶ A375 CT83-Luc | 1 × 10⁷ | 1 | 43-E07-UCHT1v9 |
| 8 | 1 × 10⁶ A375 CT83-Luc | 1 × 10⁷ | 0.1 | 43-E07-UCHT1v9 |
| 9 | 1 × 10⁶ A375 CT83-Luc | 1 × 10⁷ | 0.01 | 43-E07-UCHT1v9 |
| 10 | 1 × 10⁶ A375 CT83-Luc | 1 × 10⁷ | 1 | 41-G5(1C12)-hOKT3a |
| 11 | 1 × 10⁶ A375 CT83-Luc | 1 × 10⁷ | 0.1 | 41-G5(1C12)--hOKT3a |
| 12 | 1 × 10⁶ A375 CT83-Luc | 1 × 10⁷ | 0.01 | 41-G5(1C12)--hOKT3a |
| 13 | 1 × 10⁶ A375 CT83-Luc | 1 × 10⁷ | 1 | 41-31E07-hSP34x |
| 14 | 1 × 10⁶ A375 CT83-Luc | 1 × 10⁷ | 0.1 | 41-31E07-hSP34x |
| 15 | 1 × 10⁶ A375 CT83-Luc | 1 × 10⁷ | 0.01 | 41-31E07-hSP34x |

### Results

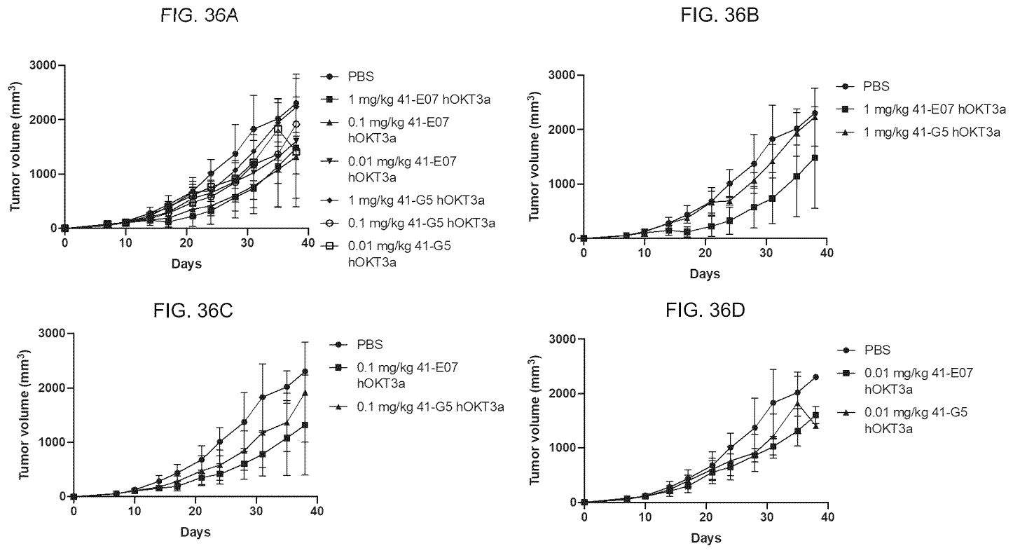

Mice treated with 1 mg/kg 41-E07 hOKT3a showed a reduction in A375-CT83 tumor growth when compared to mice treated with the control antibody, 1 mg/kg 41-G5(1C12) hOKT3a (FIGS. 36A and B). Mice treated with 0.1 mg/kg E07 hOKT3a demonstrated a decreasing trend in tumor growth compared 0.1 mg/kg 41-G5(1C12) hOKT3a but was not significantly different (FIG. 36C). Additionally, there was no significant difference in tumor growth between mice treated with 0.01 mg/kg 41-E07 hOKT3a when compared to mice treated with 0.01 mg/kg 41-G5(1C12) hOKT3a (FIG. 36D). Mice treated with 1 mg/kg and 0.1 mg/kg 41-E07 hOKT3a showed a decreased in A375-CT83 tumor growth compared to PBS control group (FIG. 36A).

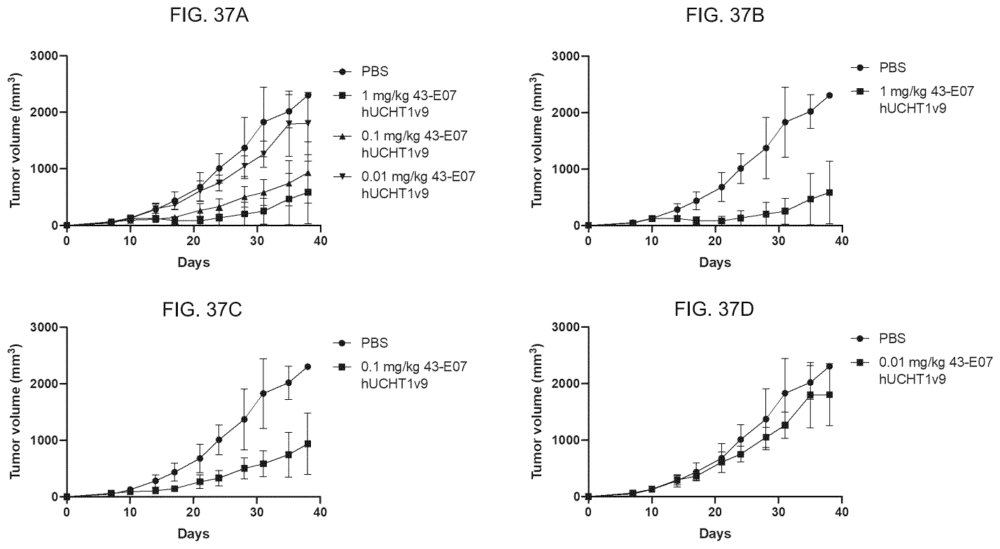

Mice treated with 1 and 0.1 mg/kg 43-E07 UCHT1v9 demonstrated a significant reduction in tumor growth compared to mice treated with PBS (FIGS. 37A, B, and C). However, mice treated with 0.01 mg/kg 43-E07 UCHT1v9 did not show a significant difference in tumor growth compared to PBS group (FIG. 37D).

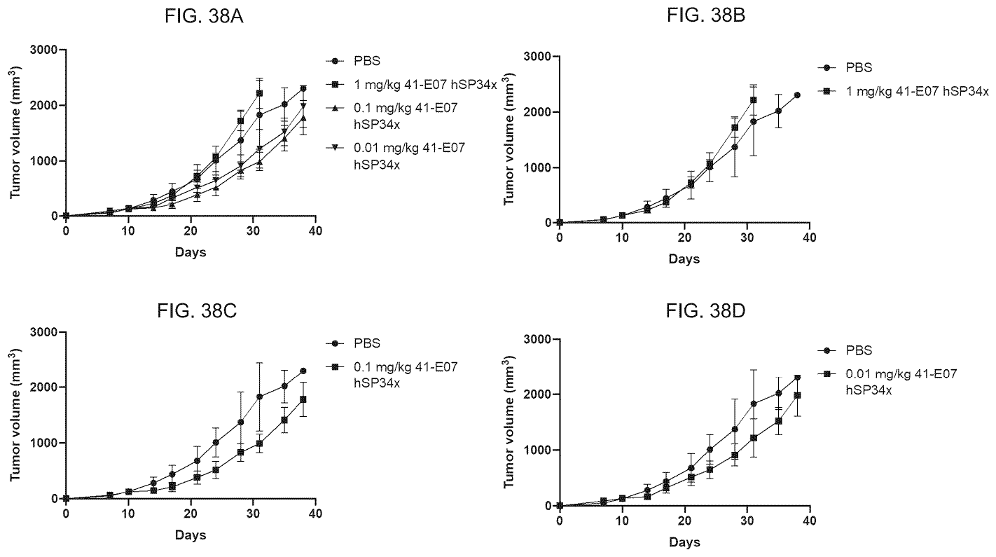

Mice treated with 1 and 0.01 mg/kg 41-E07 hSP34x did not have a significant reduction in tumor growth when compared to PBS control group (FIGS. 38A, B, and D). In contrast, mice treated with 0.1 mg/kg 41-E07 hSP34x demonstrated a slight reduction in tumor growth compared to mice treated with PBS (FIGS. 38A and C).

In sum, mice treated with 1 mg/kg 41-E07 hOKT3a had reduced tumor growth compared to mice treated with 1 mg/kg 41-G5(1C12) hOKT3a. Mice treated with 1 and 0.1 mg/kg 43-E07 UCHT1v9 demonstrated a reduction in tumor growth compared to mice treated with PBS. Similarly, mice treated with 0.1 mg/kg 41-E07 hSP34x demonstrated a reduction in tumor growth compared to mice treated with PBS. Mice treated with all concentrations of 41-G5(1C12) hOKT3a did not have a significant impact on tumor growth when compared to PBS control group.

### Example 13: In Vivo Efficacy of 41-E07 UCHT1v9 Bispecific Antibody

### Methods

Eight-week-old female NOD.Cg-Prkd^{scid}Il2rg^{tm1Wjl}/SzJ (JAX) mice were used to assess in vivo efficacy of the Format 41 and 43 antibodies. The A375 tumor cell line (ATCC) transduced to overexpress the CT83 gene and luciferase. 1×10⁶ A375-CT83-Luc (A375 CT83) cells were injected subcutaneously in the flank of each mouse on day 0. 1×10⁷ human PBMCs were injected intravenously (i.v.) on day 0. Tumors were allowed to grow to 50-100 mm³ prior to antibody dosing. Tumors were measured by caliper and mice were randomized into groups based on tumor volumes. Mice were bled on day 7 prior to antibody dosing and PBMC engraftment was monitored via flow cytometry. Mice were bled on day 14 and day 21 post PBMC injection to assess PBMC engraftment and expansion. 41-E07 UCHT1v9 or 41-G5(1C12) UCHT1v9 antibody was diluted in PBS and administered via intravenous (i.v.) route at the indicated concentration on day 7 (Table 10). Mice were monitored by caliper measurements of tumors as well as body weight twice a week. Mice were sacrificed at humane endpoints or by day 40.

TABLE 10

| | | PBMC | Dugs IV | |
|---|---|---|---|---|
| Group | Tumor cells s.c. | IV | (mg/kg) | Antibody |
| 1 | 1 × 10⁶ A375 CT83-Luc | 0 PBMC | 0 | |
| 2 | 0 | 1 × 10⁷ | 0 | |
| 3 | 1 × 10⁶ A375 CT83-Luc | 1 × 10⁷ | 0 | PBS |
| 4 | 1 × 10⁶ A375 CT83-Luc | 1 × 10⁷ | 2 | 41-E07-UCHT1v9 |
| 5 | 1 × 10⁶ A375 CT83-Luc | 1 × 10⁷ | 1 | 41-E07-UCHT1v9 |
| 6 | 1 × 10⁶ A375 CT83-Luc | 1 × 10⁷ | 0.1 | 41-E07-UCHT1v9 |
| 7 | 1 × 10⁶ A375 CT83-Luc | 1 × 10⁷ | 0.01 | 41-E07-UCHT1v9 |
| 8 | 1 × 10⁶ A375 CT83-Luc | 1 × 10⁷ | 2 | 41-G5(1C12)-UCHT1v9 |
| 9 | 1 × 10⁶ A375 CT83-Luc | 1 × 10⁷ | 1 | 41-G5(1C12)-UCHT1v9 |
| 10 | 1 × 10⁶ A375 CT83-Luc | 1 × 10⁷ | 0.1 | 41-G5(1C12)-UCHT1v9 |
| 11 | 1 × 10⁶ A375 CT83-Luc | 1 × 10⁷ | 0.01 | 41-G5(1C12)-UCHT1v9 |

### Results

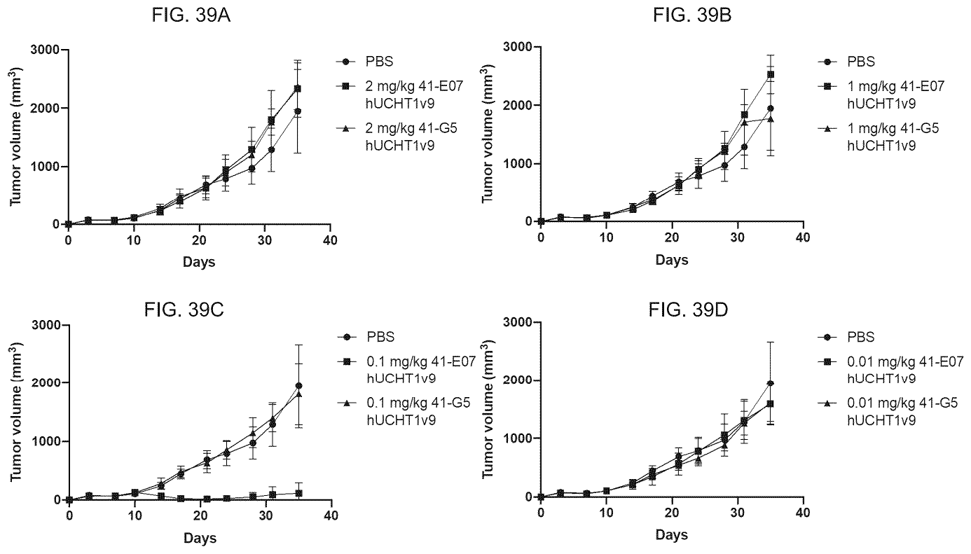

Mice treated with 1 and 0.01 mg/kg 41-E07 UCHT1v9 did not have a significant reduction in tumor growth compared to mice treated with 41-G5 UCHT1v9 (FIGS. 39A, B, and D). However, mice treated with 0.1 mg/kg 41-E07 UCHT1v9 demonstrated a significant reduction in tumor growth compared to mice treated with 0.1 mg/kg 41-G5(1C12) UCHT1v9 (FIG. 39C). However, mice treated with 1 and 0.01 mg/kg 41-E07 UCHT1v9 had very little to no PBMC engraftment while the mice treated with 0.1 mg/kg 41-E07 UCHT1v9 did have PBMC engraftment.

In sum, mice treated with 0.1 mg/kg 41-E07 UCHT1v9 demonstrated an 80-100% tumor inhibition as compared to mice treated with 41-G5(1C12) UCHT1v9 or PBS (FIG. 39C). Three of the six mice had complete tumor regression while two of the six mice had 80-90% tumor inhibition. However, the mice dosed with 2 mg/kg and 1 mg/kg 41-E07-v9 had no PBMC engraftment. In addition, no signs of GvHD was observed in the mice treated with 41-E07-UCHT1v9 and 41-G5-UCHT1v9.

### Example 14: Off-Target Liabilities Assessment (OTLA) of E07-hOKT3a

### Materials

### HLA Refolding

HLA A*01:01 binding peptides of interest for the OTLA positional scanning were synthesized with a purity of >95% (Genscript Inc). The alpha chain of HLA-A*01:01 and beta chain β2-microglobulin, separately expressed in E. coli as inclusion bodies, were purified and then solubilized in 8 M Urea containing 10 mM DTT. Peptides resuspended in DMSO at 10 mg/ml were used in the refolding process. Refolding of the pHLA complex was initiated by rapid dilution of 20 mg β2-microglobulin, 10 mg peptide of interest and 20 mg HLA-A*01:01 alpha chain in 1 L of 100 mM Tris-HCl pH 8.0 buffer containing 400 mM L-arginine, 2 mM EDTA, 1.5 mM L-glutathione reduced and 0.5 mM of L-glutathione oxidized. The 1L refolding reaction was concentrated using tangential flow filtration process (Sarotorius Corporation) and purified by size exclusion method. Fractions containing the correctly folded pHLA complex were collected and biotinylated (Avidity). Quality of the pHLA complexes were analyzed by analytical HPLC-SEC (Agilent), SDS-PAGE under denaturing conditions, and ELISA using the W6/32 antibody (BioLegend).

### Off-Target Liabilities Assessment in Alanine Scan

The Off-Target Liabilities Assessment (OTLA) of E07-hOKT3a was evaluated substituting each amino acid position in the G2 peptide, NTDNNLAVY (SEQ ID NO: 214), with alanine or a conserved or a divergent single amino acid substitution (valine or glutamic acid). The resulting peptides were pulsed on K562-HLA cells to evaluate the binding property of the 41-E07-hOKT3a antibody. The alanine and valine or glutamic acid substituted peptide sequences are provided in Table 11.

TABLE 11

| SEQ ID | | |
|---|---|---|
| NO | Name | Peptide |
| 120 | ALA 1 | ATDNNLAVY |
| 121 | ALA 2 | NADNNLAVY |
| 122 | ALA 3 | NTANNLAVY |
| 123 | ALA 4 | NTDANLAVY |
| 124 | ALA 5 | NTDNALAVY |
| 125 | ALA 6 | NTDNNAAVY |
| 126 | ALA 7 | NTDNNLAAY |
| 127 | ALA 8 | NTDNNLAVA |
| 128 | Vor E 1 | NTENNLAVY |
| 129 | Vor E 2 | NTDENLAVY |
| 130 | Vor E 3 | NTDNVLAVY |
| 131 | Vor E 4 | NTDNNVAVY |
| 132 | Vor E 5 | NTDNNLVVY |
| 133 | Vor E 6 | NTDNNLVEY |

### EDGE Prediction

EDGE prediction was done as described in Example 6.

### Pulsing Method

Cells were pulsed with the resulting peptides as described in Examples 2 and 6 to evaluate the binding property of the 41-E07-hOKT3a antibody.

IP-MS Immunoprecipitation of HLA/Peptide Complexes from Cell Lysates

2×10⁸ cells expressing A*01:01/CT83 (NCI-H1703, A375±CT83) were re-suspended in 5 ml lysis buffer (1% CHAPS, 150 mM sodium chloride, 20 mM Tris pH 8) in the presence of phosphatase, and protease inhibitors. The cells were homogenized by sonication, rotated at 4° C. for 1 hr, and spun at 21,000×g for 30 min. to pellet cell debris. To the cleared lysates, 3 mg of ProteinA-conjugated 41-E07 hOKT3a, W6/32, or control IgG antibodies, were added, and the lysate-antibody mixture was rotated at 4° C. overnight.

After immunoprecipitation, the IP beads were isolated using filter plates, then washed to remove non-specific binding using Tris pH 8 buffers with increasing levels of NaCl. The HLA/peptide complex was eluted from the beads using 2N acetic acid. The protein components were removed from the peptides using C18 fractionation. The resultant peptides were taken to dryness by SpeedVac evaporation and stored at −20° C. prior to MS analysis.

Dried peptides were reconstituted along with the relevant stable isotopically labeled peptides in an HPLC buffer suitable for reverse phase chromatography and loaded onto a C-18 microcapillary HPLC column for gradient elution in a Fusion Lumos mass spectrometer (Thermo). MS1 spectra of peptide mass/charge (m/z) were collected in the Orbitrap detector at high resolution followed by MS2 lower resolution scans collected in the ion trap detector after HCD fragmentation of the selected ion. MS2 spectra were measured with high resolution mass accuracy in the Orbitrap detector with targeted method known as parallel reaction monitoring. In targeted PRM, specific peptide precursor ions are isolated in the Orbitrap detector and all resulting HCD fragmentation ions are scanned across the elution of the peptide peak. This enables both peptide identification and quantitation of endogenous peptide in the presence of a co-injected stable isotopically labeled peptide standard. Targeted MS1 and MS2 spectra obtained from the endogenous and synthetic standard peptides were processed through Skyline software (University of Washington).

### Affinity Measurements

The affinities of the monovalent Format 3-E07-hOKT3a antibody with HLA complexes refolded with different peptides of interest was measured using Octet HTX (ForteBio) in 96-channel mode with biolayer interferometry (BLI) detection. The binding constants (KD) of the interactions were measured using the buffer A (0.02% Tween-20 and 0.1% BSA in PBS). All the assay conditions were prepared in a volume of 200 μL. The pHLA complex biotinylated at the C-terminal AviTag was used to ensure uniform directionality of the protein. Biotinylated-pHLA was diluted into assay buffer at 0.6 μg/mL and immobilized onto High Precision Streptavidin SAX biosensors (P/N 18-5117 to a minimum response value of 1 nm on the Octet System (ForteBio). A baseline response was established in assay buffer (120 s) prior to each association. The purified monovalent Format 3-E07 hOKT3a antibody was diluted in the assay buffer at the specified concentrations (typically 500-0 nM, diluted 2-fold). The monovalent Format 3-E07 hOKT3a antibody was allowed to associate for 120 s followed by dissociation for 120 s in the same baseline wells. The assay included one Referencing biosensor with no immobilized ligand dipped into analytes. Using the ForteBio Data Analysis suite, the data was normalized to the association curves followed by background normalization. Curve fitting using global fitting of the sensor data with the Octet™ software using 1:1 kinetic model with errors within 10%, X2below 3, and R2above 0.9 to determine the association and dissociation constants.

### Results

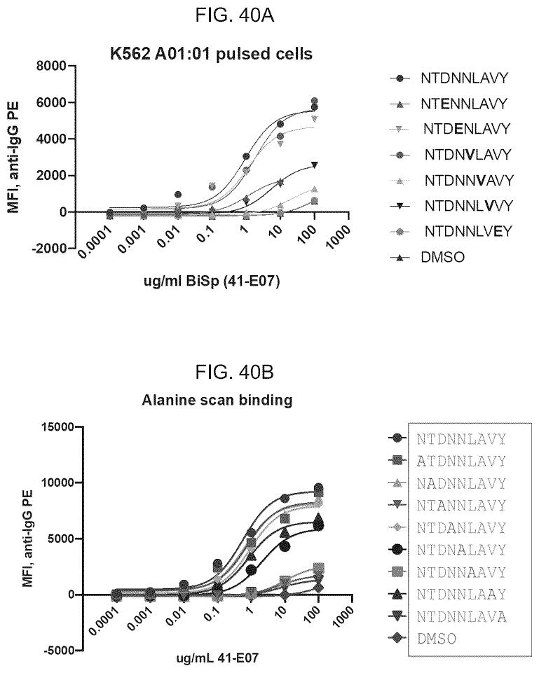

In the valine and glutamic acid replacement assay using 41-E07-hOKT3a, the flow cytometry binding results indicate that replacement of residues 3, 6, 7, and 9 with valine or glutamic acid resulted in a significant reduction in binding of 41-E07-hOKT3a (FIG. 40A). Substitution of residues 2, 4, 5, and 8 with valine or glutamic acid showed minimal impact on binding (FIG. 40A). Results are also shown in Table 12.

TABLE 12

| | Max | Fold change in | | Fold change |
|---|---|---|---|---|
| Position | Binding | max binding | EC50 | in EC50 |
| WT | 5563 | | 0.9353 | |
| V or E 1 | 1885 | 0.3388 | 1.128 | 1.206 |
| V or E 2 | 4683 | 0.8418 | 1.14 | 1.218 |
| V or E 3 | 5685 | 1.0219 | 1.788 | 1.911 |
| V or E 4 | 1558 | 0.2800 | 20.24 | 21.640 |
| V or E 5 | 2634 | 0.4734 | 5.435 | 5.810 |
| V or E 6 | 1401 | 0.2518 | 91.7 | 98.043 |

In the alanine scan assay using 41-E07-hOKT3a, the flow cytometry binding results indicate that replacement of residues 3, 6, and 9 with alanine resulted in a significant reduction in binding, while substitution of 5^{th} residue with alanine increased the EC50 by approximately 4-fold (FIG. 40B). Substitution of residues 1, 2, 4, and 8 with alanine showed minimal impact on binding (FIG. 40B).

The alanine scan results were substantiated with affinity measurement of peptide-HLA complex with the monovalent Format 3 E07-hOKT3a antibody using Bio-layer interferometry affinity measurement methods (Table 13).

TABLE 13

| | | | | | Fold | | |
|---|---|---|---|---|---|---|---|
| | Max | | | | change | | Fold |
| | Response | KD | Fold change | Fold change | in max | | change |
| Position | (nm) | (nM) | in Response | in affinity | binding | EC50 | in EC50 |
| WT | 0.24 | 1.2 | — | — | — | 0.5536 | |
| Ala 1 | 0.26 | 3 | — | 2.5 decrease | 0.90 | 0.7489 | 1.35 |
| Ala 2 | 0.24 | 1.5 | — | — | 0.88 | 0.6301 | 1.14 |
| Ala 3 | 0.15 | 17 | 1.6 decrease | 14.2 decrease | 0.19 | 7.355 | 13.29 |
| Ala 4 | 0.24 | 8.7 | — | 7.3 decrease | 0.86 | 0.9113 | 1.65 |
| Ala 5 | 0.21 | 16 | — | 13.3 decrease | 0.64 | 2.062 | 3.72 |
| Ala 6 | 0.09 | 16 | 2.7 decrease | 13.3 decrease | 0.28 | 9.866 | 17.86 |
| Ala 8 | 0.24 | 8 | — | 6.7 decrease | 0.71 | 0.8319 | 1.50 |
| Ala 9 | 0 | n/a | Complete loss | n/a | 0.15 | 7.648 | 13.82 |

Based on highest response (nm) on the Octet, Positions 3, 6, and 9, showed the least response to non-binding of the peptide complex to the peptide. Substitution at position 9 resulted in complete loss of binding and therefore is a critical residue for binding.

Based on the cytometry and affinity measurements, close analogues of G2 peptide (up to five mismatches in the human genome, 800 peptides) were chosen to evaluate the cross reactivity of the 41-E07-hOKT3a antibody. Using the EDGE prediction, 66 peptides with highest predicted binding to A*01:01 allele were identified (Table 14).

TABLE 14

| | Peptides | |
|---|---|---|
| | with | SEQ |
| | positional | ID |
| Gene | change | NO |
| AAMP | YLDGTLAIY | 134 |
| ABCB4 | FSDKELAAY | 135 |
| ALDH5A1 | AADVGLAGY | 136 |
| AP4S1 | DTENEMAIY | 137 |
| ARRDC4 | RVDYSLAVY | 138 |
| ATP8B1 | YADGKLAFY | 140 |
| BCOR | GADPTLATY | 141 |
| CC2D2B | QTEFALAVY | 142 |
| (C10orf131) | | |
| CCND2 | ATDFKFAMY | 143 |
| CCND3 | ATDYTFAMY | 144 |
| CD2 | GTDPELNLY | 145 |
| CFAP47 | WTDVFLQIY | 146 |
| (CXorf22) | | |
| CNP | RLDEDLAAY | 147 |
| CYP2J2 | TVDTTLAGY | 148 |
| DMD | FIDKQLAAY | 149 |
| DNAH1 | QTDFPLQAY | 150 |
| DNAH7 | YIDIFLNVY | 151 |
| DSG3 | YTDNWLAVY | 152 |
| DYRK1A | AMDVNLTVY | 153 |
| E2F2 | RTEDNLQIY | 154 |
| ERICH3 | LMDKHLAGY | 155 |
| (C1orf173) | | |
| F13B | VADGILASY | 156 |
| FBXO21 | SLDLYLAMY | 157 |
| GABRB3 | NMDYTLTMY | 158 |
| GABRD | NLDGLIAGY | 159 |
| GFAP | EAENNLAAY | 160 |
| HYDIN | VTDFKFALY | 161 |
| JAK2 | QIDPVLQVY | 162 |
| LEP | KMDQTLAVY | 163 |
| LMLN | NMDRPIAGY | 164 |
| LRPPRC | NLDFVLSFY | 165 |
| MCM3 | ETEYGLSVY | 166 |
| MKKS | CTETHLAAY | 167 |
| MMP8 | EADINIAFY | 168 |
| NAV2 | MTDGGLGLY | 169 |
| NAV3 | MTDGGLNLY | 170 |
| NHLRC2 | NTEEPISVY | 171 |
| NID2 | NVDRVFALY | 172 |
| NT5C2 | DMDYTLAVY | 173 |
| OARD1 | ATDIKITVY | 174 |
| (C6orf130) | | |
| OTULIN | NTEEFITVY | 175 |
| (FAM105B) | | |
| PASK | RLDEPLASY | 176 |
| PPAN-P2RY11 | TADHGLAAY | 177 |
| PSME1 | KTENLLGSY | 178 |
| PTPRO | WTDYLLAFY | 179 |
| RNF212B | QTDLLIAFY | 180 |
| (C14orf164) | | |
| ROBO1 | NSDSNLTTY | 190 |
| SH2B3 | KTDHFLSCY | 191 |
| SLC16A8 | CTDRAFAVY | 192 |
| SLC25A10 | RTDGILALY | 193 |
| SND1 | CVDWSIAVY | 194 |
| STIP1 | NIDDALQCY | 195 |
| SYN3 | FSELNLAAY | 196 |
| TLE6 | GMDDFLGVY | 197 |
| TMEM232 | NTDSDMGGY | 198 |
| TRIM47 | YADRALAFY | 199 |
| ZNF503 | GTDKLLSGY | 200 |
| ZNF507 | QVDSTLAAY | 201 |
| ZNF653 | WSDAKLAAY | 202 |
| DSG4 | GTDNWLAQY | 203 |
| PTS | ETDNNIVVY | 204 |
| KDM7A | PTDENLARY | 205 |
| (JHDM1D) | | |
| ICE1 | NTDNLLTEY | 206 |
| (KIAA0947) | | |
| RBM4 | LTEQYNEQY | 207 |
| ERGIC3 | TTEVHPELY | 208 |
| UBR2 | ATDLTREVY | 209 |

Immunoprecipitation combined with targeted mass spectrometry (IPMS) method was used to assess the presentation of these 66 peptides by the HLA (allele A*01:01) that could be recognized by the 41-E07-hOKT3a antibody. The 41-E07-hOKT3a antibody as well as pan HLA1 (b2m specific) antibody was used to assess two cancer cell lines. K562-A*01:01 (engineered to express HLA A*01:01) and NCI-H1703 (naturally harbor HLA A*01:01). These two cell lines express the majority of these OTLA genes (expression >1 TPM per CCLE, Cancer Cell Line Encyclopedia). Six of those peptides presented by HLA, were identified by the IPMS experiment as possible off target interactions (Table 15). Where targets of the OTLA peptides were detected, target densities as given by copies per cell are shown.

TABLE 15
Target densities of G2, OTLA peptides in A*01:01-expressing cell lines

| | NCI-H1703 | | | K562-A*01:01 | | | |
|---|---|---|---|---|---|---|---|
| | 41E07 | IgG | W6/32 | | | | Potential |
| Peptide (Gene) | Rep1:Rep2 | Rep1:Rep2 | Rep1:Rep2 | 41E07 | IgG | W6/32 | OTLA |
| NTDNNLAVY | 2958; 2959 | ND; ND | 3789; 4040 | ND | ND | ND | |
| (SEQ ID NO: | | | | | | | |
| 214) (CT83) | | | | | | | |
| ATDLTREVY | 146; 147 | 6; 4 | 891; 876 | 6 | ND | 6 | Yes |
| (SEQ ID NO: | | | | | | | |
| 209) (UBR2) | | | | | | | |
| LTEQYNEQY | 1146; 1127 | 113; 79 | 16727; 16746 | 60 | ND | 1004 | Yes |
| (SEQ ID NO: | | | | | | | |
| 207) (RBM4) | | | | | | | |
| TTEVHPELY | 34; 37 | 25; 15 | 3358; 3352 | ND | ND | 96 | Yes |
| (SEQ ID NO: | | | | | | | |
| 208) (ERGIC3) | | | | | | | |
| ETDNNIVVY | 40; 42 | 32; 25 | 2927; 2980 | 12 | ND | 91 | Yes |
| (SEQ ID NO: | | | | | | | |
| 204) (PTS) | | | | | | | |
| PTDENLARY | 0; 0 | 0; 0 | 2; 2 | ND | ND | ND | No |
| (SEQ ID NO: | | | | | | | |
| 205) (KDM7A) | | | | | | | |
| NTDNLLTEY | 33; 32 | 35; 21 | 2661; 2745 | ND | ND | ND | Yes |
| (SEQ ID NO: | | | | | | | |
| 206) (IC1) | | | | | | | |
| GTDNWLAQY | 0; 0 | 0; 0 | 0; 0 | ND | ND | ND | No |
| (SEQ ID NO: | | | | | | | |
| 203) (DGS4) | | | | | | | |
| NSDSNLTTY | 85; 79 | 0; 0 | 554; 517 | ND | ND | ND | Yes |
| (SEQ ID NO: | | | | | | | |
| 190) (ROBO1) | | | | | | | |
| YTDNWLAVY | 0; 0 | 0; 0 | 0; 0 | ND | ND | ND | Yes |
| (SEQ ID NO: | | | | | | | |
| 152) (DSG3) | | | | | | | |

ND: Not detected

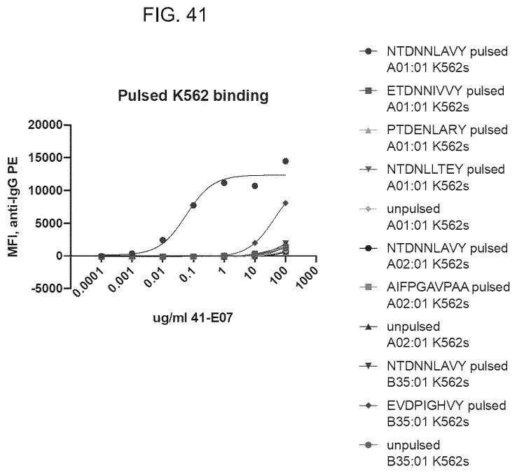

Peptides identified by IPMS experiment were de-risked by in vitro assays. The A01:01 HLA presenting cells pulsed with the OTLA peptides of interest show no or minimal interactions (FIG. 41). Some binding to EVDPIGHVY (SEQ ID NO: 233) pulsed β35:01 cells was observed, but there was a more than 3 log difference in EC50 as compared to the G2 peptide NTDNNLAVY (SEQ ID NO: 214).

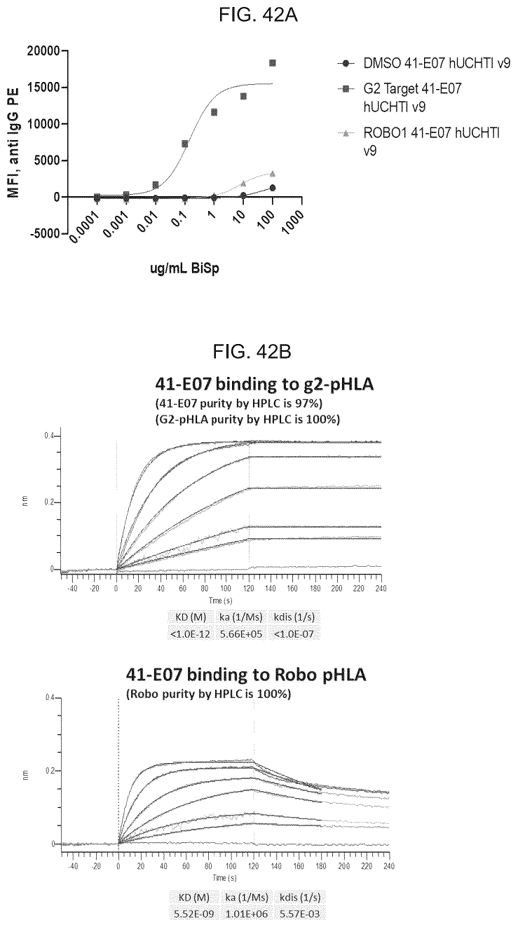

Of these OTLA peptides, only the peptide from ROBO1 gene, when presented on HLA showed a prominent interaction with 41-E07-UCHT1v9 (FIG. 42A). The EC50 for 41-E07-UCHT1v9 to the G2 target peptide was 0.1461, while the EC50 for 41-E07-UCHT1v9 to the ROBO1 target was 7.331. However, when this peptide was refolded into A*01:011b2m complex, it showed 5000 times less binding affinity to 41-E07-UCHT1v9, as compared to the G2-peptide A*01:011b2m complex, as measured by octet experiment (FIG. 42B).

### Example 15: SEC-HPLC Analysis of Format 4 Bispecific Antibodies Reveal Presence of an Alternative Isomer

### Methods

Analytical SEC-HPLC was performed on an Agilent 1200 series HPLC system equipped with a degasser (G1379B), binary pump (G1312B), high performance autosampler (G1367D), and wide range diode array detector (DAD, G7115A). Approximately 50-100 ug of Format 4 G5(1C12) protein A eluate, neutralized to pH 7 using 1M Tris buffer pH 7.5, was loaded onto a TSKgel SuperSW mAb HTP column (4.6 mm ID×15 cm) with the TSKgel Guardcolumn SuperSW mAb guard column in line, or TSKgel G3000 SWxl column (7.8 mm ID×30 cm) with the TSKgel G2000SWxl-G4000SWx1 Guard Column in line from Tosoh Bioscience. The TSKgel SuperSW mAb HTP column was operated at 0.35 ml/min for 7 min in PBS pH 7.4. The TSKgel G3000 SWxl column was operated at 0.5 ml/min for 35 min in PBS, pH 7.4. The DAD was set to collect absorbance at 280 nm for both methods.

### Results:

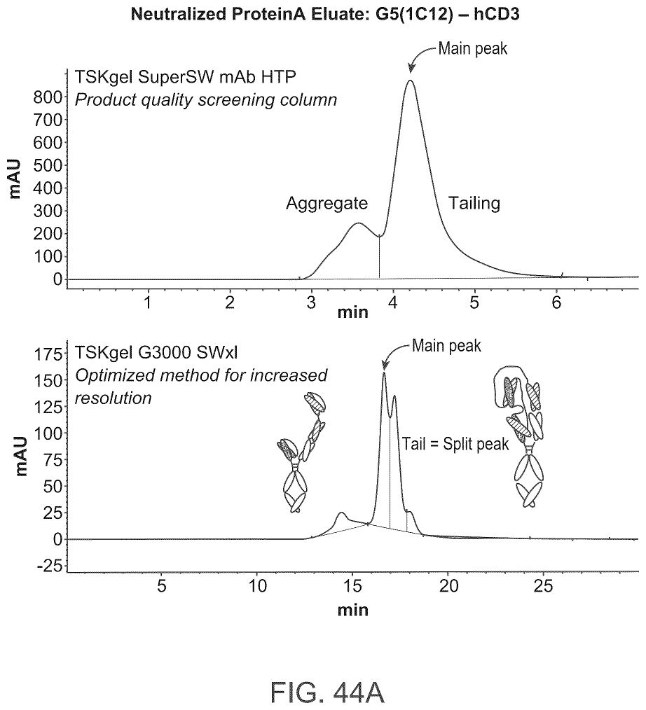

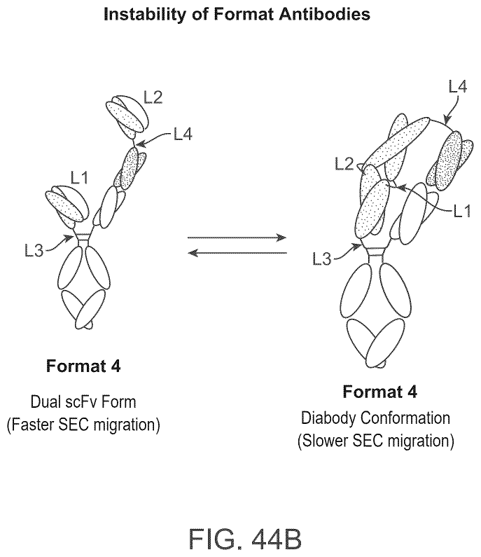

Analysis of Format 4-G5(1C12) hOKT3a proteinA eluate using the TSKgel SuperSW mAb HTP column (FIG. 44A, top), used for quick product quality screening of antibodies, revealed the presence of aggregates between 3-4 min, a main peak, and an unexpected significant tailing between 4.5-5.5 minutes. The observed tailing suggested the presence of an additional antibody moiety that either interacts more with the SEC column, or is more compacted and thus migrates slower than the main antibody conformation. Analyzing the same proteinA eluate using the TSKgel G3000SWxl column, which has greater resolving power than the shorter TSKgel SuperSW mAb HTP column, shows that the tailing initially observed resolves into a “split peak” (FIG. 44A, bottom). Mass spectrometry analysis of the Format 4-G5(1C12) hOKT3a antibody suggested no fragmentation (data not shown). Accordingly, the “split peak” was hypothesized to be a diabody isoform of the Format 4 antibody, where the VH of one of the scFvs interacts with the VL of the other scFv and vice versa. (FIG. 44B, bottom panel, bispecific diagram on the right).

These results indicated that the antibodies can exist in two conformations. They further indicated that in solution, the antibody will be in equilibrium between (1) a dual scFv conformation and (2) a diabody conformation (see FIG. 5).

### Example 16: Determination of Alternate Diabody Isoform

### Materials and Methods

### Antibody Digestion Experiment

0.4 mg each of purified G5(1C12) hOKT3a format 3, 4 and 5 bispecific antibodies were buffer exchanged from PBS pH 7.4 into 150 mM sodium phosphate buffer at pH 7.0. The samples were then concentrated to a volume of approximately 100 μL, with corresponding concentrations ranging from 3-4 mg/mL, loaded onto FabALACTICA microspin columns (Genovis), and incubated for 16 hr with end-over-end mixing. FabALACTICA antibody digestion involves a cysteine protease that digests human IgG1 at one specific site above the hinge (KSCDKT/HTCPPC (SEQ ID NO: 211)), generating intact Fab and Fc fragments. The name of the enzyme is derived from the pathogen Streptococcus agalactiae, where it was first discovered. Spoerry, Christian & Hessle, Pontus & Lewis, Melanie & Paton, Lois & Woof, Jenny & Pawel-Rammingen, Ulrich. (2016). Novel IgG-Degrading Enzymes of the IgdE Protease Family Link Substrate Specificity to Host Tropism of Streptococcus Species. PLoS ONE. 11. e0164809. 10.1371/journal.pone.0164809), which is hereby incorporated by reference in its entirety. To collect the digested products, the columns were centrifuged at 1000×g for 1 min, followed by two additional rounds of elution using 100 μL PBS pH 7.4. The elution fractions were pooled and subsequently loaded onto a CaptureSelect (Genovis) column, and incubated for 30 min with end-over-end mixing. The flowthrough was collected by centrifugation at 200×g for 1 min, followed by two wash steps with 100 μL PBS (200×g for 1 min, and 100×g for 1 min, respectively). The flowthrough and wash fractions were pooled, and are henceforth referred to as “ProteinA Flowthrough”. The ProteinA bound fragments were eluted using 100 μL of 0.1M Glycine, pH 3 by centrifugation at 200×g for 1 min, and immediately neutralized with 50 μL 1M tris pH 7.5. A second elution step was performed by centrifugation at 1000×g for 1 min, and neutralized immediately as described. The elution fractions were pooled and are henceforth referred to as “ProteinA bound/Eluted”

### SEC-HPLC Analysis

Analytical SEC-HPLC was performed on an Agilent 1200 series HPLC system equipped with a degasser (G1379B), binary pump (G1312B), high performance autosampler (G1367D), and wide range diode array detector (DAD, G7115A. Approximately 40 μL of each of untreated antibody, digested proteinA flowthrough, and digested ProteinA bound/eluted was loaded onto a TSKgel G3000 SWxl column (7.8 mm ID×30 cm) with the TSKgel G2000SWxl-G4000SWxl Guard Column in line from Tosoh Bioscience. The column was operated at 0.5 ml/min for 60 min in PBS, pH 7.4. The DAD was set to collect absorbance at 280 nm.

### Results

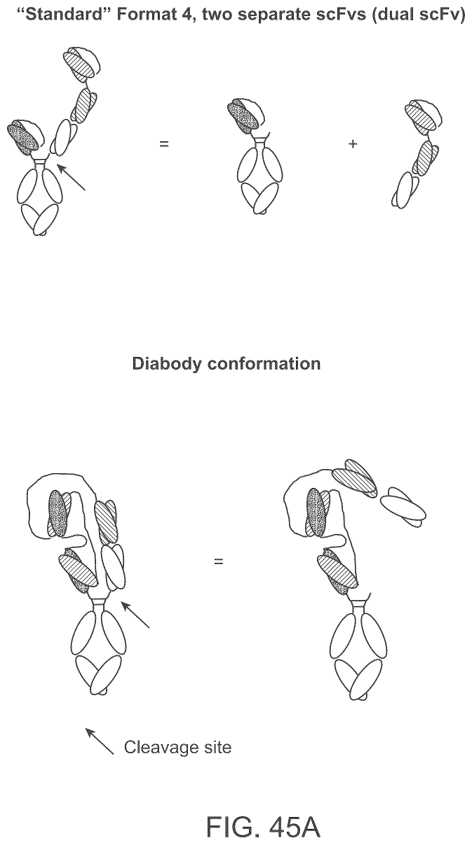

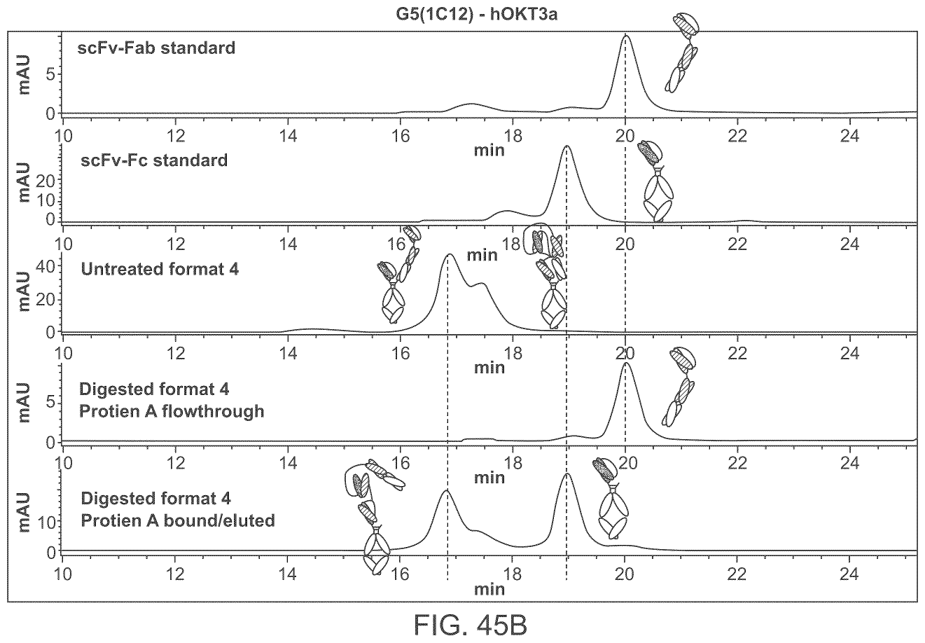

FIG. 45A depicts expected protein digestion fragments of “standard” Format 4 antibodies and a “diabody” isomer of Format 4. FabALACTICA digestion of “standard” Format 4 conformation (scFv/scFv-Fab) antibodies with two separate scFvs, without presence of any alternative “diabody” isoforms, would be expected to yield two peaks: one corresponding to the scFv-Fc fragment and one corresponding to the scFv-Fab fragment. Presence of a Format 4 “alternative diabody” conformation would be expected to reveal presence of a third peak that aligns with the undigested Format 4 main peak.

SEC-HPLC results are depicted in FIG. 45B. Digested format 5 ProteinA flowthrough is used as the scFv Fab standard, and digested format 3 Protein A bound/Eluted is used as the ScFv-Fc standard. The undigested Format 4 SEC-HPLC profile shows the previously described split peak. Digested Format 4 ProteinA flowthrough showed a peak with a retention time that aligned with the scFv-Fab standard. Digested Format 4 ProteinA bound/Eluted SEC-HPLC profile showed a peak that aligned with the scFv-Fc standard expected to be seen for the “standard” Format 4, as well as a peak that aligned with the undigested format 4. The presence of the latter peak indicated the presence of the alternate diabody conformation.

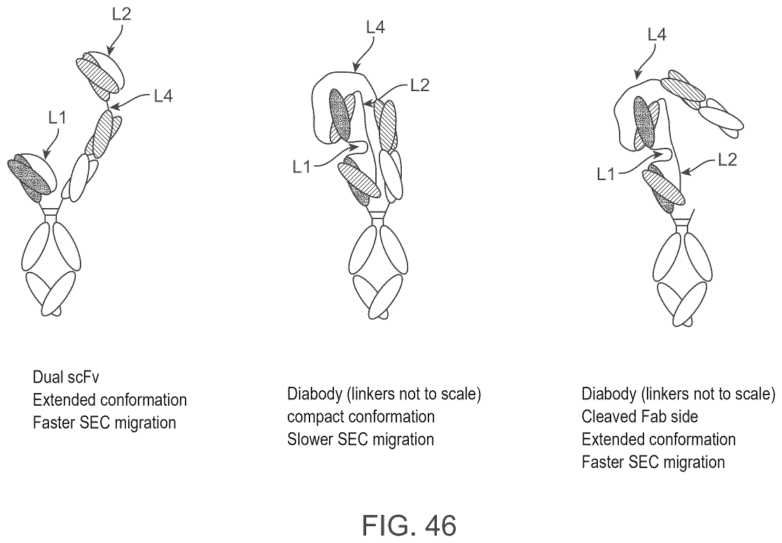

FIG. 46 depicts a diagram representation of the undigested Format 4 “separate scFv” conformation (left), the alternate diabody conformation without digestion (middle), and the alternate diabody conformation with digestion (right).

### Example 17: Negative Stain Electron Microscopy and 2D Class Averaging

### Materials and Methods

### Grid Preparation

A sample of Format 4-G5(1C12) hOKT3 bispecific antibody was diluted to 18 μg/mL using PBS prior to imaging. The sample was imaged over a layer of continuous carbon supported by nitro-cellulose on a 400-mesh copper grid. The grids were prepared by applying 3 μl of sample suspension to a cleaned grid, blotting away with filter paper, and immediately staining with uranyl formate.

### EM Imaging

Electron microscopy was performed using an FEI Tecnai T12 electron microscope (serial number D1100), operating at 120 keV equipped with an FEI Eagle 4 k×4 k CCD camera. Negative stain grids were transferred into the electron microscope using a room temperature stage.

Images of each grid were acquired at multiple scales to assess the overall distribution of the specimen. After identifying potentially suitable target areas for imaging at lower magnifications, high magnification images were acquired at nominal magnifications of 110,000× (0.10 nm/pixel) and 67,000× (0.16 nm/pixel). The images were acquired at a nominal underfocus of −1.6 μm to −0.8 μm and electron doses of −25 e/Å.

### 2D Averaging Analysis

Particles were identified in the high magnification images prior to alignment and classification. The individual particles were then selected, boxed out, and individual sub-images are combined into a stack to be processed using reference-free classification.

Particle Selection: Individual particles in the 67,000× high magnification images were selected using automated picking protocols described in Lander, G. C., S. M. Stagg, et al. (2009). “Appion: an integrated, database-driven pipeline to facilitate EM image processing.” J Struct Biol 166(1): 95-102, which is hereby incorporated by reference in its entirety, and manual picking. An initial round of alignments was done on each sample and from that alignment class averages that appeared to contain recognizable particles were selected for additional rounds of alignment.

Particle Alignment and Classification: A reference-free alignment strategy based on the XMIPP (Sorzano, Marabini et al. 2004) processing package, described in Sorzano, C., R. Marabini, et al. (2004). XMIPP: a new generation of an open-source image-processing package for electron microscopy. J Struct Biol. 148: 194-204, which is hereby incorporated by reference in its entirety, was used. Algorithms in this package align the selected particles and sort them into self-similar groups of classes.

### Results

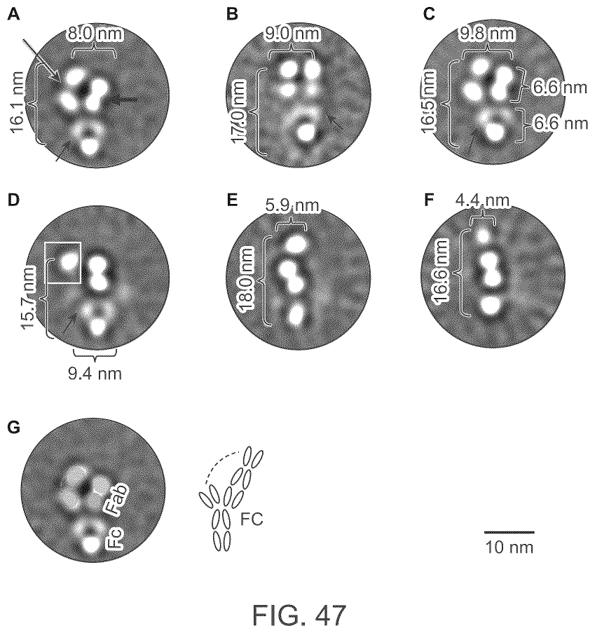

FIG. 47 depicts electron microscopy results. Visible in the sample were particles that displayed different sizes and morphologies. Particles ranged from ˜16-22 nm in their longest dimension and had a wide range of conformations; some particles had a branched appearance and others were irregular in shape. Class averages showed particles that ranged from ˜5 to 10 nm in width and ˜16 to 18 nm in length (see FIG. 47). The majority of the class averages contained features that resembled those seen for IgG molecules: a single Fc domain and two antibody arms. However, there were aspects that distinguished these particles from a typical antibody sample: 1. One of the antibody arms contained a peanut-shaped moiety closely resembling a typical Fab (FIG. 47, panel A, black arrow). The other arm appeared to contain two spherical domains, but at a greater distance from each other when compared to that seen in a standard Fab arm (FIG. 47, panel A, light gray arrow). Based on the model of this bispecific antibody, it is likely that only one of these two spherical domains was connected to the Fc region, whereas the other was in fact connected to the end of the neighboring arm. It seems to be flexibly linked, as it can bend down and interact with the tip of the neighboring Fab arm. These interacting spherical domains are mostly likely the two scFv domains of the Format 4 antibody. Thus the EM revealed visual evidence of the alternative diabody isomer.

It should be noted that in a few class averages, the Fc and Fab domains were stacked in a straight line making it impossible to distinguish between them (FIG. 47, panels E and F). These are likely side views of the particle described above.

Averages were generally well-defined, with some portions of the Fc domain not as clearly resolved as others.

### Example 18: Introduction of DSB44/100 Removes Putative Diabody Peak

### Materials and Methods

### DSB Engineering

Position 44 of the VH (Kabat) is often in close proximity to position 100 of VL (Kabat). By introducing Cys residues at both of these positions, a disulfide bond (DSB) can be formed that stabilizes the VH/VL interactions within each scFv, prior to assembly of the bispecific antibody chains. Such a stabilizing DSB would be expected to reduce the probability that the two scFvs of the Format 4 antibodies interact to form the alternative diabody isomer.

Gene fragments incorporating the H44-L100 DSB mutations (Kabat numbering) were ordered through Genewiz, incorporating 18-base pair overlaps with digested vector. Fragments were cloned using In-Fusion homologous recombination (Takara) according to manufacturer's instructions. Clones were confirmed to be correct by sequencing (Elim Biopharmaceuticals). Molecules were generated by transfection of Expi293F cells according to manufacturer's recommended protocols (Life Technologies). Molecules were purified on Akta AVANT using protein A and Kappa Select Light columns (GE Healthcare) and polished using CHT (Bio-Rad) for aggregate removal.

### SEC-HPLC

Analytical SEC-HPLC was performed on an Agilent 1200 series HPLC system equipped with a degasser (G1379B), binary pump (G1312B), high performance autosampler (G1367D), and wide range diode array detector (DAD, G7115A). Purified Format 4 G5(1C12) and G2(1H11) antibodies, with and without the DSB were loaded onto a TSKgel G3000 SWxl column (7.8 mm ID×30 cm) with the TSKgel G2000SWxl-G4000SWx1 Guard Column in line from Tosoh Bioscience. The column was operated at 0.5 ml/min for 30 min in PBS, pH 7.4. The DAD was set to collect absorbance at 280 nm.

### Results

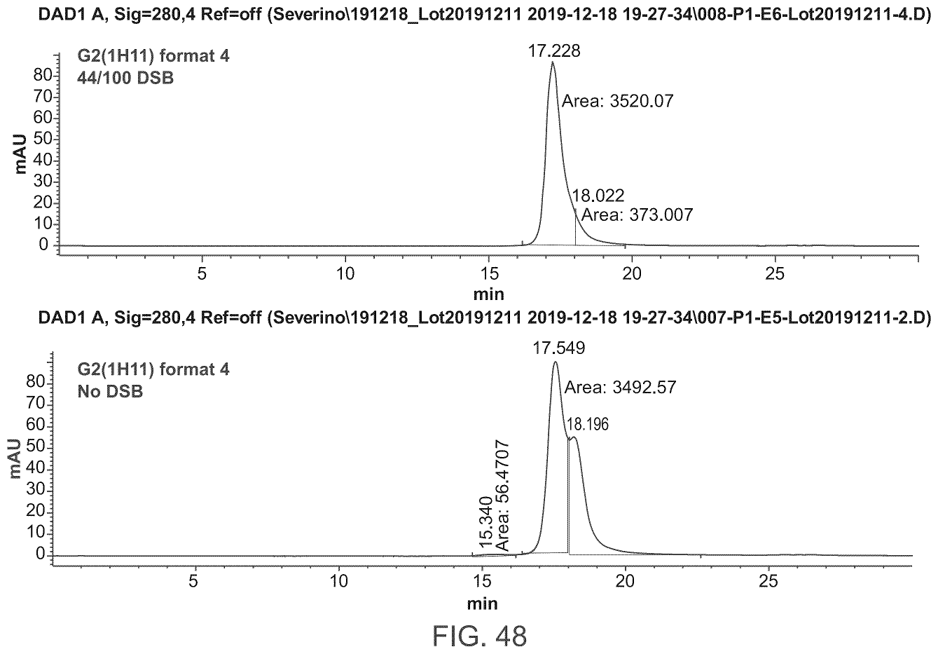

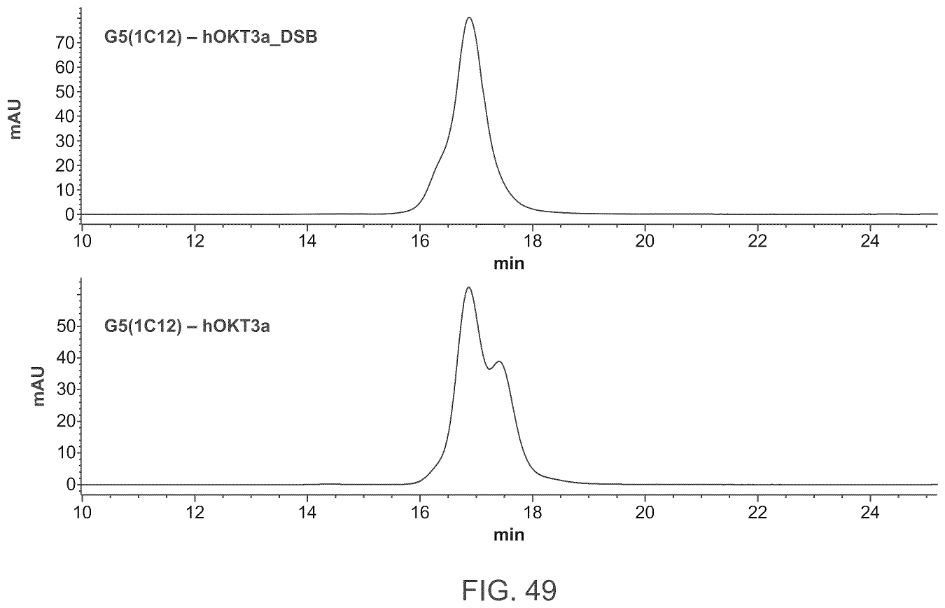

Bottom panels of FIG. 48 and FIG. 49 show the previously observed split peak for both Format 4-G5 and G2 molecules, indicating the presence of both “standard” (with two separate scFvs) and alternate diabody conformation across all Format 4 molecules. Introduction of a stabilizing disulfide bond within the scFv regions of both molecules is shown to remove the split peak (top panels of FIG. 48 and FIG. 49). A retention time that aligns with that of the Format 4 “standard” conformation suggests that the introduction of a disulfide bond stabilizes the standard conformation with two separate scFvs for both G5 and G2 molecules and reduces their isomerization into the alternative diabody format.

### Example 19: Digestion of Format 4 Antibody with DSB44/100

### Materials and Methods

### Proteolysis by FabALACTICA:

0.3-0.5 mg of Format 4 G5(1C12)-hOKT3_DSBH44/L100 and Format 4 G2(1H11)-hOKT3_DSBH44/L100 antibodies (both having non-shortened linkers: L1=L2=(G4S)×4 (SEQ ID NO: 228); L3=L4=(G4S)×2 (SEQ ID NO: 111)) were buffer exchanged from PBS pH 7.4 into 150 mM sodium phosphate buffer at pH 7.0. The samples were then concentrated to a volume of approximately 100 μL, with corresponding concentrations ranging from 3-5 mg/mL, loaded onto FabALACTICA microspin columns (Genovis), and incubated for 18 hr with end-over-end mixing. To collect the digested products, the columns were centrifuged at 1000×g for 1 min, followed by three additional rounds of elution using 100 μL PBS pH 7.4. The elution fractions were pooled (referred to as “digested pool”), and subsequently loaded onto a CaptureSelect ProteinA (Genovis) column, and incubated for a minimum of 30 min with end-over-end mixing. The flowthrough was collected by centrifugation at 200×g for 1 min, followed by three wash steps with 100 μL PBS pH 7.4. The flowthrough and wash fractions were pooled, and are henceforth referred to as “‘Fab’ Fraction”. The Protein A bound fragments were eluted using 100 μL of 0.1 M Glycine, pH 3, by centrifugation at 200×g for 1 min, and immediately neutralized with 10 μL 1M Tris pH 8. Four additional elution steps were performed by centrifugation at 1000×g for 1 min, and neutralized immediately as described. The elution fractions are henceforth referred to as “‘Fc’ fraction”.

### SEC-HPLC

Analytical SEC-HPLC was performed on an Agilent 1260 series HPLC system equipped with a degasser (G4225A), binary pump (G1312B), autosampler (G1329B), and diode array detector (DAD, G4212B). Approximately 60 to 100 μg of each untreated antibody, and 100 μL of the “Fab” fraction, and “Fc” fraction were loaded onto a TSKgel G3000 SWxl column (7.8 mm ID×30 cm) with the TSKgel G2000SWxl-G4000SWxl Guard Column in line from Tosoh Bioscience. The column was operated at 0.5 mL/min for 30 min in PBS, pH 7.4. The DAD was set to collect absorbance at 280 nm.

### CE-SDS

Capillary gel electrophoresis was performed using the LabChip GXII Touch HT system (PerkinElmer), and samples were analyzed using the ProteinExpress 200 High Sensitivity assay (PerkinElmer, #CLS960008) under reducing and non-reducing conditions. 2 μg of each untreated antibody, and 5 μL of each of the digested pool, “Fab” fraction, and “Fc” fraction were mixed with 7 μL of reducing or non-reducing denaturing solution, and incubated at 70° C. for 10-12 min. The reducing denaturing solution was prepared by adding 24.5 μL of 1 M DTT to 700 μL of non-reducing denaturing solution provided in the kit. Denatured samples were diluted with 32 μL of MilliQ water, mixed well, and spun down prior to analysis. The Protein Express LabChip (PerkinElmer, #760499), and ladder were prepared according to manufacturer instructions.

### Results

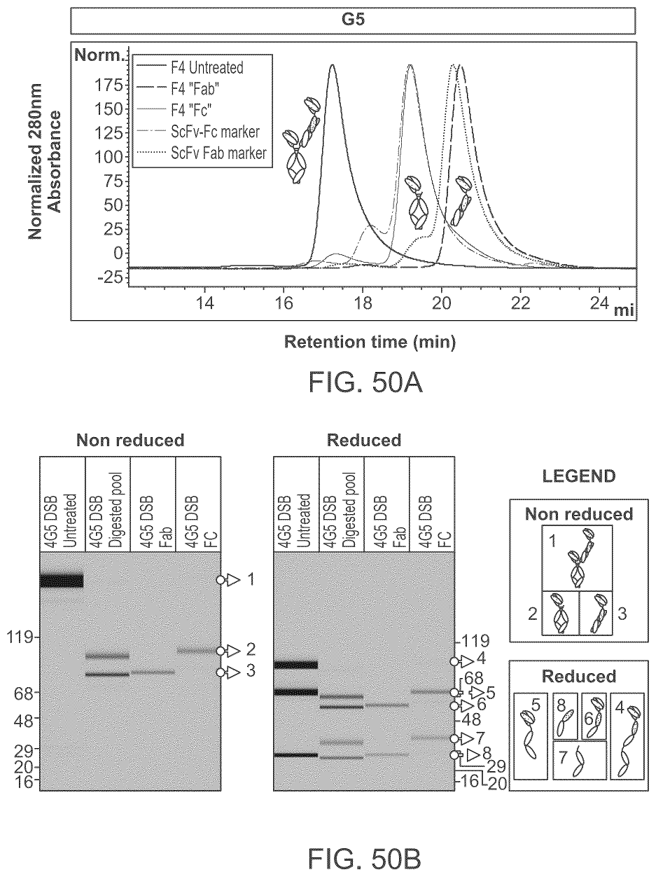

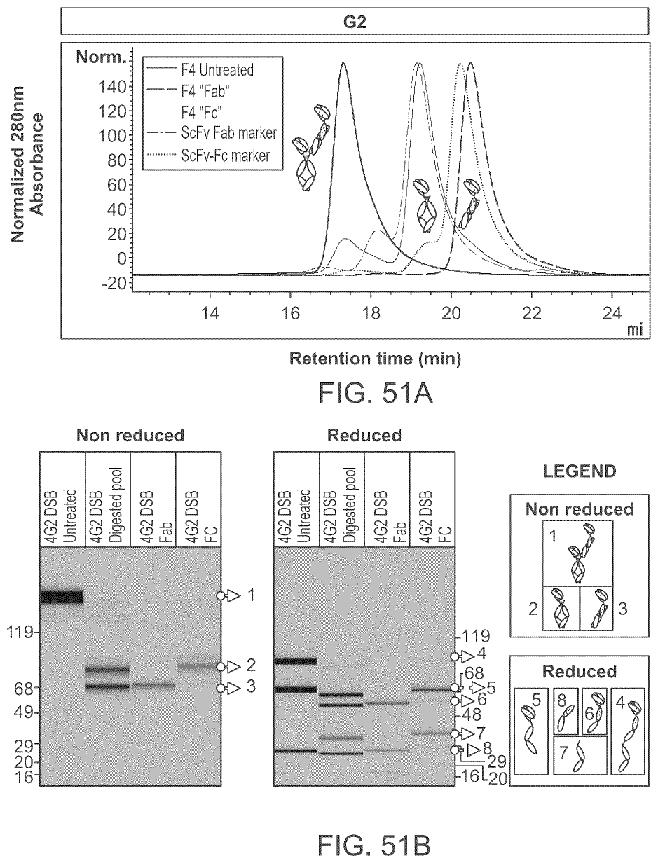

Format 4 molecules stabilized in the 2×ScFv conformation were expected to exhibit a single peak when analyzed by SEC-HPLC. Both undigested G2 and G5 molecules containing the DSB H44/L100 mutation were observed to migrate as single peaks, with a retention time of approximately 17.3 minutes (FIG. 50A and FIG. 51A), suggesting that the putative diabody formation had been eliminated via disulfide bond stabilization. Further, proteolysis of these Format 4 molecules resulted in two fragments, namely the scFv-Fc fragment (#2 in FIG. 50A and FIG. 51A), and the scFv-Fab fragment (#3 in FIG. 50B and FIG. 51B), as observed in the non-reducing gel. Complete separation of these fragments is observed upon purification of the digested pool by protein A when analyzed by both, CE-SDS and SEC-HPLC. The absence of residual scFv-Fab related bands in the “Fc” fraction lanes under both reducing and non-reducing conditions indicated stabilization of the 2×scFv conformation for both the G2 and G5 antibodies. Additionally, the absence of a significant peak that aligns with the undigested samples in the Fc-fraction when analyzed by SEC-HPLC also indicated that the 2×scFv conformation has been successfully stabilized in both G2 (FIG. 51A) and G5 (FIG. 50A) molecules.

### Example 20: Effect of Engineered DSB on Apparent Affinity as Measured by BLI

Format 4 bispecific antibodies with or without DSB mutations as described in Example 18 were generated. The affinity of wildtype and DSB mutants were analyzed on the ForteBio Octet HTX in 96-channel mode with biolayer interferometry (BLI) detection. High Precision Streptavidin SAX biosensors (P/N 18-5117) were loaded into the instrument. Biotinylated G2-pHLA or G5-pHLA was captured on the SAX biosensor at 2 μg/mL and ran for 120 s in the assay buffer composed of 0.02% Tween-20 and 0.1% BSA. The biosensors were then dipped in assay buffer for a baseline. Subsequently, the biosensors were dipped into wells containing varying concentrations of the bispecific antibody samples (3.125, 6.25, 12.5, 25, 50, 100 and 200 nM) to measure the association rate for 50 seconds. The biosensors were finally dipped into wells containing assay buffer to measure the dissociation rate for another 50 seconds. Referencing was completed by having a biosensor with no immobilized ligand dipped into analyte. Kinetic data was processed with Octet™ software using a 1:1 kinetic model with errors within 10%, X² below 3, and R² above 0.9.

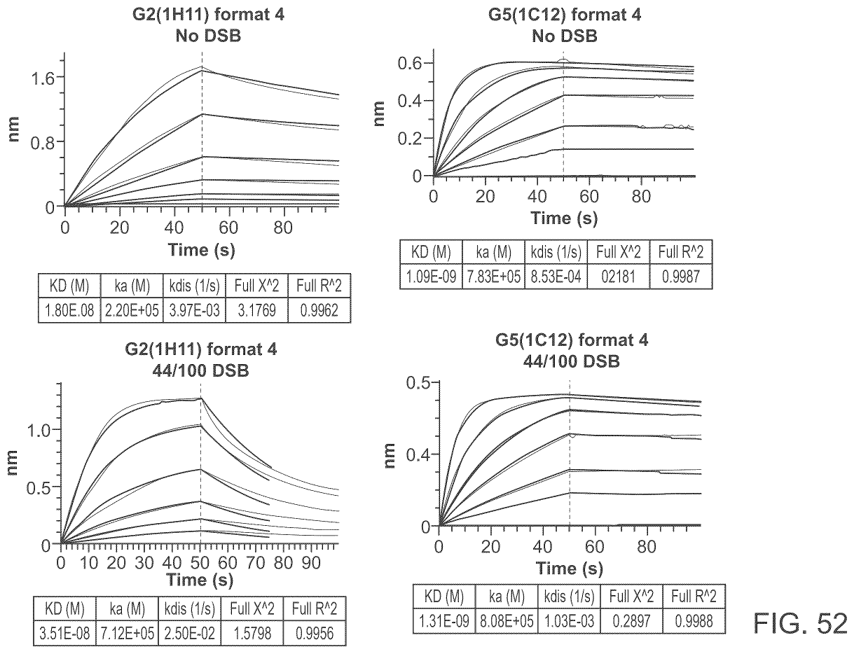

Results are depicted in FIG. 52. Introduction of the DSB mutation increased the K_{D} of the G2 Format 4 bispecific from 18 nM to 35.1 nM. Introduction of the DSB mutation increased the K_{D} of the G5 Format 4 bispecific from 1.09 nM to 1.35 nM.

### Example 21: Effect of DSB on Apparent Affinity as Measured by MSD

The effect of the stabilizing DSB on cell binding of Format 4 G2 and G5 antibodies was assessed using the Meso Scale Discovery (MSD) U-PLEX Development Pack, 9-assay (cat. No. K15234N). Biotinylated pHLA and biotinylated Protein A were each diluted to 33 nM using PBS+0.5% BSA. For each plate, 200 μL of the diluted pHLA or protein A was mixed with 300 μL Linker and incubated at room temperature for 30 minutes.

Following the 30 minute incubation, 200 μL Stop solution was added to each linker-pHLA solution. They were again incubated for 30 minutes at room temperature. These volumes were scaled based on the number of plates. At this point, the linker-pHLA solutions were a 10× solution. They were then pooled together and further diluted with stop solution to the final 1× concentration. All volumes were scaled for additional plates. The pooled linker-pHLA solutions were then coated onto the 10-spot plate as 50 μL/well, the plate sealed and stored at 4° C. overnight.

Format 4-G2(1H11) hOKT3a and Format 4-G5(1C12) hOKT3a antibodies, with or without the DSB described in Example 18, were serially diluted 3-fold with PBS+1% BSA. The plate was washed 3 times with PBS+0.05% Tween and samples added as 50 μL/well. Plates were incubated at room temperature shaking for 2 hours. The plates were washed as before and 50 μL of 1 μg/mL SulfoTag donkey anti-human Fc, (Jackson ImmunoResearch 709-005-098) was added to each well. The anti-human Fc antibody was sulfo-tag labeled using the MSD Gold Sulfo-tag NHS-Ester Conjugation kit (Meso Scale Discovery, R31AA-2) at a challenge ratio of 10. The plates were incubated for 1 hour shaking at room temperature. The plate wash was repeated and 150 μL 2× Read Buffer T (Meso Scale Discovery, R92TC-2) was added to all wells and the plate read immediately on the Quickplex SQ 120.

### Results

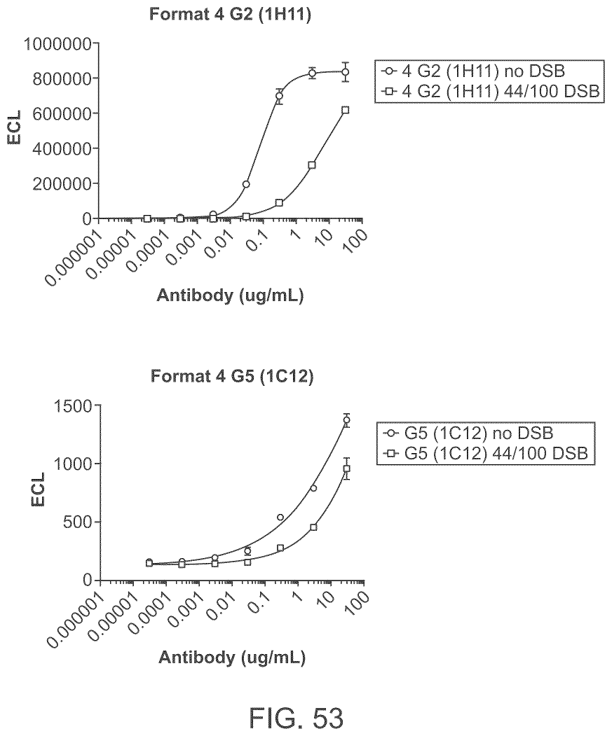

Results are depicted in FIG. 53. G2 Format 4 binding as measured by MSD was 0.546 nM without the DSB and 46.42 nM with the DSB. The G5 data did not fit a curve. However, the G5 dose-response curve without the DSB was leftward shifted as compared to the G5 dose-response curve with the DSB.

### Example 22: Effect of Engineered DSB on Cell Binding

Format 4 bispecific antibodies with and without the stabilizing DSB as described in Example 18 were tested for their ability to specifically bind to the HLA-PEPTIDE targets on the surface of antigen presenting cells.

The cell lines used to express the desired HLA-PEPTIDE targets were as follows: A375 cells (which express HLA subtype *01:01) engineered to express the G2 restricted peptide NTDNNLAVY (SEQ ID NO: 214), LN229 (which express HLA subtype B*35:01) engineered to express the G5 restricted peptide EVDPIGHVY (SEQ ID NO: 233). All cell lines were also engineered to express luciferase.

Tumor cells engineered to express target peptide were harvested, washed in PBS, and stained with eBioscience Fixable Viability Dye eFluor 450 for 15 minutes at room temperature. Following another wash in PBS+1-2% FBS, cells were resuspended with the indicated molecules at varying concentrations and incubated for 1 hour at 4° C. After another wash, PE-conjugated goat anti-human IgG secondary antibody (Jackson ImmunoResearch) was added at 1:100 to 1:200 for 30 minutes at 4° C. After washing in PBS+1-2% FBS, cells were resuspended in PBS+1-2% FBS and analyzed by flow cytometry. Flow cytometric analysis was performed on the Attune NxT Flow Cytometer (ThermoFisher) using the Attune NxT Software. Data was analyzed using FlowJo.

### Results

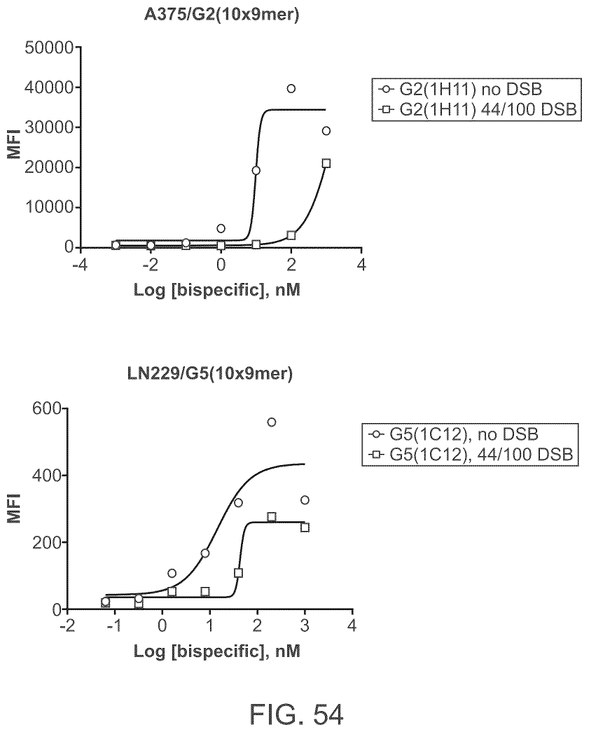

Results are depicted in FIG. 54. Introduction of the stabilizing H44/L100 DSB reduces cell binding for 4-G2(1H11) hOKT3a as measured by an EC50 shift from 9.8 nM without the DSB to 1.75 μM with the DSB. For 4-G5(1C12) hOKT3a, addition of the DSB shifted the EC50 from 14.3 nM to 43.2 nM.

### Example 23: In Vitro Cytotoxicity for G2 Format 4+/−DSB

### Materials and Methods

### Spheroid Toxicity

The cell lines used to express the desired HLA-PEPTIDE targets were as follows: A375 cells (which express HLA subtype *01:01) engineered to express the G2 restricted peptide NTDNNLAVY (SEQ ID NO: 214). The cell line was also engineered to express luciferase.

Luciferase expressing cells were plated in 100 μL at 10,000-15,000 cells/well in Corning ultra-low attachment plates (Corning #4515) in corresponding culture medium without selection. Plates were incubated for two days at 37° C. and 5% CO2 to allow spheroid formation. Antibody (Format 4-G2(1H11) hOKT3a), plus or minus the stabilizing disulfide bond described in Example 12), was titrated at and added as 10 μL/well. Normal human PBMCs were thawed and rested for 4-6 hours at 37° C. and added as 100,000 cells/well in 50 μL giving an Effector:Target ratio of 10:1. Plates were then incubated for 4 days at 37° C. and 5% CO2. At the end of the incubation period 100 μL Luciferin (Pierce #88292) at 300 μg/mL was added to the plate. Luciferase was read on the SpectraMax iE3. Percent cytotoxicity was calculated as (Media control-sample signal)/(Media control-maximum lysis)*100.

### 2D Cytotoxicity

Target and control cells were plated at 40,000 cells per well of 96 well plate. For the G2 molecules the target cell line with A375 transduced with a 10×9mer cassette expressing the target peptide and luciferase. A375 s transduced with luciferase alone serve as a negative control. After allowing the cells to adhere for 30 minutes, human PBMCs (Stem Cell Technologies) were added at a ratio of 5:1 effector to target cells. Bispecific antibody was added to the well at indicated final concentration. Each concentration was performed in duplicate. Cultures were incubated for three days. Luciferase signal was assessed using Promega's Bio-Glo assay system (Cat. #G7941) according to manufacturer's instructions and read on the SpectraMax M5. Signal was normalized to control wells to determine the percent of cytotoxicity. Loss of luciferase signal is interpreted as loss of cell viability.

### Results

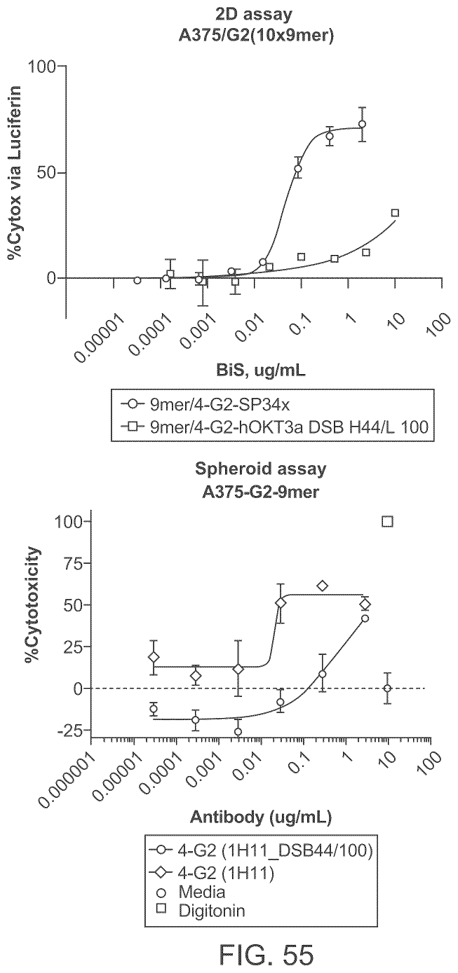

Results for G2 are depicted in FIG. 55. G2 Format 4 antibodies with the stabilizing disulfide bond resulted in lower cytotoxicity, as indicated by the rightward shift in the dose-response curve.

### Example 24: Preparation of Samples for Diabody Experiments

Samples/molecules used in Examples 24-29 and corresponding linker lengths are provided in Table 16 below.

TABLE 16

| | | | | L1 = L2 | L3 = L4 |
|---|---|---|---|---|---|
| | Sample | | | (Linker | (Linker |
| Target | # | Format | Molecule ID | Length) | Length) |
| G5 | 1 | 3 | 3-G5(1C12)-hOKT3a_N97G | | |
| | 2 | 5 | 5-G5(1C12)-hOKT3A DSB | | |
| | | | H44/L100 | | |
| | 3 | 4 | 4-G5(1C12)-hOKT3a | (G4S) × 4 (SEQ | (G4S) × 2 (SEQ |
| | | | | ID NO: 228) | ID NO: 111) |
| | 4 | 4_DSB | 4-G5(1C12) hOKT3a_DSB | (G4S) × 4 (SEQ | (G4S) × 2 (SEQ |
| | | | H44/L100 | ID NO: 228) | ID NO: 111) |
| | | | TM(+)cys; N97G; 153R; Y100dV | | |
| | 5 | 41(Diabody) | 4-G5(1C12)_10AAL hOKT3a | (G4S) × 4 (SEQ | (G4S) × 2 (SEQ |
| | | | | ID NO: 228) | ID NO: 111) |
| G2 | 6 | 4 | 4-G2(1H11)-hOKT3a | (G4S) × 4 (SEQ | (G4S) × 2 (SEQ |
| | | | | ID NO: 228) | ID NO: 111) |
| | 7 | 4-DSB | 4-G2(1H11)-H44/L100 | (G4S) × 4 (SEQ | (G4S) × 2 (SEQ |
| | | | hOKT3a-TM | ID NO: 228) | ID NO: 111) |
| | 8 | 41(Diabody) | 4-G2(1H11)-10AAL hOKT3a | (G4S) × 2 (SEQ | (G4S) × 2 (SEQ |
| | | | | ID NO: 111) | ID NO: 111) |
| | 9 | 41(Diabody) | 4-G2(1H11)-10AAL -DSB | (G4S) × 2 (SEQ | (G4S) × 2 (SEQ |
| | | X DSB | H44/L100 hOKT3a-TM | ID NO: 111) | ID NO: 111) |

* TM denotes the TM mutations (L234F/L235E/P331S) in the variant CH2-CH3 domains (both arms of the antibody).

### Example 25: Effect of Shortened Linker on Diabody Formation

### Materials and Methods

### Proteolysis by FabALACTICA

0.3-0.5 mg of G5(1C12)-hOKT3a and G2(1H11)-hOKT3a Format 4 antibodies with and without shortened linkers in L1 and L2 were buffer exchanged from PBS pH 7.4 into 150 mM sodium phosphate buffer at pH 7.0. Format 4 antibodies with shortened L1 and L2 linkers had 10 amino acid residues at each of the L1 and L2 linkers, specifically (GGGGS)₂ (SEQ ID NO: 111), The samples were then concentrated to a volume of approximately 100 μL, with corresponding concentrations ranging from 35 mg/mL, loaded onto FabALACTICA microspin columns (Genovis), and incubated for 16 hr with end-over-end mixing. To collect the digested products, the columns were centrifuged at 1000×g for 1 min, followed by two additional rounds of elution using 100 μL PBS pH 7.4. The elution fractions were pooled (referred to as “digested pool”), and subsequently loaded onto a CaptureSelect ProteinA (Genovis) column, and incubated for a minimum of 30 min with end-over-end mixing. The flowthrough was collected by centrifugation at 200×g for 1 min, followed by two wash steps with 100 μL PBS pH 7.4. The flowthrough and wash fractions were pooled, and are henceforth referred to as “‘Fab’ fraction”. The ProteinA bound fragments were eluted using 100 μL of 0.1 M Glycine, pH 3 by centrifugation at 200×g for 1 min, and immediately neutralized with 10 μL 1M tris pH 8. A second elution step was performed by centrifugation at 1000×g for 1 min, and neutralized immediately as described. The elution fractions are henceforth referred to as “‘ Fc’ Fraction”

### SEC-HPLC

For the Format 4-G5(1C12) hOKT3a molecule with L1=L2=(G4S)×4 (SEQ ID NO: 228), analytical SEC-HPLC was performed on an Agilent 1200 series HPLC system equipped with a degasser (G1379B), binary pump (G1312B), high performance autosampler (G1367D), and wide range diode array detector (DAD, G7115A). Approximately 40 μL of each of the untreated antibody, “Fab” Fraction, and “Fc” fraction was loaded onto a TSKgel G3000 SWxl column (7.8 mm ID×30 cm) with the TSKgel G2000SWxl-G4000SWxl Guard Column in line from Tosoh Bioscience. The column was operated at 0.5 mL/min for 60 min in PBS, pH 7.4. The DAD was set to collect absorbance at 280 nm.

For all other molecules, analytical SEC-HPLC was performed on an Agilent 1260 series HPLC system equipped with a degasser (G4225A), binary pump (G1312B), autosampler (G1329B), and diode array detector (DAD, G4212B). Approximately 60 to 100 μg of each untreated antibody, and 100 μL of the “Fab” fraction, and “Fc” fraction were loaded onto a TSKgel G3000 SWxl column (7.8 mm ID×30 cm) with the TSKgel G2000SWxl-G4000SWxl Guard Column in line from Tosoh Bioscience. The column was operated at 0.5 mL/min for 30 min in PBS, pH 7.4. The DAD was set to collect absorbance at 280 nm.

### SDS-PAGE

For all molecules tested, 2 μg of undigested antibody, and 5-10 μL of digested pool, “Fab” fraction, and “Fc” fraction were denatured under non-reducing and reducing conditions using NuPage 4×LDS sample buffer (Invitrogen). Samples analyzed under non-reducing conditions were left at ambient temperature. Samples analyzed under reducing conditions, using 2 μL of 1M DTT, were incubated at 70° C. for 5-10 minutes. All samples were analyzed using NuPage 4-12% Bis-Tris gradient gels (Invitrogen) against Precision Plus Protein™ Dual Color Standards (Bio-Rad), with NuPage MOPS SDS running buffer (Invitrogen). Gels were visualized after staining with InstantBlue stain (Expedeon).

### Results

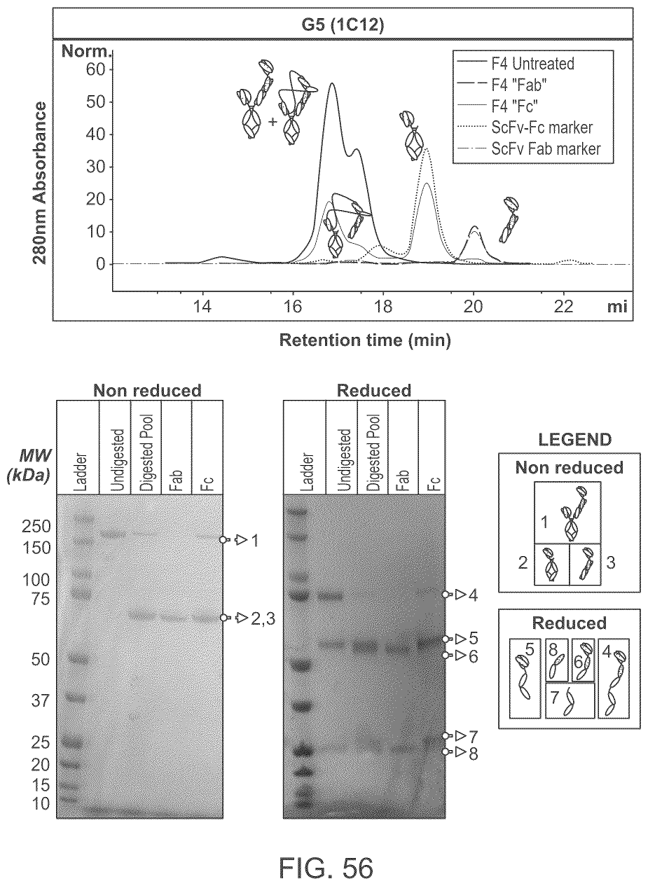

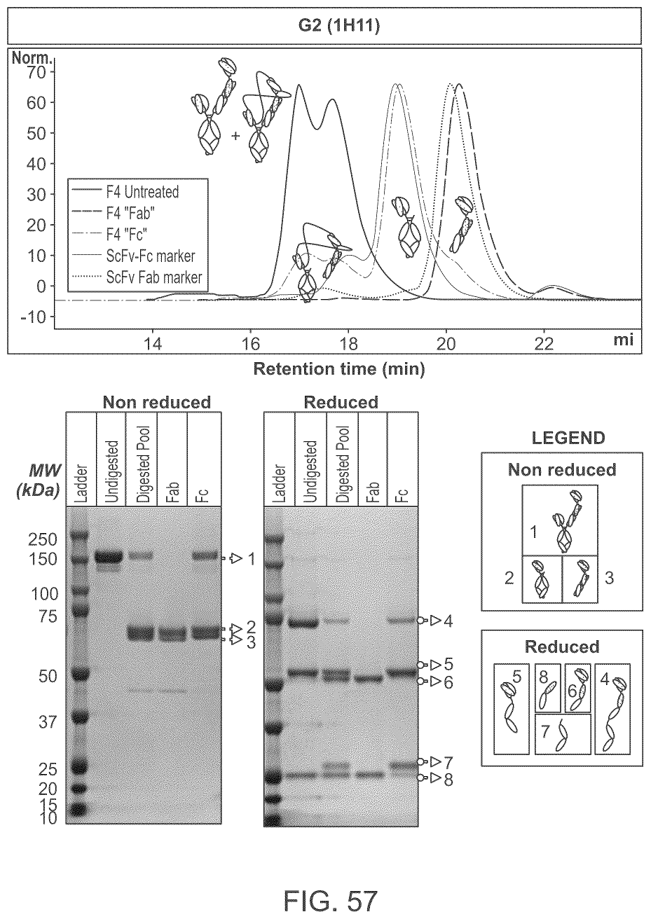

SEC-HPLC analysis of undigested Format 4-G2(1H11) hOKT3a and G5(1C12) hOKT3a molecules with L1=L2=(G4S)×4 (SEQ ID NO: 228) showed the characteristic “split-peak” profile described previously (FIG. 56 and FIG. 57), where the peak with retention time around 17.3 minutes corresponds to the 2×scFv conformation, and the peak with retention time around 17.7 minutes corresponds to the putative diabody conformation. This “split-peak” was more pronounced for the G2(1H11) hOKT3a molecule, indicating that the scFv of this molecule undergoes more “breathing”, resulting in more frequent shuffling between the two conformations.

SEC-HPLC analysis of the “Fab” fraction for both molecules revealed a single peak that aligns with the retention time of scFv-Fab marker. The “Fc” fractions had a peak with retention time that aligned with the scFv-Fc marker, as well as a second peak with retention time that aligned with the undigested antibody. The absence of an intense band migrating around 150 kDa under non-reducing conditions, and around 75 kDa under reducing conditions in the “Fc” fraction lane for the G5 molecule indicated that the digestion was near complete. Yet, the second peak on the SEC-HPLC chromatogram that aligned with the retention time of the undigested antibody appeared in roughly equal proportion to the ScFv-Fc peak. The presence of this second peak, which likely corresponds to a clipped diabody, further indicated that the Format 4-G5(1C12) hOKT3a molecule with a 20 amino acid-long linker at locations L1 and L2 exists as a mixture of the 2×ScFv and diabody conformations. The observation that the second peak for the G2(1H11) hOKT3a molecule in the “Fc” fraction was not as significant as was observed for G5(1C12) hOKT3a is likely explained by more frequent shuffling between the diabody and 2×scFv conformations mentioned supra. During the 16 hr digestion period, the clipped diabody form can more readily dissociate into the scFv-Fc and scFv-Fab fragments, contributing to the results obtained for this molecule (FIG. 56 and FIG. 57).

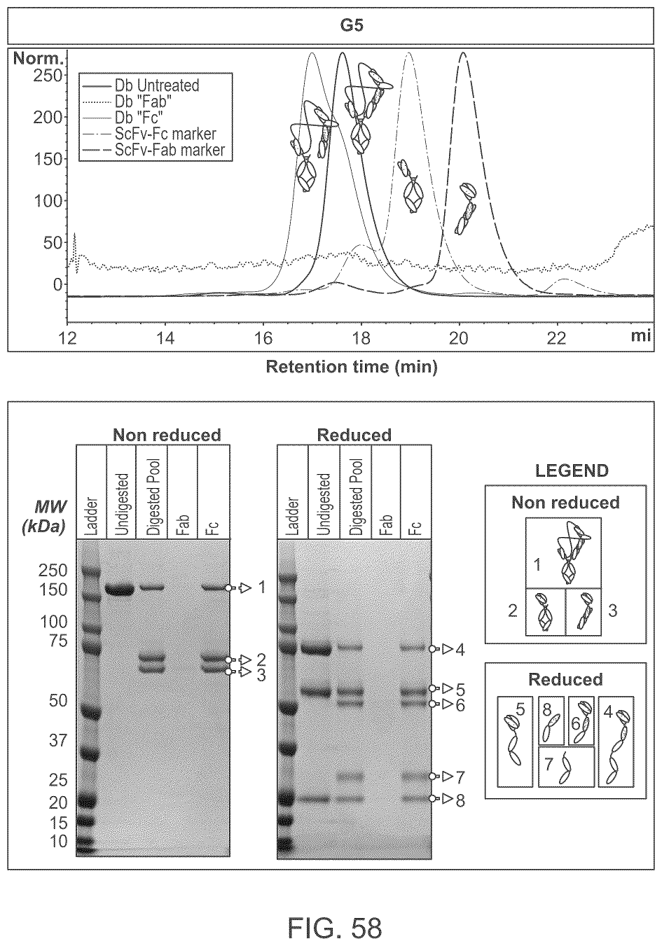

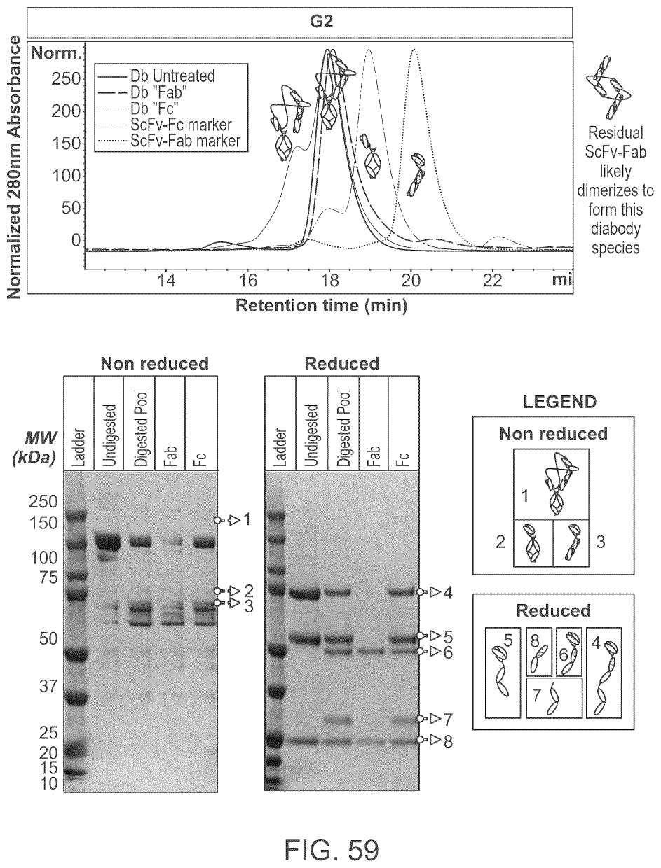

On the other hand, SEC-HPLC analysis of undigested format 4 G2(1H11) hOKT3a and G5(1C12) hOKT3a molecules with shortened L1 and L2 linkers (L1=L2=(G4S)×2 (SEQ ID NO: 111)) showed a single peak with retention time around 17.7-18 minutes (FIG. 58 and FIG. 59). This retention time aligns with what we hypothesized to be the peak corresponding to the diabody conformation in the split-peak profile observed prior to shortening the linker. Therefore, this indicated that shortening the linker from 20 amino acids to 10 amino acids forced both Format 4 molecules into the diabody conformation.

To further support this, no protein was recovered in the G5(1C12) hOKT3a “Fab” fraction, as shown in the SEC-HPLC chromatogram, and reducing and non-reducing gels. Additionally, the “Fc” fraction that resulted from digestion of this molecule resulted in a single peak on the SEC-HPLC chromatogram which aligned with the retention time of the undigested molecule, corresponding to a clipped diabody. The newly formed “split peak” in the Fe-fraction is likely due to the clipped diabody existing in compact and extended conformations. Furthermore, the reducing and non-reducing gels showed that any bands corresponding the scFv-Fab fragment, which would be expected to be present in the “Fab” fraction lane in the absence of diabody, were present in the scFv-Fc fraction instead (FIG. 58).

Similar analysis of the “Fc” fraction for the G2(1H11) hOKT3a molecule supported diabody formation, where the SEC-HPLC chromatogram showed a “split peak” corresponding to the clipped diabody conformation, and bands corresponding to scFc-Fab fragment present in the “Fc” lane of the reducing and non-reducing gel. The “Fab” fraction of the G2(1H11) hOKT3a molecule, however did contain some residual scFv-Fab, which likely dimerized into a diabody, as the SEC-HPLC trace for this fraction had an earlier retention time than would be expected for the scFv-Fab fragment (FIG. 59). This is likely a result of the G2(1H11) hOKT3a scFv being more prone to “breathing”, as mentioned above, where the clipped diabody can readily fall apart into the scFv-Fc and scFv-Fab fractions. The increased “breathing” observed for the G2(1H11) hOKT3a molecule indicated that the G2(1H11) hOKT3a molecule with shortened linker might not form the diabody conformation as stably as the G5(1C12) hOKT3a molecule.

### Example 26: Effect of Combining DSB with Shortened Linker

### Materials and Methods

### Proteolysis by FabALACTICA:

0.3-0.5 mg of G2(1H11)-hOKT3a Format 4 with L1=L2=(G4S)×2 (SEQ ID NO: 111) and DSB H44/L100 mutation, was buffer exchanged from PBS pH 7.4 into 150 mM sodium phosphate buffer at pH 7.0. The samples were then concentrated to a volume of approximately 100 μL, with corresponding concentrations ranging from 35 mg/mL, loaded onto FabALACTICA microspin columns (Genovis), and incubated for 18 hr with end-over-end mixing. To collect the digested products, the columns were centrifuged at 1000×g for 1 min, followed by three additional rounds of elution using 100 μL PBS pH 7.4. The elution fractions were pooled (referred to as “digested pool”), and subsequently loaded onto a CaptureSelect ProteinA (Genovis) column, and incubated for a minimum of 30 min with end-over-end mixing. The flowthrough was collected by centrifugation at 200×g for 1 min, followed by three wash steps with 100 μL PBS pH 7.4. The flowthrough and wash fractions were pooled, and are henceforth referred to as “‘Fab’ fraction”. The ProteinA bound fragments were eluted using 10 μL of 0.1 M Glycine, pH 3 by centrifugation at 200×g for 1 min, and immediately neutralized with 10 μL 1 M Tris pH 8. Four additional elution steps were performed by centrifugation at 1000×g for 1 min, and neutralized immediately as described. The elution fractions are henceforth referred to as “‘Fc’ Fraction”

### SEC-HPLC

Analytical SEC-HPLC was performed on an Agilent 1260 series HPLC system equipped with a degasser (G4225A), binary pump (G1312B), autosampler (G1329B), and diode array detector (DAD, G4212B). Approximately 60 μg to 100 μg of each untreated antibody, and 100 μL of the “Fab” fraction, and “Fc” fraction were loaded onto a TSKgel G3000 SWxl column (7.8 mm ID×30 cm) with the TSKgel G2000SWxl-G4000SWxl Guard Column in line from Tosoh Bioscience. The column was operated at 0.5 mL/min for 30 min in PBS, pH 7.4. The DAD was set to collect absorbance at 280 nm.

### CE-SDS

Capillary gel electrophoresis was performed using the LabChip GXII Touch HT system (PerkinElmer), and samples were analyzed using the ProteinExpress 200 High Sensitivity assay (PerkinElmer, #CLS960008) under reducing and non-reducing conditions. 2 μg of each untreated antibody, and 5 μL of each of digested pool, digested “Fab” fraction, and digested “Fc” fraction were mixed with 7 μL of reducing or non-reducing denaturing solution, and incubated at 70° C. for 10-12 min. The reducing denaturing solution was prepared by adding 24.5 μL of 1M DTT to 700 μL of non-reducing denaturing solution provided in the kit. Denatured samples were diluted with 32 μL of MilliQ water, mixed well, and spun down prior to analysis. The Protein Express LabChip (PerkinElmer, #760499), and ladder were prepared according to manufacturer instructions.

### Results

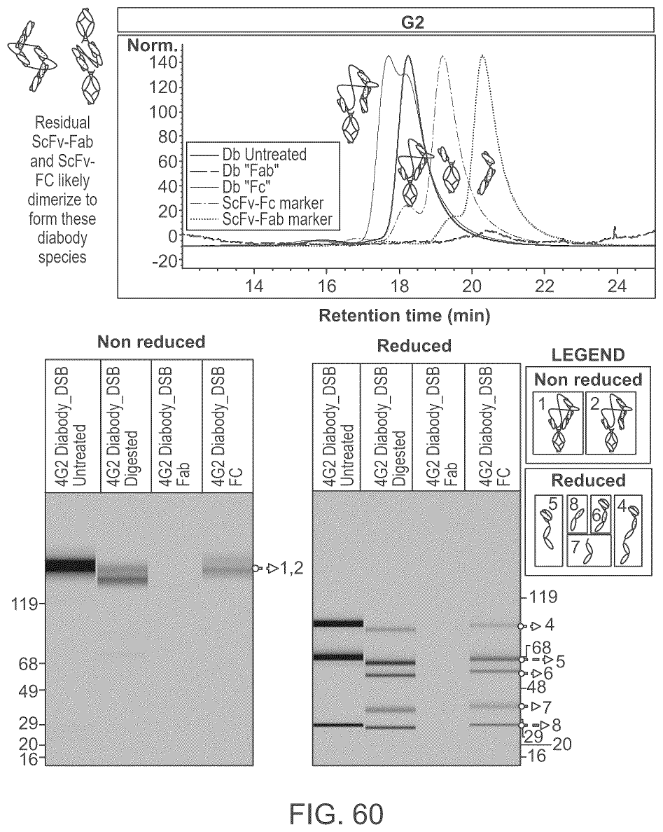

SEC-HPLC analysis of undigested Format 4 G2(1H11) hOKT3a with shortened L1 and L2 linkers (L1=L2=(G4S)×2 (SEQ ID NO: 111)), along with the incorporation of the DSB H44/L100 mutation showed a single peak with retention time around 17.7-18 minutes (FIG. 60). This retention time aligned with what was hypothesized to be the peak corresponding to the diabody conformation in the split-peak profile observed prior to shortening the linker. Contrary to what was seen in the absence of the DSB H44/L100 stabilizing mutation (Example 25), no protein was recovered in the “Fab” fraction, as was observed in the SEC-HPLC chromatogram, and reducing and non-reducing gels. Absence of bands corresponding to scFv-Fc and scFv-Fab fragments following digestion under non-reducing conditions indicated that the disulfide bond formation was complete, and was effective at stabilizing the diabody conformation and preventing “breathing” (FIG. 60).

Additionally, the “Fc” fraction that resulted from digestion of this molecule resulted in a single peak on the SEC-HPLC chromatogram which aligned with the retention time of the undigested molecule, corresponding to a clipped diabody. The newly formed “split peak” in this “Fc” fraction was likely due to the clipped diabody existing in compact and extended conformations. Furthermore, the reducing gel showed that any bands corresponding to the scFv-Fab fragment, which would be expected in the “Fab” fraction lane in the absence of diabody, was instead present only in the scFv-Fc fraction (FIG. 60). These results indicate that shortening the linker and introduction of a disulfide bond together force and stabilize diabody conformation for the Format 4-G2(1H11) hOKT3a molecule.

### Example 27: Effect of Diabody on Affinity as Measured by BLI

### Materials and Methods

The affinity of 2×scFv-conformed ABPs and diabody-conformed ABPs (i.e. G5(1C12) hOKT3a and G2(1H11) hOKT3a Format 4 antibodies with and without shortened linkers L1 and L2) was evaluated using the ForteBio Octet HTX in 96-channel mode with biolayer interferometry (BLI) detection. The experiment utilized G2(1H11)-hOKT3a and G5(1C12)-hOKT3a Format 4 antibodies with shortened linkers (L1=L2=(GGGGS)₂ (SEQ ID NO: 111), “DAB”) and with non-shortened linkers (L1=L2=(GGGGS)₄ (SEQ ID NO: 228), “2×scFv”). High Precision Streptavidin SAX biosensors (P/N 18-5117) were loaded into the instrument. Biotinylated G2-pHLA or G5-pHLA was captured on the SAX biosensor at 2 μg/mL and ran for 120 s in the assay buffer composed of 0.02% Tween-20 and 0.1% BSA. The biosensors were then dipped in assay buffer for a baseline. Subsequently, the biosensors were dipped into wells containing varying concentrations of the bispecific antibody samples (3.125, 6.25, 12.5, 25, 50, 100 and 200 nM) to measure the association rate for 50 seconds. The biosensors were finally dipped into wells containing assay buffer to measure the dissociation rate for another 50 seconds. Referencing was completed by having a biosensor with no immobilized ligand dipped into analyte. Kinetic data was processed with Octet™ software using a 1:1 kinetic model with errors within 10%, X² below 3, and R² above 0.9.

### Results

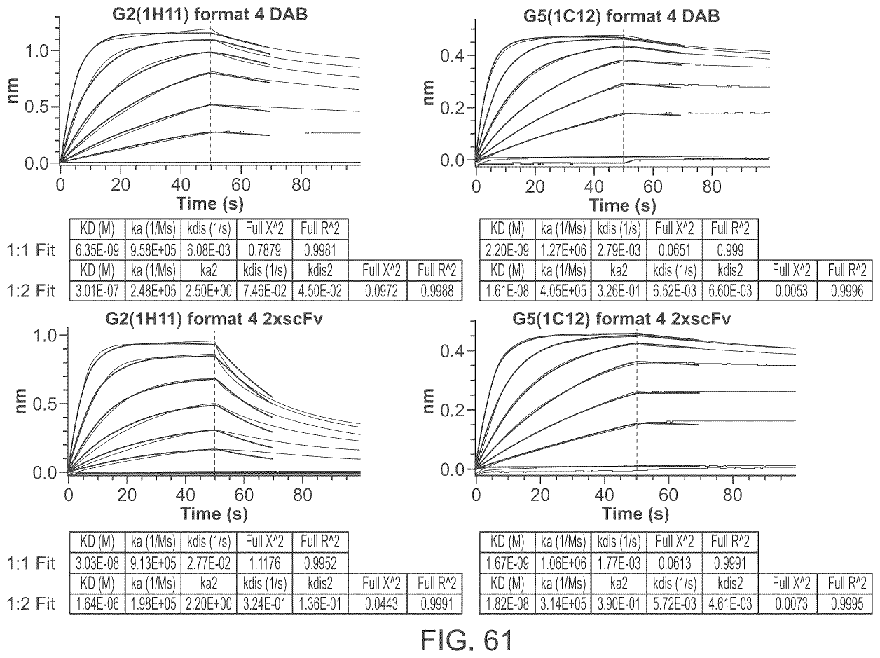

Results are shown in FIG. 61. The results revealed a significantly lower K_{D} value for the Format 4-G2(1H11) hOKT3a antibody with shortened linker than with the non-shortened linker.

### Example 28: Effect of Diabody Formation on Cell Binding

For G2 cell binding, the experiment utilized Format 4-G2(1H11) hOKT3a antibody having L1=L2=(GGGGS)₄ (SEQ ID NO: 228) with/or without the DSB44/100. It also utilized a Format 4-G2(1H11) hOKT3a antibody with the shortened linker (10AAL=L1=L2=(GGGGS)₂ (SEQ ID NO: 111)). For the G5 cell binding, the experiment utilized Format 4-G5(1C12) hOKT3a antibody having L1=L2=(GGGGS)₄ (SEQ ID NO: 228) with/or without the DSB44/100. It also utilized a Format 4-G5(1C12) hOKT3a antibody with the shortened linker (10AAL=L1=L2=(GGGGS)₂ (SEQ ID NO: 111)). The cell lines used to express the desired HLA-PEPTIDE targets were as follows: A375 cells (which express HLA subtype *01:01) engineered to express the G2 restricted peptide NTDNNLAVY (SEQ ID NO: 214), LN229 (which express HLA subtype B*35:01) engineered to express the G5 restricted peptide EVDPIGHVY (SEQ ID NO: 233). All cell lines were also engineered to express luciferase, using a lentivirus transduction of a cassette containing a 10× repeat of the peptide, Luciferase, and puromycin-resistance. Cassette-expressing cells were selected using 0.5 μg/mL of puromycin. Jurkat E6-1 (ATCC TIB-152) and Jurkat T3.5 (ATCC TIB-153) cells were grown under standard tissue culture conditions. Cells were harvested, washed in PBS, and stained with eBioscience Fixable Viability Dye eFluor 450 for 15 minutes at room temperature. Following another wash in PBS+2% FBS, cells were resuspended with bispecifics at varying concentrations. Cells were incubated with bispecifics for 1 hour at 4° C. After another wash, PE-conjugated goat anti-human IgG secondary antibody (Jackson ImmunoResearch) was added at 1:100. After incubating at 4° C. for 45 minutes and washing in PBS+2% FBS, cells were resuspended in PBS+2% FBS and analyzed by flow cytometry. Flow cytometric analysis was performed on the Attune NxT Flow Cytometer (ThermoFisher) using the Attune NxT Software. Data was analyzed using FlowJo.

### Results

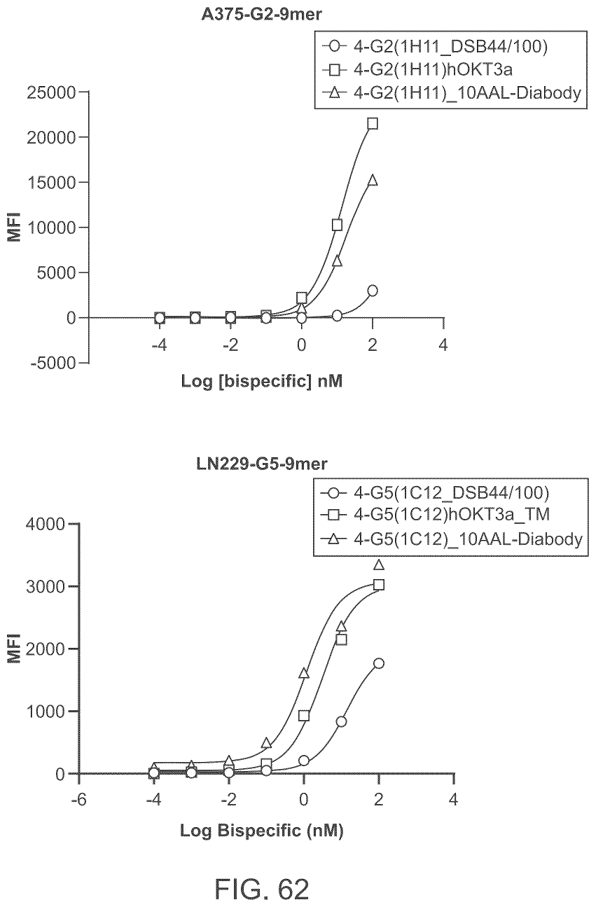

Results are shown in FIG. 62. All the G2 and G5 molecule bound their pHLA targets. For both G2 and G5 groups, the cell binding to molecule with the shortened linker (diabody conformed) exhibited higher levels of cell binding than the non-shortened linker+DSB44/100 groups (FIG. 62).

### Example 29: Effect of Diabody Formation on Cytotoxicity

For G2 cell binding, the experiment utilized Format 4-G2(1H11) hOKT3a antibody having L1=L2=(GGGGS)₄ (SEQ ID NO: 228) with/or without the DSB44/100. It also utilized a Format 4-G2(1H11) hOKT3a antibody with the shortened linker (10AAL=L1=L2=(GGGGS)₂ (SEQ ID NO: 111)). For the G5 cell binding, the experiment utilized Format 4-G5(1C12) hOKT3a antibody having L1=L2=(GGGGS)₄ (SEQ ID NO: 228) with/or without the DSB44/100. It also utilized a Format 4-G5(1C12) hOKT3a antibody with the shortened linker (10AAL=L1=L2=(GGGGS)₂ (SEQ ID NO: 111)). The cell lines used to express the desired HLA-PEPTIDE targets were as follows: A375 cells (which express HLA subtype *01:01) engineered to express the G2 restricted peptide NTDNNLAVY (SEQ ID NO: 214), LN229 (which express HLA subtype B*35:01) engineered to express the G5 restricted peptide EVDPIGHVY (SEQ ID NO: 233). All cell lines were also engineered to express luciferase, using a lentivirus transduction of a cassette containing a 10× repeat of the peptide, Luciferase, and puromycin-resistance. Cassette-expressing cells were selected using 0.5 μg/mL of puromycin. For the assay, cells were pelleted, washed in PBS, and re-suspended at 2E6/mL in RPMI with 10% FBS. 25 μL of target cells were plated in opaque white 96-well plates. Serial dilutions of the bispecific molecules were added as described above. T cells were added to the plates to give a 10:1 T cell: target ratio as described above. Following 24-hour incubation, Bio-Glo luciferase substrate (Promega cat #G7941) was added and plate incubated and read according to manufacturer's instructions. To calculate % killing, RPMI background RLU was first subtracted from all values. % killing was determined as % cytotoxicity w/Ab-% cytotoxicity w/o Ab, where % cytotoxicity was calculated as 100%-% viability. % viability was calculated as % of RLU in experimental wells normalized against target cells alone.

### Results

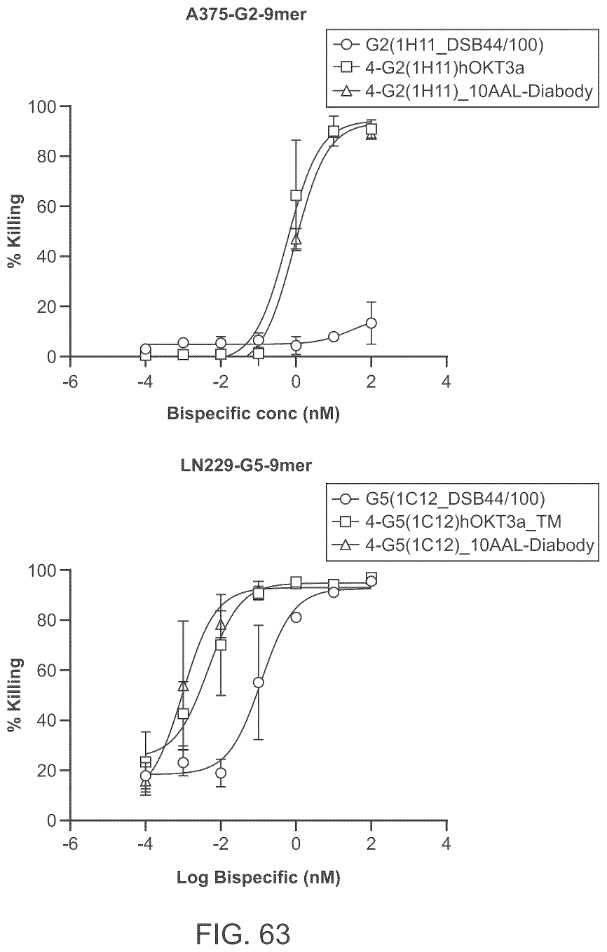

The results are shown in FIG. 63. All the G2 and G5 molecule exhibited varying degrees of cytotoxic potency. In the case of the G2 molecules, the G2(1H11)hOKT3a (non-shortened linkers; 2×scFv) and the G2(1H11)_10AAL (shortened linkers; diabody) groups showed significantly more cytotoxic potential than the DSB group. In the case of the G5 molecules, the cytotoxic potential of the G5(1C12)hOKT3a (non-shortened linkers; 2×scFv) and the G5(1C12)_10AAL (shortened linkers; diabody) groups showed significantly higher cytotoxic potential.

### Example 30: Digestion of Format 4 Antibodies with Engineered External DSBs

In order to stabilize molecules in the diabody format, without Fv modification, linkers were engineered that contain Cys residues to introduce disulfide bonds (DSBs) downstream of the diabody domains. First, the diabody conformation of the molecule was forced by shortening the VH-VL linker of the Fv on both chains to 10 amino acids (10AAL). It is known in the art that the C termini of the two chains that dimerize to form a diabody can exist in close or distal conformations relative to each other. See Olafsen, Tove, et al. Protein Engineering Design and Selection 17.1 (2004): 21-27, which is incorporated by reference in its entirety. By introducing a DSB immediately downstream of the second domain of each half-diabody construct, the proximal conformation is forced, and the overall assembly is stabilized. Constructs were generated that introduced Cys residues 3 amino acids (GGC) or 4 amino acids (GGGC (SEQ ID NO: 210)) downstream of the end of the diabody sequences. In the case of format 4-like molecules, after forming a DSB, the linkers both continue, either in the knob-Fc chain, or into hole-Fab-Fc chain as depicted in FIG. 64 (top right). Additionally, we designed constructs that disconnect one of the ABRs from the hole-Fab-Fc side and provide it as a separate chain in trans (FIG. 64; bottom). Similarly, we introduced Cys residues 3 amino acids (GGC) or 4 amino acids (GGGC (SEQ ID NO: 210)) downstream of the end of the diabody sequences, but in this case, the new 4th chain terminated after the Cys rather than continuing into the hole-Fab-Fc. Schematic diagrams of the assembled constructs are presented in FIG. 64, linkers are presented in Tables 17 and 18. In order to demonstrate that these sequences form diabody, they are subjected to FabALACTICA digestion and analyzed by SEC-HPLC and CE-SDS.

TABLE 17
Cysteine mutations in the linkers of ABP
constructs that result in external DSBs.
Cysteines that form the DSBs are underlined.

| Construct | L1 & L2 | L3 | L4 |
|---|---|---|---|
| D- | GGGGSGGGGS | GGGGSEPKSSDKTHT | GGGGSGGGGS |
| 1H11xhOKT3 | (SEQ ID NO: | (SEQ ID NO: 112) | (SEQ ID NO: 111) |
| | 111) | | |
| D-1H11-LL- | GGGGSGGGGS | GGCGGGGSGSEPKSSDKTHT | GGCGGGGSGSGGGGS |
| GGCxhOKT3 | (SEQ ID NO: | (SEQ ID NO: 113) | (SEQ ID NO: 114) |
| | 111) | | |
| D-1H11-LS- | GGGGSGGGGS | GGCGSEPKSSDKTHT (SEQ ID | GGCGGGGSGSGGGGS |
| GGCxhOKT3 | (SEQ ID NO: | NO: 115) | (SEQ ID NO: 114) |
| | 111) | | |
| D-1H11-SL- | GGGGSGGGGS | GGCGGGGSGSEPKSSDKTHT | GGCGS (SEQ ID NO: |
| GGCxhOKT3 | (SEQ ID NO: | (SEQ ID NO: 113) | 116) |
| | 111) | | |
| D-1H11-LL- | GGGGSGGGGS | GGGCGGGGSGSEPKSSDKTHT | GGGCGGGGSGSGGGGS |
| GGGCxhOKT3 | (SEQ ID NO: | (SEQ ID NO: 117) | (SEQ ID NO: 118) |
| | 111) | | |
| D-1H11-LS- | GGGGSGGGGS | GGGCGSEPKSSDKTHT (SEQ | GGGCGGGGSGSGGGGS |
| GGGCxhOKT3 | (SEQ ID NO: | ID NO: 234) | (SEQ ID NO: 118) |
| | 111) | | |
| D-1H11-SL- | GGGGSGGGGS | GGGCGGGGSGSEPKSSDKTHT | GGGCGS (SEQ ID NO: |
| GGGCxhOKT3 | (SEQ ID NO: | (SEQ ID NO: 117) | 119) |
| | 111) | | |

TABLE 18
Cysteine mutations in the linkers of ABP
constructs that result in external DSBs.
Cysteines that form the DSBs are underlined.

| Construct | L1 & L2 | L3 | L4 |
|---|---|---|---|
| D- | GGGGSGGGGS | GGGGSEPKSSDKTHT | GGGGSGGGGS |
| 1H11xhOKT3 | (SEQ ID NO: | (SEQ ID NO: 112) | (SEQ ID NO: 111) |
| | 111) | | |
| D-1H11-LL- | GGGGSGGGGS | GGCGGGGSGSEPKSSDKTHT | GGCGGGGSGSGGGGS |
| GGCxhOKT3 | (SEQ ID NO: | (SEQ ID NO: 113) | (SEQ ID NO: 114) |
| | 111) | | |
| D-1H11-LS- | GGGGSGGGGS | GGCGSEPKSSDKTHT | GGCGGGGSGSGGGGS |
| GGCxhOKT3 | (SEQ ID NO: | (SEQ ID NO: 115) | (SEQ ID NO: 114) |
| | 111) | | |
| D-1H11-SL- | GGGGSGGGGS | GGCGGGGSGSEPKSSDKTHT | GGCGS |
| GGCxhOKT3 | (SEQ ID NO: | (SEQ ID NO: 113) | (SEQ ID NO: 116) |
| | 111) | | |
| D-1H11-LL- | GGGGSGGGGS | GGGCGGGGSGSEPKSSDKTHT | GGGCGGGGSGSGGGGS |
| GGGCxhOKT3 | (SEQ ID NO: | (SEQ ID NO: 117) | (SEQ ID NO: 118) |
| | 111) | | |
| D-1H11-LS- | GGGGSGGGGS | GGGCGSEPKSSDKTHT | GGGCGGGGSGSGGGGS |
| GGGCxhOKT3 | (SEQ ID NO: | (SEQ ID NO: 234) | (SEQ ID NO: 118) |
| | 111) | | |
| D-1H11-SL- | GGGGSGGGGS | GGGCGGGGSGSEPKSSDKTHT | GGGCGS (SEQ ID NO: |
| GGGCxhOKT3 | (SEQ ID NO: | (SEQ ID NO: 117) | 119) |
| | 111) | | |

### Materials and Methods

### Proteolysis by FabALACTICA:

0.3-0.5 mg of Format 4-G2(1H11) hOKT3a with L1=L2=(G4S)×2 (SEQ ID NO: 111) and DSB introduced into linker regions as described above, is buffer exchanged from PBS pH 7.4 into 150 mM sodium phosphate buffer at pH 7.0. The samples are then concentrated to a volume of approximately 100 μL, with corresponding concentrations ranging from 35 mg/mL, loaded onto FabALACTICA microspin columns (Genovis), and incubated for 18 hr with end-over-end mixing. To collect the digested products, the columns are centrifuged at 1000×g for 1 min, followed by three additional rounds of elution using 100 μL PBS pH 7.4. The elution fractions are pooled (referred to as “digested pool”), and subsequently loaded onto a CaptureSelect ProteinA (Genovis) column and incubated for a minimum of 30 min with end-over-end mixing. The flowthrough is collected by centrifugation at 200×g for 1 min, followed by three wash steps with 100 μL PBS pH 7.4. The flowthrough and wash fractions are pooled, and are henceforth referred to as “‘Fab’ fraction”. The ProteinA bound fragments are eluted using 100 μL of 0.1M Glycine, pH 3 by centrifugation at 200×g for 1 min, and immediately neutralized with 10 μL 1M tris pH 8. Four additional elution steps are performed by centrifugation at 1000×g for 1 min, and neutralized immediately as described. The elution fractions are henceforth referred to as “‘FC’ Fraction”

### SEC-HPLC

Analytical SEC-HPLC are performed on an Agilent 1260 series HPLC system equipped with a degasser (G4225A), binary pump (G1312B), autosampler (G1329B), and diode array detector (DAD, G4212B). Approximately 60 to 100 μg of each untreated antibody, and 100 μL of the “Fab” fraction, and “Fc” fraction are loaded onto a TSKgel G3000 SWxl column (7.8 mm ID×30 cm) with the TSKgel G2000SWxl-G4000SWxl Guard Column in line from Tosoh Bioscience. The column is operated at 0.5 ml/min for 30 min in PBS, pH 7.4. The DAD is set to collect absorbance at 280 nm.

### CE-SDS

Capillary gel electrophoresis is performed using the LabChip GXII Touch HT system (PerkinElmer), and samples are analyzed using the ProteinExpress 200 High Sensitivity assay (PerkinElmer, #CLS960008) under reducing and non-reducing conditions. 2 μg of each untreated antibody, and 5 μL of each of digested pool, digested “Fab” fraction, and digested “Fc” fraction are mixed with 7 μL of reducing or non-reducing denaturing solution, and incubated at 70° C. for 10-12 min. The reducing denaturing solution is prepared by adding 24.5 μL of 1M DTT to 700 μL of non-reducing denaturing solution provided in the kit. Denatured samples are diluted with 32 μL of MilliQ water, mixed well, and spun down prior to analysis. The Protein Express LabChip (PerkinElmer, #760499), and ladder were prepared according to manufacturer instructions.

### Results

SEC-HPLC analysis of undigested Format 4-G2(1H11) hOKT3a with shortened L1 and L2 linkers (L1=L2=(G4S)×2 (SEQ ID NO: 111)), along with the incorporation of the DSB introduced into the linkers downstream of the diabody sequences are expected to show a single peak with retention time around 17.7-18 minutes. This retention time aligns with what is hypothesized to be the peak corresponding to the diabody conformation in the split-peak profile observed prior to shortening the linker. Due to stabilization, no protein will be recovered in the “Fab” fraction for the Format 4-like constructs, as shown in SEC-HPLC chromatograms, and reducing and non-reducing gels. By contrast, in the 4 chain constructs, the Fab will be liberated by digestion and the diabody will remain with the proteinA bindable fraction. Absence of bands corresponding to subassemblies or partially digested products following digestion under non-reducing conditions (other than free Fab in the case of the 4-chain constructs) will indicate that the disulfide bond formation was complete, and is effective at stabilizing the diabody conformation and preventing breathing. Additionally, the “Fc” fraction that results from digestion of this molecule will result in a single peak on the SEC-HPLC chromatogram which aligns with the retention time of the undigested molecule, corresponding to a clipped diabody for the format 4-like molecules and will be slightly right-shifted for the 4-chain constructs, due to removal of the Fab domain.

### Example 31: Effect of Engineered External DSBs on the Activity of Format 4 Antibodies

The ABPs described in Example 30 are further analyzed to determine functional activity.

First, their affinity to G2 pHLA is measured using a ForteBio Octet HTX in 96-channel mode with biolayer interferometry (BLI) detection. High Precision Streptavidin SAX biosensors (P/N 18-5117) are loaded into the instrument. Biotinylated G2-pHLA is captured on the SAX biosensor at 2 μg/mL and ran for 120 s in the assay buffer composed of 0.02% Tween-20 and 0.1% BSA. The biosensors are then dipped in assay buffer for a baseline. Subsequently, the biosensors are dipped into wells containing varying concentrations of the bispecific antibody samples (3.125, 6.25, 12.5, 25, 50, 100 and 200 nM) to measure the association rate for 50 seconds. The biosensors are finally dipped into wells containing assay buffer to measure the dissociation rate for another 50 seconds. Referencing is completed by having a biosensor with no immobilized ligand dipped into analyte. Kinetic data is processed with Octet™ software using a 1:1 kinetic model with errors within 10%, X2 below 3, and R2 above 0.9.

Second, their binding to cells expressing the G2 pHLA target or the CD3 target is measured by flow cytometry. A375 cells, which express HLA-A*01:01, are engineered to express the restricted peptide NTDNNLAVY (SEQ ID NO: 214) using a lentivirus transduction of a cassette containing a 10× repeat of the peptide, Luciferase, and puromycin-resistance. Cassette-expressing cells are selected using 0.5 μg/mL of puromycin. Jurkat E6-1 (ATCC TIB-152) and Jurkat T3.5 (ATCC TIB-153) cells are grown under standard tissue culture conditions. Cells are harvested, washed in PBS, and stained with eBioscience Fixable Viability Dye eFluor 450 for 15 minutes at room temperature. Following another wash in PBS+2% FBS, cells are resuspended with bispecifics at varying concentrations. Cells are incubated with bispecifics for 1 hour at 4° C. After another wash, PE-conjugated goat anti-human IgG secondary antibody (Jackson ImmunoResearch) is added at 1:100. After incubating at 4° C. for 45 minutes and washing in PBS+2% FBS, cells are resuspended in PBS+2% FBS and analyzed by flow cytometry. Flow cytometric analysis is performed on the Attune NxT Flow Cytometer (ThermoFisher) using the Attune NxT Software. Data is analyzed using FlowJo.

Finally, cytotoxicity is measured. The cell lines used to express the desired HLA-PEPTIDE targets are as follows: A375 cells (which express HLA subtype A*01:01) engineered to express the G2 restricted peptide NTDNNLAVY (SEQ ID NO: 214), LN229 (which express HLA subtype B*35:01) engineered to express the G5 restricted peptide EVDPIGHVY (SEQ ID NO: 233). All cell lines are also engineered to express luciferase, using a lentivirus transduction of a cassette containing a 10× repeat of the peptide, Luciferase, and puromycin-resistance. Cassette-expressing cells are selected using 0.5 μg/mL of puromycin. For the assay, cells are pelleted, washed in PBS, and re-suspended at 2×10⁶ cells/mL in RPMI with 10% FBS. 25 μL of target cells are plated in opaque white 96-well plates. Serial dilutions of the bispecific molecules are added as described above. T cells are added to the plates to give a 10:1 T cell: target ratio as described above. Following 24-hour incubation, Bio-Glo luciferase substrate (Promega cat #G7941) is added and plate incubated and read according to manufacturer's instructions. To calculate % killing, RPMI background RLU is first subtracted from all values. % killing is determined as % cytotoxicity w/Ab-% cytotoxicity w/o Ab, where % cytotoxicity is calculated as 100%-% viability. % viability is calculated as % of RLU in experimental wells normalized against target cells alone.

While the invention has been particularly shown and described with reference to a preferred embodiment and various alternate embodiments, it will be understood by persons skilled in the relevant art that various changes in form and details can be made therein without departing from the spirit and scope of the invention.

All references, issued patents and patent applications cited within the body of the instant specification are hereby incorporated by reference in their entirety, for all purposes.

TABLE 19
VH and VL sequences for G2 scFv Selective Binders, selective for
HLA-PEPTIDE Target HLA-A*01:01 NTDNNLAVY (SEQ ID NO: 214)

| Target | Clone | | |
|---|---|---|---|
| group | name | VH | VL |
| G2 | C11 | QVQLVQSGAEVKKPGASVKVSCK | DIQMTQSPSSLSASVGDRVTITCRAS |
| | (2F10) | ASGYTFTTYNIHWVRQAPGQGLE | QEIRRWLAWYQQKPGKAPKLLIYAA |
| | | WMGWINPNSGGTNYAQKFQGRVT | SNLETGVPSRFSGSGSGTDFTLTIS |
| | | MTRDTSTSTVYMELSSLRSEDTA | SLQPEDFATYYCQQSYSIPYTFGQG |
| | | VYYCARANWLDYWGQGTLVTVS | TKLEIK (SEQ ID NO: 2) |
| | | S (SEQ ID NO: 1) | |
| G2 | D5 | QVQLVQSGAEVKKPGASVKVSCK | DIQMTQSPSSLSASVGDRVTITCRAS |
| | (2H03) | ASGYTFTNYPIHWVRQAPGQGLE | QEIRRWLAWYQQKPGKAPKLLIYAA |
| | | WMGWINPNSGGTNYAQKLQGRVT | SNLQSGVPSRFSGSGSGTDFTLTIS |
| | | MTRDTSTSTVYMELSSLRSEDTA | SLQPEDFATYYCQQSYSTPYTFGQG |
| | | VYYCARDQGAGWDYWGQGTLVT | TKLEIK (SEQ ID NO: 4) |
| | | VSS (SEQ ID NO: 3) | |
| G2 | 52C11 | QVQLVQSGAEVKKPGASVKVSCK | DIQMTQSPSSLSASVGDRVTITCRAS |
| | | ASGATFTGYTIHWVRQAPGQGLE | QEIRRWLAWYQQKPGKAPKLLIYAA |
| | | WMGWINPNSGGTNYAQSFQGRVT | SNLETGVPSRFSGSGSGTDFTLTIS |
| | | MTRDTSTSTVYMELSSLRSEDTA | SLQPEDFATYYCQQSYSIPYTFGQG |
| | | VYYCARAEWLDKWGQGTLVTVSS | TKLEIK (SEQ ID NO: 2) |
| | | (SEQ ID NO: 5) | |
| G2 | E07 | QVQLVQSGAEVKKPGASVKVSCK | DIQMTQSPSSLSASVGDRVTITCRAS |
| | (31E07) | ASGFTFTNYLIHWVRQAPGQGLE | QEIRRWLAWYQQKPGKAPKLLIYAA |
| | | WMGWINPNSGGTNYAQRLQGRVT | SNLQSGVPSRFSGSGSGTDFTLTIS |
| | | MTRDTSTSTVYMELSSLRSEDTA | SLQPEDFATYYCQQSYSTPYTFGQG |
| | | VYYCARDYGAGNDYWGQGTLVT | TKLEIK (SEQ ID NO: 4) |
| | | VSS (SEQ ID NO: 6) | |
| G2 | G2(1H11) | QVQLVQSGAEVKKPGASVKVSCK | DIQMTQSPSSLSASVGDRVTITCQAS |
| | | ASGYTFTNYYMHWVRQAPGQGLE | QDISNYLNWYQQKPGKAPKLLIYAA |
| | | WMGMINPSGGGTSYAQKFQGRVT | SSLQSGVPSRFSGSGSGTDFTLTIS |
| | | MTRDTSTSTVYMELSSLRSEDTA | SLQPEDFATYYCQQYYSYPFTFGPG |
| | | VYYCARGNPWELRLDYWGQGTLV | TKVDIK (SEQ ID NO: 61) |
| | | TVSS (SEQ ID NO: 60) | |

TABLE 20
CDR sequences for G2 scFv Selective Binders, selective for HLA-
PEPTIDE Target HLA-A*01:01_NTDNNLAVY (SEQ ID NO: 214) (according
to Kabat numbering scheme)

| Clone name | HCDR1 | HCDR2 | HCDR3 | LCDR1 | LCDR2 | LCDR3 |
|---|---|---|---|---|---|---|
| C11 (2F10) | TYNIH | WINPNSGG | ANWLDY | RASQEIRR | AASNLET | QQSYSIPY |
| | (SEQ ID | TNYAQKF | (SEQ ID | WLA (SEQ | (SEQ ID | T (SEQ ID |
| | NO: 7) | QG (SEQ ID | NO: 9) | ID NO: 21) | NO: 10) | NO: 11) |
| | | NO: 8) | | | | |
| D5 (2H03) | NYPIH | WINPNSGG | DQGAGWD | RASQEIRR | AASNLQS | QQSYSTPY |
| | (SEQ ID | TNYAQKL | Y (SEQ ID | WLA (SEQ | (SEQ ID | T (SEQ ID |
| | NO: 12) | QG | NO: 14) | ID NO: 21) | NO: 22) | NO: 23) |
| | | (SEQ ID | | | | |
| | | NO: 13) | | | | |
| 52C11 | GYTIH | WINPNSGG | AEWLDK | RASQEIRR | AASNLET | QQSYSIPY |
| | (SEQ ID | TNYAQSFQ | (SEQ ID | WLA (SEQ | (SEQ ID | T (SEQ ID |
| | NO: 15) | G (SEQ ID | NO: 17) | ID NO: 21) | NO: 10) | NO: 11) |
| | | NO: 16) | | | | |
| E07 (31E07) | NYLIH | WINPNSGG | DYGAGND | RASQEIRR | AASNLQS | QQSYSTPY |
| | (SEQ ID | TNYAQRL | Y (SEQ ID | WLA (SEQ | (SEQ ID | T (SEQ ID |
| | NO: 18) | QG (SEQ ID | NO: 20) | ID NO: 21) | NO: 22) | NO: 23) |
| | | NO: 19) | | | | |
| G2(1H11) | YTFTNYY | GMINPSGG | CARGNPW | QASQDISN | AASSLQS | CQQYYSY |
| | MH (SEQ | GTSYA | ELRLDYW | YLN (SEQ | (SEQ ID | PFTF (SEQ |
| | ID NO: 67) | (SEQ ID | (SEQ ID | ID NO: 70) | NO: 71) | ID NO: 72) |
| | | NO: 68) | NO: 69) | | | |

TABLE 21
VH and VL sequences for Anti-CD3 Binder

| Clone name | VH | VL |
|---|---|---|
| Anti-CD3 | QVQLVQSGAEVKKPGASVKVSCKASG | DIQMTQSPSSLSASVGDRVTITCSASSSV |
| (hOKT3a) | YTFTRYTMHWVRQAPGQGLEWMGYIN | SYMNWYQQKPGKAPKRLIYDTSKLASG |
| | PSRGYTNYNQKFKDRVTLTTDKSSST | VPSRFSGSGSGTDFTLTISSLQPEDFATY |
| | AYMELSSLRSEDTAVYYCARYYDDHY | YCQQWSSNPFTFGQGTKLEIK (SEQ ID |
| | SLDYWGQGTLVTVSS | NO: 25) |
| | (SEQ ID NO: 24) | |
| Anti-CD3 | EVQLVESGGGLVQPGGSLRLSCAASG | DIQMTQSPSSLSASVGDRVTITCRASQD |
| (UCHT1v9) | YSFTGYTMNWVRQAPGKGLEWVALIN | IRNYLNWYQQKPGKAPKLLIYYTSRLES |
| | PYKGVSTYNQKFKDRFTISVDKSKNT | GVPSRFSGSGSGTDYTLTISSLQPEDFA |
| | LAYQMNSLRAEDTAVYYCARSGYYGD | TYYCQQGNTLPWTFGQGTKVEIK (SEQ |
| | SDWYFDVWGQGTLVTVSS | ID NO: 33) |
| | (SEQ ID NO: 32) | |
| Anti-CD3 | EVQLVESGGGLVQPGGSLRLSCAASGF | QAVVTQEPSLTVSPGGTVTLTCGSSTGA |
| (hSP34) | TFSTYAMNWVRQAPGKGLEWVGRIRS | VTTSNYANWVQQKPGKSPRGLIGGTNK |
| | KYNNYATYYADSVKGRFTISRDDSKNT | RAPGVPARFSGSLLGGKAALTISGAQPE |
| | LYLQMNSLRAEDTAVYYCVRHGNFGD | DEADYYCALWYSNHWVFGGGTKLTVL |
| | SYVSWFAYWGQGTLVTVSS (SEQ ID | (SEQ ID NO: 35) |
| | NO: 34) | |

TABLE 22
CDR sequences for Anti-CD3 Binder (according to Kabat numbering
scheme)

| Clone name | HCDR1 | HCDR2 | HCDR3 | LCDR1 | LCDR2 | LCDR3 |
|---|---|---|---|---|---|---|
| Anti-CD3 | RYTMH | YINPSRGYTN | YYDDHYS | SASSSVSY | DTSKLAS | QQWSSNPF |
| (hOKT3a) | (SEQ ID | YNQKFKD | LDY (SEQ | MN (SEQ | (SEQ ID | T (SEQ ID |
| | NO: 26) | (SEQ ID | ID NO: 28) | ID NO: 29) | NO: 30) | NO: 31) |
| | | NO: 27) | | | | |
| Anti-CD3 | GYTMN | LINPYKGVSTY | SGYYGDS | RASQDIRN | YTSRLES | QQGNTLP |
| (UCHT1v9) | (SEQ ID | NQKFKD (SEQ | DWYFDV | YLN (SEQ | (SEQ ID | WT (SEQ ID |
| | NO: 36) | ID NO: 37) | (SEQ ID | ID NO: 39) | NO: 40) | NO: 41) |
| | | | NO: 38) | | | |
| Anti-CD3 | TYAMN | RIRSKYNNYA | GSSTGAVT | GSSTGAVT | GTNKRAP | ALWYSNH |
| (hSP34x) | (SEQ ID | TYYADSVKG | TSNYAN | TSNYAN | (SEQ ID | WV (SEQ |
| | NO: 42) | (SEQ ID | (SEQ ID | (SEQ ID | NO: 46) | ID NO: 47) |
| | | NO: 43) | NO: 44) | NO: 45) | | |

TABLE 23
Sequence of Format 41 31E07-CD3 bispecific antibodies

| | | L4 Linker-VH-CH1- | | ABR |
|---|---|---|---|---|
| | L3 Linker-Fc | hinge-CH2- | LC | (Chain 4; VH- |
| Name | (Chain1) | CH3 (Chain2) | (Chain 3) | Linker-VL) |
| 41-31E0 | GGGGSEPKSSD | GGGGSGGGGSQVQLVQSG | DIQMTQSPSSLS | QVQLVQSGAEV |
| -hOKT3a | KTHTCPPCPAP | AEVKKPGASVKVSCKASG | ASVGDRVTITC | KKPGASVKVSC |
| | EFEGGPSVFLFP | YTFTRYTMHWVRQAPGQ | SASSSVSYMN | KASGFTFTNYLI |
| | PKPKDTLMISR | GLEWMGYINPSRGYTNYN | WYQQKPGKAP | HWVRQAPGQG |
| | TPEVTCVVVDV | QKFKDRVTLTTDKSSSTAY | KRLIYDTSKLA | LEWMGWINPN |
| | SHEDPEVKFNW | MELSSLRSEDTAVYYCARY | SGVPSRFSGSGS | SGGTNYAQRLQ |
| | YVDGVEVHNA | YDDHYSLDYWGQGTLVTV | GTDFTLTISSLQ | GRVTMTRDTST |
| | KTKPREEQYNS | SSASTKGPSVFPLAPSSKSTSG | PEDFATYYCQQ | STVYMELSSLR |
| | TYRVVSVLTVL | GTAALGCLVKDYFPEPVTVSW | WSSNPFTFGQG | SEDTAVYYCAR |
| | HQDWLNGKEY | NSGALTSGVHTFPAVLQSSGLY | TKLEIKRTVAA | DYGAGNDYWG |
| | KCKVSNKALPA | SLSSVVTVPSSSLGTQTYICNVN | PSVFIFPPSDEQ | QGTLVTVSSGG |
| | SIEKTISKAKGQ | HKPSNTKVDKRVepkscdktht | LKSGTASVVCL | GGSGGGGSDIQ |
| | PREPQVYTLPP | cpPCPAPEFEGGPSVFLFPPKP | LNNFYPREAKV | MTQSPSSLSAS |
| | CREEMTKNQV | KDTLMISRTPEVTCVVVDVS | QWKVDNALQS | VGDRVTITCRA |
| | SLWCLVKGFYP | HEDPEVKFNWYVDGVEVHN | GNSQESVTEQD | SQEIRRWLAW |
| | SDIAVEWESNG | AKTKPREEQYNSTYRVVSVL | SKDSTYSLSSTL | YQQKPGKAPK |
| | QPENNYKTTPP | TVLHQDWLNGKEYKCKVSN | TLSKADYEKHK | LLIYAASNLQS |
| | VLDSDGSFFLY | KALPASIEKTISKAKGQPREP | VYACEVTHQG | GVPSRFSGSGS |
| | SKLTVDKSRW | QVCTLPPSREEMTKNQVSLSC | LSSPVTKSFNR | GTDFTLTISSL |
| | QQGNVFSCSV | AVKGFYPSDIAVEWESNGQPE | GEC (SEQ ID | QPEDFATYYC |
| | MHEALHNHYT | NNYKTTPPVLDSDGSFFLVSK | NO: 50) | QQSYSTPYTFG |
| | QKSLSLSPGK | LTVDKSRWQQGNVFSCSVMH | | QGTKLEIK |
| | (SEQ ID NO: 48) | EALHNRFTQKSLSLSPGK | | (SEQ ID NO: 55) |
| | | (SEQ ID NO: 49) | | |
| 41- | GGGGSEPKSSD | GGGGSGGGGSEVQLVESGG | DIQMTQSPSSLS | QVQLVQSGAEV |
| 31E07- | KTHTCPPCPAP | GLVQPGGSLRLSCAASGYS | ASVGDRVTITC | KKPGASVKVSC |
| UCHT1v | EFEGGPSVFLFP | FTGYTMNWVRQAPGKGL | RASQDIRNYLN | KASGFTFTNYLI |
| 9 | PKPKDTLMISR | EWVALINPYKGVSTYNQK | WYQQKPGKAP | HWVRQAPGQG |
| | TPEVTCVVVDV | FKDRFTISVDKSKNTAYLQ | KLLIYYTSRLES | LEWMGWINPN |
| | SHEDPEVKFNW | MNSLRAEDTAVYYCARSG | GVPSRFSGSGS | SGGTNYAQRLQ |
| | YVDGVEVHNA | YYGDSDWYFDVWGQGTL | GTDYTLTISSLQ | GRVTMTRDTST |
| | KTKPREEQYNS | VTVSSASTKGPSVFPLAPSSKS | PEDFATYYCQQ | STVYMELSSLR |
| | TYRVVSVLTVL | TSGGTAALGCLVKDYFPEPVT | GNTLPWTFGQ | SEDTAVYYCAR |
| | HQDWLNGKEY | VSWNSGALTSGVHTFPAVLQSS | GTKVEIKRTVA | DYGAGNDYWG |
| | KCKVSNKALPA | GLYSLSSVVTVPSSSLGTQTYI | APSVFIFPPSDE | QGTLVTVSSGG |
| | SIEKTISKAKGQ | CNVNHKPSNTKVDKRVepkscd | QLKSGTASVVC | GGSGGGGSDIQ |
| | PREPQVYTLPP | kthtcpPCPAPEFEGGPSVFLF | LLNNFYPREAK | MTQSPSSLSAS |
| | CREEMTKNQV | PPKPKDTLMISRTPEVTCVV | VOWKVDNALQ | VGDRVTITCRA |
| | SLWCLVKGFYP | VDVSHEDPEVKFNWYVDGV | SGNSQESVTEQ | SQEIRRWLAW |
| | SDIAVEWESNG | EVHNAKTKPREEQYNSTYRV | DSKDSTYSLSS | YQQKPGKAPK |
| | QPENNYKTTPP | VSVLTVLHQDWLNGKEYKC | TLTLSKADYEK | LLIYAASNLQS |
| | VLDSDGSFFLY | KVSNKALPASIEKTISKAKGQ | HKVYACEVTH | GVPSRFSGSGS |
| | SKLTVDKSRW | PREPQVCTLPPSREEMTKNQV | QGLSSPVTKSE | GTDFTLTISSL |
| | QQGNVFSCSV | SLSCAVKGFYPSDIAVEWESN | NRGEC (SEQ ID | QPEDFATYYC |
| | MHEALHNHYT | GQPENNYKTTPPVLDSDGSFF | NO: 52) | QQSYSTPYTFG |
| | QKSLSLSPGK | LVSKLTVDKSRWQQGNVFSCS | | QGTKLEIK |
| | (SEQ ID NO: 48) | VMHEALHNRFTQKSLSLSPGK | | (SEQ ID |
| | | (SEQ ID NO: 51) | | NO: 55) |
| 41- | GGGGSEPKSSD | GGGGSGGGGS | QAVVTQEPSLT | QVQLVQSGAEV |
| 31E07- | KTHTCPPCPAP | EVQLVESGGGLVQPGGSL | VSPGGTVTLTC | KKPGASVKVSC |
| hSP34x | EFEGGPSVFLFP | RLSCAASGFTFSTYAMNW | GSSTGAVTTSN | KASGFTFTNYLI |
| | PKPKDTLMISR | VRQAPGKGLEWVGRIRSK | YANWVQQKPG | HWVRQAPGQG |
| | TPEVTCVVVDV | YNNYATYYADSVKGRFTIS | KSPRGLIGGTN | LEWMGWINPN |
| | SHEDPEVKFNW | RDDSKNTLYLQMNSLRAE | KRAPGVPARFS | SGGTNYAQRLQ |
| | YVDGVEVHNA | DTAVYYCVRHGNFGDSYV | GSLLGGKAALT | GRVTMTRDTST |
| | KTKPREEQYNS | SWFAYWGQGTLVTVSSAS | ISGAQPEDEAD | STVYMELSSLR |
| | TYRVVSVLTVL | TKGPSVFPLAPSSKSTSG | YYCALWYSNH | SEDTAVYYCAR |
| | HQDWLNGKEY | GTAALGCLVKDYFPEPVT | WVFGGGTKLT | DYGAGNDYWG |
| | KCKVSNKALPA | VSWNSGALTSGVHTFPAV | VL | QGTLVTVSSGG |
| | SIEKTISKAKGQ | LQSSGLYSLSSVVTVPSS | RTVAAPSVFIFP | GGSGGGGSDIQ |
| | PREPQVYTLPP | SLGTQTYICNVNHKPSNT | PSDEQLKSGTA | MTQSPSSLSAS |
| | CREEMTKNQV | TKVDKRVEPKSCDKTHCP | SVVCLLNNFYP | VGDRVTITCRA |
| | SLWCLVKGFYP | PCPAPEFEGGPSVFLFPPKP | REAKVQWKVD | SQEIRRWLAW |
| | SDIAVEWESNG | KDTLMISRTPEVTCVVVDVS | NALQSGNSQES | YQQKPGKAPK |
| | QPENNYKTTPP | HEDPEVKFNWYVDGVEVHN | VTEQDSKDSTY | LLIYAASNLQS |
| | VLDSDGSFFLY | AKTKPREEQYNSTYRVVSVL | SLSSTLTLSKAD | GVPSRFSGSGS |
| | SKLTVDKSRW | TVLHQDWLNGKEYKCKVSN | YEKHKVYACE | GTDFTLTISSL |
| | QQGNVFSCSV | KALPASIEKTISKAKGQPREP | VTHQGLSSPVT | QPEDFATYYC |
| | MHEALHNHYT | QVCTLPPSREEMTKNQVSLSC | KSFNRGEC | QQSYSTPYTFG |
| | QKSLSLSPGK | AVKGFYPSDIAVEWESNGQPE | (SEQ ID | QGTKLEIK |
| | (SEQ ID NO: 48) | NNYKTTPPVLDSDGSFFLVSK | NO: 54) | (SEQ ID |
| | | LTVDKSRWQQGNVFSCSVMH | | NO: 55) |
| | | EALHNRFTQKSLSLSPGK | | |
| | | (SEQ ID NO: 53) | | |

Fragments of the Formats 41, 42, and 43 antibody herein have text formatted differently. In Chain 1, the L3 linker is non-bold and underlined, the Fc is non-bold. In Chain 2, the L4 linker is non-bold and underlined, the VH is bold, the CH1 is non-bold, italics and underlined, the hinge is lower case, CH2 is bold and italics, CH3 is non-bold and italics. In Chain 3, the CL is underlined. In the ABR (Chain 4), the VH is non-bold and italics, the linker (L1 or L2) is non-bold and underlined, and the VL is bold.

TABLE 24
Sequences of Format 42 31E07-CD3 bispecific antibodies

| | | Linker-VH- | | Mixed ABR | Mixed ABR |
|---|---|---|---|---|---|
| | Linker-Fc | CH1-hinge- | LC | (chain 4) | (chain 4) |
| Name | (Chain1) | CH2-CH3 (Chain2) | (Chain 3) | VH-VL | VH-VL |
| 42-31E07 | GGGGSEPKS | GGGGSGGGGSQVQLV | DIQMTQSP | QVQLVQSGA | EVQLVESGGGL |
| UCHT1v9 | SDKTHTCPP | QSGAEVKKPGASVK | SSLSASVG | EVKKPGASV | VQPGGSLRLSC |
| | CPAPEFEGG | VSCKASGFTFTNYLI | DRVTITCR | KVSCKASGFT | AASGYSFTGYT |
| | PSVFLFPPKP | HWVRQAPGQGLEW | ASQEIRRW | FTNYLIHWV | MNWVRQAPGK |
| | KDTLMISRT | MGWINPNSGGTNYA | LAWYQQK | RQAPGQGLE | GLEWVALINPY |
| | PEVTCVVVD | QRLQGRVTMTRDTS | PGKAPKLLI | WMGWINPNS | KGVSTYNQKFK |
| | VSHEDPEVK | TSTVYMELSSLRSED | YAASNLQS | GGTNYAQRL | DRFTISVDKSKN |
| | FNWYVDGV | TAVYYCARDYGAGN | GVPSRFSGS | QGRVTMTRD | TAYLQMNSLRA |
| | EVHNAKTKP | DYWGQGTLVTVSSAST | GSGTDFTL | TSTSTVYMEL | EDTAVYYCARS |
| | REEQYNSTY | KGPSVFPLAPSSKSTSG | TISSLQPED | SSLRSEDTAV | GYYGDSDWYF |
| | RVVSVLTVL | GTAALGCLVKDYFPEPV | FATYYCQQ | YYCARDYGA | DVWGQGTLVT |
| | HQDWLNGK | TVSWNSGALTSGVHTFP | SYSTPYTFG | GNDYWGQG | VSSGGGGSGGG |
| | EYKCKVSN | AVLQSSGLYSLSSVVTVP | QGTKLEIK | TLVTVSSGGG | GSDIQMTQSPS |
| | KALPASIEK | SSSLGTQTYICNVNHKPS | RTVAAPSV | GSGGGGSDI | SLSASVGDRVT |
| | TISKAKGQP | NTKVDKRVepkscdktht | FIFPPSDEQ | QMTQSPSSL | ITCRASQEIRR |
| | REPQVYTLP | cpPCPAPEFEGGPSVFLF | LKSGTASV | SASVGDRVTI | WLAWYQQKP |
| | PCREEMTKN | PPKPKDTLMISRTPEV | VCLLNNFY | TCRASQDIR | GKAPKLLIYAA |
| | QVSLWCLV | TCVVVDVSHEDPEVK | PREAKVQ | NYLNWYQQ | SNLQSGVPSRF |
| | KGFYPSDIA | FNWYVDGVEVHNAKT | WKVDNAL | KPGKAPKLL | SGSGSGTDFTL |
| | VEWESNGQ | KPREEQYNSTYRVVSV | QSGNSQES | IYYTSRLESG | TISSLQPEDFAT |
| | PENNYKTTP | LTVLHQDWLNGKEYK | VTEQDSKD | VPSRFSGSGS | YYCQQSYSTPY |
| | PVLDSDGSF | CKVSNKALPASIEKTIS | STYSLSSTL | GTDYTLTISS | TFGQGTKLEIK |
| | FLYSKLTVD | KAKGQPREPQVCTLPP | TLSKADYE | LQPEDFATY | (SEQ ID NO: 58) |
| | KSRWQQGN | SREEMTKNQVSLSCAVK | KHKVYACE | YCQQGNTLP | |
| | VFSCSVMHE | GFYPSDIAVEWESNGQP | VTHQGLSS | WTFGQGTK | |
| | ALHNHYTQ | ENNYKTTPPVLDSDGSF | PVTKSFNR | VEIK | |
| | KSLSLSPGK | FLVSKLTVDKSRWQQG | GEC | (SEQ ID | |
| | (SEQ ID | NVFSCSVMHEALHNRF | (SEQ ID | NO: 59) | |
| | NO: 48) | TQKSLSLSPGK | NO: 57) | | |
| | | (SEQ ID NO: 56) | | | |

TABLE 25
Sequences of Format 43 31E07-CD3 bispecific antibodies

| | | Linker-VH- | | Mixed ABR | Mixed ABR |
|---|---|---|---|---|---|
| | Linker-Fc | CH1-hinge- | LC | (chain 4) | (chain 4) |
| Name | (Chain1) | CH2-CH3 (Chain2) | (Chain 3) | VH-VL | VH-VL |
| 43-31E07- | GGGGSEPKS | GGGGSGGGGSQVQL | DIQMTQSPS | EVQLVESGGG | QVQLVQSGAE |
| GGGGS- | SDKTHTCPP | VQSGAEVKKPGASV | SLSASVGDR | LVQPGGSLRL | VKKPGASVKV |
| UCHT1v9 | CPAPEFEGG | KVSCKASGFTFTNY | VTITCRASQ | SCAASGYSFT | SCKASGFTFTN |
| | PSVFLFPPKP | LIHWVRQAPGQGLE | EIRRWLAW | GYTMNWVRQ | YLIHWVRQAP |
| | KDTLMISRT | WMGWINPNSGGTN | YQQKPGKA | APGKGLEWVA | GQGLEWMGWI |
| | PEVTCVVVD | YAQRLQGRVTMTR | PKLLIYAAS | LINPYKGVST | NPNSGGTNYA |
| | VSHEDPEVK | DTSTSTVYMELSSLR | NLQSGVPSR | YNQKFKDRFT | QRLQGRVTMT |
| | FNWYVDGV | SEDTAVYYCARDYG | FSGSGSGTD | ISVDKSKNTA | RDTSTSTVYM |
| | EVHNAKTKP | AGNDYWGQGTLVT | FTLTISSLQP | YLQMNSLRAE | ELSSLRSEDTA |
| | REEQYNSTY | VSSASTKGPSVFPLAPS | EDFATYYCQ | DTAVYYCARS | VYYCARDYGA |
| | RVVSVLTVL | SKSTSGGTAALGCLVK | QSYSTPYTF | GYYGDSDWY | GNDYWGQGTL |
| | HQDWLNGK | DYFPEPVTVSWNSGAL | GQGTKLEIK | FDVWGQGTL | VTVSSGGGGS |
| | EYKCKVSN | TSGVHTFPAVLQSSGL | RTVAAPSVE | VTVSSGGGGS | GGGGSDIQMT |
| | KALPASIEK | YSLSSVVTVPSSSLGTQ | IFPPSDEQLK | GGGGSDIQMT | QSPSSLSASVG |
| | TISKAKGQP | TYICNVNHKPSNTKVD | SGTASVVCL | QSPSSLSASV | DRVTITCRAS |
| | REPQVYTLP | KRVepkscdkthtcp | LNNFYPREA | GDRVTITCRA | QDIRNYLNWY |
| | PCREEMTKN | PCPAPEFEGGPSVFLF | KVQWKVDN | SQEIRRWLA | QQKPGKAPK |
| | QVSLWCLV | PPKPKDTLMISRTPE | ALQSGNSQE | WYQQKPGKA | LLIYYTSRLES |
| | KGFYPSDIA | VTCVVVDVSHEDPEV | SVTEQDSKD | PKLLIYAASN | GVPSRFSGSGS |
| | VEWESNGQ | KFNWYVDGVEVHNAK | STYSLSSTLT | LQSGVPSRFS | GTDYTLTISSL |
| | PENNYKTTP | TKPREEQYNSTYRVV | LSKADYEKH | GSGSGTDFTL | QPEDFATYYC |
| | PVLDSDGSF | SVLTVLHQDWLNGKEY | KVYACEVT | TISSLQPEDFA | QQGNTLPWT |
| | FLYSKLTVD | KCKVSNKALPASIEKT | HQGLSSPVT | TYYCQQSYST | FGQGTKVEIK |
| | KSRWQQGN | ISKAKGQPREPQVCTL | KSFNRGEC | PYTFGQGTK | (SEQ ID |
| | VFSCSVMHE | PPSREEMTKNQVSLSC | (SEQ ID | LEIK | NO: 59) |
| | ALHNHYTQ | AVKGFYPSDIAVEWES | NO: 57) | (SEQ ID | |
| | KSLSLSPGK | NGQPENNYKTTPPVLD | | NO: 58) | |
| | (SEQ ID | SDGSFFLVSKLTVDKS | | | |
| | NO: 48) | RWQQGNVFSCSVMHE | | | |
| | | ALHNRFTQKSLSLSPG | | | |
| | | K | | | |
| | | (SEQ ID NO: 56) | | | |

TABLE 26
CDR sequences of identified scFvs to G5-pHLA,
numbered according to the Kabat
numbering scheme
CDR sequences of identified scFvs to G5,
numbered according to the Kabat numbering
scheme

| Target | Clone | | | | | | |
|---|---|---|---|---|---|---|---|
| group | name | HCDR1 | HCDR2 | HCDR3 | LCDR1 | LCDR2 | LCDR3 |
| G5 | G5 | GTF | GGII | CATP | RASQ | AAS | CQQ |
| | (1C1 | SNF | PILG | TNSG | SISS | TLQS | SYS |
| | 2) | GVS | TA | YYGP | WLA | (SEQ | IPL |
| | | (SEQ | NYA | YYYY | (SEQ | ID | TF |
| | | ID | (SEQ | GMDV | ID | NO: | (SEQ |
| | | NO: | ID | W | NO: | 77) | ID |
| | | 73) | NO: | (SEQ | 76) | | NO: |
| | | | 74) | ID | | | 78) |
| | | | | NO: | | | |
| | | | | 75) | | | |

TABLE 27
VH and VL sequences of scFv hits
that bind target G5-pHLA
VH and VL sequences of scFv
hits that bind target G5
VH and VL sequences of scFv
hits that bind target G5

| Target | Clone | | |
|---|---|---|---|
| group | name | V_{H} | V_{L} |
| G5 | G5(1C12) | QVQLVQSGAEVKK | DIQMTQSPSSLSA |
| | | PGSSVKVSCKASG | SVGDRVTITCRAS |
| | | GTFSNFGVSWLRQ | QSISSWLAWYQQK |
| | | APGQGLEWMGGII | PGKAPKLLIYAAS |
| | | PILGTANYAQKFQ | TLQSGVPSRFSGS |
| | | GRVTITADESTST | GSGTDFTLTISSL |
| | | AYMELSSLRSEDT | QPEDFATYYCQQS |
| | | AVYYCATPTNSGY | YSIPLTFGGGTKV |
| | | YGPYYYYGMDVWG | EIK |
| | | QGTTVTVSS | (SEQ ID |
| | | (SEQ ID | NO: 80) |
| | | NO: 79) | |

TABLE 28
Exemplary Bispecific Format 4 Constructs

| | Linker- | scFv | | VH | CH1-CH2- | |
|---|---|---|---|---|---|---|
| | Fc | (Chains | Linker | (Chain | CH3 | LC |
| Name | (Chain1) | 1 and 2) | (Chain2) | 2) | (Chain2) | (Chain 3) |
| 4-G2 | GGGGSEPK | QVQLVQSG | GGGGSG | QVQL | ASTKGPSVFPL | DIQMTQSP |
| (1H11)- | SSDKTHTC | AEVKKPGAS | GGGS | VQSG | APSSKSTSGGT | SSLSASVG |
| hOKT3 | PPCPAPELL | VKVSCKASG | (SEQ ID | AEVK | AALGCLVKDY | DRVTITCS |
| | GGPSVFLF | YTFTNYYM | NO: 111) | KPGAS | FPEPVTVSWNS | ASSSVSYM |
| | PPKPKDTL | HWVRQAPG | | VKVS | GALTSGVHTFP | NWYQQKP |
| | MISRTPEV | QGLEWMGM | | CKAS | AVLQSSGLYSL | GKAPKRLI |
| | TCVVVDVS | INPSGGGTS | | GYTFT | SSVVTVPSSSL | YDTSKLAS |
| | HEDPEVKF | YAQKFQGR | | RYTM | GTQTYICNVNH | GVPSRFSG |
| | NWYVDGV | VTMTRDTST | | HWVR | KPSNTKVDKR | SGSGTDFT |
| | EVHNAKT | STVYMELSS | | QAPG | VEPKSCDKTHT | LTISSLQPE |
| | KPREEQYQ | LRSEDTAVY | | QGLE | CPPCPAPELLG | DFATYYC |
| | STYRVVSV | YCARGNPW | | WMGY | GPSVFLFPPKPK | QQWSSNPF |
| | LTVLHQD | ELRLDYWG | | INPSR | DTLMISRTPEV | TFGQGTKL |
| | WLNGKEY | QGTLVTVSS | | GYTN | TCVVVDVSHE | EIKRTVAA |
| | KCKVSNK | GGGGSGGG | | YNQK | DPEVKFNWYV | PSVFIFPPS |
| | ALPAPIEKT | GSGGGGSG | | FKDR | DGVEVHNAKT | DEQLKSGT |
| | ISKAKGQP | GGGSDIQMT | | VTLTT | KPREEQYQSTY | ASVVCLLN |
| | REPQVYTL | QSPSSLSASV | | DKSSS | RVVSVLTVLHQ | NFYPREAK |
| | PPCREEMT | GDRVTITCQ | | TAYM | DWLNGKEYKC | VQWKVDN |
| | KNQVSLW | ASQDISNYL | | ELSSL | KVSNKALPAPI | ALQSGNSQ |
| | CLVKGFYP | NWYQQKPG | | RSEDT | EKTISKAKGQP | ESVTEQDS |
| | SDIAVEWE | KAPKLLIYA | | AVYY | REPQVCTLPPS | KDSTYSLS |
| | SNGQPENN | ASSLQSGVP | | CARY | REEMTKNQVS | STLTLSKA |
| | YKTTPPVL | SRFSGSGSG | | YDDH | LSCAVKGFYPS | DYEKHKV |
| | DSDGSFFL | TDFTLTISSL | | YSLD | DIAVEWESNGQ | YACEVTH |
| | YSKLTVDK | QPEDFATYY | | YWGQ | PENNYKTTPPV | QGLSSPVT |
| | SRWQQGN | CQQYYSYPF | | GTLVT | LDSDGSFFLVS | KSFNRGEC |
| | VFSCSVMH | TFGPGTKVD | | VSS | KLTVDKSRWQ | (SEQ ID |
| | EALHNHYT | IK (SEQ ID | | (SEQ | QGNVFSCSVM | NO: 85) |
| | QKSLSLSP | NO: 82) | | ID NO: | HEALHNRFTQK | |
| | GK (SEQ ID | | | 83) | SLSLSPGK (SEQ | |
| | NO: 81) | | | | ID NO: 84) | |
| 4-G2 | GGGGSEPK | QVQLVQSG | GGGGSG | EVQL | ASTKGPSVFPL | QAVVTQE |
| (1H11) | SSDKTHTC | AEVKKPGAS | GGGS | VESG | APSSKSTSGGT | PSLTVSPG |
| hSP34x | PPCPAPELL | VKVSCKASG | (SEQ ID | GGLV | AALGCLVKDY | GTVTLTCG |
| | GGPSVFLF | YTFTNYYM | NO: 111) | QPGGS | FPEPVTVSWNS | SSTGAVTT |
| | PPKPKDTL | HWVRQAPG | | LRLSC | GALTSGVHTFP | SNYANWV |
| | MISRTPEV | QGLEWMGM | | AASGF | AVLQSSGLYSL | QQKPGKSP |
| | TCVVVDVS | INPSGGGTS | | TFSTY | SSVVTVPSSSL | RGLIGGTN |
| | HEDPEVKF | YAQKFQGR | | AMNW | GTQTYICNVNH | KRAPGVP |
| | NWYVDGV | VTMTRDTST | | VRQA | KPSNTKVDKR | ARFSGSLL |
| | EVHNAKT | STVYMELSS | | PGKG | VEPKSCDKTHT | GGKAALTI |
| | KPREEQYQ | LRSEDTAVY | | LEWV | CPPCPAPELLG | SGAQPEDE |
| | STYRVVSV | YCARGNPW | | GRIRS | GPSVFLFPPKPK | ADYYCAL |
| | LTVLHQD | ELRLDYWG | | KYNN | DTLMISRTPEV | WYSNHWV |
| | WLNGKEY | QGTLVTVSS | | YATY | TCVVVDVSHE | FGGGTKLT |
| | KCKVSNK | GGGGSGGG | | YADS | DPEVKFNWYV | VLRTVAAP |
| | ALPAPIEKT | GSGGGGSG | | VKGR | DGVEVHNAKT | SVFIFPPSD |
| | ISKAKGQP | GGGSDIQMT | | FTISR | KPREEQYQSTY | EQLKSGTA |
| | REPQVYTL | QSPSSLSASV | | DDSK | RVVSVLTVLHQ | SVVCLLNN |
| | PPCREEMT | GDRVTITCQ | | NTLYL | DWLNGKEYKC | FYPREAKV |
| | KNQVSLW | ASQDISNYL | | QMNS | KVSNKALPAPI | QWKVDNA |
| | CLVKGFYP | NWYQQKPG | | LRAE | EKTISKAKGQP | LQSGNSQE |
| | SDIAVEWE | KAPKLLIYA | | DTAV | REPQVCTLPPS | SVTEQDSK |
| | SNGQPENN | ASSLQSGVP | | YYCV | REEMTKNQVS | DSTYSLSS |
| | YKTTPPVL | SRFSGSGSG | | RHGN | LSCAVKGFYPS | TLTLSKAD |
| | DSDGSFFL | TDFTLTISSL | | FGDSY | DIAVEWESNGQ | YEKHKVY |
| | YSKLTVDK | QPEDFATYY | | VSWF | PENNYKTTPPV | ACEVTHQ |
| | SRWQQGN | CQQYYSYPF | | AYWG | LDSDGSFFLVS | GLSSPVTK |
| | VFSCSVMH | TFGPGTKVD | | QGTL | KLTVDKSRWQ | SFNRGEC |
| | EALHNHYT | IK (SEQ ID | | VTVSS | QGNVFSCSVM | (SEQ ID |
| | QKSLSLSP | NO: 87) | | (SEQ | HEALHNRFTQK | NO: 90) |
| | GK (SEQ ID | | | ID NO: | SLSLSPGK (SEQ | |
| | NO: 86) | | | 88) | ID NO: 89) | |
| 4-G5 | GGGGSEPK | QVQLVQSG | GGGGSG | QVQL | ASTKGPSVFPL | DIQMTQSP |
| (1C12) | SSDKTHTC | AEVKKPGSS | GGGS | VQSG | APSSKSTSGGT | SSLSASVG |
| hOKT3a | PPCPAPELL | VKVSCKASG | (SEQ ID | AEVK | AALGCLVKDY | DRVTITCS |
| | GGPSVFLF | GTFSNFGVS | NO: 111) | KPGAS | FPEPVTVSWNS | ASSSVSYM |
| | PPKPKDTL | WLRQAPGQ | | VKVS | GALTSGVHTFP | NWYQQKP |
| | MISRTPEV | GLEWMGGII | | CKAS | AVLQSSGLYSL | GKAPKRLI |
| | TCVVVDVS | PILGTANYA | | GYTFT | SSVVTVPSSSL | YDTSKLAS |
| | HEDPEVKF | QKFQGRVTI | | RYTM | GTQTYICNVNH | GVPSRFSG |
| | NWYVDGV | TADESTSTA | | HWVR | KPSNTKVDKR | SGSGTDFT |
| | EVHNAKT | YMELSSLRS | | QAPG | VEPKSCDKTHT | LTISSLQPE |
| | KPREEQYQ | EDTAVYYC | | QGLE | CPPCPAPELLG | DFATYYC |
| | STYRVVSV | ATPTNSGYY | | WMGY | GPSVFLFPPKPK | QQWSSNPF |
| | LTVLHQD | GPYYYYGM | | INPSR | DTLMISRTPEV | TFGQGTKL |
| | WLNGKEY | DVWGQGTT | | GYTN | TCVVVDVSHE | EIKRTVAA |
| | KCKVSNK | VTVSSGGGG | | YNQK | DPEVKFNWYV | PSVFIFPPS |
| | ALPAPIEKT | SGGGGSGG | | FKDR | DGVEVHNAKT | DEQLKSGT |
| | ISKAKGQP | GGSGGGGS | | VTLTT | KPREEQYQSTY | ASVVCLLN |
| | REPQVYTL | DIQMTQSPS | | DKSSS | RVVSVLTVLHQ | NFYPREAK |
| | PPCREEMT | SLSASVGDR | | TAYM | DWLNGKEYKC | VQWKVDN |
| | KNQVSLW | VTITCRASQ | | ELSSL | KVSNKALPAPI | ALQSGNSQ |
| | CLVKGFYP | SISSWLAWY | | RSEDT | EKTISKAKGQP | ESVTEQDS |
| | SDIAVEWE | QQKPGKAP | | AVYY | REPQVCTLPPS | KDSTYSLS |
| | SNGQPENN | KLLIYAAST | | CARY | REEMTKNQVS | STLTLSKA |
| | YKTTPPVL | LQSGVPSRF | | YDDH | LSCAVKGFYPS | DYEKHKV |
| | DSDGSFFL | SGSGSGTDF | | YSLD | DIAVEWESNGQ | YACEVTH |
| | YSKLTVDK | TLTISSLQPE | | YWGQ | PENNYKTTPPV | QGLSSPVT |
| | SRWQQGN | DFATYYCQ | | GTLVT | LDSDGSFFLVS | KSFNRGEC |
| | VFSCSVMH | QSYSIPLTFG | | VSS | KLTVDKSRWQ | (SEQ ID |
| | EALHNHYT | GGTKVEIK | | (SEQ | QGNVFSCSVM | NO: 95) |
| | QKSLSLSP | (SEQ ID NO: | | ID NO: | HEALHNRFTQK | |
| | GK (SEQ ID | 92) | | 93) | SLSLSPGK (SEQ | |
| | NO: 91) | | | | ID NO: 94) | |
| 4-G5 | GGGGSEPK | QVQLVQSG | GGGGSG | EVQL | ASTKGPSVFPL | QAVVTQE |
| (1C12) | SSDKTHTC | AEVKKPGSS | GGGS | VESG | APSSKSTSGGT | PSLTVSPG |
| hSP34x | PPCPAPELL | VKVSCKASG | (SEQ ID | GGLV | AALGCLVKDY | GTVTLTCG |
| | GGPSVFLF | GTFSNFGVS | NO: 111) | QPGGS | FPEPVTVSWNS | SSTGAVTT |
| | PPKPKDTL | WLRQAPGQ | | LRLSC | GALTSGVHTFP | SNYANWV |
| | MISRTPEV | GLEWMGGII | | AASGF | AVLQSSGLYSL | QQKPGKSP |
| | TCVVVDVS | PILGTANYA | | TFSTY | SSVVTVPSSSL | RGLIGGTN |
| | HEDPEVKF | QKFQGRVTI | | AMNW | GTQTYICNVNH | KRAPGVP |
| | NWYVDGV | TADESTSTA | | VRQA | KPSNTKVDKR | ARFSGSLL |
| | EVHNAKT | YMELSSLRS | | PGKG | VEPKSCDKTHT | GGKAALTI |
| | KPREEQYQ | EDTAVYYC | | LEWV | CPPCPAPELLG | SGAQPEDE |
| | STYRVVSV | ATPTNSGYY | | GRIRS | GPSVFLFPPKPK | ADYYCAL |
| | LTVLHQD | GPYYYYGM | | KYNN | DTLMISRTPEV | WYSNHWV |
| | WLNGKEY | DVWGQGTT | | YATY | TCVVVDVSHE | FGGGTKLT |
| | KCKVSNK | VTVSSGGGG | | YADS | DPEVKFNWYV | VLRTVAAP |
| | ALPAPIEKT | SGGGGSGG | | VKGR | DGVEVHNAKT | SVFIFPPSD |
| | ISKAKGQP | GGSGGGGS | | FTISR | KPREEQYQSTY | EQLKSGTA |
| | REPQVYTL | DIQMTQSPS | | DDSK | RVVSVLTVLHQ | SVVCLLNN |
| | PPCREEMT | SLSASVGDR | | NTLYL | DWLNGKEYKC | FYPREAKV |
| | KNQVSLW | VTITCRASQ | | QMNS | KVSNKALPAPI | QWKVDNA |
| | CLVKGFYP | SISSWLAWY | | LRAE | EKTISKAKGQP | LQSGNSQE |
| | SDIAVEWE | QQKPGKAP | | DTAV | REPQVCTLPPS | SVTEQDSK |
| | SNGQPENN | KLLIYAAST | | YYCV | REEMTKNQVS | DSTYSLSS |
| | YKTTPPVL | LQSGVPSRF | | RHGN | LSCAVKGFYPS | TLTLSKAD |
| | DSDGSFFL | SGSGSGTDF | | FGDSY | DIAVEWESNGQ | YEKHKVY |
| | YSKLTVDK | TLTISSLQPE | | VSWF | PENNYKTTPPV | ACEVTHQ |
| | SRWQQGN | DFATYYCQ | | AYWG | LDSDGSFFLVS | GLSSPVTK |
| | VFSCSVMH | QSYSIPLTFG | | QGTL | KLTVDKSRWQ | SFNRGEC |
| | EALHNHYT | GGTKVEIK | | VTVSS | QGNVFSCSVM | (SEQ ID |
| | QKSLSLSP | (SEQ ID NO: | | (SEQ | HEALHNRFTQK | NO: 100) |
| | GK | 97) | | ID NO: | SLSLSPGK | |
| | (SEQ ID | | | 98) | (SEQ | |
| | NO: 96) | | | | ID NO: 99) | |

TABLE 29
Exemplary sequences of Format 4 bispecific antibodies with
engineered disulfide bond (DSB)
bold text indicates the engineered cysteine residue

| | Linker-Fc | scFv (Chains | Linker | VH | CH1-CH2-CH3 | LC |
|---|---|---|---|---|---|---|
| Name | (Chain1) | 1 and 2) | (Chain2) | (Chain2) | (Chain2) | (Chain 3) |
| 4- | GGGGSEPKSS | QVQLVQSG | GGGGS | QVQLVQ | ASTKGPSVFPLA | DIQMTQSP |
| G2 | DKTHTCPPCP | AEVKKPGAS | GGGGS | SGAEVK | PSSKSTSGGTAA | SSLSASVG |
| (1H11) | APELLGGPSV | VKVSCKASG | (SEQ ID | KPGASVK | LGCLVKDYFPEP | DRVTITCS |
| hOKT | FLFPPKPKDT | YTFTNYYM | NO: 111) | VSCKASG | VTVSWNSGALT | ASSSVSYM |
| 3 | LMISRTPEVT | HWVRQAPG | | YTFTRYT | SGVHTFPAVLQS | NWYQQKP |
| | CVVVDVSHE | QCLEWMGM | | MHWVRQ | SGLYSLSSVVTV | GKAPKRLI |
| | DPEVKFNWY | INPSGGGTS | | APGQGLE | PSSSLGTQTYIC | YDTSKLAS |
| | VDGVEVHNA | YAQKFQGR | | WMGYIN | NVNHKPSNTKV | GVPSRFSG |
| | KTKPREEQYQ | VTMTRDTST | | PSRGYTN | DKRVEPKSCDK | SGSGTDFT |
| | STYRVVSVLT | STVYMELSS | | YNQKFK | THTCPPCPAPEL | LTISSLQPE |
| | VLHQDWLNG | LRSEDTAVY | | DRVTLTT | LGGPSVFLFPPK | DFATYYC |
| | KEYKCKVSN | YCARGNPW | | DKSSSTA | PKDTLMISRTPE | QQWSSNP |
| | KALPAPIEKTI | ELRLDYWG | | YMELSSL | VTCVVVDVSHE | FTFGQGTK |
| | SKAKGQPREP | QGTLVTVSS | | RSEDTAV | DPEVKFNWYVD | LEIKRTVA |
| | QVYTLPPCRE | GGGGSGGG | | YYCARY | GVEVHNAKTKP | APSVFIFPP |
| | EMTKNQVSL | GSGGGGSG | | YDDHYS | REEQYQSTYRV | SDEQLKSG |
| | WCLVKGFYP | GGGSDIQMT | | LDYWGQ | VSVLTVLHQDW | TASVVCLL |
| | SDIAVEWESN | QSPSSLSASV | | GTLVTVS | LNGKEYKCKVS | NNFYPREA |
| | GQPENNYKTT | GDRVTITCQ | | S (SEQ ID | NKALPAPIEKTIS | KVQWKVD |
| | PPVLDSDGSF | ASQDISNYL | | NO: 103) | KAKGQPREPQV | NALQSGN |
| | FLYSKLTVDK | NWYQQKPG | | | CTLPPSREEMTK | SQESVTEQ |
| | SRWQQGNVF | KAPKLLIYA | | | NQVSLSCAVKG | DSKDSTYS |
| | SCSVMHEAL | ASSLQSGVP | | | FYPSDIAVEWES | LSSTLTLS |
| | HNHYTQKSLS | SRFSGSGSG | | | NGQPENNYKTT | KADYEKH |
| | LSPGK (SEQ | TDFTLTISSL | | | PPVLDSDGSFFL | KVYACEV |
| | ID NO: 101) | QPEDFATYY | | | VSKLTVDKSRW | THQGLSSP |
| | | CQQYYSYPF | | | QQGNVFSCSVM | VTKSFNRG |
| | | TFGCGTKVD | | | HEALHNRFTQK | EC (SEQ ID |
| | | IK (SEQ ID | | | SLSLSPGK (SEQ | NO: 105) |
| | | NO: 102) | | | ID NO: 104) | |
| 4- | GGGGSEPKSS | QVQLVQSG | GGGGS | QVQLVQ | ASTKGPSVFPLA | DIQMTQSP |
| G5 | DKTHTCPPCP | AEVKKPGSS | GGGGS | SGAEVK | PSSKSTSGGTAA | SSLSASVG |
| (1C12) | APELLGGPSV | VKVSCKASG | (SEQ ID | KPGASVK | LGCLVKDYFPEP | DRVTITCS |
| hOKT | FLFPPKPKDT | GTFSNFGVS | NO: 111) | VSCKASG | VTVSWNSGALT | ASSSVSYM |
| 3 | LMISRTPEVT | WLRQAPGQ | | YTFTRYT | SGVHTFPAVLQS | NWYQQKP |
| | CVVVDVSHE | CLEWMGGII | | MHWVRQ | SGLYSLSSVVTV | GKAPKRLI |
| | DPEVKFNWY | PILGTANYA | | APGQGLE | PSSSLGTQTYIC | YDTSKLAS |
| | VDGVEVHNA | QKFQGRVTI | | WMGYIN | NVNHKPSNTKV | GVPSRFSG |
| | KTKPREEQYQ | TADESTSTA | | PSRGYTN | DKRVEPKSCDK | SGSGTDFT |
| | STYRVVSVLT | YMELSSLRS | | YNQKFK | THTCPPCPAPEL | LTISSLQPE |
| | VLHQDWLNG | EDTAVYYC | | DRVTLTT | LGGPSVFLFPPK | DFATYYC |
| | KEYKCKVSN | ATPTNSGYY | | DKSSSTA | PKDTLMISRTPE | QQWSSNP |
| | KALPAPIEKTI | GPYYYYGM | | YMELSSL | VTCVVVDVSHE | FTFGQGTK |
| | SKAKGQPREP | DVWGQGTT | | RSEDTAV | DPEVKFNWYVD | LEIKRTVA |
| | QVYTLPPCRE | VTVSSGGGG | | YYCARY | GVEVHNAKTKP | APSVFIFPP |
| | EMTKNQVSL | SGGGGSGG | | YDDHYS | REEQYQSTYRV | SDEQLKSG |
| | WCLVKGFYP | GGSGGGGS | | LDYWGQ | VSVLTVLHQDW | TASVVCLL |
| | SDIAVEWESN | DIQMTQSPS | | GTLVTVS | LNGKEYKCKVS | NNFYPREA |
| | GQPENNYKTT | SLSASVGDR | | S | NKALPAPIEKTI | KVQWKVD |
| | PPVLDSDGSF | VTITCRASQ | | (SEQ ID | SKAKGQPREPQV | NALQSGN |
| | FLYSKLTVDK | SISSWLAWY | | NO: 108) | CTLPPSREEMTK | SQESVTEQ |
| | SRWQQGNVF | QQKPGKAP | | | NQVSLSCAVKG | DSKDSTYS |
| | SCSVMHEAL | KLLIYAAST | | | FYPSDIAVEWES | LSSTLTLS |
| | HNHYTQKSLS | LQSGVPSRF | | | NGQPENNYKTT | KADYEKH |
| | LSPGK (SEQ | SGSGSGTDF | | | PPVLDSDGSFFL | KVYACEV |
| | ID NO: 106) | TLTISSLQPE | | | VSKLTVDKSRW | THQGLSSP |
| | | DFATYYCQ | | | QQGNVFSCSVM | VTKSFNRG |
| | | QSYSIPLTFG | | | HEALHNRFTQK | EC (SEQ ID |
| | | GGTKVEIK | | | SLSLSPGK (SEQ | NO: 110) |
| | | (SEQ ID NO: | | | ID NO: 109) | |
| | | 107) | | | | |

1-95. (canceled)

96. An isolated antigen binding protein (ABP) that comprises a first antigen binding region (ABR) and a second ABR which each specifically bind a first target antigen, a Fab that specifically binds CD3, and an Fc domain, wherein the ABP comprises:
(a) a first polypeptide comprising, in an N→C direction, the first ABR-a first hinge-a first CH2-CH3;
(b) a second polypeptide comprising, in an N→C direction, the second ABR-a variable heavy chain (VH) domain of the Fab-a CH1 domain of the Fab-a second hinge-a second CH2-CH3; and
(c) a third polypeptide comprising, in an N→C direction, a variable light chain (VL) domain of the Fab-a CL domain of the Fab;
wherein:
the first ABR and second ABR each comprise, in an N→C direction:
(i) a VH domain-a VL domain, or
(ii) a VL domain-a VH domain;
the VH domain of the first ABR is attached to the VL domain of the first ABR via a first linker;
the VH domain of the second ABR is attached to the VL domain of the second ABR via a second linker;
the first linker and second linker are each about 5-15 amino acids in length;
the first target antigen is an HLA-PEPTIDE target comprising an HLA-restricted peptide complexed with an HLA Class I molecule, wherein the HLA Class I molecule is HLA subtype HLA-A*01:01 and the HLA-restricted peptide comprises the sequence NTDNNLAVY (SEQ ID NO: 214);
the VH of the first and second ABRs comprises the sequence set forth in SEQ ID NO: 6; and
the VL of the first and second ABRs comprises the sequence set forth in SEQ ID NO:4.

97. The isolated ABP according to claim 96, wherein the first linker and second linker are each individually 8-12 amino acids.

98. The isolated ABP according to claim 96, wherein the first linker and the second linker each consist of 10 amino acids.

99. The isolated ABP according to claim 96, wherein the Fab that specifically binds CD3 comprises an OKT3, UCHT1v9, or hSP34x binding fragment.

100. The isolated ABP according to claim 96, wherein the VH and VL of the Fab comprise the sequences set forth in:
(i) SEQ ID NOs:32 and 33, respectively,
(ii) SEQ ID NOs:24 and 25, respectively, or
(iii) SEQ ID NOs:34 and 35, respectively.

101. The isolated ABP according to claim 96, wherein the first polypeptide comprises the sequence(s) set forth in:
(i) SEQ ID NO: 55, or
(ii) SEQ ID NOs: 55 and 48.

102. The isolated ABP according to claim 96, wherein the second polypeptide comprises the sequence(s) set forth in:
(i) SEQ ID NO: 55,
(ii) SEQ ID NOs: 55 and 51,
(iii) SEQ ID NOs: 55 and 49, or
(iv) SEQ ID NOs: 55 and 53.

103. The isolated ABP according to claim 96, wherein the third polypeptide comprises the sequence set forth in SEQ ID NOs: 52, 50, or 54.

104. The isolated ABP according to claim 96, wherein the first polypeptide comprises the sequence set forth in SEQ ID NOs: 55 and 48, the second polypeptide comprises the sequence set forth in SEQ ID NOs: 55 and 51, and the third polypeptide comprises the sequence set forth in SEQ ID NO: 52.

105. The isolated ABP according to claim 96, wherein the first polypeptide comprises the sequence set forth in SEQ ID NOs: 55 and 48, the second polypeptide comprises the sequence set forth in SEQ ID NOs: 55 and 49, and the third polypeptide comprises the sequence set forth in SEQ ID NO:50.

106. The isolated ABP according to claim 96, wherein the first polypeptide comprises the sequence set forth in SEQ ID NOs: 55 and 48, the second polypeptide comprises the sequence set forth in SEQ ID NOs: 55 and 53, and the third polypeptide comprises the sequence set forth in SEQ ID NO:54.

107. The isolated ABP according to claim 96, wherein the VL domain of the first ABR interacts with the VH domain of the second ABR, and the VH domain of the first ABR interacts with the VL domain of the second ABR.

108. The isolated ABP according to claim 96, wherein:
(i) the antigen binding protein is linked to a scaffold;
(ii) the antigen binding protein is linked to a scaffold, wherein the scaffold comprises serum albumin; or
(iii) the antigen binding protein is linked to a scaffold, wherein the scaffold comprises Fc.

109. The isolated ABP according to claim 108, wherein the scaffold comprises Fc, and the Fc is human Fc and is an IgG, an IgA, an IgD, an IgE, or an IgM.

110. The isolated ABP according to claim 108, wherein the scaffold comprises Fc, and the Fc is human Fc and is an IgG1, IgG2, IgG3, IgG4, IgA1, or IgA2.

111. The isolated ABP according to claim 96, wherein the antigen binding protein comprises an Fv fragment, a Fab fragment, a F(ab′)₂ fragment, a Fab′ fragment, an scFv fragment, an scFv-Fc fragment, and/or a single-domain antibody or antigen binding fragment thereof.

112. The isolated ABP according to claim 96, comprising a variant CH2-CH3 domain, wherein the variant CH2-CH3 domain of the first or second polypeptide comprises a knob-in-hole modification.

113. The isolated ABP according to claim 96, wherein the first CH2-CH3 is a first variant CH2-CH3 comprising a T366W mutation and the second CH2-CH3 is a second variant CH2-CH3 comprising T366S, L368A, and Y407V mutations, according to EU numbering.

114. The isolated ABP according to claim 96, wherein the ABP comprises a G1m17,1 allotype.

115. An isolated polynucleotide or set of polynucleotides encoding the antigen binding protein according to claim 96 or an antigen-binding portion thereof.

116. A vector or set of vectors comprising the polynucleotide or set of polynucleotides according to claim 115.

117. A host cell comprising the polynucleotide or set of polynucleotides according to claim 115.

118. The host cell according to claim 117, wherein the host cell is CHO, HEK293, or a T cell.

119. A method of producing an antigen binding protein comprising expressing the antigen binding protein with the host cell according to claim 117 and isolating the expressed antigen binding protein.

120. A pharmaceutical composition comprising the antigen binding protein according to claim 96 and a pharmaceutically acceptable excipient.

121. A method of treating cancer in a subject in need thereof, comprising administering to the subject an effective amount of the isolated ABP according to claim 96.

122. A method of increasing an immune response in a subject, comprising administering to the subject the ABP according to claim 96.

123. The method according to claim 122, wherein the subject has cancer.

124. An isolated antigen binding protein (ABP) that comprises a first antigen binding region (ABR) and a second ABR which each specifically bind a first target antigen, a Fab that specifically binds CD3, and an Fc domain, wherein the ABP comprises:
(a) a first polypeptide comprising, in an N→C direction, the first ABR-a first hinge-a first CH2-CH3;
(b) a second polypeptide comprising, in an N→C direction, the second ABR-a variable heavy chain (VH) domain of the Fab-a CH1 domain of the Fab-a second hinge-a second CH2-CH3; and
(c) a third polypeptide comprising, in an N→C direction, a variable light chain (VL) domain of the Fab-a CL domain of the Fab;
wherein:
the first ABR and second ABR each comprise, in an N→C direction:
(i) a VH domain-a VL domain, or
(ii) a VL domain-a VH domain;
the VH domain of the first ABR is attached to the VL domain of the first ABR via a first linker;
the VH domain of the second ABR is attached to the VL domain of the second ABR via a second linker;
the first linker and second linker are each about 5-15 amino acids in length;
the first target antigen is an HLA-PEPTIDE target comprising an HLA-restricted peptide complexed with an HLA Class I molecule, wherein the HLA Class I molecule is HLA subtype HLA-A*01:01 and the HLA-restricted peptide comprises the sequence NTDNNLAVY (SEQ ID NO: 214); and
the VH domains of the first and second ABRs each comprise complementarity-determining regions (CDR) H1 (CDR-H1), CDR-H2, and CDR-H3 comprising the sequences set forth in SEQ ID NOS:18, 19, and 20, respectively, and the VL domains of the first and second ABRs each comprise CDR-L1, CDR-L2, and CDR-L3 comprising the sequences set forth in SEQ ID NOS:21, 22, and 23, respectively.

125. An isolated antigen binding protein (ABP) that comprises a first antigen binding region (ABR) and a second ABR which each specifically bind a first target antigen, a Fab that specifically binds CD3, and an Fc domain, wherein the ABP comprises:
(a) a first polypeptide comprising, in an N→C direction, the first ABR-a first hinge-a first CH2-CH3;
(b) a second polypeptide comprising, in an N→C direction, the second ABR-a variable heavy chain (VH) domain of the Fab-a CH1 domain of the Fab-a second hinge-a second CH2-CH3; and
(c) a third polypeptide comprising, in an N→C direction, a variable light chain (VL) domain of the Fab-a CL domain of the Fab;
wherein:
the first ABR and second ABR each comprise, in an N→C direction:
(i) a VH domain-a VL domain, or
(ii) a VL domain-a VH domain;
the VH domain of the first ABR is attached to the VL domain of the first ABR via a first linker;
the VH domain of the second ABR is attached to the VL domain of the second ABR via a second linker;
the first linker and second linker comprise
(i) a (GS)n sequence, wherein n is 1, 2, 3, 4, 5, 6, or 7;
(ii) a (GGS)n sequence, wherein n is 1, 2, 3, 4, or 5;
(iii) a (GGGS)n sequence, wherein n is 1, 2, or 3;
(iv) a (GGSG)n sequence, wherein n is 1, 2, or 3;
(v) a (GGSGG)n sequence, wherein n is 1, 2, or 3; or
(vi) a (GGGGS)n sequence, wherein n is 1, 2, or 3
the first target antigen is an HLA-PEPTIDE target comprising an HLA-restricted peptide complexed with an HLA Class I molecule, wherein the HLA Class I molecule is HLA subtype HLA-A*01:01 and the HLA-restricted peptide comprises the sequence NTDNNLAVY (SEQ ID NO: 214);
the VH of the first and second ABRs comprises the sequence set forth in SEQ ID NO: 6; and
the VL of the first and second ABRs comprises the sequence set forth in SEQ ID NO:4.


SEQUENCE LISTING (237 sequences)

SEQ ID NO: 1 - protein, 115 aa, synthetic construct
QVQLVQSGAEVKKPGASVKVSCKASGYTFTTYNIHWVRQAPGQGLEWMGWINPNSGGTNYAQKFQGRVTMTRDTSTSTVYMELSSLRSEDTAVYYCARANWLDYWGQGTLVTVSS

SEQ ID NO: 2 - protein, 107 aa, synthetic construct
DIQMTQSPSSLSASVGDRVTITCRASQEIRRWLAWYQQKPGKAPKLLIYAASNLETGVPSRFSGSGSGTDFTLTISSLQPEDFATYYCQQSYSIPYTFGQGTKLEIK

SEQ ID NO: 3 - protein, 117 aa, synthetic construct
QVQLVQSGAEVKKPGASVKVSCKASGYTFTNYPIHWVRQAPGQGLEWMGWINPNSGGTNYAQKLQGRVTMTRDTSTSTVYMELSSLRSEDTAVYYCARDQGAGWDYWGQGTLVTVSS

SEQ ID NO: 4 - protein, 107 aa, synthetic construct
DIQMTQSPSSLSASVGDRVTITCRASQEIRRWLAWYQQKPGKAPKLLIYAASNLQSGVPSRFSGSGSGTDFTLTISSLQPEDFATYYCQQSYSTPYTFGQGTKLEIK

SEQ ID NO: 5 - protein, 115 aa, synthetic construct
QVQLVQSGAEVKKPGASVKVSCKASGATFTGYTIHWVRQAPGQGLEWMGWINPNSGGTNYAQSFQGRVTMTRDTSTSTVYMELSSLRSEDTAVYYCARAEWLDKWGQGTLVTVSS

SEQ ID NO: 6 - protein, 117 aa, synthetic construct
QVQLVQSGAEVKKPGASVKVSCKASGFTFTNYLIHWVRQAPGQGLEWMGWINPNSGGTNYAQRLQGRVTMTRDTSTSTVYMELSSLRSEDTAVYYCARDYGAGNDYWGQGTLVTVSS

SEQ ID NO: 7 - protein, 5 aa, synthetic construct
TYNIH

SEQ ID NO: 8 - protein, 17 aa, synthetic construct
WINPNSGGTNYAQKFQG

SEQ ID NO: 9 - protein, 6 aa, synthetic construct
ANWLDY

SEQ ID NO: 10 - protein, 7 aa, synthetic construct
AASNLET

SEQ ID NO: 11 - protein, 9 aa, synthetic construct
QQSYSIPYT

SEQ ID NO: 12 - protein, 5 aa, synthetic construct
NYPIH

SEQ ID NO: 13 - protein, 17 aa, synthetic construct
WINPNSGGTNYAQKLQG

SEQ ID NO: 14 - protein, 8 aa, synthetic construct
DQGAGWDY

SEQ ID NO: 15 - protein, 5 aa, synthetic construct
GYTIH

SEQ ID NO: 16 - protein, 17 aa, synthetic construct
WINPNSGGTNYAQSFQG

SEQ ID NO: 17 - protein, 6 aa, synthetic construct
AEWLDK

SEQ ID NO: 18 - protein, 5 aa, synthetic construct
NYLIH

SEQ ID NO: 19 - protein, 17 aa, synthetic construct
WINPNSGGTNYAQRLQG

SEQ ID NO: 20 - protein, 8 aa, synthetic construct
DYGAGNDY

SEQ ID NO: 21 - protein, 11 aa, synthetic construct
RASQEIRRWLA

SEQ ID NO: 22 - protein, 7 aa, synthetic construct
AASNLQS

SEQ ID NO: 23 - protein, 9 aa, synthetic construct
QQSYSTPYT

SEQ ID NO: 24 - protein, 119 aa, synthetic construct
QVQLVQSGAEVKKPGASVKVSCKASGYTFTRYTMHWVRQAPGQGLEWMGYINPSRGYTNYNQKFKDRVTLTTDKSSSTAYMELSSLRSEDTAVYYCARYYDDHYSLDYWGQGTLVTVSS

SEQ ID NO: 25 - protein, 106 aa, synthetic construct
DIQMTQSPSSLSASVGDRVTITCSASSSVSYMNWYQQKPGKAPKRLIYDTSKLASGVPSRFSGSGSGTDFTLTISSLQPEDFATYYCQQWSSNPFTFGQGTKLEIK

SEQ ID NO: 26 - protein, 5 aa, synthetic construct
RYTMH

SEQ ID NO: 27 - protein, 17 aa, synthetic construct
YINPSRGYTNYNQKFKD

SEQ ID NO: 28 - protein, 10 aa, synthetic construct
YYDDHYSLDY

SEQ ID NO: 29 - protein, 10 aa, synthetic construct
SASSSVSYMN

SEQ ID NO: 30 - protein, 7 aa, synthetic construct
DTSKLAS

SEQ ID NO: 31 - protein, 9 aa, synthetic construct
QQWSSNPFT

SEQ ID NO: 32 - protein, 122 aa, synthetic construct [sequence omitted]

SEQ ID NO: 33 - protein, 107 aa, synthetic construct [sequence omitted]

SEQ ID NO: 34 - protein, 125 aa, synthetic construct
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFAYWGQGTLVTVSS

SEQ ID NO: 35 - protein, 109 aa, synthetic construct
QAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVL

SEQ ID NO: 36 - protein, 5 aa, synthetic construct
GYTMN

SEQ ID NO: 37 - protein, 17 aa, synthetic construct
LINPYKGVSTYNQKFKD

SEQ ID NO: 38 - protein, 13 aa, synthetic construct
SGYYGDSDWYFDV

SEQ ID NO: 39 - protein, 11 aa, synthetic construct
RASQDIRNYLN

SEQ ID NO: 40 - protein, 7 aa, synthetic construct
YTSRLES

SEQ ID NO: 41 - protein, 9 aa, synthetic construct
QQGNTLPWT

SEQ ID NO: 42 - protein, 5 aa, synthetic construct
TYAMN

SEQ ID NO: 43 - protein, 19 aa, synthetic construct
RIRSKYNNYATYYADSVKG

SEQ ID NO: 44 - protein, 14 aa, synthetic construct
GSSTGAVTTSNYAN

SEQ ID NO: 45 - protein, 14 aa, synthetic construct
GSSTGAVTTSNYAN

SEQ ID NO: 46 - protein, 7 aa, synthetic construct
GTNKRAP

SEQ ID NO: 47 - protein, 9 aa, synthetic construct
ALWYSNHWV

SEQ ID NO: 48 - protein, 237 aa, synthetic construct
GGGGSEPKSSDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 49 - protein, 459 aa, synthetic construct
GGGGSGGGGSQVQLVQSGAEVKKPGASVKVSCKASGYTFTRYTMHWVRQAPGQGLEWMGYINPSRGYTNYNQKFKDRVTLTTDKSSSTAYMELSSLRSEDTAVYYCARYYDDHYSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 50 - protein, 213 aa, synthetic construct
DIQMTQSPSSLSASVGDRVTITCSASSSVSYMNWYQQKPGKAPKRLIYDTSKLASGVPSRFSGSGSGTDFTLTISSLQPEDFATYYCQQWSSNPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 51 - protein, 462 aa, synthetic construct
GGGGSGGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 52 - protein, 214 aa, synthetic construct
DIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 53 - protein, 465 aa, synthetic construct
GGGGSGGGGSEVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 54 - protein, 216 aa, synthetic construct
QAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 55 - protein, 234 aa, synthetic construct
QVQLVQSGAEVKKPGASVKVSCKASGFTFTNYLIHWVRQAPGQGLEWMGWINPNSGGTNYAQRLQGRVTMTRDTSTSTVYMELSSLRSEDTAVYYCARDYGAGNDYWGQGTLVTVSSGGGGSGGGGSDIQMTQSPSSLSASVGDRVTITCRASQEIRRWLAWYQQKPGKAPKLLIYAASNLQSGVPSRFSGSGSGTDFTLTISSLQPEDFATYYCQQSYSTPYTFGQGTKLEIK

SEQ ID NO: 56 - protein, 457 aa, synthetic construct
GGGGSGGGGSQVQLVQSGAEVKKPGASVKVSCKASGFTFTNYLIHWVRQAPGQGLEWMGWINPNSGGTNYAQRLQGRVTMTRDTSTSTVYMELSSLRSEDTAVYYCARDYGAGNDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 57 - protein, 214 aa, synthetic construct
DIQMTQSPSSLSASVGDRVTITCRASQEIRRWLAWYQQKPGKAPKLLIYAASNLQSGVPSRFSGSGSGTDFTLTISSLQPEDFATYYCQQSYSTPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 58 - protein, 239 aa, synthetic construct
EVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGGSGGGGSDIQMTQSPSSLSASVGDRVTITCRASQEIRRWLAWYQQKPGKAPKLLIYAASNLQSGVPSRFSGSGSGTDFTLTISSLQPEDFATYYCQQSYSTPYTFGQGTKLEIK

SEQ ID NO: 59 - protein, 234 aa, synthetic construct
QVQLVQSGAEVKKPGASVKVSCKASGFTFTNYLIHWVRQAPGQGLEWMGWINPNSGGTNYAQRLQGRVTMTRDTSTSTVYMELSSLRSEDTAVYYCARDYGAGNDYWGQGTLVTVSSGGGGSGGGGSDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIK

SEQ ID NO: 60 - protein, 119 aa, synthetic construct
QVQLVQSGAEVKKPGASVKVSCKASGYTFTNYYMHWVRQAPGQGLEWMGMINPSGGGTSYAQKFQGRVTMTRDTSTSTVYMELSSLRSEDTAVYYCARGNPWELRLDYWGQGTLVTVSS

SEQ ID NO: 61 - protein, 107 aa, synthetic construct
DIQMTQSPSSLSASVGDRVTITCQASQDISNYLNWYQQKPGKAPKLLIYAASSLQSGVPSRFSGSGSGTDFTLTISSLQPEDFATYYCQQYYSYPFTFGPGTKVDIK

SEQ ID NO: 62-66 - skipped (5 entries)

SEQ ID NO: 67 - protein, 9 aa, synthetic construct
YTFTNYYMH

SEQ ID NO: 68 - protein, 13 aa, synthetic construct
GMINPSGGGTSYA

SEQ ID NO: 69 - protein, 14 aa, synthetic construct
CARGNPWELRLDYW

SEQ ID NO: 70 - protein, 11 aa, synthetic construct
QASQDISNYLN

SEQ ID NO: 71 - protein, 7 aa, synthetic construct
AASSLQS

SEQ ID NO: 72 - protein, 11 aa, synthetic construct [sequence omitted]

SEQ ID NO: 73 - protein, 9 aa, synthetic construct
GTFSNFGVS

SEQ ID NO: 74 - protein, 13 aa, synthetic construct
GGIIPILGTANYA

SEQ ID NO: 75 - protein, 21 aa, synthetic construct
CATPTNSGYYGPYYYYGMDVW

SEQ ID NO: 76 - protein, 11 aa, synthetic construct
RASQSISSWLA

SEQ ID NO: 77 - protein, 7 aa, synthetic construct
AASTLQS

SEQ ID NO: 78 - protein, 11 aa, synthetic construct [sequence omitted]

SEQ ID NO: 79 - protein, 126 aa, synthetic construct
QVQLVQSGAEVKKPGSSVKVSCKASGGTFSNFGVSWLRQAPGQGLEWMGGIIPILGTANYAQKFQGRVTITADESTSTAYMELSSLRSEDTAVYYCATPTNSGYYGPYYYYGMDVWGQGTTVTVSS

SEQ ID NO: 80 - protein, 107 aa, synthetic construct
DIQMTQSPSSLSASVGDRVTITCRASQSISSWLAWYQQKPGKAPKLLIYAASTLQSGVPSRFSGSGSGTDFTLTISSLQPEDFATYYCQQSYSIPLTFGGGTKVEIK

SEQ ID NO: 81 - protein, 237 aa, synthetic construct
GGGGSEPKSSDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 82 - protein, 246 aa, synthetic construct
QVQLVQSGAEVKKPGASVKVSCKASGYTFTNYYMHWVRQAPGQGLEWMGMINPSGGGTSYAQKFQGRVTMTRDTSTSTVYMELSSLRSEDTAVYYCARGNPWELRLDYWGQGTLVTVSSGGGGSGGGGSGGGGSGGGGSDIQMTQSPSSLSASVGDRVTITCQASQDISNYLNWYQQKPGKAPKLLIYAASSLQSGVPSRFSGSGSGTDFTLTISSLQPEDFATYYCQQYYSYPFTFGPGTKVDIK

SEQ ID NO: 83 - protein, 119 aa, synthetic construct; identical to SEQ ID NO: 24

SEQ ID NO: 84 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 85 - protein, 213 aa, synthetic construct; identical to SEQ ID NO: 50

SEQ ID NO: 86 - protein, 237 aa, synthetic construct; identical to SEQ ID NO: 81

SEQ ID NO: 87 - protein, 246 aa, synthetic construct; identical to SEQ ID NO: 82

SEQ ID NO: 88 - protein, 125 aa, synthetic construct; identical to SEQ ID NO: 34

SEQ ID NO: 89 - protein, 330 aa, synthetic construct; identical to SEQ ID NO: 84

SEQ ID NO: 90 - protein, 216 aa, synthetic construct; identical to SEQ ID NO: 54

SEQ ID NO: 91 - protein, 237 aa, synthetic construct; identical to SEQ ID NO: 81

SEQ ID NO: 92 - protein, 253 aa, synthetic construct
QVQLVQSGAEVKKPGSSVKVSCKASGGTFSNFGVSWLRQAPGQGLEWMGGIIPILGTANYAQKFQGRVTITADESTSTAYMELSSLRSEDTAVYYCATPTNSGYYGPYYYYGMDVWGQGTTVTVSSGGGGSGGGGSGGGGSGGGGSDIQMTQSPSSLSASVGDRVTITCRASQSISSWLAWYQQKPGKAPKLLIYAASTLQSGVPSRFSGSGSGTDFTLTISSLQPEDFATYYCQQSYSIPLTFGGGTKVEIK

SEQ ID NO: 93 - protein, 119 aa, synthetic construct; identical to SEQ ID NO: 24

SEQ ID NO: 94 - protein, 330 aa, synthetic construct; identical to SEQ ID NO: 84

SEQ ID NO: 95 - protein, 213 aa, synthetic construct; identical to SEQ ID NO: 50

SEQ ID NO: 96 - protein, 237 aa, synthetic construct; identical to SEQ ID NO: 81

SEQ ID NO: 97 - protein, 253 aa, synthetic construct; identical to SEQ ID NO: 92

SEQ ID NO: 98 - protein, 125 aa, synthetic construct; identical to SEQ ID NO: 34

SEQ ID NO: 99 - protein, 330 aa, synthetic construct; identical to SEQ ID NO: 84

SEQ ID NO: 100 - protein, 216 aa, synthetic construct; identical to SEQ ID NO: 54

SEQ ID NO: 101 - protein, 237 aa, synthetic construct; identical to SEQ ID NO: 81

SEQ ID NO: 102 - protein, 246 aa, synthetic construct
QVQLVQSGAEVKKPGASVKVSCKASGYTFTNYYMHWVRQAPGQCLEWMGMINPSGGGTSYAQKFQGRVTMTRDTSTSTVYMELSSLRSEDTAVYYCARGNPWELRLDYWGQGTLVTVSSGGGGSGGGGSGGGGSGGGGSDIQMTQSPSSLSASVGDRVTITCQASQDISNYLNWYQQKPGKAPKLLIYAASSLQSGVPSRFSGSGSGTDFTLTISSLQPEDFATYYCQQYYSYPFTFGCGTKVDIK

SEQ ID NO: 103 - protein, 119 aa, synthetic construct; identical to SEQ ID NO: 24

SEQ ID NO: 104 - protein, 330 aa, synthetic construct; identical to SEQ ID NO: 84

SEQ ID NO: 105 - protein, 213 aa, synthetic construct; identical to SEQ ID NO: 50

SEQ ID NO: 106 - protein, 237 aa, synthetic construct; identical to SEQ ID NO: 81

SEQ ID NO: 107 - protein, 253 aa, synthetic construct
QVQLVQSGAEVKKPGSSVKVSCKASGGTFSNFGVSWLRQAPGQCLEWMGGIIPILGTANYAQKFQGRVTITADESTSTAYMELSSLRSEDTAVYYCATPTNSGYYGPYYYYGMDVWGQGTTVTVSSGGGGSGGGGSGGGGSGGGGSDIQMTQSPSSLSASVGDRVTITCRASQSISSWLAWYQQKPGKAPKLLIYAASTLQSGVPSRFSGSGSGTDFTLTISSLQPEDFATYYCQQSYSIPLTFGGGTKVEIK

SEQ ID NO: 108 - protein, 119 aa, synthetic construct; identical to SEQ ID NO: 24

SEQ ID NO: 109 - protein, 330 aa, synthetic construct; identical to SEQ ID NO: 84

SEQ ID NO: 110 - protein, 213 aa, synthetic construct; identical to SEQ ID NO: 50

SEQ ID NO: 111 - protein, 10 aa, synthetic construct
GGGGSGGGGS

SEQ ID NO: 112 - protein, 15 aa, synthetic construct
GGGGSEPKSSDKTHT

SEQ ID NO: 113 - protein, 20 aa, synthetic construct
GGCGGGGSGSEPKSSDKTHT

SEQ ID NO: 114 - protein, 15 aa, synthetic construct
GGCGGGGSGSGGGGS

SEQ ID NO: 115 - protein, 15 aa, synthetic construct
GGCGSEPKSSDKTHT

SEQ ID NO: 116 - protein, 5 aa, synthetic construct
GGCGS

SEQ ID NO: 117 - protein, 21 aa, synthetic construct
GGGCGGGGSGSEPKSSDKTHT

SEQ ID NO: 118 - protein, 16 aa, synthetic construct
GGGCGGGGSGSGGGGS

SEQ ID NO: 119 - protein, 6 aa, synthetic construct
GGGCGS

SEQ ID NO: 120 - protein, 9 aa, synthetic construct
ATDNNLAVY

SEQ ID NO: 121 - protein, 9 aa, synthetic construct
NADNNLAVY

SEQ ID NO: 122 - protein, 9 aa, synthetic construct
NTANNLAVY

SEQ ID NO: 123 - protein, 9 aa, synthetic construct
NTDANLAVY

SEQ ID NO: 124 - protein, 9 aa, synthetic construct
NTDNALAVY

SEQ ID NO: 125 - protein, 9 aa, synthetic construct
NTDNNAAVY

SEQ ID NO: 126 - protein, 9 aa, synthetic construct
NTDNNLAAY

SEQ ID NO: 127 - protein, 9 aa, synthetic construct
NTDNNLAVA

SEQ ID NO: 128 - protein, 9 aa, synthetic construct
NTENNLAVY

SEQ ID NO: 129 - protein, 9 aa, synthetic construct
NTDENLAVY

SEQ ID NO: 130 - protein, 9 aa, synthetic construct
NTDNVLAVY

SEQ ID NO: 131 - protein, 9 aa, synthetic construct
NTDNNVAVY

SEQ ID NO: 132 - protein, 9 aa, synthetic construct
NTDNNLVVY

SEQ ID NO: 133 - protein, 9 aa, synthetic construct
NTDNNLVEY

SEQ ID NO: 134 - protein, 9 aa, Homo sapiens
YLDGTLAIY

SEQ ID NO: 135 - protein, 9 aa, Homo sapiens
FSDKELAAY

SEQ ID NO: 136 - protein, 9 aa, Homo sapiens
AADVGLAGY

SEQ ID NO: 137 - protein, 9 aa, Homo sapiens
DTENEMAIY

SEQ ID NO: 138 - protein, 9 aa, Homo sapiens
RVDYSLAVY

SEQ ID NO: 139 - skipped

SEQ ID NO: 140 - protein, 9 aa, Homo sapiens
YADGKLAFY

SEQ ID NO: 141 - protein, 9 aa, Homo sapiens
GADPTLATY

SEQ ID NO: 142 - protein, 9 aa, Homo sapiens
QTEFALAVY

SEQ ID NO: 143 - protein, 9 aa, Homo sapiens
ATDFKFAMY

SEQ ID NO: 144 - protein, 9 aa, Homo sapiens
ATDYTFAMY

SEQ ID NO: 145 - protein, 9 aa, Homo sapiens
GTDPELNLY

SEQ ID NO: 146 - protein, 9 aa, Homo sapiens
WTDVFLQIY

SEQ ID NO: 147 - protein, 9 aa, Homo sapiens
RLDEDLAAY

SEQ ID NO: 148 - protein, 9 aa, Homo sapiens
TVDTTLAGY

SEQ ID NO: 149 - protein, 9 aa, Homo sapiens
FIDKQLAAY

SEQ ID NO: 150 - protein, 9 aa, Homo sapiens
QTDFPLQAY

SEQ ID NO: 151 - protein, 9 aa, Homo sapiens
YIDIFLNVY

SEQ ID NO: 152 - protein, 9 aa, Homo sapiens
YTDNWLAVY

SEQ ID NO: 153 - protein, 9 aa, Homo sapiens
AMDVNLTVY

SEQ ID NO: 154 - protein, 9 aa, Homo sapiens
RTEDNLQIY

SEQ ID NO: 155 - protein, 9 aa, Homo sapiens
LMDKHLAGY

SEQ ID NO: 156 - protein, 9 aa, Homo sapiens
VADGILASY

SEQ ID NO: 157 - protein, 9 aa, Homo sapiens
SLDLYLAMY

SEQ ID NO: 158 - protein, 9 aa, Homo sapiens
NMDYTLTMY

SEQ ID NO: 159 - protein, 9 aa, Homo sapiens
NLDGLIAGY

SEQ ID NO: 160 - protein, 9 aa, Homo sapiens
EAENNLAAY

SEQ ID NO: 161 - protein, 9 aa, Homo sapiens
VTDFKFALY

SEQ ID NO: 162 - protein, 9 aa, Homo sapiens
QIDPVLQVY

SEQ ID NO: 163 - protein, 9 aa, Homo sapiens
KMDQTLAVY

SEQ ID NO: 164 - protein, 9 aa, Homo sapiens
NMDRPIAGY

SEQ ID NO: 165 - protein, 9 aa, Homo sapiens
NLDFVLSFY

SEQ ID NO: 166 - protein, 9 aa, Homo sapiens
ETEYGLSVY

SEQ ID NO: 167 - protein, 9 aa, Homo sapiens
CTETHLAAY

SEQ ID NO: 168 - protein, 9 aa, Homo sapiens
EADINIAFY

SEQ ID NO: 169 - protein, 9 aa, Homo sapiens
MTDGGLGLY

SEQ ID NO: 170 - protein, 9 aa, Homo sapiens
MTDGGLNLY

SEQ ID NO: 171 - protein, 9 aa, Homo sapiens
NTEEPISVY

SEQ ID NO: 172 - protein, 9 aa, Homo sapiens
NVDRVFALY

SEQ ID NO: 173 - protein, 9 aa, Homo sapiens
DMDYTLAVY

SEQ ID NO: 174 - protein, 9 aa, Homo sapiens
ATDIKITVY

SEQ ID NO: 175 - protein, 9 aa, Homo sapiens
NTEEFITVY

SEQ ID NO: 176 - protein, 9 aa, Homo sapiens
RLDEPLASY

SEQ ID NO: 177 - protein, 9 aa, Homo sapiens
TADHGLAAY

SEQ ID NO: 178 - protein, 9 aa, Homo sapiens
KTENLLGSY

SEQ ID NO: 179 - protein, 9 aa, Homo sapiens
WTDYLLAFY

SEQ ID NO: 180 - protein, 9 aa, Homo sapiens
QTDLLIAFY

SEQ ID NO: 181-189 - skipped (9 entries)

SEQ ID NO: 190 - protein, 9 aa, Homo sapiens
NSDSNLTTY

SEQ ID NO: 191 - protein, 9 aa, Homo sapiens
KTDHFLSCY

SEQ ID NO: 192 - protein, 9 aa, Homo sapiens
CTDRAFAVY

SEQ ID NO: 193 - protein, 9 aa, Homo sapiens
RTDGILALY

SEQ ID NO: 194 - protein, 9 aa, Homo sapiens
CVDWSIAVY

SEQ ID NO: 195 - protein, 9 aa, Homo sapiens
NIDDALQCY

SEQ ID NO: 196 - protein, 9 aa, Homo sapiens
FSELNLAAY

SEQ ID NO: 197 - protein, 9 aa, Homo sapiens
GMDDFLGVY

SEQ ID NO: 198 - protein, 9 aa, Homo sapiens
NTDSDMGGY

SEQ ID NO: 199 - protein, 9 aa, Homo sapiens
YADRALAFY

SEQ ID NO: 200 - protein, 9 aa, Homo sapiens
GTDKLLSGY

SEQ ID NO: 201 - protein, 9 aa, Homo sapiens
QVDSTLAAY

SEQ ID NO: 202 - protein, 9 aa, Homo sapiens
WSDAKLAAY

SEQ ID NO: 203 - protein, 9 aa, Homo sapiens
GTDNWLAQY

SEQ ID NO: 204 - protein, 9 aa, Homo sapiens
ETDNNIVVY

SEQ ID NO: 205 - protein, 9 aa, Homo sapiens
PTDENLARY

SEQ ID NO: 206 - protein, 9 aa, Homo sapiens
NTDNLLTEY

SEQ ID NO: 207 - protein, 9 aa, Homo sapiens
LTEQYNEQY

SEQ ID NO: 208 - protein, 9 aa, Homo sapiens
TTEVHPELY

SEQ ID NO: 209 - protein, 9 aa, Homo sapiens
ATDLTREVY

SEQ ID NO: 210 - protein, 4 aa, synthetic construct
GGGC

SEQ ID NO: 211 - protein, 12 aa, Homo sapiens
KSCDKTHTCPPC

SEQ ID NO: 212 - protein, 4 aa, Homo sapiens
ELLG

SEQ ID NO: 213 - protein, 4 aa, Homo sapiens
EFLG

SEQ ID NO: 214 - protein, 9 aa, Homo sapiens
NTDNNLAVY

SEQ ID NO: 215 - protein, 15 aa, synthetic construct
  VARIANT 6..15: note = GGGGS repeats may be deleted
GGGGSGGGGSGGGGS

SEQ ID NO: 216 - protein, 10 aa, synthetic construct
GSGGGGSGGG

SEQ ID NO: 217 - protein, 5 aa, synthetic construct
GGGGS

SEQ ID NO: 218 - protein, 40 aa, synthetic construct
  VARIANT 3..40: note = GS repeats may be deleted
GSGSGSGSGSGSGSGSGSGSGSGSGSGSGSGSGSGSGSGS

SEQ ID NO: 219 - protein, 60 aa, synthetic construct
  VARIANT 4..60: note = GGS repeats may be deleted
GGSGGSGGSGGSGGSGGSGGSGGSGGSGGSGGSGGSGGSGGSGGSGGSGGSGGSGGSGGS

SEQ ID NO: 220 - protein, 80 aa, synthetic construct
  VARIANT 5..80: note = GGGS repeats may be deleted
GGGSGGGSGGGSGGGSGGGSGGGSGGGSGGGSGGGSGGGSGGGSGGGSGGGSGGGSGGGSGGGSGGGSGGGSGGGSGGGS

SEQ ID NO: 221 - protein, 80 aa, synthetic construct
  VARIANT 5..80: note = GGSG repeats may be deleted
GGSGGGSGGGSGGGSGGGSGGGSGGGSGGGSGGGSGGGSGGGSGGGSGGGSGGGSGGGSGGGSGGGSGGGSGGGSGGGSG

SEQ ID NO: 222 - protein, 100 aa, synthetic construct
  VARIANT 6..100: note = GGSGG repeats may be deleted
GGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGG

SEQ ID NO: 223 - protein, 100 aa, synthetic construct
  VARIANT 6..100: note = GGGGS repeats may be deleted
GGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGS

SEQ ID NO: 224 - protein, 100 aa, synthetic construct
  VARIANT 6..100: note = GSGGG repeats may be deleted
GSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGG

SEQ ID NO: 225 - protein, 100 aa, synthetic construct
  VARIANT 6..100: note = GGGSG repeats may be deleted
GGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSG

SEQ ID NO: 226 - protein, 100 aa, synthetic construct
  VARIANT 6..100: note = GGGGG repeats may be deleted
GGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGG

SEQ ID NO: 227 - protein, 50 aa, synthetic construct
  VARIANT 16..50: note = GGGGS repeats may be deleted
GGGGSGGGGSGGGGSGGGGSGGGGSGGGGSGGGGSGGGGSGGGGSGGGGS

SEQ ID NO: 228 - protein, 20 aa, synthetic construct
GGGGSGGGGSGGGGSGGGGS

SEQ ID NO: 229 - DNA, 22 bp, synthetic construct
  source 1..22: mol_type = other DNA; note = primer
ctcgcggcccagccggccatgg

SEQ ID NO: 230 - DNA, 31 bp, synthetic construct
  source 1..31: mol_type = other DNA; note = primer
gttggcctcccgggccactagttttgatctc

SEQ ID NO: 231 - protein, 6 aa, synthetic construct
GSGGGG

SEQ ID NO: 232 - protein, 17 aa, synthetic construct
GGGGSEPKSSDKTHTCP

SEQ ID NO: 233 - protein, 9 aa, Homo sapiens
EVDPIGHVY

SEQ ID NO: 234 - protein, 16 aa, synthetic construct
GGGCGSEPKSSDKTHT

SEQ ID NO: 235 - protein, 117 aa, synthetic construct
QVQLVQSGAEVKKPGASVKVSCKASGFTFTNYLIHWVRQAPGQGLEWMGWINPNSGGTNYAQRLQGRVTMTRDTSTSTVYMELSSLRSEDTACYYCARDYGAGNDYWGQGTLVTVSS

SEQ ID NO: 236 - protein, 10 aa, Homo sapiens
AIFPGAVPAA

SEQ ID NO: 237 - protein, 8 aa, synthetic construct
GSGGGGCP